SEQUENCE LISTING (52 sequences)

SEQ ID NO: 1 - protein, 443 aa, synthetic construct
MSYYHHHHHHDYDIPTTENLTRSSENLYFQSGGTQTQLAIDNVLASAESTIQLNELPKVVLDFITGEQTSVARSGGIFTKEDLINLKLYVRKGLSLPTRQDEVEAYLGYKKIDVAGLEPKDIKLLFDEIHNHALNWNDVEQAVLQQSLDLDIAAKNIISTGNEIINLINQMPITLRVKTLLGDITDKQLENITYESADHEVASALKDILDDMKGDINRHQTTTENVRKKVSDYRITLTGGELSSGDKVNGLEPQVKTKYDLMEKSNMRKSIKELDEKIKEKRQRIEQLKKDYDKFVGLSFTGAIGGIIAMAITGGIFGAKAENARKEKNALISEVAELESKVSSQRALQTALEALSLSFSDIGIRMVDAESALNHLDFMWLSVLNQITESQIQFAMINNALRLTSFVNKFQQVITPWQSVGDSARQLVDIFDEAIKEYKKVYG

SEQ ID NO: 2 - protein, 6 aa, synthetic construct
HHHHHH

SEQ ID NO: 3 - protein, 7 aa, synthetic construct
ENLYFQS

SEQ ID NO: 4 - protein, 40 aa, synthetic construct
TQTQLAIDNVLASAESTIQLNELPKVVLDFITGEQTSVAR

SEQ ID NO: 5 - protein, 384 aa, synthetic construct
MHHHHHHENLYFQSSGGIFTKEDLINLKLYVRKGLSLPTRQDEVEAYLGYKKIDVAGLEPKDIKLLFDEIHNHALNWNDVEQAVLQQSLDLDIAAKNIISTGNEIINLINQMPITLRVKTLLGDITDKQLENITYESADHEVASALKDILDDMKGDINRHQTTTENVRKKVSDYRITLTGGELSSGDKVNGLEPQVKTKYDLMEKSNMRKSIKELDEKIKEKRQRIEQLKKDYDKFVGLSFTGAIGGIIAMAITGGIFGAKAENARKEKNALISEVAELESKVSSQRALQTALEALSLSFSDIGIRMVDAESALNHLDFMWLSVLNQITESQIQFAMINNALRLTSFVNKFQQVITPWQSVGDSARQLVDIFDEAIKEYKKVYG

SEQ ID NO: 6 - protein, 367 aa, synthetic construct
MSYYHHHHHHDYDIPTTENLYFQGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 7 - protein, 7 aa, synthetic construct
ENLYFQG

SEQ ID NO: 8 - protein, 378 aa, synthetic construct
MSYYHHHHHHDYDIPTTENLYFQGWSHPQFEKGSSAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 9 - skipped

SEQ ID NO: 10 - protein, 378 aa, synthetic construct
MSYYHHHHHHDYDIPTTENLYFQGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLIGSSWSHPQFEK

SEQ ID NO: 11 - protein, 385 aa, synthetic construct
MSYYHHHHHHDYDIPTTENLYFQGWSHPQFEKGSSGSAGSAGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 12 - protein, 10 aa, synthetic construct
GSSGSAGSAG

SEQ ID NO: 13 - protein, 385 aa, synthetic construct
MSYYHHHHHHDYDIPTTENLYFQGWSHPQFEKGSDGDEGSEGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 14 - protein, 10 aa, synthetic construct
GSDGDEGSEG

SEQ ID NO: 15 - protein, 385 aa, synthetic construct
MSYYHHHHHHDYDIPTTENLYFQGWSHPQFEKGSRKSRGSKGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 16 - protein, 10 aa, synthetic construct
GSRKSRGSKG

SEQ ID NO: 17 - protein, 391 aa, synthetic construct
MSYYHHHHHHDYDIPTTENLYFQGWSHPQFEKGSSDSDGSSGEAGDEGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 18 - protein, 16 aa, synthetic construct
GSSDSDGSSGEAGDEG

SEQ ID NO: 19 - protein, 405 aa, synthetic construct
MSYYHHHHHHDYDIPTTENLYFQGWSHPQFEKGSSGSTSNASAGTGSATGSNSTAGASSGNSAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 20 - protein, 30 aa, synthetic construct
GSSGSTSNASAGTGSATGSNSTAGASSGNS

SEQ ID NO: 21 - protein, 425 aa, synthetic construct
MSYYHHHHHHDYDIPTTENLYFQGWSHPQFEKGSSGSTSNASAGTGSATGSNSTAGASSGNSAGTGTSSGSTGSANSSAGTGAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 22 - protein, 50 aa, synthetic construct
GSSGSTSNASAGTGSATGSNSTAGASSGNSAGTGTSSGSTGSANSSAGTG

SEQ ID NO: 23 - protein, 445 aa, synthetic construct
MSYYHHHHHHDYDIPTTENLYFQGWSHPQFEKGSSGSTSNASAGTGSATGSNSTAGASSGNSAGTGTSSGSTGSANSSAGTGSGSATASAGNSGTSTAGNSTAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAIIKIDQEKSAVVVEAEKLSRAWHIFIHEITALQGTSLNEVELSKPLIKQQIYLESLIKQLI

SEQ ID NO: 24 - protein, 70 aa, synthetic construct
GSSGSTSNASAGTGSATGSNSTAGASSGNSAGTGTSSGSTGSANSSAGTGSGSATASAGNSGTSTAGNST

SEQ ID NO: 25 - protein, 411 aa, Yersinia enterocolitica
MTQTQLAIDNVLASAENTIQLNELPKVVLDFITGEQTSVARSGGIFTKEDLINLKLYVRKGLSLPTRQDEVEAYLGYKKIDVAGLEPKDIKLLFDEIHNHALNWNDVEQAVLQQSLDLDIAAKNIISTGNEIINLINQMPITLRVKTLLRDITDKQLENITYESADHEVASALKDILDDMKGDINRHQTTTENVRKKVSDYRITLTGGELSSGDKVNGLEPQVKTKYDLMEKSNMRKSIKELDEKIKEKKQRIEQLKKDYDKFVGLSFTGAIGGIIAMAITSGIFGAKAENARKEKNALISEVAELESKVSSQRALQTALEALSLSFSDIGIRMVDAESALNHLDFMWLSVLNQITESQIQFAMINNALRLTSFVNKFQQVITPWQSVGDSARQLVDIFDEAIKEYKKVYG

SEQ ID NO: 26 - protein, 344 aa, Yersinia enterocolitica
MAEISTFPHSGLSYPDINFKIFSQGVKNISHLAQFKTTGVEVLQEKALRVSLYSQRLDVIVRESLSSLQVKLENTLALTYFTTLEEIDEALISQDIDEESKSEMRKERINIIKNLSNDITQLKQLFIEKTELLDKSSSDLHNVVIIEGTDKVLQAEQLRQKQLTEDIATKELERKEIEKKRDKIIEALDVIREHNLVDAFKDLIPTGENLSELDLAKPEIELLKQSLEITKKLLGQFSEGLKYIDLTDARKKLDNQIDTASTRLTELNRQLEQSEKLIAGVNAVIKIDQEKSAVVVEAEKLSRAWHIFIHEITALQGTSLNEVELSKPLIKQQIYLESLIKQLI

SEQ ID NO: 27 - protein, 403 aa, Providencia alcalifaciens
MNTQFNYDRIIEKESIGDATLALLTNQDSLSARSAGIFTLEDLISIHRHVQFALALPVKDSDILKWFGINDENSASLPAIELMDIIIDIRKHANSWNNVELKVKEQSVDLSLTSRNIMQTGGQVLEYINHMPIIKKISDTLEDLSEESLSQITYQNDDQQIATELLSILHLIKGDIKQQSIKTIHIKNTVSDFRAYITGGYLSDYTHVESLLFTLKGLYQQLESKEDPSNNESFLKELITFKKNELHQLEQEYSHFVKLCFTGLAGGIIGLIITGSIFGSKAENIRHRKNMLIKEIEEINHKLHQEQLLQKTIFDIQINLQKIDGLFKDARLAIDHLDYMWLVILTEIKQSIDIFARINNAEKLIQFITQFKRIIMSWSAIQDYSIHLIKLFDELQISEKNSQ

SEQ ID NO: 28 - protein, 336 aa, Providencia alcalifaciens
MHDNLDLPMLPSIDIYQLRCISNSISSFNKNILNYDYIIYGRIQKIAAKVERLNELIRNSVPILKIKLNYIIKNDAYELLTLEDNNDDYSDIRRKIVMTGFIDDLLDIKSELEKLIAKTRYALESLLVFHLNEPNKLKHSQYLKQKEFLVNSKNGKQSKINELNNHLSIILAAEEIILKHRITDFFDKYFQGKELIDSIDIQPNKKDILKAAISYIRNLLTMVDDGLEFSQLVDVRIYVSDQIIEAREEINTIDNNIFRISQIISYANDISQIESHKQTIITQTGALKSYWESWCGFMSEKISEECLNLEHIKTMSQMFMTYLNDIEYQYQRQLPD

SEQ ID NO: 29 - protein, 403 aa, Pseudomonas syringae
MLTPAALLENEDIARVTLKPVEYLNVIFDDSGGRSAGLVLTKEDILSLKRYERHALNIPTSLSRVEQQGFTKSGIPGLEPEDMLKTYKAINSHGKSWSGVEDGIKRTGFNIDLFAAQFSVQGQQIIDRIEKMDFARQLDLTVADLIIEEVRNTPPVLEKNDQRVCSTLAEFLKKTASQIKNHQHAAETLEQHIDTFSSVLSVTLIPGINDKVKLAARSDLDQQIKELEKDIEQLTTNIEQKNKEYKTALNNIAWGGFGGPIGVAITGGIFGAKAEKIRKEKNRMVASKAQKVQLLKEKVPLTAAVRSLQLMFDDMYIRMLDAHKGATHLKDLWLLLATYIERSAHELAAIKDDQALLIFAMQFQGVVMPWLEIKGKTTELLKIFDSALDQFKREQQPAIGIGR

SEQ ID NO: 30 - protein, 338 aa, Pseudomonas syringae
MTMSTETLGKPMVLTPPDTEIMNAASQNILAQVNTLKLDFLPAMKEKMLSLQNALTRADNAYREALADITVQLNNVNLQPIDLRQQHIEADARLSDKQKKQAISLLNGERIRLLSNLTAVLRSSAHAIAERSDDMAQINLTQEDNRLQDILQQQIDGMTQRSAALESAMSVIAEDRRLLDTTIKTYEKYNLADLFKDMLPTQEELQIVAMPSPELALITAGIARLGKLLDKISSALTYLDLTEERDRLRSRYNALLAESRTVAQEAKSINGRLDELTGLADISKSKALWVKEARKVYHSLYKFLDQITSQDDASAPISTPVEKLKTYIKSFYDIRRLV

SEQ ID NO: 31 - protein, 403 aa, Proteus mirabilis
MDTTTLDHIDKVIEQYQLPEVTLSLILGKNIDHGRSAGIFTPYDLNKIKSYIDKVLKLPYQKDTIIELLGYEDINISQISAETIQTLFLNMRTHAFEWQKIEYSVKQQAIDLEITGREITSTGEQLLAFINKMPIIKKINRKIEDITPEELSSIKYSTQDKKVSKQLVAILESMKNDINEQYNNTLKIKNMIYDFRIEIVGGENSKGNICHSIYYDIINKKKYINDFFDNSDDSLNEQRILLENEIEILKKEYQHFVKLAFTGLAFGIIGVIITGGIFGSKAEKIRKKRNETIEKLHNINKGIKQQSTISECLQRFQVDLDEIEQYIEDADMSVEHLLYMWQTILTEINASLINFKKIDNAMELIRFSIYLEKIIAPWYMVVGYSKEMMAVFDEALSSFYSSK

SEQ ID NO: 32 - protein, 335 aa, Proteus mirabilis
MNSVTSQLQEFDCNQFFHTIKSIRKITSELFEHDNILNHNLITISINNNKVYDEIIIFSILLKSRLNQKLLTGIFKNINEINHAMADRELTQQLSEKLLQEKMFIEQLFKDELTESIEIIEKHISFINDKKQLLTTTTIISEIDKLIRNKEEISIEINEKIAKQKLSINEIKEQLDIITKSEDIIYNKNILDFFTSHLPSKQTISKLQLASSEKDILSVFIDLLKQFFTHLEKGFSYQKLVETRHTLIDNYLIQIKNLTKLEQKKQALLFTLSRYYAVTEINQYIDMLNQQLSLLEQYWHTLIEQLIQLKRNLSEAKTVLSPHLVFLNHFSHHYQ

SEQ ID NO: 33 - protein, 399 aa, Morganella morganii
MHTQTMPADRLITADEIGPATLKLLLNEEEDTFRAAGIFTHNDLVHIKRYINYGMTLPREKEDVIEFIGYRDTELPGFEPHNIQSLFIHVHQHALSWDHVESMTKQQAIDLEIAGSAITATGNYILDAINEMPVIRRAEISLQDATADKLRDITYRRQDNIISGELTKILSAMKDDIAEERRKTQKVKNIISRFRLEMIGGADESGNEIPSLIYDVKRKQKILLKNQDSDATNDLLSEIRVRNEEIEILKREYNQFVKLSFSGLIGGIIGLIITGGIFGYRAEQVRRRKNKLLEEVAELEEQIISRQAIQQLIIRLDKELSTLDGYFTDAHVAVDHLDFMWQVMLTEITESLNTFMQINDAYSLLQFSLHLKKITLPWQRVRGYAKELVAVFDTANLAA

SEQ ID NO: 34 - protein, 328 aa, Morganella morganii
MTHSDITPEHITLAHLRAKYHDISNDAEILSLPAADLHFQIRDIKKWQTKILNMISAESAIIYSHLHNFDLENLLGTFSPDTGMNLFSLPHEKQIYEFLTSQMNTIYHSVNSINTLFNTEFDATAIPLLQGGLLHHQSYLDKAISNARPDIDKFKCDKLYWEEKLAVIIQSEEIIHQRGFQSLFGTTTLPTTEQLKNVEMSSSERFIMDELHRIISAVINTLTEGLSYVQLVETRTTLSQRIYDLSKLIRNLKKDLKQLQDHMQEISSALTLLPKLREFNNRISAVLLFWLQSVRQYEPYFSQNVPLPGLDALILAQRRYFSAFIGMA

SEQ ID NO: 35 - protein, 408 aa, Photorhabdus luminescens
MEKYMLLAQLPAEKTLNETDIPSATLQLLTGKQEGVARPGGIFTKEDLINIKLYVKKGLSLPFNLEEVKSYLGYQKIDIAGLEPEDIHSLFEEIRTHSFSWSDVENDILQQSIDLEVVGKQITETGENIISIINEMPIITRIKRRLGEISHRQLANITYTHEDREVSSALGEILDTMKNDINKQREKTEKVKTEVSDFKLKLIGGRLSNGSIALGLQPQVENKRKLMRDNKISVSIGDLDDKITEKKEEITQLKKDYDRFVGLAFSGIAGGLIGLAITGGIFGSRAEEVRKRKNMLIEEVRSLEESIKGKRALQKSITSLSLDFSDIDTRLLDAEVALNHLDYMWQSMLTQISASKDKFTQINDALNLTSFITKFQQVINPWKEVEGSAKQLVKVFDEALKEYKHRYN

SEQ ID NO: 36 - protein, 353 aa, Photorhabdus luminescens
MSEIITFPQQTVVYPEINVKTLSQAVKNIWRLSHQQKSGIEIIQEKTLRISLYSRDLDEAARASVPQLQTVLRQLPPQDYFLTLTEIDTELEDPELDDETRNTLLEARSEHIRNLKKDVKGVIRSLRKEANLMASRIADVSNVVILERLESSLKEEQERKAEIQADIAQQEKNKAKLVVDRNKIIESQDVIRQYNLADMFKDYIPNISDLDKLDLANPKKELIKQAIKQGVEIAKKILGNISKGLKYIELADARAKLDERINQINKDCDDLKIQLKGVEQRIAGIEDVHQIDKERTTLLLQAAKLEQAWNIFAKQLQNTIDGKIDQQDLTKIIHKQLDFLDDLALQYNSMLLS

SEQ ID NO: 37 - protein, 408 aa, Xenorhabdus nematophila
MENDMSSNQTLAEKKIPVSEVPSATLKMLTSQAEGVARPGGIFTKGDLINIKLYVKHSLELPFTLEGVKEYIGYNDIDIDGLKPAKMATLFKEIHDHALSWSGVESKVQQQSIDLENAGKQITLTGDEIISVIDQMPIIERVKNKLGDLTDKQLAEITYTNDDKEIAVELGNILESMKKDIKKQQENTQKVKTAVSDFKLKLIGGELSDGTIAQGLQPQISSKKKLMDDNNLSTTIKDLQSKIDEKNKEIDQLQKDYNKYVGLAFSGMVGGIISWAITGGIFGDKAEKARKQKNKLIDEVKDLQSQVKDKSALQTSVQNLSLSFAGIHTSMVDAEEALNHLDFMWNTMLTQITTSRDKFDDINDALKLTSFVIAFKQVIEPWRDVQGSAAQLIQTFDEALAEYKKLYH

SEQ ID NO: 38 - protein, 350 aa, Xenorhabdus nematophila
MSDNTLSQKENMYPEINIKAMNQAVNTIWLLAQRQTSGIEIINDKVKRISLYSREFDEMMRDSLAQLAPVLKQLTSDAAFQTIAQIDEALADPSLSKDDREALTLERNNLLQNLSKDIDNVIVSFTGRTNKLTNKISDISDMVIAERLQDLVTQAESQKTELQSDIDPKTEKRNKLDADREKIIESQDVIRQNNIADMFKDFIPSAKDIDGLDFTQPKKEAIKQAIKQGAEIARKILGKVSEGLKYIDLADARMKLSDQIDQLITETDELKAKIREVELRLSGLKDVMQIDTERTTLLTEAVKIEQVWISFAEQLHKLSNDEINQQDLSNLINGQLDFLNNLTLQYNKLK

SEQ ID NO: 39 - protein, 8 aa, synthetic construct
WSHPQFEK

SEQ ID NO: 40 - protein, 4 aa, synthetic construct
MSYY

SEQ ID NO: 41 - protein, 7 aa, synthetic construct
DYDIPTT

SEQ ID NO: 42 - protein, 5 aa, synthetic construct
EAAAK

SEQ ID NO: 43 - protein, 5 aa, synthetic construct
  REGION 1..5: note = this sequence can be repeated 1, 2, 3, 4, 5, 6, 7, 8, 9, or 10 times
GGGGS

SEQ ID NO: 44 - protein, 4 aa, synthetic construct
  SITE 4: note = this Gly residue can be repeated 1, 2, 3, 4, 5, 6, or 7 times
GGGG

SEQ ID NO: 45 - protein, 5 aa, synthetic construct
  REGION 1..5: note = this sequence can be repeated 1, 2, 3, 4, 5, 6, 7, 8, 9, or 10 times
EAAAK

SEQ ID NO: 46 - protein, 16 aa, synthetic construct
  REGION 2..6: note = this sequence can be repeated 1, 2, 3, 4, 5, 6, 7, 8, 9, or 10 times
  REGION 11..15: note = this sequence can be repeated 1, 2, 3, 4, 5, 6, 7, 8, 9, or 10 times
AEAAAKALEAEAAAKA

SEQ ID NO: 47 - protein, 4 aa, synthetic construct
  REGION 3..4: note = this AP sequence can be repeated 1, 2, 3, 4, 5, 6, 7, 8, 9, 10, 11, 12, 13, 14, or 19 times
APAP

SEQ ID NO: 48 - protein, 10 aa, synthetic construct
  SITE 1: note = coupled to StrepII tag
  SITE 10: note = coupled to YaxB
GDSGDEGSEG

SEQ ID NO: 49 - protein, 40 aa, synthetic construct
  SITE 1: note = coupled to Strep II tag
TPQQNKTPFSFGTANNNSNTTNQNSSTGAGAFGTGQSTFG

SEQ ID NO: 50 - protein, 4 aa, synthetic construct
TQTQ

SEQ ID NO: 51 - protein, 6 aa, synthetic construct
SGGIFT

SEQ ID NO: 52 - protein, 5 aa, synthetic construct
AEIST

(19) United States
(12) Patent Application Publication
(10) Pub. No.: US 2026/0176683 A1
(43) Pub. Date: Jun. 25, 2026

(54) LARGE CONICAL NANOPORES AND USES THEREOF IN ANALYTE SENSING

(71) Applicant: Rijksuniversiteit Groningen, Groningen (NL)

(72) Inventors: Giovanni MAGLIA, Glimmen (NL); Maaruthy YELLESWARAPU, Nijmengen (NL); Carsten WLOKA, Bergfelde (DE); Sabine STRAATHOF, Groningen (NL)

(21) Appl. No.: 19/341,112

(22) Filed: Sep. 26, 2025

Related U.S. Application Data
(63) Continuation of application No. PCT/NL2024/050161, filed on Apr. 2, 2024.

(30) Foreign Application Priority Data
Mar. 30, 2023 (EP) 23165582.0

(51) Int. Cl. C12Q 1/6869

(52) U.S. Cl. CPC C12Q 1/6869

(57) ABSTRACT

The invention relates to proteinaceous nanopores, nanopore systems and devices, and their application in single molecule analysis, such as detecting the presence, concentration and/or identity of a clinically relevant analyte in a complex sample. Provided is a sensor system comprising a nanopore embedded in an amphipathic or hydrophobic membrane separating a fluid filled chamber into a cis side and a trans side, wherein the nanopore is a conical shaped proteinaceous nanopore having a cis entrance of at least 11 nm, preferably about 12 to 20 nm, and a trans constriction of less than 5 nm, preferably about 2 to 4 nm.

### CROSS-REFERENCE TO RELATED APPLICATIONS

This application is a continuation of International Patent Application No. PCT/NL2024/050161, filed Apr. 2, 2024, which claims the benefit of European Application No. EP23165582.0, filed Mar. 30, 2023, each of which is herein incorporated by reference in its entirety.

### SEQUENCE LISTING

The instant application contains a Sequence Listing which has been submitted electronically in XML file format and is hereby incorporated by reference in its entirety. Said XML copy, created on Aug. 20, 2024, is named 64828-712-601_SL.xml and is 59,052 bytes in size.

### BACKGROUND

Determination of analytes is an important part of scientific studies. Improvements in the characterization of analytes can be important for further scientific studies or clinical aspects.

### SUMMARY

In an aspect, the present disclosure provides a biological nanopore comprising (i) a first opening of at least 10 nanometers (nm) and (ii) a second opening of less than 10 nm, wherein the biological nanopore is coupled to one or more recognition elements, wherein the one or more recognition elements are configured to interact with a non-nucleic acid based polymer analyte.

In some embodiments, the first opening comprises a widest dimension of at least 11 nm. In some embodiments, the first opening comprises a widest dimension of at least 15 nm. In some embodiments, the second opening comprises a widest dimension of less than 5 nm.

In some embodiments, the biological nanopore comprises at least a portion of an alpha-helical pore forming protein or peptide. In some embodiments, the biological nanopore comprises at least a portion of a beta-barrel pore forming protein or peptide. In some embodiments, the biological nanopore does not comprise a portion of an alpha-hemolysin. In some embodiments, the biological nanopore does not comprise a portion of a MspA.

In some embodiments, the first opening of the biological nanopore comprises a length that is greater than the second opening of the biological nanopore.

In some embodiments, the non-nucleic acid based polymer analyte comprises a size of at least about 20 kilodaltons (kDa). In some embodiments, the non-nucleic acid based polymer analyte comprises a size of at least about 50 kDa. In some embodiments, the non-nucleic acid based polymer analyte comprises a length of at least about 2 nm. In some embodiments, the non-nucleic acid based polymer analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the non-nucleic acid based polymer analyte comprises a diameter of at least 20 angstroms (Å). In some embodiments, the non-nucleic acid based polymer analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, or any combination thereof. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the biological nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the biological nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxBT. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the biological nanopore comprises one or more YaxA and YaxB heterodimers. In some embodiments, the biological nanopore comprises at least seven YaxA and YaxB heterodimers. In some embodiments, the biological nanopore comprises at least ten YaxA and YaxB heterodimers. In some embodiments, the biological nanopore comprises 20 YaxA and YaxB heterodimers.

In some embodiments, the non-nucleic acid based polymer analyte is smaller than 2 nm in size. In some embodiments, the non-nucleic acid based polymer analyte is coupled to a binder protein. In some embodiments, the non-nucleic acid based polymer analyte is smaller than the binder protein. In some embodiments, the binder protein is larger than 2 nm in size. In some embodiments, the binder protein has diameter greater than 20 Å. In some embodiments, one or more non-nucleic acid based polymer analytes couple to the binder protein. In some embodiments, the one or more non-nucleic acid based polymer analytes are the same. In some embodiments, the one or more non-nucleic acid based polymer analytes are different.

In some embodiments, the binder protein is configured to couple to the one or more recognition elements coupled to the biological nanopore. In some embodiments, the one or more recognition elements comprises protein, peptide, small molecules, nucleic acid, or any combination thereof. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements couple to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the biological nanopore. In some embodiments, the one or more recognition elements are indirectly coupled to the biological nanopore via one or more linkers. In some embodiments, the one or more linkers comprise flexible linkers. In some embodiments, the one or more linkers comprise polymer linkers. In some embodiments, the one or more recognition elements are directly coupled to the biological nanopore.

In some embodiments, the one or more recognition elements are coupled to the nanopore at the first opening. In some embodiments, the biological nanopore comprises one or more monomers. In some embodiments, a subunit of the one or more monomers is coupled to the one or more recognition elements.

In another aspect, the present disclosure provides a system comprising: a fluid chamber; and a membrane comprising a nanopore, wherein the membrane separates the fluid chamber into (1) a first side and (2) a second side, wherein the nanopore comprises (i) a first opening of at least 11 nm and (ii) a second opening of less than 11 nm, wherein the nanopore is configured to contact a non-nucleic acid based polymer analyte.

In some embodiments, the first opening comprises a widest dimension of at least 15 nm. In some embodiments, the second opening comprises a widest dimension of less than 5 nm.

In some embodiments, the nanopore comprises at least a portion of an alpha-helical pore forming protein or peptide. In some embodiments, the nanopore comprises at least a portion of a beta-barrel pore forming protein or peptide. In some embodiments, the nanopore does not comprise a portion of an alpha-hemolysin. In some embodiments, the nanopore does not comprise a portion of a MspA. In some embodiments, the first opening of the biological nanopore comprises a length that is greater than the second opening of the biological nanopore. In some embodiments, the non-nucleic acid based polymer analyte comprises a size of at least about 20 kilodaltons (kDa). In some embodiments, the non-nucleic acid based polymer analyte comprises a size of at least about 50 kDa. In some embodiments, the non-nucleic acid based polymer analyte comprises a length of at least about 2 nm. In some embodiments, the non-nucleic acid based polymer analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the non-nucleic acid based polymer analyte comprises a diameter of at least 20 angstroms (Å). In some embodiments, the non-nucleic acid based polymer analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, or any combination thereof. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the biological nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the biological nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxB. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the nanopore comprises one or more YaxA and YaxB heterodimers. In some embodiments, the nanopore comprises at least seven YaxA and YaxB heterodimers. In some embodiments, the nanopore comprises at least ten YaxA and YaxB heterodimers. In some embodiments, the nanopore comprises 20 YaxA and YaxB heterodimers.

In some embodiments, the non-nucleic acid based polymer analyte is smaller than 2 nm in size. In some embodiments, the non-nucleic acid based polymer analyte is coupled to a binder protein. In some embodiments, the non-nucleic acid based polymer analyte is smaller than the binder protein. In some embodiments, the binder protein is larger than 2 nm in size. In some embodiments, the binder protein has diameter greater than 20 Å. In some embodiments, the one or more non-nucleic acid based polymer analytes are coupled to the binder protein. In some embodiments, the one or more non-nucleic acid based polymer analytes are the same. Wherein the one or more non-nucleic acid based polymer analytes are different.

In some embodiments, the binder protein is configured to couple to one or more recognition elements coupled to the nanopore. In some embodiments, the nanopore comprises a biological nanopore. In some embodiments, the nanopore is coupled to one or more recognition elements. In some embodiments, the one or more recognition elements comprises protein, peptide, small molecules, nucleic acid, or any combination thereof. In some embodiments, the one or more recognition elements is configured to couple to the non-nucleic acid based polymer analyte. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements is coupled to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the nanopore. In some embodiments, the one or more recognition elements are indirectly coupled to the nanopore via one or more linkers. In some embodiments, the one or more linkers are flexible linkers. In some embodiments, the one or more linkers are polymer linkers. In some embodiments, the one or more recognition elements are directly coupled to the nanopore. In some embodiments, the one or more recognition elements are coupled to the nanopore at the first opening.

In some embodiments, the nanopore comprises one or more monomers. In some embodiments, a subunit of the one or more monomers is coupled to the one or more recognition elements.

In some embodiments, the system further comprises a pair of electrodes. In some embodiments, the system further comprises a controller. In some embodiments, the controller is configured to use the pair of electrodes to detect one or more signals associated with one or more characteristics of an analyte. In some embodiments, the first side of the fluid chamber comprises a first solution and the second side of the fluid chamber comprises a second solution. In some embodiments, the first solution comprises a first concentration of a solute and the second solution comprises a second concentration of the solute. In some embodiments, the solute comprises an ion or an osmolyte. In some embodiments, a difference between the first concentration of the solute and the second concentration of the solute is configured to generate an electro-osmotic force in a presence of an applied potential.

In another aspect, the present disclosure provides a method comprising: providing a nanopore system, wherein the nanopore system comprises (1) a fluid chamber and (2) a membrane comprising a nanopore, wherein the membrane separates the fluid chamber into a first side and a second side, wherein the nanopore comprises (i) a first opening of at least 11 nanometers (nm) and (ii) a second opening of less than 11 nm; and contacting the nanopore with a non-nucleic acid based polymer analyte.

In some embodiments, the first opening comprises a widest dimension at least 15 nm. In some embodiments, the second opening comprises a widest dimension less than 5 nm.

In some embodiments, the nanopore comprises at least a portion of an alpha-helix pore forming protein. In some embodiments, the nanopore comprises at least a portion of a beta-barrel pore forming protein. In some embodiments, the nanopore does not comprise a portion of an alpha-hemolysin. In some embodiments, the nanopore does not comprise a portion of a MspA. In some embodiments, the first opening of the nanopore comprises a length that is greater than the second opening of the nanopore. In some embodiments, the non-nucleic acid based polymer analyte comprises a size of at least about 20 kilodaltons (kDa). In some embodiments, the non-nucleic acid based polymer analyte comprises a size of at least about 50 kDa. In some embodiments, the non-nucleic acid based polymer analyte comprises a length of at least about 2 nm.

In some embodiments, the non-nucleic acid based polymer analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the non-nucleic acid based polymer analyte comprises a diameter of at least 20 angstroms (Å). In some embodiments, the non-nucleic acid based polymer analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, or any combination thereof. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxB. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the nanopore comprises one or more YaxA and YaxB heterodimers. In some embodiments, the nanopore comprises at least seven YaxA and YaxB heterodimers. In some embodiments, the nanopore comprises at least ten YaxA and YaxB heterodimers. In some embodiments, the nanopore comprises 20 YaxA and YaxB heterodimers.

In some embodiments, the non-nucleic acid based polymer analyte is smaller than 2 nm in size. In some embodiments, the non-nucleic acid based polymer analyte is coupled to a binder protein. In some embodiments, the non-nucleic acid based polymer analyte is smaller than the binder protein. In some embodiments, the binder protein is larger than 2 nm in size. In some embodiments, the binder protein has diameter greater than 20 Å. In some embodiments, one or more non-nucleic acid based polymer analytes is coupled to the binder protein. In some embodiments, the one or more analytes are the same. In some embodiments, the one or more analytes are different. In some embodiments, the binder protein is added to first side of the fluid chamber. In some embodiments, the binder protein is configured to enter into the first opening of the nanopore. In some embodiments, the non-nucleic acid based polymer analyte is located in the second side of the fluid chamber. In some embodiments, the non-nucleic acid based polymer analyte couples to the binder protein inside of the nanopore. In some embodiments, the binder protein is configured to not exit through the second opening of the nanopore.

In some embodiments, the binder protein is configured to couple to one or more recognition elements coupled to the nanopore. In some embodiments, the one or more recognition elements are configured to allow entry of the binder protein into the first opening of the nanopore. In some embodiments, the one or more recognition elements are configured to prevent entry of a non-binder protein into the first opening of the nanopore. In some embodiments, the nanopore comprises a biological nanopore.

In some embodiments, the nanopore is coupled to one or more recognition elements. In some embodiments, the one or more recognition elements comprises protein, peptide, small molecules, nucleic acid, or any combination thereof. In some embodiments, the one or more recognition elements couple to the non-nucleic acid based polymer analyte. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements couple to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the nanopore. In some embodiments, the one or more recognition elements are indirectly coupled to the nanopore via one or more linkers. In some embodiments, the one or more linkers are flexible linkers. In some embodiments, the one or more linkers are polymer linkers. In some embodiments, the one or more recognition elements are directly coupled to the nanopore. In some embodiments, the one or more recognition elements are configured to allow entry of the non-nucleic acid based polymer analyte into the first opening of the nanopore. In some embodiments, the one or more recognition elements are configured to prevent entry of a non-target non-nucleic acid based polymer analyte into the first opening of the nanopore. In some embodiments, the one or more recognition elements are coupled to the nanopore at the first opening.

In some embodiments, the nanopore comprises one or more monomers. In some embodiments, a subunit of the one or more monomers is coupled to the one or more recognition elements. In some embodiments, the first side of the fluid chamber comprises a first solution and the second side of the fluid chamber comprises a second solution. In some embodiments, the first solution comprises a first concentration of a solute and the second solution comprises a second concentration of the solute. In some embodiments, the solute comprises an ion or an osmolyte. In some embodiments, a difference between the first concentration of the solute and the second concentration of the solute is configured to generate an electro-osmotic force.

In some embodiments, the method further comprises measuring a signal generated by contacting the non-nucleic acid based polymer analyte to the nanopore. In some embodiments, the measuring the signal comprises measuring a signal for a state of (a) an open channel of the nanopore; (b) capture of the non-nucleic acid based polymer analyte by the first opening of the nanopore; or (c) exit of the non-nucleic acid based polymer analyte through the first opening of the nanopore. In some embodiments, the measuring comprises detecting differences in the signal between states (a), (b), and (c). In some embodiments, the signal comprises an ionic current, a change in ionic current, or derivations thereof. In some embodiments, the measuring comprises detecting a presence of the non-nucleic acid based polymer analyte, a concentration of the non-nucleic acid based polymer analyte, or any combination thereof. In some embodiments, the measuring comprises detecting one or more characteristics of the non-nucleic acid based polymer analyte. In some embodiments, the one or more characteristics of the non-nucleic acid based polymer analyte comprise a shape of the non-nucleic acid based polymer analyte, a structure of the non-nucleic acid based polymer analyte, one or more mutations of the non-nucleic acid based polymer analyte, a surface charge of the non-nucleic acid based polymer analyte, one or more post-translation modifications of the non-nucleic acid based polymer analyte, one or more ligands coupled to the non-nucleic acid based polymer analyte, or any combination thereof.

In some embodiments, (b) comprises contacting the non-nucleic acid based polymer analyte with the first side of the fluid chamber. In some embodiments, (b) comprises contacting the non-nucleic acid based polymer analyte with the second side of the fluid chamber. In some embodiments, the nanopore system further comprises a pair of electrodes. In some embodiments, the pair of electrodes is configured to provide an applied voltage to generate the electrophoretic force. In some embodiments, the applied voltage is a negative voltage on the first side of the fluid chamber. In some embodiments, the applied voltage is a positive voltage on the second side of the fluid chamber. In some embodiments, the non-nucleic acid based polymer analyte enters the nanopore through the first opening. In some embodiments, the non-nucleic acid based polymer analyte exits the nanopore through the first opening. In some embodiments, the non-nucleic acid based polymer analyte does not exit the nanopore through the second opening.

In another aspect, the present disclosure provides a membrane comprising a nanopore comprising (i) a first opening of at least 10 nm and (ii) a second opening of less than 10 nm.

In some embodiments, the first opening comprises a widest dimension at least 15 nm. In some embodiments, the second opening comprises a widest dimension less than 5 nm.

In some embodiments, the biological nanopore comprises at least a portion of an alpha-helical pore forming protein or peptide. In some embodiments, the biological nanopore comprises at least a portion of a beta-barrel pore forming protein or peptide. In some embodiments, the biological nanopore does not comprise a portion of an alpha-hemolysin. In some embodiments, the biological nanopore does not comprise a portion of a MspA. In some embodiments, the first opening of the biological nanopore comprises a length that is greater than the second opening of the biological nanopore.

In some embodiments, the non-nucleic acid based polymer analyte comprises a size of at least about 20 kilodaltons (kDa). In some embodiments, the non-nucleic acid based polymer analyte comprises a size of at least about 50 kDa. In some embodiments, the non-nucleic acid based polymer analyte comprises a length of at least about 2 nm. In some embodiments, the non-nucleic acid based polymer analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the non-nucleic acid based polymer analyte comprises a diameter of at least 20 angstroms (Å). In some embodiments, the non-nucleic acid based polymer analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, or any combination thereof. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the biological nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the biological nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxB. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the biological nanopore comprises one or more YaxA and YaxB heterodimers. In some embodiments, the biological nanopore comprises at least seven YaxA and YaxB heterodimers. In some embodiments, the biological nanopore comprises at least ten YaxA and YaxB heterodimers. In some embodiments, the biological nanopore comprises 20 YaxA and YaxB heterodimers.

In some embodiments, the non-nucleic acid based polymer analyte is smaller than 2 nm in size. In some embodiments, the non-nucleic acid based polymer analyte is coupled to a binder protein. In some embodiments, the non-nucleic acid based polymer analyte is smaller than the binder protein. In some embodiments, the binder protein is larger than 2 nm in size. In some embodiments, the binder protein has diameter greater than 20 Å. In some embodiments, one or more non-nucleic acid based polymer analytes are coupled to the binder protein. In some embodiments, the one or more non-nucleic acid based polymer analytes are the same. In some embodiments, the one or more non-nucleic acid based polymer analytes are different. In some embodiments, the binder protein is configured to couple to the one or more recognition elements coupled to the biological nanopore. In some embodiments, the one or more recognition elements comprises protein, peptide, small molecules, nucleic acid, or any combination thereof. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements couple to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the biological nanopore. In some embodiments, the one or more recognition elements are indirectly coupled to the biological nanopore via one or more linkers. In some embodiments, the one or more linkers comprise flexible linkers. In some embodiments, the one or more linkers comprise polymer linkers. In some embodiments, the one or more recognition elements are directly coupled to the biological nanopore. In some embodiments, the one or more recognition elements are coupled to the nanopore at the first opening.

In some embodiments, the biological nanopore comprises one or more monomers. In some embodiments, a subunit of the one or more monomers is coupled to the one or more recognition elements.

In another aspect, the present disclosure provides a biological nanopore comprising (i) a first opening of at least 10 nm and (ii) a second opening of less than 10 nm.

In some embodiments, the first opening comprises a widest dimension at least 15 nm. In some embodiments, the second opening comprises a widest dimension less than 5 nm.

In some embodiments, the biological nanopore comprises at least a portion of an alpha-helical pore forming protein or peptide. In some embodiments, the biological nanopore comprises at least a portion of a beta-barrel pore forming protein or peptide. In some embodiments, the biological nanopore does not comprise a portion of an alpha-hemolysin. In some embodiments, the biological nanopore does not comprise a portion of a MspA. In some embodiments, the first opening of the biological nanopore comprises a length that is greater than the second opening of the biological nanopore.

In some embodiments, the biological nanopore is configured to contact an analyte. In some embodiments, the analyte comprises a size of at least about 20 kilodaltons (kDa). In some embodiments, the analyte comprises a size of at least about 50 kDa. In some embodiments, the analyte comprises a length of at least about 2 nm. In some embodiments, the analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the analyte comprises a diameter of at least 20 angstroms (Å). In some embodiments, the analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, a non-nucleic acid based polymer analyte, or any combination thereof. In some embodiments, the analyte comprises a non-nucleic acid based polymer analyte. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the biological nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the biological nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxB. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the biological nanopore comprises one or more YaxA and YaxB heterodimers. In some embodiments, the biological nanopore comprises at least seven YaxA and YaxB heterodimers. In some embodiments, the biological nanopore comprises at least ten YaxA and YaxB heterodimers. In some embodiments, the biological nanopore comprises 20 YaxA and YaxB heterodimers.

In some embodiments, the biological nanopore is configured to contact an analyte. In some embodiments, the analyte is smaller than 2 nm in size. In some embodiments, the analyte is coupled to a binder protein. In some embodiments, the analyte is smaller than the binder protein. In some embodiments, the binder protein is larger than 2 nm in size. In some embodiments, the binder protein has diameter greater than 20 Å. In some embodiments, one or more analytes are configured to couple to the binder protein. In some embodiments, the one or more analytes are the same. In some embodiments, the one or more analytes are different in sequence and structure. In some embodiments, the binder protein is configured to couple to one or more recognition elements coupled to the biological nanopore.

In some embodiments, the biological nanopore is coupled to one or more recognition elements. In some embodiments, the one or more recognition elements are configured to interact with an analyte. In some embodiments, the one or more recognition elements comprises protein, peptide, small molecules, nucleic acid, or any combination thereof. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements couple to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the biological nanopore. In some embodiments, the one or more recognition elements are indirectly coupled to the biological nanopore via one or more linkers. In some embodiments, the one or more linkers comprise flexible linkers. In some embodiments, the one or more linkers comprise polymer linkers. In some embodiments, the one or more recognition elements are directly coupled to the biological nanopore. In some embodiments, the one or more recognition elements are coupled to the nanopore at the first opening. In some embodiments, the biological nanopore comprises one or more monomers. In some embodiments, a subunit of the one or more monomers is coupled to the one or more recognition elements.

In another aspect, the present disclosure provides a system comprising: a fluid chamber; and a membrane comprising a nanopore, wherein the membrane separates the fluid chamber into (1) a first side and (2) a second side, wherein the nanopore comprises (i) a first opening of at least 10 nm and (ii) a second opening of less than 10 nm.

In some embodiments, the first opening comprises a widest dimension at least 15 nm. In some embodiments, the second opening comprises a widest dimension less than 5 nm.

In some embodiments, the nanopore comprises at least a portion of an alpha-helical pore forming protein or peptide. In some embodiments, the nanopore comprises at least a portion of a beta-barrel pore forming protein or peptide. In some embodiments, the nanopore does not comprise a portion of an alpha-hemolysin. In some embodiments, the nanopore does not comprise a portion of a MspA. In some embodiments, the first opening of the nanopore comprises a length that is greater than the second opening of the nanopore.

In some embodiments, the nanopore is configured to contact an analyte. In some embodiments, the analyte comprises a size of at least about 20 kilodaltons (kDa). In some embodiments, the analyte comprises a size of at least about 50 kDa. In some embodiments, the analyte comprises a length of at least about 2 nm. In some embodiments, the analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the analyte comprises a diameter of at least 20 angstroms (Å). In some embodiments, the analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, a non-nucleic acid based polymer analyte, or any combination thereof. In some embodiments, the analyte comprises a non-nucleic acid based polymer analyte. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxB. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the nanopore comprises one or more YaxA and YaxB heterodimers. In some embodiments, the nanopore comprises at least seven YaxA and YaxB heterodimers. In some embodiments, the nanopore comprises at least ten YaxA and YaxB heterodimers. In some embodiments, the nanopore comprises 20 YaxA and YaxB heterodimers.

In some embodiments, the nanopore is configured to contact an analyte. In some embodiments, the analyte is smaller than 2 nm in size. In some embodiments, the analyte is coupled to a binder protein. In some embodiments, the analyte is smaller than the binder protein. In some embodiments, the binder protein is larger than 2 nm in size. In some embodiments, the binder protein has diameter greater than 20 Å. In some embodiments, one or more analytes couple to the binder protein. In some embodiments, the one or more analytes are the same. In some embodiments, the one or more analytes are different in sequence and structure.

In some embodiments, the binder protein is configured to couple to one or more recognition elements coupled to the nanopore. In some embodiments, the nanopore is coupled to one or more recognition elements. In some embodiments, the one or more recognition elements are configured to interact with an analyte. In some embodiments, the one or more recognition elements comprises protein, peptide, small molecules, nucleic acid, or any combination thereof. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements couple to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the nanopore. In some embodiments, the one or more recognition elements are indirectly coupled to nanopore via one or more linkers. In some embodiments, the one or more linkers comprise flexible linkers. In some embodiments, the one or more linkers comprise polymer linkers. In some embodiments, the one or more recognition elements are directly coupled to the nanopore. In some embodiments, the one or more recognition elements are coupled to the nanopore at the first opening. In some embodiments, the nanopore comprises one or more monomers. In some embodiments, a subunit of the one or more monomers is coupled to the one or more recognition elements. In some embodiments, the nanopore comprises a biological nanopore.

In some embodiments, the system further comprises a pair of electrodes. In some embodiments, the system further comprises a controller. In some embodiments, the controller is configured to use the pair of electrodes to detect one or more signals associated with one or more characteristics of an analyte. In some embodiments, the first side of the fluid chamber comprises a first solution and the second side of the fluid chamber comprises a second solution. In some embodiments, the first solution comprises a first concentration of a solute and the second solution comprises a second concentration of the solute. In some embodiments, the solute comprises an ion or an osmolyte. In some embodiments, a difference between the first concentration of the solute and the second concentration of the solute is configured to generate an electro-osmotic force in a presence of an applied potential.

In another aspect, the present disclosure provides a method comprising: providing a nanopore system, wherein the nanopore system comprises (1) a fluid chamber and (2) a membrane comprising a nanopore, wherein the membrane separates the fluid chamber into a first side and a second side, wherein the nanopore comprises (i) a first opening of at least 10 nanometers (nm) and (ii) a second opening of less than 10 nm; and contacting the nanopore with an analyte.

In some embodiments, the first opening comprises a widest dimension at least 15 nm. In some embodiments, the second opening comprises a widest dimension less than 5 nm.

In some embodiments, the nanopore comprises at least a portion of an alpha-helical pore forming protein or peptide. In some embodiments, the nanopore comprises at least a portion of a beta-barrel pore forming protein or peptide. In some embodiments, the nanopore does not comprise a portion of an alpha-hemolysin. In some embodiments, the nanopore does not comprise a portion of a MspA. In some embodiments, the first opening of the nanopore comprises a length that is greater than the second opening of the nanopore. In some embodiments, the nanopore is configured to contact an analyte. In some embodiments, the analyte comprises a size of at least about 20 kilodaltons (kDa). In some embodiments, the analyte comprises a size of at least about 50 kDa. In some embodiments, the analyte comprises a length of at least about 2 nm. In some embodiments, the analyte originates from a complex sample. In some embodiments, the complex sample comprises a clinical sample. In some embodiments, the clinical sample comprises whole blood, plasma, blood serum, urine, feces, saliva, cerebrospinal fluid, nasopharyngeal swab, breast milk, sputum, or any combination thereof.

In some embodiments, the analyte comprises a diameter of at least 20 angstroms (Å). In some embodiments, the analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, a non-nucleic acid based polymer analyte, or any combination thereof. In some embodiments, the analyte comprises a non-nucleic acid based polymer analyte. In some embodiments, the non-nucleic acid based polymer analyte is a peptide, a protein, or a polypeptide. In some embodiments, the non-nucleic acid based polymer comprises a folded protein, a protein biomarker, a peptide, a polypeptide, a pathogenic protein, or a cell surface protein.

In some embodiments, the nanopore comprises a conical shaped nanopore. In some embodiments, the conical shaped nanopore comprises one or more monomers. In some embodiments, the conical shaped nanopore comprises at least seven monomers. In some embodiments, the conical shaped nanopore comprises at least ten monomers. In some embodiments, a subunit of the one or more monomers comprises the same protein. In some embodiments, a subunit of the one or more monomers comprises different proteins.

In some embodiments, the nanopore comprises one or more subunits from an alpha-xenorhabdolysin family of binary toxins. In some embodiments, a subunit of the one or more subunits comprises one or more proteins or peptides from the alpha-xenorhabdolysin family of binary toxins. In some embodiments, the one or more proteins or peptides of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof. In some embodiments, the one or more proteins or peptides of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homologs, functional orthologs, functional paralogs, or any combination thereof. In some embodiments, the subunit of the one or more subunits of the biological nanopore comprises YaxA and YaxB, functional homologs, functional paralogs, or functional orthologs of YaxA and YaxB. In some embodiments, the YaxA is a truncated YaxA with at least 20 residues removed from a N-terminal region of a wild-type YaxA.

In some embodiments, the YaxA comprises one or more mutations. In some embodiments, the one or more mutations are at a position of R150, N12, N17, or any combination thereof of a wild-type YaxA. In some embodiments, the YaxB comprises one or more mutations. In some embodiments, the one or more mutations are at a position of V284, E208, E212, D214, E208, E212, or any combination thereof of a wild-type YaxB. In some embodiments, the nanopore comprises one or more YaxA and YaxB heterodimers. In some embodiments, the nanopore comprises at least seven YaxA and YaxB heterodimers. In some embodiments, the nanopore comprises at least ten YaxA and YaxB heterodimers. In some embodiments, the nanopore comprises 20 YaxA and YaxB heterodimers.

In some embodiments, the nanopore is configured to contact an analyte. In some embodiments, the analyte is smaller than 2 nm in size. In some embodiments, the analyte is coupled to a binder protein. In some embodiments, the analyte is smaller than the binder protein. In some embodiments, the binder protein is larger than 2 nm in size. In some embodiments, the binder protein has diameter greater than 20 Å.

In some embodiments, one or more analytes couple to the binder protein. In some embodiments, the one or more analytes are the same. In some embodiments, the one or more analytes are different in sequence and structure. In some embodiments, the binder protein is configured to couple to one or more recognition elements coupled to the nanopore. In some embodiments, the nanopore is coupled to one or more recognition elements. In some embodiments, the one or more recognition elements are configured to interact with an analyte. In some embodiments, the one or more recognition elements comprises protein, peptide, small molecules, nucleic acid, or any combination thereof. In some embodiments, each recognition element of the one or more recognition elements is coupled to a non-nucleic acid based polymer analyte, wherein the non-nucleic acid based polymer analyte on each recognition element is identical in sequence and structure. In some embodiments, each recognition element of the one or more recognition elements couple to the same non-nucleic acid based polymer analyte. In some embodiments, each recognition element of the one or more recognition elements is coupled to a non-nucleic acid based polymer analyte, wherein the non-nucleic acid based polymer analyte on each recognition element is different in sequence and structure.

In some embodiments, the one or more recognition elements are indirectly coupled to the nanopore. In some embodiments, the one or more recognition elements are indirectly coupled to nanopore via one or more linkers. In some embodiments, the one or more linkers comprise flexible linkers. In some embodiments, the one or more linkers comprise polymer linkers. In some embodiments, the one or more recognition elements are directly coupled to the nanopore. In some embodiments, the one or more recognition elements are coupled to the nanopore at the first opening. In some embodiments, the nanopore comprises one or more monomers. In some embodiments, a subunit of the one or more monomers is coupled to the one or more recognition elements. In some embodiments, the nanopore comprises a biological nanopore.

In some embodiments, the first side of the fluid chamber comprises a first solution and the second side of the fluid chamber comprises a second solution. In some embodiments, the first solution comprises a first concentration of a solute and the second solution comprises a second concentration of the solute. In some embodiments, the solute comprises an ion or an osmolyte. In some embodiments, a difference between the first concentration of the solute and the second concentration of the solute is configured to generate an electro-osmotic force.

In some embodiments, the method further comprises measuring a signal generated by contacting the non-nucleic acid based polymer analyte to the nanopore. In some embodiments, the measuring the signal comprises measuring a signal for a state of (a) an open channel of the nanopore; (b) capture of the non-nucleic acid based polymer analyte by the first opening of the nanopore; or (c) exit of the non-nucleic acid based polymer analyte through the first opening of the nanopore. In some embodiments, the measuring comprises detecting differences in the signal between states (a), (b), and (c). In some embodiments, the signal comprises an ionic current, a change in ionic current, or derivations thereof. In some embodiments, the measuring comprises detecting a presence of the non-nucleic acid based polymer analyte, a concentration of the non-nucleic acid based polymer analyte, or any combination thereof.

In some embodiments, the measuring comprises detecting one or more characteristics of the non-nucleic acid based polymer analyte. In some embodiments, the one or more characteristics of the non-nucleic acid based polymer analyte comprise a shape of the non-nucleic acid based polymer analyte, a structure of the non-nucleic acid based polymer analyte, one or more mutations of the non-nucleic acid based polymer analyte, a surface charge of the non-nucleic acid based polymer analyte, one or more post-translation modifications of the non-nucleic acid based polymer analyte, one or more ligands coupled to the non-nucleic acid based polymer analyte, or any combination thereof. In some embodiments, (b) comprises contacting the non-nucleic acid based polymer analyte with the first side of the fluid chamber. In some embodiments, (b) comprises contacting the non-nucleic acid based polymer analyte with the second side of the fluid chamber.

In some embodiments, the nanopore system further comprises a pair of electrodes. In some embodiments, the pair of electrodes is configured to provide an applied voltage to generate the electrophoretic force. In some embodiments, the applied voltage is a negative voltage on the first side of the fluid chamber. In some embodiments, the applied voltage is a positive voltage on the second side of the fluid chamber. In some embodiments, the non-nucleic acid based polymer analyte enters the nanopore through the first opening. In some embodiments, the non-nucleic acid based polymer analyte exits the nanopore through the first opening. In some embodiments, the non-nucleic acid based polymer analyte does not exit the nanopore through the second opening.

In another aspect, the present disclosure provides a method comprising: (a) providing a mixture containing or suspected of containing a polypeptide or protein, and (b) using a nanopore to generate a measure of a concentration or relative amount of said polypeptide or protein in said mixture at an accuracy of greater than 80%.

In some embodiments, the mixture contains or is suspected of containing an additional polypeptide or protein. In some embodiments, the method further comprises using the nanopore to generate a measure of a concentration or relative amount of the additional polypeptide or protein in the mixture at an accuracy of greater than 80%. In some embodiments, the nanopore is a conical nanopore. In some embodiments, the polypeptide or protein has a size greater than 3 kDa. In some embodiments, the polypeptide or protein has a size greater than 20 kDa. In some embodiments, the polypeptide or protein has a size greater than 60 kDa. In some embodiments, the measure of the concentration or relative amount of the polypeptide or protein in the mixture is generated at the accuracy of greater than 90%. In some embodiments, the measure of the concentration or relative amount of the polypeptide or protein in the mixture is generated at the accuracy of greater than 95%.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1B show schematic representations of the nanopores of the present disclosure. FIG. 1A shows a conical nanopore with unstructured N-terminal tails (i). FIG. 1B shows the truncated YaxA_{Δ40}B nanopore. The nanopore has a first opening (e.g., cis entrance) (101) and a second opening (e.g., trans entrance) (102). The nanopore can be disposed in a membrane (104) and have an edge (103). An outer edge can comprise an edge facing away from an interior channel (e.g., lumen) of the nanopore and an inner edge can comprise an edge facing the interior channel (e.g., lumen). Subunits of the nanopore can have untruncated N-termini (i) or truncated termini (ii).

FIGS. 2A-2B show representations of open-pore currents for YaxAB nanopores. FIG. 2A shows currents for (i) the full-length YaxAB pores and (ii) the truncated YaxA_{Δ40}B pores. FIG. 2B shows reverse potential current-voltage curves, demonstrating the pores are cation selective.

FIGS. 3A-3B show schematic models of a target analyte (301) in a cis chamber captured by a nanopore. FIG. 3A shows the analyte (301) in the first side (e.g., cis chamber). FIG. 3B shows the electroosmotic flow and ionic current moves the analyte to the first opening (302) of the nanopore. Once captured by the nanopore, the analyte (301) resides in the constriction region (303) allowing characterization.

FIG. 4 shows a model showing capture and characterization of different target analytes in nanopores. Abbreviations are: bovine thrombin (BT); streptavidin A (SA); haemoglobin (HG); C-reactive protein (CRP).

FIGS. 5A-5C show electrophysiology data for the sampled target analytes: bovine thrombin (BT); streptavidin A (SA); haemoglobin (HG); C-reactive protein (CRP). FIG. 5A shows the current output for each analyte individually as well as a mixed sample containing all four different sized analytes (bottom row). FIG. 5B shows the dwell time (in milliseconds) on the y-axis plotted against the residual current (I_{RES} (%)) for each analyte as well as the complex (e.g., mixed) sample. FIG. 5C shows the blockade noise (σblockade) measured in picoamp (pA) on the y-axis plotted against the residual current (I_{RES} (%)) for each analyte as well as the complex (e.g., mixed) sample.

FIGS. 6A-6B show graphs depicting average residual current blockade for target analytes based on molecular weight (FIG. 6A) or hydrodynamic radius (FIG. 6B).

FIGS. 7A-7B show electrophysiological data for detection of CRP in depleted human serum. FIG. 7A shows panel (i) showing depleted human serum alone and was recorded for 10 minutes. Panels (ii) to (v) show increasing levels of CRP and were recorded for 2 minutes. FIG. 7B shows the correlation between event frequency of the CRP blockade events and concentration of CRP.

FIGS. 8A-8B show schematic models showing the capture method with the binder protein-analyte (BP-A) complex. FIG. 8A shows in panel (i) the binder protein (801) and analyte (802) on opposite sides of the nanopore (803), with the nanopore disposed in a membrane (804); (ii) the binder protein (801) entering the nanopore (803); and (iii) the binder protein (801) binding to analyte (802) in the nanopore (803). FIG. 8B shows (i) the binder protein (801) and analyte (802) on the same side of the nanopore; (ii) the binder protein (801) entering the nanopore (803); and (iii) the binder protein (801) binding to analyte (802) in the nanopore (803).

FIGS. 9A-9B show recordings of unmodified Streptavidin A (SA) in nanopore. FIG. 9A shows capture of target analyte SA (901) in nanopore (903). As the analyte (901) resides in a constriction region (904) of the nanopore (903), recordings may be taken of ionic current. FIG. 9B shows addition of biotin (902) to the analyte SA (901) and effect on recordings. As the analyte resides, the open-pore current (I_{O}) can spike, indicating a blockage event from the analyte and shown by the blockade current (I_{SA} for SA analyte). Current is measured in pA and measured over time.

FIGS. 10A-10F show a schematic model of the nanopore with conjugated recognition elements and linkers. The recognition element (i) is attached with various length linker (ii) and connected to the first opening (e.g., cis entrance) (iii) of the nanopore (1000). The linker (ii) may be attached to a nanopore (1000) disposed in a membrane (1010). The recognition element of a StrepII-tag is conjugated to a linker that is 3 amino acid residues (FIG. 10A), 10 amino acid residues (FIG. 10B), 20 amino acid residues (FIG. 10C), 30 amino acid residues (FIG. 10D), 50 amino acid residues (FIG. 10E), and 70 amino acid residues (FIG. 10F).

FIGS. 11A-11B show a schematic model of the capture and/or filtration method of a target analyte into a nanopore. FIG. 11A shows (i) the target analyte (111) approaching a nanopore with conjugated recognition element (117) attached by a linker (118) and the analyte is present on a first side (120) of a membrane; (ii) the recognition element assists in capture of the analyte; and (iii) the analyte is characterized in the nanopore. FIG. 11B shows (i) filtering of the target analyte (111) in the presence of a non-target analyte (112); (ii) the recognition element assists in capture of the target analyte; and (iii) the target analyte is characterized in the nanopore.

FIGS. 12A-12C show exemplary YaxAB nanopores for capture of Streptavidin A (SA). FIG. 12A shows unmodified YaxA_{Δ40}B*80 nanopore to capture target analyte SA (1201). FIG. 12B shows addition of N-terminal StrepII-tag (1202) to the nanopore to assist in capture of SA (1201). The StrepII-tag may be attached to the nanopore by a linker segment (1204). FIG. 12C shows addition of biotin (1203) and changes to recordings.

FIGS. 13A-13C show exemplary YaxAB nanopores for capture of Streptavidin A (SA). FIG. 13A shows unmodified YaxAΔ₄₀B*80 nanopore to capture target analyte SA (1301). FIG. 13B shows addition of C-terminal StrepII-tag (1302) to the nanopore to assist in capture of SA (1301). The StrepII-tag may be attached to the nanopore by a linker segment (1304). FIG. 13C shows addition of biotin (1303) and changes to recordings.

FIGS. 14A-14B show representative examples of YaxAΔ₄₀B*80 untruncated nanopore with C-reactive protein (CRP; 1401) in FIG. 14A and Streptavidin A (SA; 1402) in FIG. 14B. The CRP approaches a first opening (1403) of a nanopore (1404) disposed in a membrane (1405) and is captured for characterization. The difference in binding in the nanopore can be seen in the recorded current measurements.

FIGS. 15A-15C show N-termini Functionalized YaxA_{Δ40}B_{N-strepII-30aa-flex} with N-terminal StrepII-tag (1502) separated with 30 amino acids from YaxB. FIG. 15A shows C-reactive protein (1501) captured by nanopore and representative recordings. FIG. 15B shows capture of Streptavidin A (SA; 1503) in nanopore and prevention of CRP capture. FIG. 15C shows addition of biotin (1504) and effects on SA dwell time in nanopore.

FIGS. 16A-16C show N-termini Functionalized YaxA_{Δ40}B_{N-strepII-50aa-flex} with N-terminal StrepII-tag (1602) separated with 50 amino acids from YaxB. FIG. 16A shows C-reactive protein (1601) captured by nanopore and representative recordings. FIG. 16B shows capture of Streptavidin A (SA; 1603) in nanopore and prevention of CRP capture. FIG. 16C shows addition of biotin (1604) and effects on SA dwell time in nanopore.

FIGS. 17A-17C show N-termini Functionalized YaxA_{Δ40}B_{N-strepII-70aa-flex} with N-terminal StrepII-tag (1702) separated with 70 amino acids from YaxB. FIG. 17A shows C-reactive protein (1701) not captured by nanopore with representative recordings. FIG. 17B shows capture of Streptavidin A (SA; 1703) in nanopore and no CRP capture events. FIG. 17C shows addition of biotin (1704) and effects on SA dwell time in nanopore with no CRP capture.

FIG. 18 is a depiction of a computer system that is programmed or otherwise configured to implement the methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. As used herein, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most”, “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The invention relates generally to the field of biological nanopores and the use thereof in the detection of analytes including biopolymers. In particular, it can relate to nanopores (e.g., biological nanopores, proteinaceous nanopores), nanopore systems and devices, and their application in analyte analysis (e.g., single molecule analysis), such as detecting the presence, concentration and/or identity of a clinically relevant analyte in a sample (e.g., complex sample).

Provided herein are nanopores, systems and methods directed to biological pores (e.g., biological nanopores). Nanopores may be promising tools (e.g., single-molecule tools) for the electrical characterization and detection of biomolecules. Biological nanopore sensors can consist of a nanometer-sized, protein-based pore embedded in an insulating membrane that separates two chambers filled with an electrolyte solution. When an electrical bias is applied across the membrane, ions can flow through the pore, producing an open pore current. Molecules traversing the pore under such an external potential will temporarily block or reduce the flow of ions, with this effect being more pronounced when the traversing molecule is relatively large compared to the pore diameter. This change in ionic current can be measured, allowing single molecule identification and characterization of unlabeled analytes, in real-time and under physiological conditions. For example, biological nanopores can be used to sequence nucleic acids at the single molecule level.

The applicability of biological nanopores to study proteins (e.g., folded proteins), polypeptides, and peptides may be limited. For example, a dimension (e.g., a diameter or widest dimension) of some biological nanopores may be small for folded proteins to enter into and/or translocate through the pore. Furthermore, the identification of proteins, especially in real-time and in complex biological samples, may be complicated by the sheer variety of sizes and shapes in the proteome.

In some cases, some α-helical biological nanopores, such as fragaceatoxin C (FraC) and cytolysin A (ClyA), may be suitable for detection of peptides and small proteins. In some cases, wild type or engineered ClyA pores may be cylindrical in overall structure, and can comprise an approximately cylindrical inner vestibule (e.g., chamber) with a constriction at the trans entrance that is capable of capturing analytes. A vestibule may refer to an opening channel of a pore through which a substrate or analyte may pass through. The vestibule of a pore (e.g., nanopore) may be a same width through the entire vestibule or a vestibule may have different widths through the entire vestibule. The vestibule may comprise a constriction region in which a width of the vestibule in the constriction region is smaller than a width of the vestibule in another region of the vestibule. ClyA pores may comprise 12 ClyA monomers and may comprise a constriction diameter of about 3.3 nanometers (nm) and a maximum vestibule opening of about 6 nm in diameter. A vestibule opening (e.g., an entrance to a nanopore) may be measured and a dimension (e.g., diameter or widest dimension) can be determined from an outer edge or an inner edge of a vestibule opening. A maximum vestibule opening may be a greatest dimension (e.g., length, width, or diameter) from a first outer edge of a vestibule to a second outer edge of a vestibule. Such pores can detect folded proteins with a molecular weight up to approximately ˜40 kDa. In other cases, ClyA pores may comprise 13 or 14 monomers. In some cases, a ClyA pore may comprise at least about, at most about, or about 3, 4, 5, 6, 7, 8, 9, 10, 11, 12, 13, 14, 15, 16, 17, 18, 19, 20, 22, 24, 26, 28, 30, 40, or 50 monomers, or a range between any of these two values. In some cases, certain ClyA pores may comprise a constriction diameter of about 4.2 nm.

Huang et al. (Angew. Chem. Int. Ed. 2022, 61, e202206227; see also WO2021/182957 A1 in the name of the applicant) disclosed engineered pleurotolysin (PlyAB) nanopores, a β-barrel biological nanopore having a cylindrical trans chamber with a diameter of about 7 nm attached to a truncated cone cis chamber with a larger diameter of approximately 10.5 nm, separated by an inner constriction zone with a diameter of about 5.5 nm. The PlyAB nanopore may be capable of detecting large, folded proteins, including for example the 66.5 kDa human albumin and the 76-81 kDa human transferrin proteins.

As an alternative to biological nanopores, solid state nanopores may be used to study folded proteins. Despite being in principle capable of sensing proteins ranging in size from approximately 6 to 660 kDa, such artificial nanopores suffer from many drawbacks. Proteins, with their non-uniform charge distribution, can adsorb to the nanopore surface or translocate too quickly to be sampled properly. It can also be challenging to reproducibly manufacture solid-state nanopores of uniform size, which is essential for reliable detection. It may not be straightforward to modify the surface properties inside the pore to optimize detection. In some cases, the surface charge, which can controls the nanofluidic properties of the nanopore cannot be modified with atomic precision, and binding elements cannot be introduced with controlled stoichiometry.

The inventors recognized the need for a biological nanopore capable of detecting a wider range of analytes than existing nanopore systems. In some embodiments, the nanopore may be able to capture large (>−80 kDa) analytes (e.g., folded proteins) as well as smaller analytes. In some cases, the nanopore can also be easily and reproducibly manufactured and/or applied for commercial electrophysiological sensing applications.

In some embodiments, the nanopores, methods, and systems described herein provide a nanopore (e.g., uniformly sized nanopore) with a large diameter and/or an appropriate selectivity to allow capture of large analytes (e.g., folded proteins greater than 20 kDa or greater than 50 kDa). The nanopore system can be readily adapted to enhance selective capture of analytes (e.g., unlabeled analytes) from a mixture (e.g., complex mixture) of components, such as biomolecules (e.g., proteins). A mixture comprising an analyte (e.g., an unlabeled analyte) may be a complex mixture. The complex mixture can comprise a target analyte (e.g., protein and/or peptide) and a non-target analyte (e.g., an analyte that is not characterized). A complex mixture can comprise a mixture of proteins, peptides, small molecules, lipids, sugars, carbohydrates, or any combination thereof. The nanopore can be sufficiently stable under conditions used for electrophysiological sensing experiments. Furthermore, the nanopore can enable reliable real-time identification of various size proteins in complex biological samples.

Disclosed herein is a nanopore (e.g., conical shaped nanopore), such as the YaxAB nanopore having a large (e.g., about 15 nm for the hetero-dodecameric species) first opening (e.g., cis opening) and a smaller (e.g., about 3.5 nm) second opening (e.g., trans constriction region). This unique pore geometry may allow for the characterisation of an unprecedented wide range of (protein) analyte sizes and makes it the largest biological nanopore (e.g., proteinaceous nanopore) for molecular analysis characterized thus far.

In some cases, the analyte can be a non-nucleic acid biomolecule. The analyte can be an amino acid-based polymer (e.g., peptide, protein, or polyamino acid). In some embodiments, the analyte can be a carbohydrate-based polymer. The analyte can be a saccharide or polysaccharide molecule. The analyte can comprise one, two, three or any number of nucleotides or nucleic acid molecules. In some cases, the analyte can comprise a non-nucleic acid-based polymer, such an amino acid-based polymer (e.g., peptide, protein, or polyamino acid).

Molecular dynamics and electrical recording showed that the resistance of the nanopore can be dominated by a constriction region (e.g., trans constriction region). In turn, the charge of nanopore, for example at the constriction, generates a strong electroosmotic flow (EOF) that promotes the capture of analytes (e.g., proteins) with a wide range of net electrostatic charges. In some cases, analytes (e.g., proteins) in at least the 25-150 kDa range can be trapped within a conical shape of the nanopore for a time that can be tuned by the external bias. An external bias can comprise an applied voltage to a nanopore and/or system as described herein. Interestingly, and contrary to the currently used cylindrical nanopores, the current blockage decreases with the size of the trapped protein, as smaller analytes (e.g., smaller proteins) penetrate deeper into the constriction region than larger analytes (e.g., larger proteins). Without wishing to be bound by theory, as an analyte (e.g., a non-nucleic acid polymer analyte) translocates to a nanopore as described herein the analyte may reside in the constriction region. The analyte residing in the constriction region may focus the ionic current and provide a measurable signal for analyte detection and characterization. This characteristic is especially useful for characterising large proteins, such as the pentameric C-reactive protein (CRP), a widely used health indicator of around 120 kDa, which shows a unique signal that could be identified in real-time in the presence of depleted blood. Analytes (e.g., proteins) may be identified by various characteristics. Characteristics of an analyte may comprise the length of the analyte (e.g., a contour length, in the case of polymeric analyte), the volume of the analyte, the mass of the analyte, the shape of the analyte, the secondary structure of the analyte, the tertiary structure of the analyte, the charge distribution of the analyte, the identity of the analyte, the sequence of the analyte, any chemical modifications of the analyte, or any combination thereof. A chemical modification to the analyte may comprise a post-translational modification (e.g., phosphorylation, glycosylation, ubiquitination, nitrosylation, methylation, acetylation, lipidation, proteolysis, or any combination thereof).

In some embodiments, the present disclosure provides a nanopore system comprising an actinoporin. In some embodiments, the nanopore system can comprise a nanopore derived from an actinoporin superfamily comprising Actinostoloidea, Actinioidea, Metridioidea, or any combination thereof. In some embodiments, the nanopore system can comprise a nanopore derived from a pore-forming toxin family comprising Actinostoloidea, Actinioidea, Metridioidea, Morganellaceae, Yersiniaceae, or any combination thereof.

### Nanopores

In some aspects, the present disclosure provides pores for detecting and/or characterizing an analyte (e.g., a protein). In some embodiments, the pore can be a biological pore. In some embodiments, the pore comprises a peptide. In some embodiments, the pore comprises a plurality of peptides. In some embodiments, the pore comprises a protein. In some embodiments, the pore comprises a plurality of proteins. In some embodiments, the pore comprises a subunit (e.g., a monomer). In some embodiments, the pore comprises at least one subunit (e.g., at least one monomer). In some embodiments, the pore comprises a plurality of subunits (e.g., a plurality of monomers).

In some embodiments, the pore may be a nanopore (e.g., a biological nanopore). In some embodiments, the pore may be disposed in a membrane. In some embodiments, the pore comprises a transmembrane region. In some embodiments, the pore comprises a hydrophilic portion. In some embodiments, the pore comprises a hydrophobic portion. In some embodiments, the pore comprises a hydrophilic and a hydrophobic portion. In some embodiments, a pore comprises an opening (e.g., an entrance). In some embodiments, a pore comprises at least one opening. In some embodiments, a pore can comprise 1, 2, 3, 4, 5, 6, 7, 8, 9, or more openings. Without wishing to be bound by theory, an entrance to a nanopore may be measured by a diameter or a circumference. An entrance to a nanopore may be defined by a widest dimension (e.g., a measure from a first edge of an entrance to a second edge of the entrance).

In some embodiments, a nanopore (e.g., a biological nanopore) can comprise a first opening. In some embodiments, a nanopore (e.g., a biological nanopore) can comprise a second opening. In some embodiments, a nanopore (e.g., a biological nanopore) can comprise a first opening and a second opening. In some embodiments a first opening of a nanopore described herein may face a first side of a fluid filled chamber. In some embodiments, a second opening of a nanopore described herein may face a second side of a fluid filled chamber. In some embodiments, a first opening of a nanopore may be a cis opening (e.g., a cis entrance). In some embodiments, a second opening of a nanopore may be a cis opening (e.g., a cis entrance). In some embodiments, a first opening of a nanopore may be a trans opening (e.g., a trans entrance). In some embodiments, a second opening of a nanopore may be a trans opening (e.g., a trans entrance).

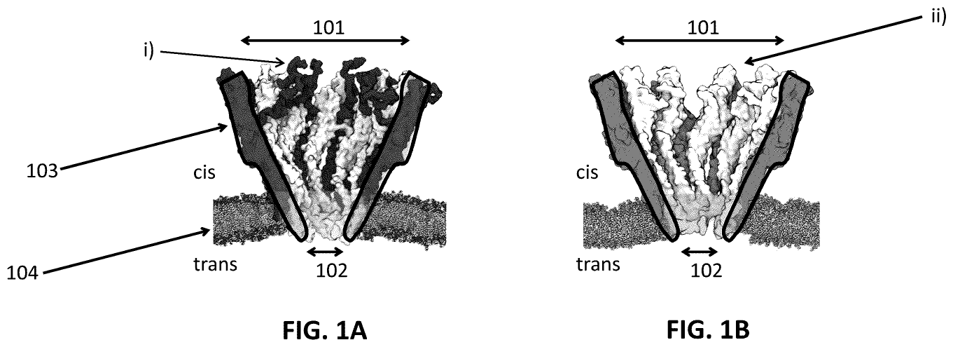

As shown in FIGS. 1A-1B, a nanopore can comprise a first opening (101) which can have a dimension (length, width, diameter, circumference, widest dimension, or any combination thereof). The first opening (101) may be larger than a second opening (102). The first opening (101) may be smaller than a second opening (102). The pore (e.g., biological nanopore) may have an edge (103) that is disposed in a membrane (104). An outer edge can comprise an edge facing away from an interior channel (e.g., lumen) of the nanopore and an inner edge can comprise an edge facing the interior channel (e.g., lumen). In some embodiments, at least one element and/or moiety may be bound to an outer edge of the nanopore, an inner edge of the nanopore, or any combination thereof. In some embodiments, no element or moiety may be bound to the edge of an outer edge of the nanopore or an inner edge of the nanopore. The membrane may have a first side (e.g., cis side) and a second side (e.g., a trans side). In some embodiments, a nanopore may comprise subunits with untruncated N-terminals (i) or truncated N-terminals (ii).

In some embodiments, a nanopore as described herein may comprise a first opening (e.g., cis entrance) of at least 10 nm and a second opening (e.g., trans entrance) of less than 10 nm. In some embodiments, the second opening (e.g., trans entrance) of the nanopore may be smaller than a first opening (e.g., cis entrance) (e.g., the second opening comprises a smaller diameter, circumference, and/or widest dimension than a first opening). A smaller second opening (e.g., trans entrance) than a first opening (e.g., cis entrance) of a nanopore may be referred to as a trans constriction. In some embodiments, the first opening (e.g., cis entrance) of the nanopore may be smaller than a second opening (e.g., trans entrance) (e.g., the first opening comprises a smaller diameter, circumference, and/or widest dimension than a second opening). A smaller first opening (e.g., cis entrance) than a second opening (e.g., trans entrance) of a nanopore may be referred to as a cis constriction. The nanopore may have a first opening (e.g., cis entrance) of about 10 to 25 nm, and/or a trans constriction of 2 to 15 nm. The nanopore may have a first opening (e.g., cis entrance) of about 10 to 25 nm, and/or a trans constriction of 2 to 15 nm.

In some embodiments, a nanopore described herein may comprise a shape. In some embodiments, the nanopore may be cylindrical. In some embodiments, the nanopore may be conical. In some embodiments, the nanopore may be ovular. Without wishing to be bound by theory, a conical nanopore may be advantageous in capturing an analyte as the channel of the conical nanopore constricts from a first opening to a second opening. A large analyte may reside in a constricted region of a conical nanopore, allowing the analyte to be characterized using the nanopores, systems, and methods described herein.

In some embodiments, a nanopore provided herein may comprise a dimension (e.g., diameter, circumference, and/or widest dimension) of a first opening (e.g., cis entrance) of at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 25 nm, at least about 30 nm, or greater than about 30 nm. In some embodiments, a nanopore provided herein may comprise a dimension (e.g., diameter, circumference, and/or widest dimension) of a first opening (e.g., cis entrance) of at most about 30 nm, at most about 25 nm, at most about 20 nm, at most about 19 nm, at most about 18 nm, at most about 17 nm, at most about 16 nm, at most about 15 nm, at most about 14 nm, at most about 13 nm, at most about 12 nm, at most about 11 nm, at most about 10 nm, at most about 9 nm, at most about 8 nm, at most about 7 nm, at most about 6 nm, at most about 5 nm, at most about 4 nm, at most about 3 nm, at most about 2 nm, at most about 1 nm, or less than about 1 nm.

In some embodiments, a nanopore provided herein may comprise a dimension (e.g., diameter, circumference, and/or widest dimension) of a first opening (e.g., cis entrance) from about 1 nm to about 8 nm. In some embodiments, a nanopore provided herein may comprise a dimension (e.g., diameter, circumference, and/or widest dimension) of a first opening (e.g., cis entrance) from about 1 nm to about 1.5 nm, about 1 nm to about 2 nm, about 1 nm to about 2.5 nm, about 1 nm to about 3 nm, about 1 nm to about 3.5 nm, about 1 nm to about 4 nm, about 1 nm to about 4.5 nm, about 1 nm to about 5 nm, about 1 nm to about 6 nm, about 1 nm to about 7 nm, about 1 nm to about 8 nm, about 1.5 nm to about 2 nm, about 1.5 nm to about 2.5 nm, about 1.5 nm to about 3 nm, about 1.5 nm to about 3.5 nm, about 1.5 nm to about 4 nm, about 1.5 nm to about 4.5 nm, about 1.5 nm to about 5 nm, about 1.5 nm to about 6 nm, about 1.5 nm to about 7 nm, about 1.5 nm to about 8 nm, about 2 nm to about 2.5 nm, about 2 nm to about 3 nm, about 2 nm to about 3.5 nm, about 2 nm to about 4 nm, about 2 nm to about 4.5 nm, about 2 nm to about 5 nm, about 2 nm to about 6 nm, about 2 nm to about 7 nm, about 2 nm to about 8 nm, about 2.5 nm to about 3 nm, about 2.5 nm to about 3.5 nm, about 2.5 nm to about 4 nm, about 2.5 nm to about 4.5 nm, about 2.5 nm to about 5 nm, about 2.5 nm to about 6 nm, about 2.5 nm to about 7 nm, about 2.5 nm to about 8 nm, about 3 nm to about 3.5 nm, about 3 nm to about 4 nm, about 3 nm to about 4.5 nm, about 3 nm to about 5 nm, about 3 nm to about 6 nm, about 3 nm to about 7 nm, about 3 nm to about 8 nm, about 3.5 nm to about 4 nm, about 3.5 nm to about 4.5 nm, about 3.5 nm to about 5 nm, about 3.5 nm to about 6 nm, about 3.5 nm to about 7 nm, about 3.5 nm to about 8 nm, about 4 nm to about 4.5 nm, about 4 nm to about 5 nm, about 4 nm to about 6 nm, about 4 nm to about 7 nm, about 4 nm to about 8 nm, about 4.5 nm to about 5 nm, about 4.5 nm to about 6 nm, about 4.5 nm to about 7 nm, about 4.5 nm to about 8 nm, about 5 nm to about 6 nm, about 5 nm to about 7 nm, about 5 nm to about 8 nm, about 6 nm to about 7 nm, about 6 nm to about 8 nm, or about 7 nm to about 8 nm. In some embodiments, a nanopore provided herein may comprise a dimension of a first opening (e.g., cis entrance) (e.g., diameter, circumference, and/or widest dimension) from about 8 nm to about 30 nm. In some embodiments, a nanopore provided herein may comprise a dimension of a first opening (e.g., cis entrance) (e.g., diameter, circumference, and/or widest dimension) from at most about 30 nm. In some embodiments, a nanopore provided herein may comprise a dimension of a first opening (e.g., cis entrance) (e.g., diameter, circumference, and/or widest dimension) from about 8 nm to about 9 nm, about 8 nm to about 10 nm, about 8 nm to about 11 nm, about 8 nm to about 12 nm, about 8 nm to about 13 nm, about 8 nm to about 14 nm, about 8 nm to about 15 nm, about 8 nm to about 20 nm, about 8 nm to about 25 nm, about 8 nm to about 30 nm, about 9 nm to about 10 nm, about 9 nm to about 11 nm, about 9 nm to about 12 nm, about 9 nm to about 13 nm, about 9 nm to about 14 nm, about 9 nm to about 15 nm, about 9 nm to about 20 nm, about 9 nm to about 25 nm, about 9 nm to about 30 nm, about 10 nm to about 11 nm, about 10 nm to about 12 nm, about 10 nm to about 13 nm, about 10 nm to about 14 nm, about 10 nm to about 15 nm, about 10 nm to about 20 nm, about 10 nm to about 25 nm, about 10 nm to about 30 nm, about 11 nm to about 12 nm, about 11 nm to about 13 nm, about 11 nm to about 14 nm, about 11 nm to about 15 nm, about 11 nm to about 20 nm, about 11 nm to about 25 nm, about 11 nm to about 30 nm, about 12 nm to about 13 nm, about 12 nm to about 14 nm, about 12 nm to about 15 nm, about 12 nm to about 20 nm, about 12 nm to about 25 nm, about 12 nm to about 30 nm, about 13 nm to about 14 nm, about 13 nm to about 15 nm, about 13 nm to about 20 nm, about 13 nm to about 25 nm, about 13 nm to about 30 nm, about 14 nm to about 15 nm, about 14 nm to about 20 nm, about 14 nm to about 25 nm, about 14 nm to about 30 nm, about 15 nm to about 20 nm, about 15 nm to about 25 nm, about 15 nm to about 30 nm, about 20 nm to about 25 nm, about 20 nm to about 30 nm, or about 25 nm to about 30 nm.

In some embodiments, a nanopore provided herein may comprise a dimension (e.g., diameter, circumference, and/or widest dimension) of a second opening (e.g., trans entrance) of at least about 1 nm, at least about 1.5 nm, at least about 2 nm, at least about 2.5 nm, at least about 3 nm, at least about 3.5 nm, at least about 4 nm, at least about 4.5 nm, at least about 5 nm, at least about 5.5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, or greater than about 15 nm. In some embodiments, a nanopore provided herein may comprise a dimension (e.g., diameter, circumference, and/or widest dimension) of a second opening (e.g., trans entrance) of at most about 15 nm, at most about 14 nm, at most about 13 nm, at most about 12 nm, at most about 11 nm, at most about 10 nm, at most about 9 nm, at most about 8 nm, at most about 7 nm, at most about 6 nm, at most about 5.5 nm, at most about 5 nm, at most about 4.5 nm, at most about 4 nm, at most about 3.5 nm, at most about 3 nm, at most about 2.5 nm, at most about 2 nm, at most about 1.5 nm, at most about 1 nm, or less than about 1 nm.

In some embodiments, a nanopore provided herein may comprise a dimension (e.g., diameter, circumference, and/or widest dimension) of a second opening (e.g., trans entrance) from about 0.5 nm to about 6 nm. In some embodiments, a nanopore provided herein may comprise a dimension (e.g., diameter, circumference, and/or widest dimension) of a second opening (e.g., trans entrance) from about 0.5 nm to about 1 nm, about 0.5 nm to about 1.5 nm, about 0.5 nm to about 2 nm, about 0.5 nm to about 2.5 nm, about 0.5 nm to about 3 nm, about 0.5 nm to about 3.5 nm, about 0.5 nm to about 4 nm, about 0.5 nm to about 4.5 nm, about 0.5 nm to about 5 nm, about 0.5 nm to about 5.5 nm, about 0.5 nm to about 6 nm, about 1 nm to about 1.5 nm, about 1 nm to about 2 nm, about 1 nm to about 2.5 nm, about 1 nm to about 3 nm, about 1 nm to about 3.5 nm, about 1 nm to about 4 nm, about 1 nm to about 4.5 nm, about 1 nm to about 5 nm, about 1 nm to about 5.5 nm, about 1 nm to about 6 nm, about 1.5 nm to about 2 nm, about 1.5 nm to about 2.5 nm, about 1.5 nm to about 3 nm, about 1.5 nm to about 3.5 nm, about 1.5 nm to about 4 nm, about 1.5 nm to about 4.5 nm, about 1.5 nm to about 5 nm, about 1.5 nm to about 5.5 nm, about 1.5 nm to about 6 nm, about 2 nm to about 2.5 nm, about 2 nm to about 3 nm, about 2 nm to about 3.5 nm, about 2 nm to about 4 nm, about 2 nm to about 4.5 nm, about 2 nm to about 5 nm, about 2 nm to about 5.5 nm, about 2 nm to about 6 nm, about 2.5 nm to about 3 nm, about 2.5 nm to about 3.5 nm, about 2.5 nm to about 4 nm, about 2.5 nm to about 4.5 nm, about 2.5 nm to about 5 nm, about 2.5 nm to about 5.5 nm, about 2.5 nm to about 6 nm, about 3 nm to about 3.5 nm, about 3 nm to about 4 nm, about 3 nm to about 4.5 nm, about 3 nm to about 5 nm, about 3 nm to about 5.5 nm, about 3 nm to about 6 nm, about 3.5 nm to about 4 nm, about 3.5 nm to about 4.5 nm, about 3.5 nm to about 5 nm, about 3.5 nm to about 5.5 nm, about 3.5 nm to about 6 nm, about 4 nm to about 4.5 nm, about 4 nm to about 5 nm, about 4 nm to about 5.5 nm, about 4 nm to about 6 nm, about 4.5 nm to about 5 nm, about 4.5 nm to about 5.5 nm, about 4.5 nm to about 6 nm, about 5 nm to about 5.5 nm, about 5 nm to about 6 nm, or about 5.5 nm to about 6 nm. In some embodiments, a nanopore provided herein may comprise a dimension (e.g., diameter, circumference, and/or widest dimension) of a second opening (e.g., trans entrance) from about 6 nm to about 15 nm. In some embodiments, a nanopore provided herein may comprise a dimension (e.g., diameter, circumference, and/or widest dimension) of a second opening (e.g., trans entrance) from about 6 nm to about 7 nm, about 6 nm to about 8 nm, about 6 nm to about 9 nm, about 6 nm to about 10 nm, about 6 nm to about 11 nm, about 6 nm to about 12 nm, about 6 nm to about 13 nm, about 6 nm to about 14 nm, about 6 nm to about 15 nm, about 7 nm to about 8 nm, about 7 nm to about 9 nm, about 7 nm to about 10 nm, about 7 nm to about 11 nm, about 7 nm to about 12 nm, about 7 nm to about 13 nm, about 7 nm to about 14 nm, about 7 nm to about 15 nm, about 8 nm to about 9 nm, about 8 nm to about 10 nm, about 8 nm to about 11 nm, about 8 nm to about 12 nm, about 8 nm to about 13 nm, about 8 nm to about 14 nm, about 8 nm to about 15 nm, about 9 nm to about 10 nm, about 9 nm to about 11 nm, about 9 nm to about 12 nm, about 9 nm to about 13 nm, about 9 nm to about 14 nm, about 9 nm to about 15 nm, about 10 nm to about 11 nm, about 10 nm to about 12 nm, about 10 nm to about 13 nm, about 10 nm to about 14 nm, about 10 nm to about 15 nm, about 11 nm to about 12 nm, about 11 nm to about 13 nm, about 11 nm to about 14 nm, about 11 nm to about 15 nm, about 12 nm to about 13 nm, about 12 nm to about 14 nm, about 12 nm to about 15 nm, about 13 nm to about 14 nm, about 13 nm to about 15 nm, or about 14 nm to about 15 nm.

In some embodiments, the first opening of the nanopore comprises a length. In some embodiments, the second opening of the nanopore comprises a length. In some embodiments, the length of the first opening is greater than the length of the second opening of the nanopore (e.g., biological nanopore). In some embodiments, a length of the first opening of the nanopore (e.g., biological nanopore) is at least 2×, 3×, 4×, 5×, 6×, 7×, 8×, 9×, 10, or greater than about 10× greater than a length of the second opening of the nanopore (e.g., biological nanopore).

In some embodiments, the nanopore comprises an alpha-helical pore-forming toxin or porin. In some embodiments, the nanopore comprises at least one alpha-helical structure. In some embodiments, the nanopore comprises an alpha-helical protein (e.g., pore-forming protein). In some embodiments, the nanopore comprises an alpha-helical peptide (e.g., pore-forming peptide). In some embodiments, the nanopore comprises at least a portion of an alpha-helical pore-forming protein or peptide. In some embodiments, the nanopore comprises at least a portion of an alpha-helical protein or peptide from the pore-forming toxin. In some embodiments, the nanopore comprises a beta-barrel pore-forming toxin or porin. In some embodiments, the nanopore comprises at least one beta-barrel structure. In some embodiments, the nanopore comprises a beta-barrel protein (e.g., pore-forming protein). In some embodiments, the nanopore comprises a beta-barrel peptide (e.g., pore-forming peptide). In some embodiments, the nanopore comprises at least a portion of a beta-barrel pore-forming protein or peptide. In some embodiments, the nanopore comprises at least a portion of a beta-barrel protein or peptide from the pore-forming toxin.

In some embodiments, the present disclosure provides a nanopore comprising an actinoporin. In some embodiments, the nanopore can comprise a nanopore derived from an actinoporin superfamily comprising Actinostoloidea, Actinioidea, Metridioidea, or any combination thereof. In some embodiments, the nanopore can comprise a nanopore derived from a pore-forming toxin family comprising Actinostoloidea, Actinioidea, Metridioidea, Yersinia enterocolitica, Xenorhabdus nematophila, or any combination thereof.

In some embodiments, the nanopore may be derived from the family Morganellaceae, Yersiniaceae, or any combination thereof. The pore may originate from a genus comprising Photorhabdus, Xenorhabdus, Yersinia, or any combination thereof. For example, a nanopore may originate from the species P. luminescens, X. nematophila, Y. enterocolitica, or any combination thereof.

In some cases, the pore may comprise a tripartite pore. The tripartite pore may stem from Aeromonas hydrophila, Bacillus cereus, or any combination thereof. For example, the nanopore may comprise an AhlABC pore from Aeromonas hydrophila, a Hb1CDA from Bacillus cereus, a NheABC pore from Bacillus cereus, or any combination thereof.

In some embodiments, a nanopore is selected from the group consisting of Aerolysin (Aer), Cytolysin K (CytK), MspA, alpha-hemolysin (aHL), CsgG, Fragaceatoxin C (FraC), Lysenin, phage derived portal proteins (Phi29, G20c, etc.), pleurotolysin (PlyA or PlyB), ClyA, or a mutant thereof. In some embodiments, the nanopore (e.g., biological nanopore) does not comprise an alpha-hemolysin. In some embodiments, the nanopore does not comprise a portion of an alpha-hemolysin. In some embodiments, the nanopore does not comprise a porin of bacteria. In some embodiments the nanopore does not comprise a porin originating from a Mycobacterium smegmatis. In some embodiments, the nanopore does not comprise a MspA. In some embodiments, the nanopore does not comprise a portion of MspA. In some embodiments, the nanopore does not comprise a Aer. In some embodiments, the nanopore does not comprise a portion of Aer. In some embodiments, the nanopore does not comprise a CsgG. In some embodiments, the nanopore does not comprise a portion of CsgG. In some embodiments, the nanopore does not comprise a CytK. In some embodiments, the nanopore does not comprise a portion of CytK. In some embodiments, the nanopore does not comprise a FraC. In some embodiments, the nanopore does not comprise a portion of FraC. In some embodiments, the nanopore does not comprise a Lysenin. In some embodiments, the nanopore does not comprise a portion of Lysenin. In some embodiments, the nanopore does not comprise a Phi29. In some embodiments, the nanopore does not comprise a portion of Phi29.

In one embodiment, the nanopore (e.g., biological nanopore) comprises one or more components (e.g., two-component or bipartite) of a heterooligomeric pore. In some embodiments, the nanopore comprises one or more components (e.g., monomers) of the alpha-xenorhabdolysin family of binary toxin or a mutant, functional homolog, functional ortholog, or functional paralog thereof. “Homologs” can refer to proteins, peptides, oligopeptides, polypeptides having amino acid substitutions, deletions, insertions, or any combination thereof relative to an unmodified (e.g., wild-type) protein and having similar biological and/or functional activity as the unmodified protein from which they are derived. “Ortholog” can refer to a gene or protein from different organisms (e.g., different species) that are derived from a common ancestral gene. “Paralog” can refer to a gene or protein from the same organism (e.g., same species) that is a product of gene duplication of a common ancestral gene. In some embodiments, the nanopore comprises one or more components (e.g., monomers) of the YaxAB toxin of Yersinia enterocolitica. In some embodiments, the nanopore comprises one or more components (e.g., monomers) of the XaxAB toxin of Xenorhabdus nematophila. The Yersinia YaxAB system represents a family of binary α-pore-forming toxins (PFTs) with orthologues in human, insect, and plant pathogens.

Disclosed herein are nanopores, systems, and methods comprising a biological nanopore (e.g., oligomeric nanopore). A nanopore may comprise one or more monomers. A monomer of the nanopore may comprises one or more portions (e.g., subunits). The one or more portions may comprise one or more proteins, polypeptides, or peptides. For example, a monomer may comprise one protein, one polypeptide, or one peptide. In another example, a subunit may comprise a first portion (e.g., a first protein, first polypeptide, or first peptide) and a second portion (e.g., a second protein, a second polypeptide, or a second peptide).

In some embodiments, the nanopore comprises a pore-forming toxin. The nanopore can comprise an α-pore-forming toxin, a β-pore-forming toxin, or any combination thereof. The nanopore can comprise a pore-forming toxin derived from a bacterium. The bacterium can be of a genus of bacteria including, but not limited to, Xenorhabdus, Yersinia, Providencia, Pseudomonas, Proteus, Morganella, or Photorhabdus. In some cases, the monomer may comprise one or more portions comprising proteins, polypeptides, or peptides of the alpha-xenorhabdolysin family of binary toxins.

In some embodiments, the monomer of a nanopore (e.g., biological nanopore) described herein may comprise one or more portions (e.g., YaxA and/or YaxB subunits) originating from Yersinia enterocolitica. In some embodiments, the monomer of a nanopore (e.g., biological nanopore) described herein may comprise one or more portions (e.g., PaYaxA and/or PaYaxB subunits) originating from Providencia alcalifaciens. In some embodiments, the monomer of a nanopore (e.g., biological nanopore) described herein may comprise one or more portions (e.g., PsYaxA and/or PsYaxB subunits) originating from Pseudomonas syringae. In some embodiments, the monomer of a nanopore (e.g., biological nanopore) described herein may comprise one or more portions (e.g., PmYaxA and/or PmYaxB subunits) originating from Proteus mirabilis. In some embodiments, the monomer of a nanopore (e.g., biological nanopore) described herein may comprise one or more portions (e.g., e.g., MmYaxA, MmYaxB subunits) originating from Morganella morganii. In some embodiments, the monomer of a nanopore (e.g., biological nanopore) described herein may comprise one or more portions (e.g., PaxA and/or PaxB subunits) originating from Photorhabdus luminescens. In some embodiments, the monomer of a nanopore (e.g., biological nanopore) described herein may comprise one or more portions (e.g., XaxA and/or XaxB subunits) originating from Xenorhabdus nematophila. Table 5 provides the amino acid sequences of alpha-xenorhabdolysin family binary toxin orthologues. In some embodiments, the monomer of a nanopore (e.g., biological nanopore) described herein may comprise one or more portions (e.g., YaxA and/or YaxB subunits) originating from Yersinia enterocolitica. In some embodiments, the monomer of a nanopore (e.g., biological nanopore) described herein may comprise one or more YaxA and/or YaxB portions (e.g., subunits) or a mutant, functional homolog, functional ortholog, or functional paralog thereof. In some embodiments, the monomer of a nanopore (e.g., biological nanopore) described herein may comprise one or more portions (e.g., PaYaxA and/or PaYaxB subunits) originating from Providencia alcalifaciens. In some embodiments, the monomer of a nanopore (e.g., biological nanopore) described herein may comprise one or more PaYaxA and/or PaYaxB portions (e.g., subunits) or a mutant, functional homolog, functional ortholog, or functional paralog thereof. In some embodiments, the monomer of a nanopore (e.g., biological nanopore) described herein may comprise one or more portions (e.g., PsYaxA and/or PsYaxB subunits) originating from Pseudomonas syringae. In some embodiments, the monomer of a nanopore (e.g., biological nanopore) described herein may comprise one or more PsYaxA and/or PsYaxB portions (e.g., subunits) or a mutant, functional homolog, functional ortholog, or functional paralog thereof. In some embodiments, the monomer of a nanopore (e.g., biological nanopore) described herein may comprise one or more portions (e.g., PmYaxA and/or PmYaxB subunits) originating from Proteus mirabilis. In some embodiments, the monomer of a nanopore (e.g., biological nanopore) described herein may comprise one or more PmYaxA and/or PmYaxB portions (e.g., subunits) or a mutant, functional homolog, functional ortholog, or functional paralog thereof. In some embodiments, the monomer of a nanopore (e.g., biological nanopore) described herein may comprise one or more portions (e.g., MmYaxA and/or MmYaxB subunits) originating from Morganella morganii. In some embodiments, the monomer of a nanopore (e.g., biological nanopore) described herein may comprise one or more MmYaxA and/or MmYaxB portions (e.g., subunits) or a mutant, functional homolog, functional ortholog, or functional paralog thereof. In some embodiments, the monomer of a nanopore (e.g., biological nanopore) described herein may comprise one or more portions (e.g., PaxA and/or PaxB subunits) originating from Photorhabdus luminescens. In some embodiments, the monomer of a nanopore (e.g., biological nanopore) described herein may comprise one or more PaxA and/or PaxB portions (e.g., subunits) or a mutant, functional homolog, functional ortholog, or functional paralog thereof. In some embodiments, the monomer of a nanopore (e.g., biological nanopore) described herein may comprise one or more portions (e.g., XaxA and/or XaxB subunits) originating from Xenorhabdus nematophila. In some embodiments, the monomer of a nanopore (e.g., biological nanopore) described herein may comprise one or more XaxA and/or XaxB portions (e.g., subunits) or a mutant, functional homolog, functional ortholog, or functional paralog thereof. In some embodiments, a monomer of a nanopore described herein may comprise an amino acid sequence with at least about 80%, 85%, 90%, 95%, 99%, or 100% sequence identity to any one of the amino acid sequences as set forth in SEQ ID NOs: 25-38.

In some embodiments, the monomer of a nanopore (e.g., biological nanopore) described herein may comprise one or more portions (e.g., YaxA and/or YaxB subunits) originating from a full-length or truncated variant of Yersinia enterocolitica. In some embodiments, the monomer of a nanopore (e.g., biological nanopore) described herein may comprise one or more portions (e.g., PaYaxA and/or PaYaxB subunits) originating from a full-length or truncated variant of Providencia alcalifaciens. In some embodiments, the monomer of a nanopore (e.g., biological nanopore) described herein may comprise one or more portions (e.g., PsYaxA and/or PsYaxB subunits) originating from a full-length or truncated variant of Pseudomonas syringae. In some embodiments, the monomer of a nanopore (e.g., biological nanopore) described herein may comprise one or more portions (e.g., PmYaxA and/or PmYaxB subunits) originating from a full-length or truncated variant of Proteus mirabilis. In some embodiments, the monomer of a nanopore (e.g., biological nanopore) described herein may comprise one or more portions (e.g., MmYaxA and/or MmYaxB subunits) originating from a full-length or truncated variant of Morganella morganii. In some embodiments, the monomer of a nanopore (e.g., biological nanopore) described herein may comprise one or more portions (e.g., PaxA and/or PaxB subunits) originating from a full-length or truncated variant of Photorhabdus luminescens. In some embodiments, the monomer of a nanopore (e.g., biological nanopore) described herein may comprise one or more portions (e.g., XaxA and/or XaxB subunits) originating from a full-length or truncated variant of Xenorhabdus nematophila. In some embodiments, the monomer of a nanopore (e.g., biological nanopore) described herein may comprise one or more portions originating from a full-length or truncated variant of Yersinia enterocolitica, Providencia alcalifaciens, Pseudomonas syringae, Proteus mirabilis, Morganella morganii, Photorhabdus luminescens, Xenorhabdus nematophila, or any combination thereof.

In some embodiments, the nanopore may comprise an assembly of monomers. In some embodiments, the nanopore may comprise an assembly of monomers of the alpha-xenorhabdolysin family of binary toxin or mutants, functional homologs, functional orthologs, or functional paralogs thereof. The nanopore may comprise a number of monomers. Monomers may be arranged vertically, horizontally, and/or layered as rings to form a nanopore described herein. In some embodiments, a nanopore (e.g., biological nanopore) comprises at least about 2, 3, 4, 5, 6, 7, 8, 9, 10, 12, 14, 16, 18, 20, 25, 30, 40, 50, or greater than 50 monomers. In some embodiments, a nanopore (e.g., biological nanopore) comprises at most about 50, 40, 30, 25, 20, 18, 16, 14, 12, 10, 9, 8, 7, 6, 5, 4, 3, 2, or less than 2 monomers. In some embodiments, a nanopore (e.g., biological nanopore) comprises from about 3 monomers to about 40 monomers. In some embodiments, a nanopore (e.g., biological nanopore) comprises from about 3 monomers to about 4 monomers, about 3 monomers to about 5 monomers, about 3 monomers to about 6 monomers, about 3 monomers to about 7 monomers, about 3 monomers to about 8 monomers, about 3 monomers to about 9 monomers, about 3 monomers to about 10 monomers, about 3 monomers to about 15 monomers, about 3 monomers to about 20 monomers, about 3 monomers to about 30 monomers, about 3 monomers to about 40 monomers, about 4 monomers to about 5 monomers, about 4 monomers to about 6 monomers, about 4 monomers to about 7 monomers, about 4 monomers to about 8 monomers, about 4 monomers to about 9 monomers, about 4 monomers to about 10 monomers, about 4 monomers to about 15 monomers, about 4 monomers to about 20 monomers, about 4 monomers to about 30 monomers, about 4 monomers to about 40 monomers, about 5 monomers to about 6 monomers, about 5 monomers to about 7 monomers, about 5 monomers to about 8 monomers, about 5 monomers to about 9 monomers, about 5 monomers to about 10 monomers, about 5 monomers to about 15 monomers, about 5 monomers to about 20 monomers, about 5 monomers to about 30 monomers, about 5 monomers to about 40 monomers, about 6 monomers to about 7 monomers, about 6 monomers to about 8 monomers, about 6 monomers to about 9 monomers, about 6 monomers to about 10 monomers, about 6 monomers to about 15 monomers, about 6 monomers to about 20 monomers, about 6 monomers to about 30 monomers, about 6 monomers to about 40 monomers, about 7 monomers to about 8 monomers, about 7 monomers to about 9 monomers, about 7 monomers to about 10 monomers, about 7 monomers to about 15 monomers, about 7 monomers to about 20 monomers, about 7 monomers to about 30 monomers, about 7 monomers to about 40 monomers, about 8 monomers to about 9 monomers, about 8 monomers to about 10 monomers, about 8 monomers to about 15 monomers, about 8 monomers to about 20 monomers, about 8 monomers to about 30 monomers, about 8 monomers to about 40 monomers, about 9 monomers to about 10 monomers, about 9 monomers to about 15 monomers, about 9 monomers to about 20 monomers, about 9 monomers to about 30 monomers, about 9 monomers to about 40 monomers, about 10 monomers to about 15 monomers, about 10 monomers to about 20 monomers, about 10 monomers to about 30 monomers, about 10 monomers to about 40 monomers, about 15 monomers to about 20 monomers, about 15 monomers to about 30 monomers, about 15 monomers to about 40 monomers, about 20 monomers to about 30 monomers, about 20 monomers to about 40 monomers, or about 30 monomers to about 40 monomers.

In some embodiments, the nanopore can comprise an assembly (e.g., an oligomeric assembly) of YaxA and YaxB subunits, or mutants, functional homologs, functional orthologs, or functional paralogs thereof. In some embodiments, a monomer comprises a first portion (e.g., subunit) and the first portion comprises a YaxA subunit. In some embodiments, a monomer comprises a first portion (e.g., subunit) and the first portion comprises a YaxB subunit. In some embodiments, a monomer comprises a first portion (e.g., first subunit) and a second portion (e.g., second subunit). The first portion of the monomer can be the same protein, polypeptide, or peptide as the second portion. The first portion can be a different protein, polypeptide, or peptide from the second portion. In some embodiments, the monomer comprises only the first portion. In some embodiments, the monomer comprises only the second portion. In some embodiments, the monomer can comprise only YaxA. In some embodiments, the monomer can comprise only YaxB.

A monomeric unit of a nanopore described herein may comprise a first subunit. A monomeric unit of a nanopore described herein may comprise a second subunit. A monomeric unit of a nanopore described herein may comprise at least a first subunit and a second subunit. The first subunit and the second subunit can be the same subunit (e.g., the same protein). In other cases, the first subunit and the second subunit can be different subunits (e.g., different proteins). In some embodiments, the monomer (e.g., monomeric unit) can comprise a dimer of a YaxA subunit and a YaxB subunit, or mutant, functional homolog, functional ortholog, or functional paralog thereof (e.g., a heterodimer or YaxAB dimer). A monomeric unit can comprise a YaxAB dimer (e.g., heterodimer) comprising a YaxA subunit (e.g. portion) and a YaxB subunit (e.g., portion). In some cases, a monomer can comprise a dimer of a YaxA subunit and a YaxA subunit (e.g., a first portion and second portion of the monomer are the same protein). In some cases, a monomer can comprise a dimer of a YaxB subunit and a YaxB subunit (e.g., a first portion and second portion of the monomer are the same protein).

In some embodiments, the nanopore (e.g., biological nanopore) may comprise different numbers of monomeric units. For example, the nanopore can be formed by an assembly (e.g., an oligomeric assembly) of 2 to 20, or 8 to 12, heterodimers of YaxA and YaxB subunits, or mutants, functional homologs, functional orthologs, or functional paralogs thereof.

In some embodiments, the nanopore may comprise a number of PaxA, PaxB, XaxA, or XaxB subunits, or any combination thereof. The nanopore may comprise a number of monomeric units. The monomeric units may originate from a Photorhabdus genus, a Xenorhabdus genus, or any combination thereof. The monomer may originate from P. luminescens and/or X. nematophila. For example, a nanopore (e.g., a biological nanopore) described herein may comprise an assembly of heterodimers (e.g., monomers) formed from PaxA subunits, PaxA subunits, PaxB subunits, XaxA subunits, XaxB subunits, or any combination thereof.

In some embodiments, a nanopore can be a tripartite pore, in which the monomeric units can comprise three subunits (e.g., portions). The subunits may originate from an Aeromonas genus, a Bacillus genus, or any combination thereof. The subunits may originate from an Aeromonas hydrophila species, a Bacillus cereus species, or any combination thereof. In some embodiments, a tripartite pore as described herein may need at least one, at least two, or three subunits. In some embodiments, the nanopore may comprise at least one AhlA subunit, at least one AhlB subunit, at least one AhlC subunit, at least one HblC subunit, at least one HblD subunit, at least one HblA subunit, at least one NheA subunit, at least one NheB subunit, at least one NheC subunit, or any combination thereof. A nanopore described herein may comprise an assembly of at least one AhlA subunit, at least one AhlB subunit, at least one AhlC subunit, or any combination thereof, and form an AhlABC nanopore. A nanopore described herein may comprise an assembly of at least one HblC subunit, at least one HblD subunit, at least one HblA subunit, or any combination thereof, and form a HblCDA nanopore. A nanopore described herein may comprise an assembly of at least one NheA subunit, at least one NheB subunit, at least one NheC subunit, or any combination thereof, and form a NheABC.

In some cases, a nanopore (e.g., biological nanopore) described herein can comprise at least about 4 YaxAB heterodimers, at least about 5 YaxAB heterodimers, at least about 6 YaxAB heterodimers, at least about 7 YaxAB heterodimers, at least about 8 YaxAB heterodimers, at least about 9 YaxAB heterodimers, at least about 10 YaxAB heterodimers, at least about 11 YaxAB heterodimers, at least about 12 YaxAB heterodimers, at least about 13 YaxAB heterodimers, at least about 14 YaxAB heterodimers, at least about 15 YaxAB heterodimers, at least about 16 YaxAB heterodimers, at least about 17 YaxAB heterodimers, at least about 18 YaxAB heterodimers, at least about 19 YaxAB heterodimers, at least about 20 YaxAB heterodimers, at least about 25 YaxAB heterodimers, at least about 30 YaxAB heterodimers, or greater than about 30 YaxAB heterodimers. In some cases, a nanopore described herein can comprise at most about 30 YaxAB heterodimers, at most about 25 YaxAB heterodimers, at most about 20 YaxAB heterodimers, at most about 19 YaxAB heterodimers, at most about 18 YaxAB heterodimers, at most about 17 YaxAB heterodimers, at most about 16 YaxAB heterodimers, at most about 15 YaxAB heterodimers, at most about 14 YaxAB heterodimers, at most about 13 YaxAB heterodimers, at most about 12 YaxAB heterodimers, at most about 11 YaxAB heterodimers, at most about 10 YaxAB heterodimers, at most about 9 YaxAB heterodimers, at most about 8 YaxAB heterodimers, at most about 7 YaxAB heterodimers, at most about 6 YaxAB heterodimers, at most about 5 YaxAB heterodimers, at most about 4 YaxAB heterodimers, or less than about 4 YaxAB heterodimers.

In some cases, a nanopore described herein can comprise from about 6 YaxAB heterodimers to about 20 YaxAB heterodimers. In some cases, a nanopore described herein can comprise from about 6 YaxAB heterodimers to about 7 YaxAB heterodimers, about 6 YaxAB heterodimers to about 8 YaxAB heterodimers, about 6 YaxAB heterodimers to about 9 YaxAB heterodimers, about 6 YaxAB heterodimers to about 10 YaxAB heterodimers, about 6 YaxAB heterodimers to about 11 YaxAB heterodimers, about 6 YaxAB heterodimers to about 12 YaxAB heterodimers, about 6 YaxAB heterodimers to about 13 YaxAB heterodimers, about 6 YaxAB heterodimers to about 14 YaxAB heterodimers, about 6 YaxAB heterodimers to about 15 YaxAB heterodimers, about 6 YaxAB heterodimers to about 18 YaxAB heterodimers, about 6 YaxAB heterodimers to about 20 YaxAB heterodimers, about 7 YaxAB heterodimers to about 8 YaxAB heterodimers, about 7 YaxAB heterodimers to about 9 YaxAB heterodimers, about 7 YaxAB heterodimers to about 10 YaxAB heterodimers, about 7 YaxAB heterodimers to about 11 YaxAB heterodimers, about 7 YaxAB heterodimers to about 12 YaxAB heterodimers, about 7 YaxAB heterodimers to about 13 YaxAB heterodimers, about 7 YaxAB heterodimers to about 14 YaxAB heterodimers, about 7 YaxAB heterodimers to about 15 YaxAB heterodimers, about 7 YaxAB heterodimers to about 18 YaxAB heterodimers, about 7 YaxAB heterodimers to about 20 YaxAB heterodimers, about 8 YaxAB heterodimers to about 9 YaxAB heterodimers, about 8 YaxAB heterodimers to about 10 YaxAB heterodimers, about 8 YaxAB heterodimers to about 11 YaxAB heterodimers, about 8 YaxAB heterodimers to about 12 YaxAB heterodimers, about 8 YaxAB heterodimers to about 13 YaxAB heterodimers, about 8 YaxAB heterodimers to about 14 YaxAB heterodimers, about 8 YaxAB heterodimers to about 15 YaxAB heterodimers, about 8 YaxAB heterodimers to about 18 YaxAB heterodimers, about 8 YaxAB heterodimers to about 20 YaxAB heterodimers, about 9 YaxAB heterodimers to about 10 YaxAB heterodimers, about 9 YaxAB heterodimers to about 11 YaxAB heterodimers, about 9 YaxAB heterodimers to about 12 YaxAB heterodimers, about 9 YaxAB heterodimers to about 13 YaxAB heterodimers, about 9 YaxAB heterodimers to about 14 YaxAB heterodimers, about 9 YaxAB heterodimers to about 15 YaxAB heterodimers, about 9 YaxAB heterodimers to about 18 YaxAB heterodimers, about 9 YaxAB heterodimers to about 20 YaxAB heterodimers, about 10 YaxAB heterodimers to about 11 YaxAB heterodimers, about 10 YaxAB heterodimers to about 12 YaxAB heterodimers, about 10 YaxAB heterodimers to about 13 YaxAB heterodimers, about 10 YaxAB heterodimers to about 14 YaxAB heterodimers, about 10 YaxAB heterodimers to about 15 YaxAB heterodimers, about 10 YaxAB heterodimers to about 18 YaxAB heterodimers, about 10 YaxAB heterodimers to about 20 YaxAB heterodimers, about 11 YaxAB heterodimers to about 12 YaxAB heterodimers, about 11 YaxAB heterodimers to about 13 YaxAB heterodimers, about 11 YaxAB heterodimers to about 14 YaxAB heterodimers, about 11 YaxAB heterodimers to about 15 YaxAB heterodimers, about 11 YaxAB heterodimers to about 18 YaxAB heterodimers, about 11 YaxAB heterodimers to about 20 YaxAB heterodimers, about 12 YaxAB heterodimers to about 13 YaxAB heterodimers, about 12 YaxAB heterodimers to about 14 YaxAB heterodimers, about 12 YaxAB heterodimers to about 15 YaxAB heterodimers, about 12 YaxAB heterodimers to about 18 YaxAB heterodimers, about 12 YaxAB heterodimers to about 20 YaxAB heterodimers, about 13 YaxAB heterodimers to about 14 YaxAB heterodimers, about 13 YaxAB heterodimers to about 15 YaxAB heterodimers, about 13 YaxAB heterodimers to about 18 YaxAB heterodimers, about 13 YaxAB heterodimers to about 20 YaxAB heterodimers, about 14 YaxAB heterodimers to about 15 YaxAB heterodimers, about 14 YaxAB heterodimers to about 18 YaxAB heterodimers, about 14 YaxAB heterodimers to about 20 YaxAB heterodimers, about 15 YaxAB heterodimers to about 18 YaxAB heterodimers, about 15 YaxAB heterodimers to about 20 YaxAB heterodimers, or about 18 YaxAB heterodimers to about 20 YaxAB heterodimers.

In some embodiments, a nanopore may comprise at least about, at most about, or about 1, 2, 3, 4, 5, 6, 7, 8, 9, 10, 12, 15, 18, 20, 22, 24, 26, 28, 30, 40, 50, PaxAB and/or XaxAB heterodimers, or a value in between any of these two values.

In some cases, the nanopore comprises at least one YaxA subunit. In some embodiments, the nanopore comprises at least one YaxB subunit. In some embodiments, the nanopore comprises an equal number of YaxA and YaxB subunits. FIGS. 1A-1B illustrates an example of a nanopore described herein. FIGS. 1A-1B shows molecular surface representations of YaxAB nanopores imbedded in a lipid membrane, comprised of a decamer of YaxA-YaxB dimers (20-mer), showing half of the nanopore as a cut-through to illustrate the conical shape of the nanopore interior. FIG. 1A shows YaxAB with YaxA unstructured N-terminal tails and FIG. 1B depicts the truncated YaxA_{Δ40}B nanopore. YaxA monomer units are shaded dark, and the YaxB monomer units are shaded white. The molecular models are obtained by using MODELLER, starting from the PDB structure 6EL1. YaxAB nanopores have a large conical shaped vestibule with an opening (e.g., cis entrance) of about 15 nanometers in diameter for the decamer of dimers arrangement of the protein, tapering to a constriction (e.g., trans entrance) of about 3 nanometers in diameter.

In some cases, the nanopore comprises at least one PaxA subunit. In some cases, the nanopore comprises at least one XaxA subunit. In some embodiments, the nanopore comprises at least one PaxB subunit. In some embodiments, the nanopore comprises at least one XaxB subunit. In some embodiments, the nanopore comprises an equal number of PaxA and PaxB subunits. In some embodiments, the nanopore comprises an equal number of XaxA and XaxB subunits.

In some embodiments, a portion of a monomer can comprise a truncation of a N-terminal region and/or C-terminal region (e.g., a N-truncated or C-truncated variant). A nanopore (e.g., biological nanopore) may comprise at least about 1, 2, 3, 4, 5, 6, 7, 8, 9, 10, 15, 20, 25, 30, or greater than about 30 monomers comprising a truncated N-terminal and/or C-terminal. In some embodiments, at least one monomer of a nanopore described herein comprises a truncated N-terminal. In some embodiments, a nanopore comprising YaxA and/or YaxB subunits comprises at least one N-truncated YaxA and/or YaxB subunit variant. In some embodiments, a nanopore can comprise a truncated variant of YaxA (e.g., a YaxA subunit lacking at an least partially unstructured N-terminal region). In some embodiments, a YaxA subunit of a monomer, or a mutant, functional homolog, functional ortholog, or functional paralog thereof may lack amino acid residues at positions 1-20, 1-30, 1-40, or 1-41, as set forth in SEQ ID NO: 25 (SEQ ID NO: 25 (ProteinID YE1984)) or the corresponding N-truncated ortholog thereof. In some cases, YaxA or its ortholog may lack one or more amino acid residues as set forth in SEQ ID NO: 25 (SEQ ID NO: 25 (ProteinID YE1984)) or the corresponding N-truncated ortholog thereof. In some cases, YaxA or its ortholog may lack amino acid residue(s) at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or any combination thereof, as set forth in SEQ ID NO: 25 (SEQ ID NO: 25 (ProteinID YE1984)) or the corresponding N-truncated ortholog thereof. In some cases, YaxA, or a mutant, functional homolog, functional ortholog, or functional paralog thereof, may lack amino acid residue(s) from positions 1 to 20 as set forth in SEQ ID NO: 25 (SEQ ID NO: 25 (ProteinID YE1984)) or the corresponding N-truncated ortholog thereof. In some cases, YaxA, or a mutant, functional homolog, functional ortholog, or functional paralog thereof, may lack amino acid residue(s) from positions 1 to 2, 1 to 3, 1 to 4, 1 to 5, 1 to 6, 1 to 7, 1 to 8, 1 to 9, 1 to 10, 1 to 15, 1 to 20, 2 to 3, 2 to 4, 2 to 5, 2 to 6, 2 to 7, 2 to 8, 2 to 9, 2 to 10, 2 to 15, 2 to 20, 3 to 4, 3 to 5, 3 to 6, 3 to 7, 3 to 8, 3 to 9, 3 to 10, 3 to 15, 3 to 20, 4 to 5, 4 to 6, 4 to 7, 4 to 8, 4 to 9, 4 to 10, 4 to 15, 4 to 20, 5 to 6, 5 to 7, 5 to 8, 5 to 9, 5 to 10, 5 to 15, 5 to 20, 6 to 7, 6 to 8, 6 to 9, 6 to 10, 6 to 15, 6 to 20, 7 to 8, 7 to 9, 7 to 10, 7 to 15, 7 to 20, 8 to 9, 8 to 10, 8 to 15, 8 to 20, 9 to 10, 9 to 15, 9 to 20, 10 to 15, 10 to 20, or 15 to 20 as set forth in SEQ ID NO: 25 (SEQ ID NO: 25 (ProteinID YE1984)) or the corresponding N-truncated ortholog thereof. In some cases, the nanopore comprises at least one full-length (e.g., non-truncated) version of a YaxA subunit.

In some embodiments, a nanopore can comprise a truncated variant of YaxB (e.g., a YaxB subunit lacking at an least partially unstructured N-terminal region). In some embodiments, a YaxB subunit of a monomer, or a mutant, functional homolog, functional ortholog, or functional paralog thereof may lack amino acid residues at positions 1-20, 1-30, 1-40, or 1-41, as set forth in SEQ ID NO: 26 (ProteinID YE1985) or the corresponding N-truncated ortholog thereof. In some cases, YaxB or its ortholog may lack one or more amino acid residues as set forth in SEQ ID NO: 26 (ProteinID YE1985) or the corresponding N-truncated ortholog thereof. In some cases, YaxB or its ortholog may lack amino acid residue(s) at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or any combination thereof, as set forth in SEQ ID NO: 26 (ProteinID YE1985) or the corresponding N-truncated ortholog thereof. In some cases, YaxB, or a mutant, functional homolog, functional ortholog, or functional paralog thereof, may lack amino acid residue(s) from positions 1 to 20 as set forth in SEQ ID NO: 26 (ProteinID YE1985) or the corresponding N-truncated ortholog thereof. In some cases, YaxB, or a mutant, functional homolog, functional ortholog, or functional paralog thereof, may lack amino acid residue(s) from positions 1 to 2, 1 to 3, 1 to 4, 1 to 5, 1 to 6, 1 to 7, 1 to 8, 1 to 9, 1 to 10, 1 to 15, 1 to 20, 2 to 3, 2 to 4, 2 to 5, 2 to 6, 2 to 7, 2 to 8, 2 to 9, 2 to 10, 2 to 15, 2 to 20, 3 to 4, 3 to 5, 3 to 6, 3 to 7, 3 to 8, 3 to 9, 3 to 10, 3 to 15, 3 to 20, 4 to 5, 4 to 6, 4 to 7, 4 to 8, 4 to 9, 4 to 10, 4 to 15, 4 to 20, 5 to 6, 5 to 7, 5 to 8, 5 to 9, 5 to 10, 5 to 15, 5 to 20, 6 to 7, 6 to 8, 6 to 9, 6 to 10, 6 to 15, 6 to 20, 7 to 8, 7 to 9, 7 to 10, 7 to 15, 7 to 20, 8 to 9, 8 to 10, 8 to 15, 8 to 20, 9 to 10, 9 to 15, 9 to 20, 10 to 15, 10 to 20, or 15 to 20 as set forth in SEQ ID NO: 26 (ProteinID YE1985) or the corresponding N-truncated ortholog thereof. In some cases, the nanopore comprises at least one full-length (e.g., non-truncated) version of a YaxB subunit.

In some embodiments, the nanopore comprises one or more N-truncated YaxA subunits, or orthologs thereof, in combination with one or more full-length (e.g., non-truncated) YaxB subunits, or orthologs thereof. In some embodiments, the nanopore comprises one or more N-truncated YaxB subunits, or orthologs thereof, in combination with one or more full-length (e.g., non-truncated) YaxA subunits, or orthologs thereof. In some embodiments, the nanopore comprises one or more N-truncated YaxA subunits, or orthologs thereof, in combination with one or more N-truncated YaxB subunits, or orthologs thereof. In some embodiments, the nanopore comprises one or more full-length (e.g., non-truncated) YaxA subunits, or orthologs thereof, in combination with one or more full-length (e.g., non-truncated) YaxB subunits, or orthologs thereof.

In some embodiments, a portion of a monomer of the nanopore described herein may comprise one or more mutations. A full-length subunit may comprise one or more mutations. A truncated subunit (e.g., a N-truncated monomer) may comprise one or more mutations. In some embodiments, a nanopore may comprise one or more mutated subunits. In some embodiments, a first portion of a monomer comprises one or more mutations. In some embodiments, a second portion of a monomer comprises one or more mutations. In some embodiments, a first portion and a second portion of a monomer comprises one or more mutations.

Without wishing to be bound by theory, a mutation to a subunit of a monomer of a nanopore described herein may alter a charge of the nanopore. A change in a charge of the nanopore may modify distribution of charges in the channel of the nanopore. In some embodiments, a mutation comprises a point mutation. In some embodiments, a point mutation can be at a non-conserved position. In some embodiments, a point mutation is a lumen-facing mutation. In some embodiments, a point mutation is a membrane-facing mutation. In some embodiments, a point mutation can alter a characteristic of a pore. In some embodiments, a point mutation can alter a pore channel charge, conductance at a set pH, ion selectivity, electro-osmotic flux, conductivity, shape, structure, or any combination thereof.

In some embodiments, the nanopore comprises one or more mutations in a subunit of a monomer of the alpha-xenorhabdolysin family of binary toxin or a mutant, functional homolog, functional ortholog, or functional paralog thereof. In some embodiments, a nanopore comprises one or more mutations of a subunit originating from Yersinia enterocolitica (e.g., YaxA or YaxB). In some embodiments, a nanopore comprises one or more mutations of a subunit originating from Providencia alcalifaciens (e.g., PaYaxA, PaYaxB). In some embodiments, a nanopore comprises one or more mutations of a subunit originating from Pseudomonas syringae (e.g., PsYaxA, PsYaxB). In some embodiments, a nanopore comprises one or more mutations of a subunit originating from Proteus mirabilis (e.g., PmYaxA, PmYaxB). In some embodiments, a nanopore comprises one or more mutations of a subunit originating from Morganella morganii (e.g., MmYaxA, MmYaxB). In some embodiments, a nanopore comprises one or more mutations of a subunit originating from Photorhabdus luminescens (e.g., PaxA, PaxB). In some embodiments, a nanopore comprises one or more mutations of a subunit originating from Xenorhabdus nematophila (e.g., XaxA, XaxB).

In some embodiments, the nanopore comprises one or more mutations of a wild-type YaxA subunit. For example, one or more amino acid substitution can be made on the basis of a sequence comparison with orthologues of YaxA, such as PaxA, MmYaxA and/or XaxA. In some embodiments, the nanopore comprises one or more mutations of a wild-type YaxB subunit. For example, one or more amino acid substitution can be made on the basis of a sequence comparison with orthologues of YaxB, such as PaxB, Mm YaxB and/or XaxB. In some cases, conserved amino acids or regions, such as the hydrophobic foot, conserved amino acid residues facing the lipid milieu as part of the transmembrane segment and/or amino acid residues engaged in YaxB-YaxB contacts, can be maintained in a nanopore described herein.

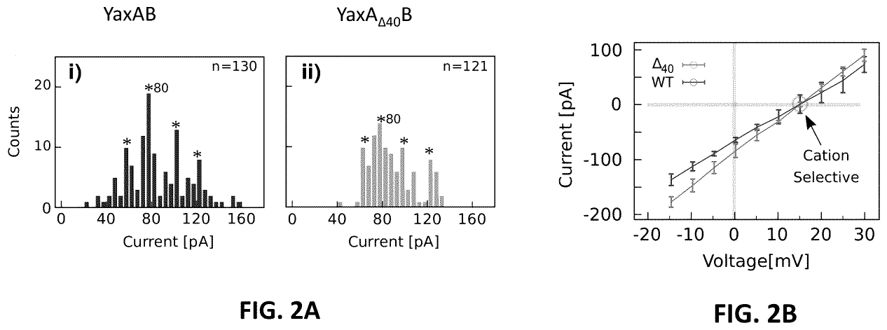

The full-length YaxAB pore and the truncated pore may comprise different open-pore currents. In some embodiments, a truncated pore can comprise a number of amino acid residue reduction from a full length pore. For example, the truncated pore may comprise a YaxAΔ₄₀B pore in which there can be a 40 amino acid residue difference between the truncated pore and a full-length (e.g., non-truncated) pore. In the YaxAΔ₄₀B monomer, the YaxA subunit may comprise a 40 amino acid residue truncation. FIG. 2A shows experimental distribution of open-pore currents for single nanopores (measured at −35 mV) for the (i) full-length YaxAB and (ii) truncated YaxAΔ₄₀B. In FIG. 2A, measurements were performed in 150 mM NaCl, 15 mM TrisHCl pH 7.5. Data was recorded with a 50 kHz sampling rate and 10 kHz Bessel filter. The histograms show the presence multiple populations of nanopore from different sized oligomeric forms. Distinct peaks correspond to the major oligomeric forms. *80 indicates the most prevalent assembly for the wild-type and YaxAΔ₄₀B pores. FIG. 2B shows reversal potential current-voltage (I-V) curves measuring the electro-osmotic ionic transport properties of the YaxAB nanopores from the *80 population under asymmetric salt conditions (300 mM NaCl in cis, 75 mM NaCl in trans). The data show that the full-length YaxAB and truncated YaxAΔ₄₀B nanopores are strongly cation selective.

Calculations of homology or sequence identity between sequences (the terms can be used interchangeably herein) are performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes can be at least 30%, at least 40%, at least 50%, 60%, or at least 70%, 80%, 90%, 100% of the length of the reference sequence. The amino acid residues or nucleotides at corresponding amino acid positions or nucleotide positions can then compared. When a position in the first sequence is occupied by the same amino acid residue or nucleotide as the corresponding position in the second sequence, then the molecules can be identical at that position (as used herein amino acid or nucleic acid “identity” is equivalent to amino acid or nucleic acid “homology”). The percent identity between the two sequences can refer to a function of the number of identical positions shared by the sequences, taking into account the number of gaps, and the length of each gap, which may need to be introduced for optimal alignment of the two sequences. The comparison of sequences and determination of percent identity between two sequences can be accomplished using a mathematical algorithm. In some embodiments, the percent identity between two amino acid sequences can be determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Yersinia enterocolitica (e.g., YaxA or YaxB). In some cases, a nanopore described herein may comprise a subunit comprising an amino acid sequence with 100% sequence identity to an amino acid sequence of a wild-type subunit originating from Yersinia enterocolitica (e.g., YaxA or YaxB).

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Providencia alcalifaciens (e.g., PaYaxA, PaYaxB). In some cases, a nanopore described herein may comprise a subunit comprising an amino acid sequence with 100% sequence identity to an amino acid sequence of a wild-type subunit originating from Providencia alcalifaciens (e.g., PaYaxA, PaYaxB).

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Pseudomonas syringae (e.g., PsYaxA, PsYaxB). In some cases, a nanopore described herein may comprise a subunit comprising an amino acid sequence with 100% sequence identity to an amino acid sequence of a wild-type subunit originating from Pseudomonas syringae (e.g., PsYaxA, PsYaxB).

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Proteus mirabilis (e.g., PmYaxA, PmYaxB). In some cases, a nanopore described herein may comprise a subunit comprising an amino acid sequence with 100% sequence identity to an amino acid sequence of a wild-type subunit originating from Proteus mirabilis (e.g., PmYaxA, PmYaxB).

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Morganella morganii (e.g., MmYaxA, MmYaxB). In some cases, a nanopore described herein may comprise a subunit comprising an amino acid sequence with 100% sequence identity to an amino acid sequence of a wild-type subunit originating from Morganella morganii (e.g., MmYaxA, MmYaxB).

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Photorhabdus luminescens (e.g., PaxA, PaxB). In some cases, a nanopore described herein may comprise a subunit comprising an amino acid sequence with 100% sequence identity to an amino acid sequence of a wild-type subunit originating from Photorhabdus luminescens (e.g., PaxA, PaxB).

In some cases, a nanopore described herein may comprise a subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence of a wild-type subunit originating from Xenorhabdus nematophila (e.g., XaxA, XaxB). In some cases, a nanopore described herein may comprise a subunit comprising an amino acid sequence with 100% sequence identity to an amino acid sequence of a wild-type subunit originating from Xenorhabdus nematophila (e.g., XaxA, XaxB).

In some cases, a nanopore described herein comprises a YaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 25 (ProteinID YE1984). In some cases, a nanopore described herein comprises a YaxA subunit comprising an amino acid sequence from about 70% to about 97% sequence identity to an amino acid sequence as set forth in SEQ ID NO: 25 (ProteinID YE1984). In some cases, a nanopore described herein comprises a YaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 25 (ProteinID YE1984). In some cases, a nanopore described herein comprises a YaxA subunit comprising an amino acid sequence as set forth in SEQ ID NO: 25 (ProteinID YE1984).

In some cases, a nanopore described herein comprises a YaxB subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 26 (ProteinID YE1985). In some cases, a nanopore described herein comprises a YaxB subunit comprising an amino acid sequence from about 70% to about 97% sequence identity to an amino acid sequence as set forth in SEQ ID NO: 26 (ProteinID YE1985). In some cases, a nanopore described herein comprises a YaxB subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 912% to about 92%, about 912% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 26 (ProteinID YE1985). In some cases, a nanopore described herein comprises a YaxB subunit comprising an amino acid sequence as set forth in SEQ ID NO: 26 (ProteinID YE1985).

In some cases, a nanopore described herein comprises a PaYaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 27. In some cases, a nanopore described herein comprises a PaYaxA subunit comprising an amino acid sequence from about 70% to about 97% sequence identity to an amino acid sequence as set forth in SEQ ID NO: 27. In some cases, a nanopore described herein comprises a PaYaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 27. In some cases, a nanopore described herein comprises a PaYaxA subunit comprising an amino acid sequence as set forth in SEQ ID NO: 27.

In some cases, a nanopore described herein comprises a PaYaxB subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 28. In some cases, a nanopore described herein comprises a PaYaxB subunit comprising an amino acid sequence from about 70% to about 97% sequence identity to an amino acid sequence as set forth in SEQ ID NO: 28. In some cases, a nanopore described herein comprises a PaYaxB subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 28. In some cases, a nanopore described herein comprises a PaYaxB subunit comprising an amino acid sequence as set forth in SEQ ID NO: 28.

In some cases, a nanopore described herein comprises a PsYaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 29. In some cases, a nanopore described herein comprises a PsYaxA subunit comprising an amino acid sequence from about 70% to about 97% sequence identity to an amino acid sequence as set forth in SEQ ID NO: 29. In some cases, a nanopore described herein comprises a PsYaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 29. In some cases, a nanopore described herein comprises a PsYaxA subunit comprising an amino acid sequence as set forth in SEQ ID NO: 29.

In some cases, a nanopore described herein comprises a PsYaxB subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 30. In some cases, a nanopore described herein comprises a PsYaxB subunit comprising an amino acid sequence from about 70% to about 97% sequence identity to an amino acid sequence as set forth in SEQ ID NO: 30. In some cases, a nanopore described herein comprises a PsYaxB subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 30. In some cases, a nanopore described herein comprises a PsYaxB subunit comprising an amino acid sequence as set forth in SEQ ID NO: 30.

In some cases, a nanopore described herein comprises a PmYaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 31. In some cases, a nanopore described herein comprises a PmYaxA subunit comprising an amino acid sequence from about 70% to about 97% sequence identity to an amino acid sequence as set forth in SEQ ID NO: 31. In some cases, a nanopore described herein comprises a PmYaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 31. In some cases, a nanopore described herein comprises a PmYaxA subunit comprising an amino acid sequence as set forth in SEQ ID NO: 31.

In some cases, a nanopore described herein comprises a PmYaxB subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 32. In some cases, a nanopore described herein comprises a PmYaxB subunit comprising an amino acid sequence from about 70% to about 97% sequence identity to an amino acid sequence as set forth in SEQ ID NO: 32. In some cases, a nanopore described herein comprises a PmYaxB subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 32. In some cases, a nanopore described herein comprises a PmYaxB subunit comprising an amino acid sequence as set forth in SEQ ID NO: 32.

In some cases, a nanopore described herein comprises a MmYaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 33. In some cases, a nanopore described herein comprises a MmYaxA subunit comprising an amino acid sequence from about 70% to about 97% sequence identity to an amino acid sequence as set forth in SEQ ID NO: 33. In some cases, a nanopore described herein comprises a MmYaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 33. In some cases, a nanopore described herein comprises a MmYaxA subunit comprising an amino acid sequence as set forth in SEQ ID NO: 33.

In some cases, a nanopore described herein comprises a MmYaxB subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 34. In some cases, a nanopore described herein comprises a MmYaxB subunit comprising an amino acid sequence from about 70% to about 97% sequence identity to an amino acid sequence as set forth in SEQ ID NO: 34. In some cases, a nanopore described herein comprises a MmYaxB subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 912% to about 93%, about 912% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 34. In some cases, a nanopore described herein comprises a MmYaxB subunit comprising an amino acid sequence as set forth in SEQ ID NO: 34.

In some cases, a nanopore described herein comprises a PaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 35. In some cases, a nanopore described herein comprises a PaxA subunit comprising an amino acid sequence from about 70% to about 97% sequence identity to an amino acid sequence as set forth in SEQ ID NO: 35. In some cases, a nanopore described herein comprises a PaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 912% to about 95%, about 912% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 35. In some cases, a nanopore described herein comprises a PaxA subunit comprising an amino acid sequence as set forth in SEQ ID NO: 35.

In some cases, a nanopore described herein comprises a PaxB subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 36. In some cases, a nanopore described herein comprises a PaxB subunit comprising an amino acid sequence from about 70% to about 97% sequence identity to an amino acid sequence as set forth in SEQ ID NO: 36. In some cases, a nanopore described herein comprises a PaxB subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 36. In some cases, a nanopore described herein comprises a PaxB subunit comprising an amino acid sequence as set forth in SEQ ID NO: 36.

In some cases, a nanopore described herein comprises a XaxA subunit comprising an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or at least about 99.9% sequence identity to an amino acid sequence as set forth in SEQ ID NO: 37. In some cases, a nanopore described herein comprises a XaxA subunit comprising an amino acid sequence from about 70% to about 97% sequence identity to an amino acid sequence as set forth in SEQ ID NO: 37. In some cases, a nanopore described herein comprises a XaxA subunit comprising an amino acid sequence from about 70% to about 75%, about 70% to about 80%, about 70% to about 85%, about 70% to about 90%, about 70% to about 91%, about 70% to about 92%, about 70% to about 93%, about 70% to about 94%, about 70% to about 95%, about 70% to about 96%, about 70% to about 97%, about 75% to about 80%, about 75% to about 85%, about 75% to about 90%, about 75% to about 91%, about 75% to about 92%, about 75% to about 93%, about 75% to about 94%, about 75% to about 95%, about 75% to about 96%, about 75% to about 97%, about 80% to about 85%, about 80% to about 90%, about 80% to about 91%, about 80% to about 92%, about 80% to about 93%, about 80% to about 94%, about 80% to about 95%, about 80% to about 96%, about 80% to about 97%, about 85% to about 90%, about 85% to about 91%, about 85% to about 92%, about 85% to about 93%, about 85% to about 94%, about 85% to about 95%, about 85% to about 96%, about 85% to about 97%, about 90% to about 91%, about 90% to about 92%, about 90% to about 93%, about 90% to about 94%, about 90% to about 95%, about 90% to about 96%, about 90% to about 97%, about 91% to about 92%, about 91% to about 93%, about 91% to about 94%, about 91% to about 95%, about 91% to about 96%, about 91% to about 97%, about 92% to about 93%, about 92% to about 94%, about 92% to about 95%, about 92% to about 96%, about 92% to about 97%, about 93% to about 94%, about 93% to about 95%, about 93% to about 96%, about 93% to about 97%, about 94% to about 95%, about 94% to about 96%, about 94% to about 97%, about 95% to about 96%, about 95% to about 97%, or about 96% to about 97% sequence identity to an amino acid sequence as set forth in SEQ ID NO: 37. In some cases, a nanopore described herein comprises a XaxA subunit comprising an amino acid sequence as set forth in SEQ ID NO: 37.

In some embodiments, variable amino acid positions can include R150, K250, S282, or any combination thereof, of the wild-type YaxA sequence as set forth in SEQ ID NO: 25 (ProteinID YE1984). For example, PaxA and XaxA can have G at position R150, MmYaxA can have recognition element at position K250, and/or six YaxA orthologues can have G at position S282. In some embodiments, mutations at amino acid positions R150, K250, N12, and/or S282 of the wild-type YaxA sequence as set forth in SEQ ID NO: 25 (ProteinID YE1984) may comprise one or more substitutions of glycine (G), alanine (A), isoleucine (I), leucine (L), proline (P), arginine (R), serine (S) or any combination thereof. In some embodiments, mutations at amino acid positions R150, K250, N12, and/or S282 of the wild-type YaxA sequence as set forth in SEQ ID NO: 25 (ProteinID YE1984) may comprise substitution to a positively-charged amino acid residue, a negatively-charged amino acid residue, a neutral amino acid residue, a hydrophobic amino acid residue, a hydrophilic amino acid residue, or any combination thereof.

In some embodiments, a N-truncated YaxA subunit may comprise one or more of the mutations R150G, K250R, or S282G with respect to the sequence of SEQ ID NO: 25 (ProteinID YE1984). In some embodiments, a non-truncated YaxA subunit may comprise one or more of the mutations R150G, K250R, or S282G with respect to the sequence of SEQ ID NO: 25 (ProteinID YE1984). In some embodiments, a nanopore described herein may comprise at least one YaxA subunit comprising one or more of the mutations R150G, K250R, or S282G with respect to the sequence of SEQ ID NO: 25 (ProteinID YE1984) and at least one of wild-type YaxA subunit.

In some embodiments, a N-truncated YaxA subunit may comprise a mutation at position N17 of the wild-type YaxA sequence as set forth in SEQ ID NO: 25 (ProteinID YE1984). In some embodiments, a non-truncated YaxA subunit may comprise a mutation at position N17 of the wild-type YaxA sequence as set forth in SEQ ID NO: 25 (ProteinID YE1984). In some embodiments, a mutation at amino acid position N17 of the wild-type YaxA sequence as set forth in SEQ ID NO: 25 (ProteinID YE1984) may comprise substitution to a positively-charged amino acid residue, a negatively-charged amino acid residue, a neutral amino acid residue, a hydrophobic amino acid residue, or a hydrophilic amino acid residue. In some embodiments, a non-truncated YaxA subunit may comprise the mutation N17S with respect to the sequence of SEQ ID NO: 25 (ProteinID YE1984). In some embodiments, a nanopore described herein may comprise at least one YaxA subunit comprising the mutation N17S with respect to the sequence of SEQ ID NO: 25 (ProteinID YE1984) and at least one of wild-type YaxA subunit.

In some cases, a YaxA subunit of a nanopore described herein can comprise a mutation comprising R150G, K250R, S282G, or N17S, or any combination thereof, with numbering respect to the sequence set forth in SEQ ID NO: 25 (ProteinID YE1984). In some embodiments, a nanopore described herein may comprise at least one YaxA subunit comprising one or more of the mutations R150G, K250R, S282G, or N17S, with respect to the sequence of ProteinID YE1984 and at least one of wild-type YaxA subunit.

In some embodiments, a N-truncated YaxB subunit may comprise a mutation at position 284 of the wild-type YaxB sequence as set forth in SEQ ID NO: 26 (ProteinID YE1985). In some embodiments, a non-truncated YaxB subunit may comprise a mutation at position 284 of the wild-type YaxB sequence as set forth in SEQ ID NO: 26 (ProteinID YE1985). In some embodiments, a N-truncated YaxB subunit may comprise a mutation at position V284 of the wild-type YaxB sequence as set forth in SEQ ID NO: 26 (ProteinID YE1985). In some embodiments, a non-truncated YaxB subunit may comprise a mutation at position V284 of the wild-type YaxB sequence as set forth in SEQ ID NO: 26 (ProteinID YE1985). In some embodiments, a mutation at amino acid position V284 of the wild-type YaxB sequence as set forth in SEQ ID NO: 26 (ProteinID YE1985) may comprise a substitution to glycine (G), alanine (A), isoleucine (I), leucine (L), proline (P), arginine (R), or serine (S). In some embodiments, a mutation at amino acid position V284 of the wild-type YaxB sequence as set forth in SEQ ID NO: 26 (ProteinID YE1985) may comprise substitution to a positively-charged amino acid residue, a negatively-charged amino acid residue, a neutral amino acid residue, a hydrophobic amino acid residue, or a hydrophilic amino acid residue. In some embodiments, a nanopore described herein comprises at least one variant YaxB subunit comprising a mutation V284I, wherein the residue numbering corresponds to SEQ ID NO: 26 (ProteinID YE1985).

In some embodiments, a nanopore described herein may comprise an electro-osmotic flow (EOF) mutant. The EOF mutant may comprise a subunit of the nanopore wherein a sequence of the subunit comprises at least one amino acid substitution. An electro-osmotic mutation may comprise mutations of one or more negatively charged amino acid residues of the nanopore. In some embodiments, the one or more negatively charged amino acid residues reside in the lumen (e.g., the channel or constriction region) of the nanopore. In some embodiments, one or more negatively charged amino acid residues of the constriction region of the nanopore can be mutated to a neutral amino acid residue. The mutation may remove the electro-osmotic force. The mutation may reduce the electro-osmotic force. In some embodiments, a nanopore described herein comprises at least one subunit comprising one or more EOF mutations. In some embodiments, a nanopore comprises at least one EOF mutations of a YaxB subunit. In some embodiments, one or more YaxB subunits of the monomers of the nanopore comprise an EOF mutation. In some embodiments, the YaxB subunit comprises a mutation at amino acid position 208, 212, 214, or any combination thereof, wherein the amino acid residue numbering corresponds to SEQ ID NO: 26 (ProteinID YE1985). In some embodiments, the YaxB subunit comprises a mutation at amino acid position E208, E212, D214, or any combination thereof, wherein the residue numbering corresponds to SEQ ID NO: 26 (ProteinID YE1985). In some embodiments, a nanopore described herein comprises at least one variant YaxB subunit comprising one or more mutations of E208N, E212N, D214N, or any combination thereof, wherein the residue numbering corresponds to SEQ ID NO: 26 (ProteinID YE1985). In some embodiments, a nanopore described herein comprises at least one variant YaxB subunit comprising one or more mutations of V284I, E208N, E212N, D214N, or any combination thereof, wherein the residue numbering corresponds to SEQ ID NO: 26 (ProteinID YE1985).

In some aspects, the present disclosure provides nanopores, systems, and methods comprising a nanopore comprising a electroosmotic flow (EOF) mutant. In some cases, the EOF mutant comprises one negatively-charged amino acid residue mutated to a neutral amino acid residue. In some cases, the EOF mutant comprises one negatively-charged amino acid residue mutated to a positively-charged residue. In some cases, the EOF mutant comprises at least one negatively-charged amino acid residue mutated to a neutral residue. In some cases, the EOF mutant comprises at least one negatively-charged amino acid residue mutated to a positively-charged residue. In some cases, the mutated residue faces a lumen of the nanopore (e.g., is within a channel of a nanopore). A lumen-facing residue may interact with an analyte that passes through the lumen. A lumen-facing residue may interact with an analyte that resides in the lumen. The mutated residue of the EOF mutant may reside in a constriction region. Without wishing to be bound by theory, a constriction region may refer to an area of the lumen with a smaller diameter, circumference, or widest dimension than another area of the lumen.

In some cases, an EOF mutant comprises an aspartic acid (D) residue and/or a glutamic acid (E) residue mutated to a positively-charged residue. In some cases, an EOF mutant comprises an aspartic acid (D) residue and/or a glutamic acid (E) residue mutated to an arginine (R) residue, a histidine (H) residue, or lysine (K) residue. In some embodiments, an EOF mutant comprises an aspartic acid (D) residue and/or a glutamic acid (E) residue mutated to a neutral residue. In some embodiments, an EOF mutant comprises an aspartic acid (D) residue and/or a glutamic acid (E) residue mutated to a serine (S) residue, a threonine (T) residue, an asparagine (N) residue, or a glutamine (Q) residue. In some embodiments, a nanopore and/or a nanopore system described herein comprises a EOF mutant of YaxA, YaxB, or a combination thereof. In some embodiments, a YaxB mutant comprises mutations E208N, E212N, D214N, or any combination thereof. In some embodiments, a YaxB mutant comprises mutations E208R, E212R, D214R or any combination thereof. In some embodiments, a YaxB mutant comprises mutations E208N, E212N, D214N, E208R, E212R, D214R or any combination thereof.

In some embodiments, the subunit of the nanopore may comprise an additional number of amino acids at an N-terminal. In some embodiments, a YaxA and/or YaxB subunit of the nanopore may comprise an additional number of amino acids at an N-terminal. In some embodiments, a YaxA and/or YaxB subunit of the nanopore comprises at least 24 amino acids at its N-terminus. The additions to the N-terminus can comprise at least one peptide tag. For example, the additions to the N-terminus may comprise a His tag, at least one spacer region, at least one protease cleavage site, or any combination thereof. The His tag can comprise a string of 2, 3, 4, 5, 6, 7, 8, 9, or 10 histidine residues. The additions to the N-terminal of a subunit of the nanopore can comprise MSYY, HHHHH (e.g., 6×His tag), DYDIPTT (e.g., a spacer region), ENLYFQG or ENLYFQS (e.g., TEV protease cleavage site), or any combination thereof. In some embodiments, a subunit of a nanopore described herein may comprise an addition to an N-terminus comprising MSYY, H HHHH (6×His tag), DYDIPTT, ENLYFQG, or any combination thereof. In some embodiments, a subunit of a nanopore described herein may comprise an addition to an N-terminus comprising MSYY, HHHHHH (6×His tag), DYDIPTT, ENLYFQS, or any combination thereof.

### Analytes

The nanopores, methods, and/or systems described herein can be readily designed to detect any analyte (or multiple analytes) of interest. The invention can be advantageously used to detect a label-free analyte. The nanopores described herein can capture a wide range of particles in a similar size range. Examples include inorganic particles (e.g. gold beads), polymeric particles such as plastics/beads/dendrimers, or oligomeric particles (e.g. micelles, liposomes and other fatty droplets).

In one embodiment, the invention provides a method for detecting an analyte/antigen selected from the group consisting of a protein, polypeptide, a protein assembly, a protein/DNA assembly, saccharide (e.g., polysaccharide), lipid, lipid membrane, lipid particle, bacterium, virus capsid, virus particle, dendrimer, polymer, inorganic particle, oligomeric particle, non-nucleic acid based polymer analyte, or any combination thereof. In some embodiments, the analyte can be a nucleic acid analyte. In some embodiments, the analyte may not be a nucleic acid analyte.

The nanopores, methods, and systems of the present disclosure can be very suitable for the analysis of a complex sample, e.g. a solution comprising a mixture of components including one or more target analytes and one or more unwanted analytes. For example, the sample can be a complex sample comprising a mixture of proteins. In some cases, the sample comprises a (diluted) clinical sample. In some cases, the sample can be a bodily fluid or sample, such as whole blood, plasma, blood serum, urine, feces, saliva, cerebrospinal fluid, nasopharyngeal swab, breast milk, sputum, or any combination thereof. In another aspect, the sample comprises (diluted) complex media. In some embodiments, a sample can be obtained from a healthy subject. In some embodiments, a sample can be obtained from a subject with a disease or condition.

In one embodiment, the target analyte can be a clinically relevant analyte, for example a clinically relevant protein or fragment thereof. In a specific embodiment, the target analyte can be a cytokine, an inflammation marker (e.g. C-reactive protein) or a cell metabolite. In some embodiments, the cytokine molecule may comprise interleukin-2 (IL-2) or a functional variant thereof, interleukin-7 (IL-7) or a functional variant thereof, interleukin-12 (IL-12) or a functional variant thereof, interleukin-15 (IL-15) or a functional variant thereof, interleukin-18 (IL-18) or a functional variant thereof, interleukin-21 (IL-21) or a functional variant thereof, or interferon gamma or a functional variant thereof, or any combination thereof. In some cases, the analyte can be a protein, for example selected from the group consisting of a folded/native protein, a protein biomarker, a pathogenic protein, a cell surface protein.

The present invention can be particularly suitable for detecting protein targets covering a very wide range of masses and dimensions, from very small proteins and peptides to very large proteins and complexes. In some embodiments, the analyte can comprise at least about 2 amino acids, at least about 5 amino acids, at least about 10 amino acids, at least about 15 amino acids, at least about 20 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 150 amino acids, at least about 200 amino acids, at least about 250 amino acids, at least about 300 amino acids, at least about 350 amino acids, at least about 400 amino acids, at least about 450 amino acids, at least about 500 amino acids, at least about 600 amino acids, at least about 700 amino acids, at least about 800 amino acids, at least about 900 amino acids, at least about 1000 amino acids, at least about 2000 amino acids, at least about 3000 amino acids, at least about 4000 amino acids, at least about 5000 amino acids, at least about 6000 amino acids, at least about 7000 amino acids, at least about 8000 amino acids, at least about 9000 amino acids, at least about 10000 amino acids, at least about 20000 amino acids, at least about 30000, at least about 34000 amino acids, or greater than about 34000 amino acids in length. In some embodiments, the analyte can be at most about 34000 amino acids, at most about 30000 amino acids, at most about 20000 amino acids, at most about 10000 amino acids, at most about 9000 amino acids, at most about 8000 amino acids, at most about 7000 amino acids, at most about 6000 amino acids, at most about 5000 amino acids, at most about 4000 amino acids, at most about 3000 amino acids, at most about 2000 amino acids, at most about 1000 amino acids, at most about 900 amino acids, at most about 800 amino acids, at most about 700 amino acids, at most about 600 amino acids, at most about 500 amino acids, at most about 450 amino acids, at most about 400 amino acids, at most about 350 amino acids, at most about 300 amino acids, at most about 250 amino acids, at most about 30000 amino acids, at most about 30000 amino acids, at most about 200 amino acids, at most about 150 amino acids, at most about 100 amino acids, at most about 90 amino acids, at most about 80 amino acids, at most about 70 amino acids, at most about 60 amino acids, at most about 50 amino acids, at most about 40 amino acids, at most about 30 amino acids, at most about 20 amino acids, at most about 15 amino acids, at most about 10 amino acids, at most about 5 amino acids, at most about 2 amino acids, or less than about 2 amino acids in length.

In some embodiments, the analyte can be from about 2 amino acids to about 1,000 amino acids in length. In some embodiments, the analyte can be from about 2 amino acids to about 10 amino acids, about 2 amino acids to about 100 amino acids, about 2 amino acids to about 200 amino acids, about 2 amino acids to about 300 amino acids, about 2 amino acids to about 400 amino acids, about 2 amino acids to about 500 amino acids, about 2 amino acids to about 600 amino acids, about 2 amino acids to about 700 amino acids, about 2 amino acids to about 800 amino acids, about 2 amino acids to about 900 amino acids, about 2 amino acids to about 1,000 amino acids, about 10 amino acids to about 100 amino acids, about 10 amino acids to about 200 amino acids, about 10 amino acids to about 300 amino acids, about 10 amino acids to about 400 amino acids, about 10 amino acids to about 500 amino acids, about 10 amino acids to about 600 amino acids, about 10 amino acids to about 700 amino acids, about 10 amino acids to about 800 amino acids, about 10 amino acids to about 900 amino acids, about 10 amino acids to about 1,000 amino acids, about 100 amino acids to about 200 amino acids, about 100 amino acids to about 300 amino acids, about 100 amino acids to about 400 amino acids, about 100 amino acids to about 500 amino acids, about 100 amino acids to about 600 amino acids, about 100 amino acids to about 700 amino acids, about 100 amino acids to about 800 amino acids, about 100 amino acids to about 900 amino acids, about 100 amino acids to about 1,000 amino acids, about 200 amino acids to about 300 amino acids, about 200 amino acids to about 400 amino acids, about 200 amino acids to about 500 amino acids, about 200 amino acids to about 600 amino acids, about 200 amino acids to about 700 amino acids, about 200 amino acids to about 800 amino acids, about 200 amino acids to about 900 amino acids, about 200 amino acids to about 1,000 amino acids, about 300 amino acids to about 400 amino acids, about 300 amino acids to about 500 amino acids, about 300 amino acids to about 600 amino acids, about 300 amino acids to about 700 amino acids, about 300 amino acids to about 800 amino acids, about 300 amino acids to about 900 amino acids, about 300 amino acids to about 1,000 amino acids, about 400 amino acids to about 500 amino acids, about 400 amino acids to about 600 amino acids, about 400 amino acids to about 700 amino acids, about 400 amino acids to about 800 amino acids, about 400 amino acids to about 900 amino acids, about 400 amino acids to about 1,000 amino acids, about 500 amino acids to about 600 amino acids, about 500 amino acids to about 700 amino acids, about 500 amino acids to about 800 amino acids, about 500 amino acids to about 900 amino acids, about 500 amino acids to about 1,000 amino acids, about 600 amino acids to about 700 amino acids, about 600 amino acids to about 800 amino acids, about 600 amino acids to about 900 amino acids, about 600 amino acids to about 1,000 amino acids, about 700 amino acids to about 800 amino acids, about 700 amino acids to about 900 amino acids, about 700 amino acids to about 1,000 amino acids, about 800 amino acids to about 900 amino acids, about 800 amino acids to about 1,000 amino acids, or about 900 amino acids to about 1,000 amino acids in length.

In some embodiments, the analyte can be from about 1,000 amino acids to about 34,000 amino acids in length. In some embodiments, the analyte can be from about 1,000 amino acids to about 2,500 amino acids, about 1,000 amino acids to about 5,000 amino acids, about 1,000 amino acids to about 7,500 amino acids, about 1,000 amino acids to about 10,000 amino acids, about 1,000 amino acids to about 15,000 amino acids, about 1,000 amino acids to about 20,000 amino acids, about 1,000 amino acids to about 25,000 amino acids, about 1,000 amino acids to about 30,000 amino acids, about 1,000 amino acids to about 34,000 amino acids, about 2,500 amino acids to about 5,000 amino acids, about 2,500 amino acids to about 7,500 amino acids, about 2,500 amino acids to about 10,000 amino acids, about 2,500 amino acids to about 15,000 amino acids, about 2,500 amino acids to about 20,000 amino acids, about 2,500 amino acids to about 25,000 amino acids, about 2,500 amino acids to about 30,000 amino acids, about 2,500 amino acids to about 34,000 amino acids, about 5,000 amino acids to about 7,500 amino acids, about 5,000 amino acids to about 10,000 amino acids, about 5,000 amino acids to about 15,000 amino acids, about 5,000 amino acids to about 20,000 amino acids, about 5,000 amino acids to about 25,000 amino acids, about 5,000 amino acids to about 30,000 amino acids, about 5,000 amino acids to about 34,000 amino acids, about 7,500 amino acids to about 10,000 amino acids, about 7,500 amino acids to about 15,000 amino acids, about 7,500 amino acids to about 20,000 amino acids, about 7,500 amino acids to about 25,000 amino acids, about 7,500 amino acids to about 30,000 amino acids, about 7,500 amino acids to about 34,000 amino acids, about 10,000 amino acids to about 15,000 amino acids, about 10,000 amino acids to about 20,000 amino acids, about 10,000 amino acids to about 25,000 amino acids, about 10,000 amino acids to about 30,000 amino acids, about 10,000 amino acids to about 34,000 amino acids, about 15,000 amino acids to about 20,000 amino acids, about 15,000 amino acids to about 25,000 amino acids, about 15,000 amino acids to about 30,000 amino acids, about 15,000 amino acids to about 34,000 amino acids, about 20,000 amino acids to about 25,000 amino acids, about 20,000 amino acids to about 30,000 amino acids, about 20,000 amino acids to about 34,000 amino acids, about 25,000 amino acids to about 30,000 amino acids, about 25,000 amino acids to about 34,000 amino acids, or about 30,000 amino acids to about 34,000 amino acids in length.

In some embodiments, the analyte can be about 2 amino acids, about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, about 100 amino acids, about 150 amino acids, about 200 amino acids, about 250 amino acids, about 300 amino acids, about 350 amino acids, about 400 amino acids, about 450 amino acids, about 500 amino acids, about 600 amino acids, about 700 amino acids, about 800 amino acids, about 900 amino acids, about 1000 amino acids, about 2000 amino acids, about 3000 amino acids, about 4000 amino acids, about 5000 amino acids, about 6000 amino acids, about 7000 amino acids, about 8000 amino acids, about 9000 amino acids, about 10000 amino acids, about 20000 amino acids, about 30000, or about 34000 amino acids in length.

As described herein, the present invention can be particularly suitable for detecting analytes (e.g., folded proteins) or protein complexes that are larger than 80 kDa, for example larger than 100 kDa, or as another example larger than 150 kDa. In some embodiments, the analyte can comprises a mass of at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, at least about 55 kDa, at least about 60 kDa, at least about 65 kDa, at least about 70 kDa, at least about 75 kDa, at least about 80 kDa, at least about 85 kDa, at least about 90 kDa, at least about 95 kDa, at least about 100 kDa, at least about 125 kDa, at least about 150 kDa, at least about 175 kDa, at least about 200 kDa, at least about 250 kDa, at least about 300 kDa, at least about 350 kDa, at least about 400 kDa, at least about 450 kDa, at least about 500 kDa, at least about 550 kDa, at least about 600 kDa, at least about 650 kDa, at least about 700 kDa, at least about 750 kDa, at least about 800 kDa, at least about 850 kDa, at least about 900 kDa, at least about 950 kDa, at least about 1000 kDa, at least about 1500 kDa, at least about 2000 kDa, at least about 2500 kDa, at least about 3000 kDa, at least about 3500 kDa, at least about 4000 kDa, or greater than about 4000 kDa.

In some embodiments, the analyte can comprises a mass of at most about 4000 kDa, at most about 3500 kDa, at most about 3000 kDa, at most about 2500 kDa, at most about 2000 kDa, at most about 1500 kDa, at most about 1000 kDa, at most about 950 kDa, at most about 900 kDa, at most about 850 kDa, at most about 800 kDa, at most about 750 kDa, at most about 700 kDa, at most about 650 kDa, at most about 600 kDa, at most about 550 kDa, at most about 500 kDa, at most about 450 kDa, at most about 400 kDa, at most about 350 kDa, at most about 300 kDa, at most about 250 kDa, at most about 200 kDa, at most about 175 kDa, at most about 150 kDa, at most about 125 kDa, at most about 100 kDa, at most about 95 kDa, at most about 90 kDa, at most about 85 kDa, at most about 80 kDa, at most about 75 kDa, at most about 70 kDa, at most about 65 kDa, at most about 60 kDa, at most about 55 kDa, at most about 50 kDa, at most about 45 kDa, at most about 40 kDa, at most about 35 kDa, at most about 30 kDa, at most about 25 kDa, at most about 20 kDa, at most about 15 kDa, at most about 10 kDa, at most about 9 kDa, at most about 8 kDa, at most about 7 kDa, at most about 6 kDa, at most about 5 kDa, at most about 4 kDa, at most about 3 kDa, at most about 2 kDa, at most about 1 kDa, or less than about 1 kDa.

In some embodiments, the analyte can comprise a mass from about 1 kDa to about 100 kDa. In some embodiments, the analyte can be from about 1 kDa to about 5 kDa, about 1 kDa to about 10 kDa, about 1 kDa to about 20 kDa, about 1 kDa to about 30 kDa, about 1 kDa to about 40 kDa, about 1 kDa to about 50 kDa, about 1 kDa to about 60 kDa, about 1 kDa to about 70 kDa, about 1 kDa to about 80 kDa, about 1 kDa to about 90 kDa, about 1 kDa to about 100 kDa, about 5 kDa to about 10 kDa, about 5 kDa to about 20 kDa, about 5 kDa to about 30 kDa, about 5 kDa to about 40 kDa, about 5 kDa to about 50 kDa, about 5 kDa to about 60 kDa, about 5 kDa to about 70 kDa, about 5 kDa to about 80 kDa, about 5 kDa to about 90 kDa, about 5 kDa to about 100 kDa, about 10 kDa to about 20 kDa, about 10 kDa to about 30 kDa, about 10 kDa to about 40 kDa, about 10 kDa to about 50 kDa, about 10 kDa to about 60 kDa, about 10 kDa to about 70 kDa, about 10 kDa to about 80 kDa, about 10 kDa to about 90 kDa, about 10 kDa to about 100 kDa, about 20 kDa to about 30 kDa, about 20 kDa to about 40 kDa, about 20 kDa to about 50 kDa, about 20 kDa to about 60 kDa, about 20 kDa to about 70 kDa, about 20 kDa to about 80 kDa, about 20 kDa to about 90 kDa, about 20 kDa to about 100 kDa, about 30 kDa to about 40 kDa, about 30 kDa to about 50 kDa, about 30 kDa to about 60 kDa, about 30 kDa to about 70 kDa, about 30 kDa to about 80 kDa, about 30 kDa to about 90 kDa, about 30 kDa to about 100 kDa, about 40 kDa to about 50 kDa, about 40 kDa to about 60 kDa, about 40 kDa to about 70 kDa, about 40 kDa to about 80 kDa, about 40 kDa to about 90 kDa, about 40 kDa to about 100 kDa, about 50 kDa to about 60 kDa, about 50 kDa to about 70 kDa, about 50 kDa to about 80 kDa, about 50 kDa to about 90 kDa, about 50 kDa to about 100 kDa, about 60 kDa to about 70 kDa, about 60 kDa to about 80 kDa, about 60 kDa to about 90 kDa, about 60 kDa to about 100 kDa, about 70 kDa to about 80 kDa, about 70 kDa to about 90 kDa, about 70 kDa to about 100 kDa, about 80 kDa to about 90 kDa, about 80 kDa to about 100 kDa, or about 90 kDa to about 100 kDa.

In some embodiments, the analyte can comprise a mass from about 100 kDa to about 4,000 kDa. In some embodiments, the analyte can be from about 100 kDa to about 250 kDa, about 100 kDa to about 500 kDa, about 100 kDa to about 1,000 kDa, about 100 kDa to about 1,500 kDa, about 100 kDa to about 2,000 kDa, about 100 kDa to about 2,500 kDa, about 100 kDa to about 3,000 kDa, about 100 kDa to about 3,500 kDa, about 100 kDa to about 4,000 kDa, about 250 kDa to about 500 kDa, about 250 kDa to about 1,000 kDa, about 250 kDa to about 1,500 kDa, about 250 kDa to about 2,000 kDa, about 250 kDa to about 2,500 kDa, about 250 kDa to about 3,000 kDa, about 250 kDa to about 3,500 kDa, about 250 kDa to about 4,000 kDa, about 500 kDa to about 1,000 kDa, about 500 kDa to about 1,500 kDa, about 500 kDa to about 2,000 kDa, about 500 kDa to about 2,500 kDa, about 500 kDa to about 3,000 kDa, about 500 kDa to about 3,500 kDa, about 500 kDa to about 4,000 kDa, about 1,000 kDa to about 1,500 kDa, about 1,000 kDa to about 2,000 kDa, about 1,000 kDa to about 2,500 kDa, about 1,000 kDa to about 3,000 kDa, about 1,000 kDa to about 3,500 kDa, about 1,000 kDa to about 4,000 kDa, about 1,500 kDa to about 2,000 kDa, about 1,500 kDa to about 2,500 kDa, about 1,500 kDa to about 3,000 kDa, about 1,500 kDa to about 3,500 kDa, about 1,500 kDa to about 4,000 kDa, about 2,000 kDa to about 2,500 kDa, about 2,000 kDa to about 3,000 kDa, about 2,000 kDa to about 3,500 kDa, about 2,000 kDa to about 4,000 kDa, about 2,500 kDa to about 3,000 kDa, about 2,500 kDa to about 3,500 kDa, about 2,500 kDa to about 4,000 kDa, about 3,000 kDa to about 3,500 kDa, about 3,000 kDa to about 4,000 kDa, or about 3,500 kDa to about 4,000 kDa.

In some embodiments, the analyte can comprise a mass about 1 kDa, about 2 kDa, about 3 kDa, about 4 kDa, about 5 kDa, about 6 kDa, about 7 kDa, about 8 kDa, about 9 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 125 kDa, about 150 kDa, about 175 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1500 kDa, about 2000 kDa, about 2500 kDa, about 3000 kDa, about 3500 kDa, or about 4000 kDa.

In one aspect, the size and geometry of the analyte may only allow entry and exit at the wide cis end into the vestibule of the conical nanopore, while it cannot pass the narrow constriction region of the pore to prevent translocation. For detecting an analyte, one dimension (e.g., length, width, height, diameter, and/or circumference) larger than the constriction region can be enough. In some embodiments, an analyte may be greater in size than a narrowed portion of the channel of a nanopore described herein. For analyte trapping, it may be preferred that multiple dimensions (e.g., length, width, height, diameter, and/or circumference) are larger than the constriction region. In some embodiments, at least one dimension (e.g., length, width, height, diameter, and/or circumference) of an analyte can be at least about 2 times, at least about 3 times, at least about 4 times, at least about 5 times, at least about 6 times, at least about 7 times, at least about 8 times, at least about 9 times, at least about 10 times, at least about 12 times, at least about 13 times, at least about 14 times, at least about 15 times, at least about 16 times, at least about 17 times, at least about 18 times, at least about 19 times, at least about 20 times, at least about 25 times, at least about 30 times, at least about 35 times, at least about 40 times, at least about 45 times, at least about 50 times, at least about 55 times, at least about 60 times, at least about 65 times, at least about 70 times, at least about 75 times, at least about 80 times, at least about 85 times, at least about 90 times, at least about 95 times, at least about 100 times, or greater than about 100 times the channel width of the nanopore. In some embodiments, at least one dimension (e.g., length, width, height, diameter, and/or circumference) of an analyte can be at most about 100 times, at most about 95 times, at most about 90 times, at most about 80 times, at most about 75 times, at most about 70 times, at most about 65 times, at most about 60 times, at most about 55 times, at most about 50 times, at most about 45 times, at most about 40 times, at most about 35 times, at most about 30 times, at most about 25 times, at most about 20 times, at most about 19 times, at most about 18 times, at most about 17 times, at most about 16 times, at most about 15 times, at most about 14 times, at most about 13 times, at most about 12 times, at most about 11 times, at most about 10 times, at most about 9 times, at most about 8 times, at most about 7 times, at most about 6 times, at most about 5 times, at most about 4 times, at most about 3 times, at most about 2 times, or less than about 2 times the channel width of the nanopore.

In some embodiments, at least one dimension (e.g., length, width, height, diameter, and/or circumference) of an analyte can be from about 2 times to about 100 times the channel width of the nanopore. In some embodiments, at least one dimension (e.g., length, width, height, diameter, and/or circumference) of an analyte can be from about 2 times to about 5 times, about 2 times to about 10 times, about 2 times to about 20 times, about 2 times to about 30 times, about 2 times to about 40 times, about 2 times to about 50 times, about 2 times to about 60 times, about 2 times to about 70 times, about 2 times to about 80 times, about 2 times to about 90 times, about 2 times to about 100 times, about 5 times to about 10 times, about 5 times to about 20 times, about 5 times to about 30 times, about 5 times to about 40 times, about 5 times to about 50 times, about 5 times to about 60 times, about 5 times to about 70 times, about 5 times to about 80 times, about 5 times to about 90 times, about 5 times to about 100 times, about 10 times to about 20 times, about 10 times to about 30 times, about 10 times to about 40 times, about 10 times to about 50 times, about 10 times to about 60 times, about 10 times to about 70 times, about 10 times to about 80 times, about 10 times to about 90 times, about 10 times to about 100 times, about 20 times to about 30 times, about 20 times to about 40 times, about 20 times to about 50 times, about 20 times to about 60 times, about 20 times to about 70 times, about 20 times to about 80 times, about 20 times to about 90 times, about 20 times to about 100 times, about 30 times to about 40 times, about 30 times to about 50 times, about 30 times to about 60 times, about 30 times to about 70 times, about 30 times to about 80 times, about 30 times to about 90 times, about 30 times to about 100 times, about 40 times to about 50 times, about 40 times to about 60 times, about 40 times to about 70 times, about 40 times to about 80 times, about 40 times to about 90 times, about 40 times to about 100 times, about 50 times to about 60 times, about 50 times to about 70 times, about 50 times to about 80 times, about 50 times to about 90 times, about 50 times to about 100 times, about 60 times to about 70 times, about 60 times to about 80 times, about 60 times to about 90 times, about 60 times to about 100 times, about 70 times to about 80 times, about 70 times to about 90 times, about 70 times to about 100 times, about 80 times to about 90 times, about 80 times to about 100 times, or about 90 times to about 100 times the channel width of the nanopore.

In some embodiments, at least one dimension (e.g., length, width, height, diameter, and/or circumference) of an analyte can be at least about 2 times, about 3 times, about 4 times, about 5 times, about 6 times, about 7 times, about 8 times, about 9 times, about 10 times, about 12 times, about 13 times, about 14 times, about 15 times, about 16 times, about 17 times, about 18 times, about 19 times, about 20 times, about 25 times, about 30 times, about 35 times, about 40 times, about 45 times, about 50 times, about 55 times, about 60 times, about 65 times, about 70 times, about 75 times, about 80 times, about 85 times, about 90 times, about 95 times, or about 100 times the channel width of the nanopore.

In some embodiments, the analyte may have a length of 2-20 nm, for example greater than about 3 nm and less than about 15 nm. In some embodiments, an analyte has at least one dimension (e.g., length, width, height, diameter, and/or circumference) that is at least about 2 nm, at least about 3 nm, at least about 4 nm, at least about 5 nm, at least about 6 nm, at least about 7 nm, at least about 8 nm, at least about 9 nm, at least about 10 nm, at least about 12 nm, at least about 15 nm, at least about 18 nm, at least about 20 nm, at least about 25 nm, or greater than about 25 nm in length. In some embodiments, an analyte has at least one dimension (e.g., length, width, height, diameter, and/or circumference) that is at most about 25 nm, at most about 20 nm, at most about 18 nm, at most about 15 nm, at most about 12 nm, at most about 10 nm, at most about 9 nm, at most about 8 nm, at most about 7 nm, at most about 6 nm, at most about 5 nm, at most about 4 nm, at most about 3 nm, at most about 2 nm, or less than about 2 nm in length. In some embodiments, an analyte has at least one dimension (e.g., length, width, height, diameter, and/or circumference) from about 3 nm to about 20 nm in length. In some embodiments, an analyte has at least one dimension (e.g., length, width, height, diameter, and/or circumference) from about 3 nm to about 4 nm, about 3 nm to about 5 nm, about 3 nm to about 6 nm, about 3 nm to about 7 nm, about 3 nm to about 8 nm, about 3 nm to about 9 nm, about 3 nm to about 10 nm, about 3 nm to about 12 nm, about 3 nm to about 15 nm, about 3 nm to about 18 nm, about 3 nm to about 20 nm, about 4 nm to about 5 nm, about 4 nm to about 6 nm, about 4 nm to about 7 nm, about 4 nm to about 8 nm, about 4 nm to about 9 nm, about 4 nm to about 10 nm, about 4 nm to about 12 nm, about 4 nm to about 15 nm, about 4 nm to about 18 nm, about 4 nm to about 20 nm, about 5 nm to about 6 nm, about 5 nm to about 7 nm, about 5 nm to about 8 nm, about 5 nm to about 9 nm, about 5 nm to about 10 nm, about 5 nm to about 12 nm, about 5 nm to about 15 nm, about 5 nm to about 18 nm, about 5 nm to about 20 nm, about 6 nm to about 7 nm, about 6 nm to about 8 nm, about 6 nm to about 9 nm, about 6 nm to about 10 nm, about 6 nm to about 12 nm, about 6 nm to about 15 nm, about 6 nm to about 18 nm, about 6 nm to about 20 nm, about 7 nm to about 8 nm, about 7 nm to about 9 nm, about 7 nm to about 10 nm, about 7 nm to about 12 nm, about 7 nm to about 15 nm, about 7 nm to about 18 nm, about 7 nm to about 20 nm, about 8 nm to about 9 nm, about 8 nm to about 10 nm, about 8 nm to about 12 nm, about 8 nm to about 15 nm, about 8 nm to about 18 nm, about 8 nm to about 20 nm, about 9 nm to about 10 nm, about 9 nm to about 12 nm, about 9 nm to about 15 nm, about 9 nm to about 18 nm, about 9 nm to about 20 nm, about 10 nm to about 12 nm, about 10 nm to about 15 nm, about 10 nm to about 18 nm, about 10 nm to about 20 nm, about 12 nm to about 15 nm, about 12 nm to about 18 nm, about 12 nm to about 20 nm, about 15 nm to about 18 nm, about 15 nm to about 20 nm, or about 18 nm to about 20 nm in length.

The analyte may have a hydrodynamic radius of at least 20 Å, for example at least 25 Å, as another example at least 28 Å or at least 30 Å. In one aspect, the analyte may comprise a hydrodynamic radius in the range of about 25 to 50 Å, for example 28 to 50 Å. In some embodiments, an analyte described herein may have a hydrodynamic radius of at least about 10 Å, at least about 15 Å, at least about 20 Å, at least about 21 Å, at least about 22 Å, at least about 23 Å, at least about 24 Å, at least about 25 Å, at least about 26 Å, at least about 27 Å, at least about 28 Å, at least about 29 Å, at least about 30 Å, at least about 35 Å, at least about 40 Å, at least about 45 Å, at least about 50 Å, or greater than about 50 Å. In some embodiments, an analyte described herein may have a hydrodynamic radius of at most about 50 Å, at most about 45 Å, at most about 40 Å, at most about 35 Å, at most about 30 Å, at most about 29 Å, at most about 28 Å, at most about 27 Å, at most about 26 Å, at most about 25 Å, at most about 24 Å, at most about 23 Å, at most about 22 Å, at most about 21 Å, at most about 20 Å, at most about 15 Å, at most about 10 Å, or less than about 10 Å. In some embodiments, an analyte described herein may have a hydrodynamic radius from about 10 Å to about 50 Å. In some embodiments, an analyte described herein may have a hydrodynamic radius from about 10 Å to about 15 Å, about 10 Å to about 20 Å, about 10 Å to about 22 Å, about 10 Å to about 24 Å, about 10 Å to about 26 Å, about 10 Å to about 28 Å, about 10 Å to about 30 Å, about 10 Å to about 35 Å, about 10 Å to about 40 Å, about 10 Å to about 45 Å, about 10 Å to about 50 Å, about 15 Å to about 20 Å, about 15 Å to about 22 Å, about 15 Å to about 24 Å, about 15 Å to about 26 Å, about 15 Å to about 28 Å, about 15 Å to about 30 Å, about 15 Å to about 35 Å, about 15 Å to about 40 Å, about 15 Å to about 45 Å, about 15 Å to about 50 Å, about 20 Å to about 22 Å, about 20 Å to about 24 Å, about 20 Å to about 26 Å, about 20 Å to about 28 Å, about 20 Å to about 30 Å, about 20 Å to about 35 Å, about 20 Å to about 40 Å, about 20 Å to about 45 Å, about 20 Å to about 50 Å, about 22 Å to about 24 Å, about 22 Å to about 26 Å, about 22 Å to about 28 Å, about 22 Å to about 30 Å, about 22 Å to about 35 Å, about 22 Å to about 40 Å, about 22 Å to about 45 Å, about 22 Å to about 50 Å, about 24 Å to about 26 Å, about 24 Å to about 28 Å, about 24 Å to about 30 Å, about 24 Å to about 35 Å, about 24 Å to about 40 Å, about 24 Å to about 45 Å, about 24 Å to about 50 Å, about 26 Å to about 28 Å, about 26 Å to about 30 Å, about 26 Å to about 35 Å, about 26 Å to about 40 Å, about 26 Å to about 45 Å, about 26 Å to about 50 Å, about 28 Å to about 30 Å, about 28 Å to about 35 Å, about 28 Å to about 40 Å, about 28 Å to about 45 Å, about 28 Å to about 50 Å, about 30 Å to about 35 Å, about 30 Å to about 40 Å, about 30 Å to about 45 Å, about 30 Å to about 50 Å, about 35 Å to about 40 Å, about 35 Å to about 45 Å, about 35 Å to about 50 Å, about 40 Å to about 45 Å, about 40 Å to about 50 Å, or about 45 Å to about 50 Å.

In certain analyte sensing applications, it may be desirable to tune the residence time of a target analyte in the vestibule of the conical nanopore. Whereas it is often sufficient, or even preferred, to have short residence of >10 milliseconds (ms) (e.g., 10 ms to 1 sec), for basic analyte detection, it is in some cases advantageous to have a much longer residence time of >1 second (sec). Depending on the analyte and/or the nanopore characteristics, if needed, the trapping time may be increased by the functionalization of the proteinaceous conical nanopore. Herewith, the functionalized nanopore enhances capture frequency of the target analyte from solution into the nanopore vestibule and/or reduces the unbinding (release) of the target analyte from the nanopore. In some embodiments, the shape of a nanopore described herein can increase a residence time of a target analyte in the lumen (e.g., channel) of the nanopore. In some embodiments, a nanopore comprises a cylindrical shape on a second side (e.g., trans side). In some embodiments, a nanopore comprises a cone shape on a first side (e.g., cis side). In some embodiments, a nanopore comprises a cylindrical shape on a second side (e.g., trans side) and a cone shape on a cis side separated by an inner constriction. In some embodiments a nanopore comprises an hourglass shape (e.g., a cone shape on a first (e.g., cis side) and a cone shape on a second (e.g., trans side), separated by an inner constriction in the channel. Without wishing to be bound by theory, alterations in a nanopore's chemical (e.g., amino acid) composition, pH, ion selectivity, electro-osmotic flux, conductivity, or any combination thereof, may affect a residence time of an analyte in a nanopore described herein.

An increase residence time in a lumen of a nanopore may provide for better characterization (e.g., sequencing determination) of the target analyte. In some embodiments, an analyte may reside in a lumen of a nanopore for at least about 10 ms, at least about 50 ms, at least about 100 ms, at least about 250 ms, at least about 500 ms, at least about 750 ms, at least about 1000 ms, at least about 1250 ms, at least about 1500 ms, at least about 1750 ms, at least about 2000 ms, at least about 2500 ms, at least about 3000 ms, at least about 4000 ms, at least about 5000 ms, or greater than 5000 ms. In some embodiments, an analyte may reside in a lumen of a nanopore for at most about 5000 ms, at most about 4000 ms, at most about 3000 ms, at most about 2500 ms, at most about 2000 ms, at most about 1750 ms, at most about 1500 ms, at most about 1250 ms, at most about 1000 ms, at most about 750 ms, at most about 500 ms, at most about 250 ms, at most about 100 ms, at most about 50 ms, at most about 10 ms, or less than about 10 ms. In some embodiments, an analyte may reside in a lumen of a nanopore for at least about 10 seconds (s), 20 s, 30 s, 1 minute, 2 minutes, 3 minutes, 4 minutes, 5 minutes, 10 minutes, 15 minutes, 30 minutes, or greater than about 30 minutes. In some embodiments, an analyte may reside in a lumen of a nanopore for at most about 30 minutes, 15 minutes, 10 minutes, 5 minutes, 4 minutes, 3 minutes, 2 minutes, 1 minute, 30 s, 20 s, 10 s, or less than about 10 s.

In some embodiments, a target analyte may reside in a lumen of a nanopore from about 10 ms to about 5,000 ms. In some embodiments, a target analyte may reside in a lumen of a nanopore from about 10 ms to about 25 ms, about 10 ms to about 50 ms, about 10 ms to about 100 ms, about 10 ms to about 250 ms, about 10 ms to about 500 ms, about 10 ms to about 750 ms, about 10 ms to about 1,000 ms, about 10 ms to about 2,000 ms, about 10 ms to about 3,000 ms, about 10 ms to about 4,000 ms, about 10 ms to about 5,000 ms, about 25 ms to about 50 ms, about 25 ms to about 100 ms, about 25 ms to about 250 ms, about 25 ms to about 500 ms, about 25 ms to about 750 ms, about 25 ms to about 1,000 ms, about 25 ms to about 2,000 ms, about 25 ms to about 3,000 ms, about 25 ms to about 4,000 ms, about 25 ms to about 5,000 ms, about 50 ms to about 100 ms, about 50 ms to about 250 ms, about 50 ms to about 500 ms, about 50 ms to about 750 ms, about 50 ms to about 1,000 ms, about 50 ms to about 2,000 ms, about 50 ms to about 3,000 ms, about 50 ms to about 4,000 ms, about 50 ms to about 5,000 ms, about 100 ms to about 250 ms, about 100 ms to about 500 ms, about 100 ms to about 750 ms, about 100 ms to about 1,000 ms, about 100 ms to about 2,000 ms, about 100 ms to about 3,000 ms, about 100 ms to about 4,000 ms, about 100 ms to about 5,000 ms, about 250 ms to about 500 ms, about 250 ms to about 750 ms, about 250 ms to about 1,000 ms, about 250 ms to about 2,000 ms, about 250 ms to about 3,000 ms, about 250 ms to about 4,000 ms, about 250 ms to about 5,000 ms, about 500 ms to about 750 ms, about 500 ms to about 1,000 ms, about 500 ms to about 2,000 ms, about 500 ms to about 3,000 ms, about 500 ms to about 4,000 ms, about 500 ms to about 5,000 ms, about 750 ms to about 1,000 ms, about 750 ms to about 2,000 ms, about 750 ms to about 3,000 ms, about 750 ms to about 4,000 ms, about 750 ms to about 5,000 ms, about 1,000 ms to about 2,000 ms, about 1,000 ms to about 3,000 ms, about 1,000 ms to about 4,000 ms, about 1,000 ms to about 5,000 ms, about 2,000 ms to about 3,000 ms, about 2,000 ms to about 4,000 ms, about 2,000 ms to about 5,000 ms, about 3,000 ms to about 4,000 ms, about 3,000 ms to about 5,000 ms, or about 4,000 ms to about 5,000 ms.

In some embodiments, a target analyte may reside in a lumen of a nanopore from about 0.5 minutes to about 45 minutes. In some embodiments, a target analyte may reside in a lumen of a nanopore from about 0.5 minutes to about 1 minute, about 0.5 minutes to about 2 minutes, about 0.5 minutes to about 3 minutes, about 0.5 minutes to about 4 minutes, about 0.5 minutes to about 5 minutes, about 0.5 minutes to about 10 minutes, about 0.5 minutes to about 15 minutes, about 0.5 minutes to about 20 minutes, about 0.5 minutes to about 25 minutes, about 0.5 minutes to about 30 minutes, about 0.5 minutes to about 45 minutes, about 1 minute to about 2 minutes, about 1 minute to about 3 minutes, about 1 minute to about 4 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 45 minutes, about 2 minutes to about 3 minutes, about 2 minutes to about 4 minutes, about 2 minutes to about 5 minutes, about 2 minutes to about 10 minutes, about 2 minutes to about 15 minutes, about 2 minutes to about 20 minutes, about 2 minutes to about 25 minutes, about 2 minutes to about 30 minutes, about 2 minutes to about 45 minutes, about 3 minutes to about 4 minutes, about 3 minutes to about 5 minutes, about 3 minutes to about 10 minutes, about 3 minutes to about 15 minutes, about 3 minutes to about 20 minutes, about 3 minutes to about 25 minutes, about 3 minutes to about 30 minutes, about 3 minutes to about 45 minutes, about 4 minutes to about 5 minutes, about 4 minutes to about 10 minutes, about 4 minutes to about 15 minutes, about 4 minutes to about 20 minutes, about 4 minutes to about 25 minutes, about 4 minutes to about 30 minutes, about 4 minutes to about 45 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 45 minutes, about 10 minutes to about 15 minutes, about 10 minutes to about 20 minutes, about 10 minutes to about 25 minutes, about 10 minutes to about 30 minutes, about 10 minutes to about 45 minutes, about 15 minutes to about 20 minutes, about 15 minutes to about 25 minutes, about 15 minutes to about 30 minutes, about 15 minutes to about 45 minutes, about 20 minutes to about 25 minutes, about 20 minutes to about 30 minutes, about 20 minutes to about 45 minutes, about 25 minutes to about 30 minutes, about 25 minutes to about 45 minutes, or about 30 minutes to about 45 minutes.

Suitably, the conical nanopore can be functionalized at, or near to, the top of its cis entrance with one or more polymeric extensions, optionally also comprising one or more recognition element(s) capable of specifically binding to a target analyte. A recognition element can but does not need to be of proteinaceous nature. A recognition element can be a protein, peptide, or polypeptide. A recognition element may be a small-molecule (e.g., a ligand to a target protein), a protein (folded or unfolded), DNA, RNA, etc. The molecular weight or size of the (proteinaceous) recognition element can vary. In one aspect, it can be small e.g. below 5 kDa. In some cases, a recognition element can be at least about 0.1 kDa, at least about 0.2 kDa, at least about 0.3 kDa, at least about 0.4 kDa, at least about 0.5 kDa, at least about 0.6 kDa, at least about 0.7 kDa, at least about 0.8 kDa, at least about 0.9 kDa, at least about 1.0 kDa, at least about 1.5 kDa, at least about 2.0 kDa, at least about 2.5 kDa, at least about 3.0 kDa, at least about 3.5 kDa, at least about 4.0 kDa, at least about 4.5 kDa, at least about 5.0 kDa, at least about 6.0 kDa, at least about 7.0 kDa, at least about 8.0 kDa, at least about 9.0 kDa, at least about 10.0 kDa, or greater than about 10.0 kDa. In some cases, a recognition element can be at most about 10.0 kDa, at most about 9.0 kDa, at most about 8.0 kDa, at most about 7.0 kDa, at most about 6.0 kDa, at most about 5.0 kDa, at most about 4.5 kDa, at most about 4.0 kDa, at most about 3.5 kDa, at most about 3.0 kDa, at most about 2.5 kDa, at most about 2.0 kDa, at most about 1.5 kDa, at most about 1.0 kDa, at most about 0.9 kDa, at most about 0.8 kDa, at most about 0.7 kDa, at most about 0.6 kDa, at most about 0.5 kDa, at most about 0.4 kDa, at most about 0.3 kDa, at most about 0.2 kDa, at most about 0.1 kDa, or less than about 0.1 kDa.

In some cases, a recognition element can be from about 0.1 kDa to about 5 kDa. In some cases, a recognition element can be from about 0.1 kDa to about 0.2 kDa, about 0.1 kDa to about 0.3 kDa, about 0.1 kDa to about 0.4 kDa, about 0.1 kDa to about 0.5 kDa, about 0.1 kDa to about 1 kDa, about 0.1 kDa to about 1.5 kDa, about 0.1 kDa to about 2 kDa, about 0.1 kDa to about 2.5 kDa, about 0.1 kDa to about 3 kDa, about 0.1 kDa to about 4 kDa, about 0.1 kDa to about 5 kDa, about 0.2 kDa to about 0.3 kDa, about 0.2 kDa to about 0.4 kDa, about 0.2 kDa to about 0.5 kDa, about 0.2 kDa to about 1 kDa, about 0.2 kDa to about 1.5 kDa, about 0.2 kDa to about 2 kDa, about 0.2 kDa to about 2.5 kDa, about 0.2 kDa to about 3 kDa, about 0.2 kDa to about 4 kDa, about 0.2 kDa to about 5 kDa, about 0.3 kDa to about 0.4 kDa, about 0.3 kDa to about 0.5 kDa, about 0.3 kDa to about 1 kDa, about 0.3 kDa to about 1.5 kDa, about 0.3 kDa to about 2 kDa, about 0.3 kDa to about 2.5 kDa, about 0.3 kDa to about 3 kDa, about 0.3 kDa to about 4 kDa, about 0.3 kDa to about 5 kDa, about 0.4 kDa to about 0.5 kDa, about 0.4 kDa to about 1 kDa, about 0.4 kDa to about 1.5 kDa, about 0.4 kDa to about 2 kDa, about 0.4 kDa to about 2.5 kDa, about 0.4 kDa to about 3 kDa, about 0.4 kDa to about 4 kDa, about 0.4 kDa to about 5 kDa, about 0.5 kDa to about 1 kDa, about 0.5 kDa to about 1.5 kDa, about 0.5 kDa to about 2 kDa, about 0.5 kDa to about 2.5 kDa, about 0.5 kDa to about 3 kDa, about 0.5 kDa to about 4 kDa, about 0.5 kDa to about 5 kDa, about 1 kDa to about 1.5 kDa, about 1 kDa to about 2 kDa, about 1 kDa to about 2.5 kDa, about 1 kDa to about 3 kDa, about 1 kDa to about 4 kDa, about 1 kDa to about 5 kDa, about 1.5 kDa to about 2 kDa, about 1.5 kDa to about 2.5 kDa, about 1.5 kDa to about 3 kDa, about 1.5 kDa to about 4 kDa, about 1.5 kDa to about 5 kDa, about 2 kDa to about 2.5 kDa, about 2 kDa to about 3 kDa, about 2 kDa to about 4 kDa, about 2 kDa to about 5 kDa, about 2.5 kDa to about 3 kDa, about 2.5 kDa to about 4 kDa, about 2.5 kDa to about 5 kDa, about 3 kDa to about 4 kDa, about 3 kDa to about 5 kDa, or about 4 kDa to about 5 kDa.

The recognition element can be conjugated to a nanopore subunit by any known means in the art, including chemical conjugation (e.g. using cysteine coupling chemistries, click chemistries, etc.) or biological attachment e.g. by genetic fusion. For example, a nanopore comprising YaxAB monomers, or mutants, functional homologs, functional orthologs, or functional paralogs thereof, can be functionalized by modification of one or more A and/or B subunits. In some embodiments, a YaxA subunit may be conjugated to a recognition element. In some embodiments, a YaxB subunit may be conjugated to a recognition element. In some embodiments, at least one YaxA subunit and at least one YaxB subunit may be conjugated to a recognition element. Individual nanopore subunits can be functionalized with the same or with different recognition elements. A recognition element can be conjugated to a nanopore at a first opening (e.g., a cis entrance). A recognition element can be conjugated to a nanopore at a second opening (e.g., a trans entrance). A nanopore can comprise one or more recognition elements. A nanopore can comprise one or more recognition elements at a first opening (e.g., cis entrance) and/or a second opening (e.g., trans entrance). In some embodiments, a nanopore described herein can comprise 1, 2, 3, 4, 5, 6, 7, 8, 9, 10, or more recognition elements. In some embodiments, a recognition element may be synthesized with a nanopore prior to inserting a nanopore into a membrane. In some embodiments, a recognition element may conjugate to a nanopore disposed in a membrane.

In some embodiments, one or more recognition elements may be coupled to an analyte (e.g., a non-nucleic acid based polymer analyte). The recognition elements may be different. The different recognition elements may comprise a different chemical composition, shape, size, ionic composition, conductance, or any combination thereof. The one or more recognition elements of a nanopore may be the same. In some embodiments, the same recognition elements may have the same sequence and structure. In some embodiments, the one or more recognition elements may bind to the same analyte in a sample of a mixture of analytes. In some embodiments, the one or more recognition elements may bind to different analytes in a sample of a mixture of analytes.

In some embodiments, nanopores with different functionalities in various stoichiometries can be obtained when the subunits are mixed. Alternatively, two or more different recognition elements can be added to one monomer of a nanopore by concatenating the different recognition elements together. In some embodiments, a first recognition element may be bound to a nanopore (e.g., conjugated to a nanopore) and a second recognition element may be concatenated to the first recognition element. In some embodiments, two or more different recognition elements can be added to one monomer by concatenating the different recognition elements together with an intervening section of linker. In some embodiments, two or more different recognition elements can be added to one monomer by concatenating the different recognition elements together without an intervening section of linker. In some embodiments, two or more recognition elements can be conjugated to a YaxA subunit. In some embodiments, two or more recognition elements can be conjugated to a YaxB subunit. In some embodiments, at least one YaxA subunit and at least one YaxB subunit of a nanopore comprise a recognition element. In some embodiments, two or more recognition elements may bind to an individual subunit of a nanopore. In some embodiments, two or more recognition elements may each bind to a different subunit of a nanopore. In some embodiments, in a collection of multiple recognition elements, a first subset of recognition elements may bind to one subunit of a nanopore and a second subset of recognition elements may bind to different subunits of the nanopore.

By building multiple different recognition elements into a single oligomeric nanopore (whether formed of differentially modified subunits or formed from a single species of subunit that contains multiple different recognition elements), it may be possible to better control the capture and binding of multiple different target analytes to a single nanopore sensor. Without wishing to be bound by theory, a sample comprising a mixture of analytes (e.g., analytes of different size, shape, sequence, chemical composition, pH, or any combination thereof) may be filtered by nanopores comprising different recognition elements. Alternatively, the multiple recognition elements on a single nanopore might bind to different regions of the same target analyte to increase the specificity for detecting the given target analyte over binding to unwanted analytes in a mixture.

In some embodiments, one or more recognition elements may be directly coupled to a nanopore. In some embodiments, one or more recognition elements may be indirectly coupled to a nanopore. In some cases, the recognition element may be indirectly coupled (e.g., not bound) to the nanopore by a linker. For example, when a recognition element is indirectly coupled to a nanopore, the recognition element may not be directly adjacent to the nanopore (e.g., separated by a linker). For example, when a recognition element is directly coupled to a nanopore, the recognition element may be directly adjacent to the nanopore. In some embodiments, the recognition element may be indirectly coupled to the nanopore by chelation-ligand coupling, biotin-streptavidin interaction, or any combination thereof.

The recognition element(s) can be coupled to the nanopore via a flexible (unstructured) linker moiety. The linker moiety can consist or comprise proteinaceous, DNA, other unstructured polymeric moieties such as polyethylene glycol (PEG) etc., or any combination thereof. The linker length can vary according to needs. For example, the linker can be at least 1 nm, or at least 3 nm, or at least 6 nm, or at least 10 nm or at least 20 nm. Longer linkers of 25 nm or more, 30 nm or more, or 50 nm or more are also envisaged. In some embodiments, a linker described herein can be at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 15 nm, at least about 20 nm, at least about 25 nm, at least about 30 nm, at least about 40 nm, at least about 50 nm, at least about 75 nm, or greater than about 75 nm in length. In some embodiments, a linker described herein can be at most about 75 nm, at most about 50 nm, at most about 40 nm, at most about 30 nm, at most about 25 nm, at most about 20 nm, at most about 15 nm, at most about 10 nm, at most about 9 nm, at most about 8 nm, at most about 7 nm, at most about 6 nm, at most about 5 nm, at most about 4 nm, at most about 3 nm, at most about 2 nm, at most about 1 nm, or less than about 1 nm.

In one aspect, the linker has a length in the range of 1-30 nm, 1-25 nm, 6-25 nm, 1-10 nm, or 10 to 30 nm. In some embodiments, a linker described herein has a length from about 1 nm to about 75 nm. In some embodiments, a linker described herein has a length from about 1 nm to about 2 nm, about 1 nm to about 3 nm, about 1 nm to about 4 nm, about 1 nm to about 5 nm, about 1 nm to about 8 nm, about 1 nm to about 10 nm, about 1 nm to about 15 nm, about 1 nm to about 20 nm, about 1 nm to about 25 nm, about 1 nm to about 50 nm, about 1 nm to about 75 nm, about 2 nm to about 3 nm, about 2 nm to about 4 nm, about 2 nm to about 5 nm, about 2 nm to about 8 nm, about 2 nm to about 10 nm, about 2 nm to about 15 nm, about 2 nm to about 20 nm, about 2 nm to about 25 nm, about 2 nm to about 50 nm, about 2 nm to about 75 nm, about 3 nm to about 4 nm, about 3 nm to about 5 nm, about 3 nm to about 8 nm, about 3 nm to about 10 nm, about 3 nm to about 15 nm, about 3 nm to about 20 nm, about 3 nm to about 25 nm, about 3 nm to about 50 nm, about 3 nm to about 75 nm, about 4 nm to about 5 nm, about 4 nm to about 8 nm, about 4 nm to about 10 nm, about 4 nm to about 15 nm, about 4 nm to about 20 nm, about 4 nm to about 25 nm, about 4 nm to about 50 nm, about 4 nm to about 75 nm, about 5 nm to about 8 nm, about 5 nm to about 10 nm, about 5 nm to about 15 nm, about 5 nm to about 20 nm, about 5 nm to about 25 nm, about 5 nm to about 50 nm, about 5 nm to about 75 nm, about 8 nm to about 10 nm, about 8 nm to about 15 nm, about 8 nm to about 20 nm, about 8 nm to about 25 nm, about 8 nm to about 50 nm, about 8 nm to about 75 nm, about 10 nm to about 15 nm, about 10 nm to about 20 nm, about 10 nm to about 25 nm, about 10 nm to about 50 nm, about 10 nm to about 75 nm, about 15 nm to about 20 nm, about 15 nm to about 25 nm, about 15 nm to about 50 nm, about 15 nm to about 75 nm, about 20 nm to about 25 nm, about 20 nm to about 50 nm, about 20 nm to about 75 nm, about 25 nm to about 50 nm, about 25 nm to about 75 nm, or about 50 nm to about 75 nm.

In some embodiments, the at least one recognition element can be attached to the nanopore via a linker sequence (e.g., a protein, peptide, or polypeptide linker sequence). Good results can be obtained with a nanopore system comprising an oligomeric assembly of subunits, wherein at least one subunit may be functionalized with a recognition element via an N- and/or C-terminal peptide extension comprising a linker sequence and recognition element. A linker can comprise a peptide linker, a flexible linker, a rigid linker, a cleavable linker, a dipeptide linker, a pyrophosphate linker, a carbohydrate linker, or a hydrazone linker. Suitably, the linker sequence (e.g., a protein, peptide, or polypeptide linker sequence) comprises at least 3 amino acids, preferably 3 to 100 amino acids, more preferably 10 to 70 amino acids. In some embodiments, the peptide linker sequences can comprise at least about 3 amino acids, at least about 4 amino acids, at least about 5 amino acids, at least about 10 amino acids, at least about 15 amino acids, at least about 20 amino acids, at least about 25 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 125 amino acids, or greater than about 125 amino acids. In some embodiments, the peptide linker sequences can comprise at most about 125 amino acids, at most about 100 amino acids, at most about 90 amino acids, at most about 80 amino acids, at most about 70 amino acids, at most about 60 amino acids, at most about 50 amino acids, at most about 40 amino acids, at most about 30 amino acids, at most about 25 amino acids, at most about 20 amino acids, at most about 15 amino acids, at most about 10 amino acids, at most about 5 amino acids, at most about 4 amino acids, at most about 3 amino acids, or less than about 3 amino acids.

In some embodiments, the linker sequence (e.g., a protein, peptide, or polypeptide linker sequence) can comprise from about 3 amino acids to about 100 amino acids. In some embodiments, the peptide linker sequences can comprise from at least about 3 amino acids. In some embodiments, the peptide linker sequences can comprise from at most about 100 amino acids. In some embodiments, the peptide linker sequences can comprise from about 3 amino acids to about 5 amino acids, about 3 amino acids to about 10 amino acids, about 3 amino acids to about 20 amino acids, about 3 amino acids to about 30 amino acids, about 3 amino acids to about 40 amino acids, about 3 amino acids to about 50 amino acids, about 3 amino acids to about 60 amino acids, about 3 amino acids to about 70 amino acids, about 3 amino acids to about 80 amino acids, about 3 amino acids to about 90 amino acids, about 3 amino acids to about 100 amino acids, about 5 amino acids to about 10 amino acids, about 5 amino acids to about 20 amino acids, about 5 amino acids to about 30 amino acids, about 5 amino acids to about 40 amino acids, about 5 amino acids to about 50 amino acids, about 5 amino acids to about 60 amino acids, about 5 amino acids to about 70 amino acids, about 5 amino acids to about 80 amino acids, about 5 amino acids to about 90 amino acids, about 5 amino acids to about 100 amino acids, about 10 amino acids to about 20 amino acids, about 10 amino acids to about 30 amino acids, about 10 amino acids to about 40 amino acids, about 10 amino acids to about 50 amino acids, about 10 amino acids to about 60 amino acids, about 10 amino acids to about 70 amino acids, about 10 amino acids to about 80 amino acids, about 10 amino acids to about 90 amino acids, about 10 amino acids to about 100 amino acids, about 20 amino acids to about 30 amino acids, about 20 amino acids to about 40 amino acids, about 20 amino acids to about 50 amino acids, about 20 amino acids to about 60 amino acids, about 20 amino acids to about 70 amino acids, about 20 amino acids to about 80 amino acids, about 20 amino acids to about 90 amino acids, about 20 amino acids to about 100 amino acids, about 30 amino acids to about 40 amino acids, about 30 amino acids to about 50 amino acids, about 30 amino acids to about 60 amino acids, about 30 amino acids to about 70 amino acids, about 30 amino acids to about 80 amino acids, about 30 amino acids to about 90 amino acids, about 30 amino acids to about 100 amino acids, about 40 amino acids to about 50 amino acids, about 40 amino acids to about 60 amino acids, about 40 amino acids to about 70 amino acids, about 40 amino acids to about 80 amino acids, about 40 amino acids to about 90 amino acids, about 40 amino acids to about 100 amino acids, about 50 amino acids to about 60 amino acids, about 50 amino acids to about 70 amino acids, about 50 amino acids to about 80 amino acids, about 50 amino acids to about 90 amino acids, about 50 amino acids to about 100 amino acids, about 60 amino acids to about 70 amino acids, about 60 amino acids to about 80 amino acids, about 60 amino acids to about 90 amino acids, about 60 amino acids to about 100 amino acids, about 70 amino acids to about 80 amino acids, about 70 amino acids to about 90 amino acids, about 70 amino acids to about 100 amino acids, about 80 amino acids to about 90 amino acids, about 80 amino acids to about 100 amino acids, or about 90 amino acids to about 100 amino acids.

Protein linkers may comprise three major types of linkers: flexible, rigid, and in vivo cleavable. Flexible linkers may consist (mainly) of many small glycine residues, giving them the ability curl into a dynamic, adaptable shape. Rigid linkers may be formed of large, cyclic proline residues, which can be helpful when highly specific spacing between domains must be maintained.

Amino acids constituting a linker sequence for use in the present invention can include a wide range of amino acids, including hydrophilic and aromatic amino acids. The linker can be mostly unstructured, but can also have rigid elements and/or α-helical elements. Amino acid sequence motifs can comprise Ala-Pro (rigid AP motif), the EAAAK motif (alpha helical rigid) and FG-motif. In a specific aspect, a peptide linker can be mainly composed of G, S, T, and very few A and N. Charged linkers may contain R and K (positively charged), or D and E (negatively charged). In some embodiments, a peptide linker sequence comprises (GGGGS)_{N}, wherein N is 1, 2, 3, 4, 5, 6, 7, 8, 9, or 10. In some embodiments, a peptide linker sequence comprises (Gly)_{N}, wherein N is 1, 2, 3, 4, 5, 6, 7, 8, 9, or 10. In some embodiments, a peptide linker sequence comprises (EAAAK)_{N}, wherein N is 1, 2, 3, 4, 5, 6, 7, 8, 9, or 10. In some embodiments, a peptide linker sequence comprises A(EAAAK)_{N}ALEA(EAAAK)_{N} Å, wherein N is 1, 2, 3, 4, 5, 6, 7, 8, 9, or 10. In some embodiments, a peptide linker sequence comprises (AP)_{N}, wherein N is 1, 2, 3, 4, 5, 6, 7, 8, 9, 10, 11, 12, 13, 14, 15, or 20.

The protein, peptide, or polypeptide extension may be attached to the full-length nanopore-forming subunit, or it may attached to one or more truncated nanopore subunits from which at least part of the N-/or C-terminal (unstructured) region has been removed. In some embodiments, an extension (e.g., a protein, peptide, or polypeptide extension) can be attached to a full-length subunit originating from Yersinia enterocolitica (e.g., YaxA or YaxB), Providencia alcalifaciens (e.g., PaYaxA, PaYaxB), Pseudomonas syringae (e.g., PsYaxA, PsYaxB), Proteus mirabilis (e.g., PmYaxA, PmYaxB), Morganella morganii (e.g., MmYaxA, MmYaxB), Photorhabdus luminescens (e.g., PaxA, PaxB), Xenorhabdus nematophila (e.g., XaxA, XaxB), or any combination thereof. In some embodiments, an extension (e.g., a protein, peptide, or polypeptide extension) can be attached to a truncated subunit originating from Yersinia enterocolitica (e.g., YaxA or YaxB), Providencia alcalifaciens (e.g., PaYaxA, PaYaxB), Pseudomonas syringae (e.g., PsYaxA, PsYaxB), Proteus mirabilis (e.g., PmYaxA, PmYaxB), Morganella morganii (e.g., MmYaxA, MmYaxB), Photorhabdus luminescens (e.g., PaxA, PaxB), Xenorhabdus nematophila (e.g., XaxA, XaxB), or any combination thereof. In some embodiments, an extension (e.g., a protein, peptide, or polypeptide extension) comprising a recognition element and a flexible linker sequence may be used to replace at least part of an unstructured terminal region of a YaxA subunit, a YaxB subunit, or a mutant, functional homolog, functional ortholog, or functional paralog thereof. In some embodiments, an extension (e.g., a protein, peptide, or polypeptide extension) comprising a recognition element and a flexible linker sequence may be used to replace all of an unstructured terminal region of a YaxA subunit, a YaxB subunit, or a mutant, functional homolog, functional ortholog, or functional paralog thereof. In some embodiments, an extension (e.g., a protein, peptide, or polypeptide extension) comprising a recognition element and a flexible linker sequence can be fused to the N- or C-terminus of YaxB or an ortholog thereof. In some aspects, the present disclosure provides a nanopore system comprising YaxAB nanopores wherein at least one YaxB monomer is functionalized (e.g., the YaxAB monomer comprises at least one recognition element). In some aspects, the present disclosure provides a nanopore system comprising YaxAB nanopores wherein two or more YaxB monomers are functionalized, wherein the YaxAB monomers comprise different recognition elements. For example, good results can be obtained wherein YaxB monomers are N- or C-terminally fused to an extension peptide comprising at its “free” terminus a proteinaceous recognition element. See Table 2 for exemplary functionalized YaxB subunits.

In some aspects, the present disclosure provides a functionalized nanopore (e.g., biological nanopore) comprising at least one recognition element conjugated to at least one monomer. In some embodiments, the functionalized nanopore comprises multiple recognition elements that are the same recognition elements. In some embodiments, the functionalized nanopore comprises multiple recognition elements that are different recognition elements. In some embodiments, the monomers of the nanopore comprise the same subunits, and each subunit can be conjugated to at least one recognition element. In some embodiments, the monomers of the nanopore comprise different subunits, and each subunit can be conjugated to at least one recognition element. In some embodiments, a first portion of a subunit can be conjugated to one or more recognition elements. In some embodiments, a second portion of a subunit can be conjugated to one or more recognition elements.

The recognition element may interact with an analyte through non-covalent binding. The recognition element may interact with an analyte through covalent binding. In some embodiments, the recognition element can interact with an analyte through electrostatic interactions, Van der Waals forces, ζ-ζ interactions, or any combination thereof. The recognition element can interact with an analyte through hydrogen bonding and/or halogen binding. In some embodiments, the recognition element can interact with an analyte through dipole-dipole interactions, dipole-induced dipole interactions, London dispersion forces, or any combination thereof.

The recognition element may comprise a small molecule (e.g., biotin). The recognition element can comprise a polynucleotide (e.g., an aptamer). The recognition element may comprise a peptide sequence. For example, the recognition element may comprise a Strep-tag. The recognition element may be polynucleotide-based. In some embodiments, the recognition element can comprise a nanobody or an antibody, or a fragment thereof.

In some embodiments, the recognition element may comprise intrinsic affinity to an analyte, allowing the element to bind to the analyte and capture it within the nanopore. A nanopore comprising a recognition element described herein may be referred to as a functionalized nanopore.

The invention also provides a functionalized YaxA polypeptide, YaxB polypeptide, or a mutant, functional homolog, functional ortholog, or functional paralog thereof, capable of forming a nanopore (e.g., a conical shaped nanopore), the functionalized polypeptide comprising a recognition element capable of specifically binding to an analyte. In some embodiments, the functionalized YaxA and/or YaxB polypeptide may not comprise a recognition element. As described herein above, the recognition element can be of proteinaceous or non-proteinaceous nature, for example the recognition element can be a small-molecule, a protein (folded or unfolded), DNA, RNA, etc. In some cases, the recognition element can be a proteinaceous moiety. The functionalized YaxA polypeptide, YaxB polypeptide, or a mutant, functional homolog, functional ortholog, or functional paralog thereof may comprise a variant, mutant and/or truncated version of YaxA polypeptide, YaxB polypeptide, or a mutant, functional homolog, functional ortholog, or functional paralog thereof as described herein.

In one aspect, the recognition element can be attached to the nanopore (e.g., biological nanopore) via a flexible linker, for example wherein the flexible linker can be a polypeptide, a polynucleotide or any other type of unstructured polymer, such as PEG. In one aspect, the recognition element can be attached to the nanopore via a rigid linker, for example wherein the rigid linker can be a polypeptide, a polynucleotide or any other type of unstructured polymer, such as PEG. In one aspect, the recognition element can be attached to the nanopore via a cleavable linker, for example wherein the cleavable linker can be a polypeptide, a polynucleotide or any other type of unstructured polymer, such as PEG. In some cases, the linker (e.g., a flexible linker, a rigid linker, or a cleavable linker) can be a polypeptide linker, e.g. a polypeptide linker comprising at least 3 amino acids, for example 3 to 100 amino acids, or 10 to 70 amino acids, e.g. 12, 15, 20, 25, 30, 35, 40, 50, 60 or 65 amino acids. In some embodiments, the linker attaching the recognition element to a variant polypeptide of a nanopore described herein can comprise at least about 3 amino acids, at least about 4 amino acids, at least about 5 amino acids, at least about 10 amino acids, at least about 15 amino acids, at least about 20 amino acids, at least about 25 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 125 amino acids, or greater than about 125 amino acids. In some embodiments, the linker attaching the recognition element to a variant polypeptide of a nanopore described herein can comprise at most about 125 amino acids, at most about 100 amino acids, at most about 90 amino acids, at most about 80 amino acids, at most about 70 amino acids, at most about 60 amino acids, at most about 50 amino acids, at most about 40 amino acids, at most about 30 amino acids, at most about 25 amino acids, at most about 20 amino acids, at most about 15 amino acids, at most about 10 amino acids, at most about 5 amino acids, at most about 4 amino acids, at most about 3 amino acids, or less than about 3 amino acids.

In some cases, a recognition element (e.g., a protein, peptide, or polypeptide recognition element) can be genetically fused to the N- and/or C-terminus of an optionally truncated YaxA polypeptide, YaxB polypeptide, or a mutant, functional homolog, functional ortholog, or functional paralog thereof (e.g., forming a functionalized YaxA polypeptide, YaxB polypeptide, or a mutant, functional homolog, functional ortholog, or functional paralog thereof). In some cases, a recognition element (e.g., a protein, peptide, or polypeptide recognition element) can be fused to said YaxA polypeptide, YaxB polypeptide, or a mutant, functional homolog, functional ortholog, or functional paralog thereof via a linker.

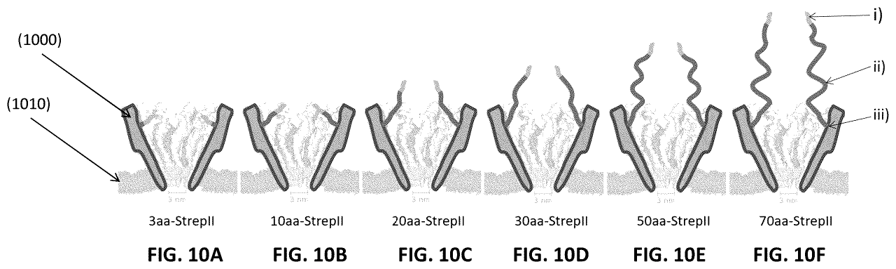

As show in FIGS. 10A-10F, a length of a linker may be modified and modification of a linker length may assist in capture and/or retention of an analyte in a nanopore. A linker (ii) may be attached to a first opening (iii) nanopore (1000) and further attached to a recognition element (i). The linker can be increased in length (e.g., number of amino acid residues) and the recognition element may remain the same length. The linker can be increased in length (e.g., number of amino acid residues) and the recognition element may also increase in length. The linker can be increased in length (e.g., number of amino acid residues) and the recognition element may decrease in length. The linker may attached on an outer edge of a first opening (iii). The linker may be attached on an inner edge (e.g., within a channel of a nanopore).

In some embodiments, a functionalized N-truncated YaxA subunit may comprise one or more of the mutations of a YaxA subunit as described herein. In some embodiments, a functionalized N-truncated YaxA subunit may comprise one or more of the mutations R150G, K250R, or S282G with respect to the sequence of SEQ ID NO: 25 (ProteinID YE1984). In some embodiments, a functionalized non-truncated YaxA subunit may comprise one or more of the mutations R150G, K250R, or S282G with respect to the sequence of SEQ ID NO: 25 (ProteinID YE1984). In some embodiments, a functionalized nanopore described herein may comprise at least one YaxA subunit comprising one or more of the mutations R150G, K250R, or S282G with respect to the sequence of SEQ ID NO: 25 (ProteinID YE1984) and at least one of wild-type YaxA subunit.

In some embodiments, a functionalized N-truncated YaxA subunit may comprise a mutation at position N17 of the wild-type YaxA sequence as set forth in SEQ ID NO: 25 (ProteinID YE1984). In some embodiments, a functionalized non-truncated YaxA subunit may comprise a mutation at position N17 of the wild-type YaxA sequence as set forth in SEQ ID NO: 25 (ProteinID YE1984). In some embodiments, a mutation at amino acid position N17 of the wild-type YaxA sequence as set forth in SEQ ID NO: 25 (ProteinID YE1984) may comprise substitution to a positively-charged amino acid residue, a negatively-charged amino acid residue, a neutral amino acid residue, a hydrophobic amino acid residue, or a hydrophilic amino acid residue. In some embodiments, a non-truncated YaxA subunit may comprise the mutation N17S with respect to the sequence of SEQ ID NO: 25 (ProteinID YE1984). In some embodiments, a nanopore described herein may comprise at least one YaxA subunit comprising the mutation N17S with respect to the sequence of SEQ ID NO: 25 (ProteinID YE1984) and at least one of wild-type YaxA subunit.

In some cases, a YaxA subunit of a functionalized nanopore described herein can comprise a mutation comprising R150G, K250R, S282G, or N17S, or any combination thereof, with numbering respect to the sequence set forth in SEQ ID NO: 25 (ProteinID YE1984). In some embodiments, a functionalized nanopore described herein may comprise at least one YaxA subunit comprising one or more of the mutations R150G, K250R, S282G, or N17S, with respect to the sequence of SEQ ID NO: 25 (ProteinID YE1984) and at least one of wild-type YaxA subunit.

In some embodiments, a functionalized N-truncated YaxB subunit may comprise one or more of the mutations of a YaxB subunit as described herein. In some embodiments, a functionalized N-truncated YaxB subunit may comprise a mutation at position 284 of the wild-type YaxB sequence as set forth in SEQ ID NO: 26 (ProteinID YE1985). In some embodiments, a functionalized non-truncated YaxB subunit may comprise a mutation at position 284 of the wild-type YaxB sequence as set forth in SEQ ID NO: 26 (ProteinID YE1985). In some embodiments, a functionalized N-truncated YaxB subunit may comprise a mutation at position V284 of the wild-type YaxB sequence as set forth in SEQ ID NO: 26 (ProteinID YE1985). In some embodiments, a non-truncated YaxB subunit may comprise a mutation at position V284 of the wild-type YaxB sequence as set forth in SEQ ID NO: 26 (ProteinID YE1985). In some embodiments, a mutation at amino acid position V284 of the wild-type YaxB sequence as set forth in SEQ ID NO: 26 (ProteinID YE1985) may comprise a substitution to glycine (G), alanine (A), isoleucine (I), leucine (L), proline (P), arginine (R), or serine (S). In some embodiments, a mutation at amino acid position V284 of the wild-type YaxB sequence as set forth in SEQ ID NO: 26 (ProteinID YE1985) may comprise substitution to a positively-charged amino acid residue, a negatively-charged amino acid residue, a neutral amino acid residue, a hydrophobic amino acid residue, or a hydrophilic amino acid residue. In some embodiments, a functionalized nanopore described herein comprises at least one variant YaxB subunit comprising a mutation V284I, wherein the residue numbering corresponds to SEQ ID NO: 26 (ProteinID YE1985). The nanopores, methods, and systems provided herein comprise conically shaped nanopore comprising at least one variant YaxA polypeptide, YaxB polypeptide, or a mutant, functional homolog, functional ortholog, or functional paralog thereof to which a recognition element capable of specifically binding to a target analyte can be attached (e.g. attached by chemical attachment, genetic fusion, and/or linker moiety). The nanopores (e.g., comprising monomers and subunits) may comprise any mutation. The mutation can be a point mutation, a silent mutation, a missense mutation, a nonsense mutation, a frameshift mutation, a truncation, or any combination thereof.

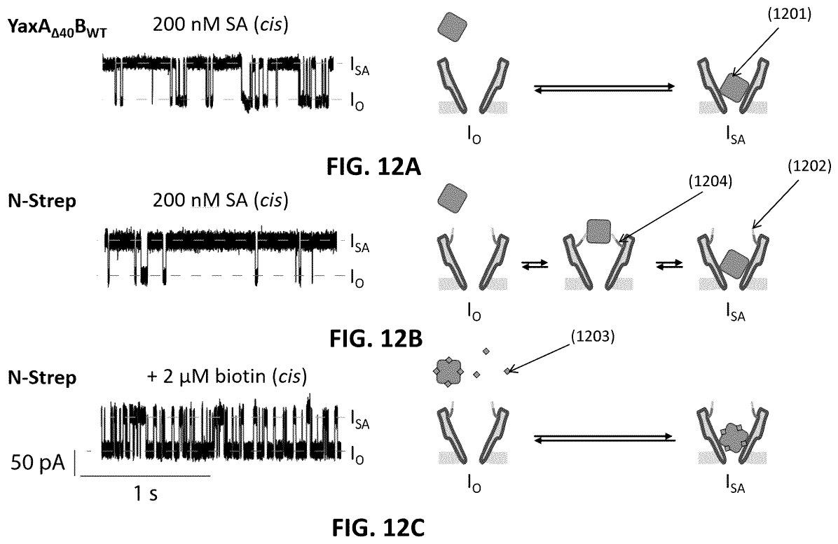

In some embodiments, a recognition element can assist in the capture of an analyte. The recognition element may provide a benefit to the nanopores, nanopore systems, methods, or any combination thereof by prolonging a dwell time of an analyte in the nanopore and allowing for longer characterization. FIGS. 12A-12C demonstrate the effect of a recognition element on a current output. For example, FIG. 12A shows an analyte, Streptavidin A (SA) being reversibly captured by a nanopore (YaxA_{Δ40}B_{WT}) in which the N-terminal of the YaxA subunit(s) of the nanopore are truncated by 40 amino acid residues and the YaxB subunit(s) are wild-type. The current output shows a I_{O} current, designating the open-pore. Once the analyte (e.g., SA) occupies the pore, the current displays peaks for the blockage current (I_{SA}) designating the captured analyte. In FIG. 12B, the nanopore comprises a recognition element comprising a peptide sequence (e.g., a Strep-tag) which has an affinity for the analyte. The Strep-tag can be attached to a N-terminal (e.g., N-Strep). The recognition elements can capture the analyte and prolong the dwell time and residence in the nanopore. This can then lead to sustained I_{SA} current and less open-pore current. FIG. 12C shows the addition of biotin to the analyte. As the biotin occupies the same binding sites as the Strep-tag, the analyte may not be bound to the recognition element and there may be a reduction in I_{SA} current. In some embodiments, the resulting current from FIG. 12C may comprise current from the conjugated biotin that can be characterized by the nanopore. The conjugation of biotin may increase or decrease a dwell time of the analyte (e.g., SA) in the pore which may reduce I_{SA} current in the current signal.

The functionalized nanopore-forming subunit advantageously comprises one or more additional sequences (motifs) that can aid in the (recombinant) production and/or purification of the variant polypeptide. These include protein purification tags, e.g. His6-tag, Strep-tag, SUMO tag, MBP tag, etc. and protease cleavage sites, such as tobacco etch virus (TEV) protease cleavage site. The additional motifs can be separated by a spacer. A spacer may comprise 1, 2, 3, 4, 5, 6, 7, 8, 9, 10, or more amino acids.

The nanopores, methods, and systems provided herein may comprise an isolated nucleic acid molecule encoding a functionalized subunit of a pore described herein. The nucleic acid molecule may encode a subunit originating from Yersinia enterocolitica (e.g., YaxA or YaxB), Providencia alcalifaciens (e.g., PaYaxA, PaYaxB), Pseudomonas syringae (e.g., PsYaxA, PsYaxB), Proteus mirabilis (e.g., PmYaxA, PmYaxB), Morganella morganii (e.g., MmYaxA, MmYaxB), Photorhabdus luminescens (e.g., PaxA, PaxB), Xenorhabdus nematophila (e.g., XaxA, XaxB), or any combination thereof. The nucleic acid molecule may comprises a sequence combination encoding a subunit of a nanopore and a recognition element described herein. In some embodiments, the present disclosure provides an isolated nucleic acid molecule encoding a functionalized and/or mutated YaxA polypeptide, YaxB polypeptide, or a mutant, functional homolog, functional ortholog, or functional paralog thereof as described herein.

Also provided is an expression vector comprising the nucleic acid molecule, and a host cell comprising such expression vector. In some aspects, the present disclosure provides nucleic acid molecules encoding nanopores and/or subunits of nanopores described herein. Nucleic acid sequences may encode a subunit of a monomer originating from Yersinia enterocolitica (e.g., YaxA or YaxB), Providencia alcalifaciens (e.g., PaYaxA, PaYaxB), Pseudomonas syringae (e.g., PsYaxA, PsYaxB), Proteus mirabilis (e.g., PmYaxA, PmYaxB), Morganella morganii (e.g., MmYaxA, MmYaxB), Photorhabdus luminescens (e.g., PaxA, PaxB), Xenorhabdus nematophila (e.g., XaxA, XaxB), or any combination thereof. In some embodiments, nucleic acid sequences may encode the YaxA or YaxB subunit to a nanopore. In some aspects, the present disclosure provides host cells and/or vectors containing the nucleic acids described herein. The nucleic acids may be present in a single vector or separate vectors. The vector or separate vectors may be present in the same host cell or separate host cell. The vector system may comprise bovine papilloma virus, polyoma virus, adenovirus, vaccinia virus, baculovirus, retroviruses (Rous Sarcoma Virus, MMTV or MOMLV) or SV40 virus. Once the expression vector or DNA sequence containing the pore construct has been prepared for expression, the expression vectors may be transfected or introduced into an appropriate host cell. In some embodiments, the host cells may be genetically engineered to comprise nucleic acid molecules encoding the pores (e.g., nanopores or conical nanopores) described herein.

### Systems

In some aspects, the present disclosure provides a sensor system comprising a pore (e.g., a nanopore). In some aspects, the present disclosure provides a sensor system comprising a nanopore embedded in a membrane. In some cases, the membrane can be an amphipathic membrane. In some cases, the membrane can be a hydrophobic membrane. In some cases, the membrane can separate a chamber into a first side and a second side. In some embodiments, the chamber can be a fluid filled chamber. In some cases, the membrane can comprise at least one nanopore. Disclosed herein is a sensor system comprising a proteinaceous nanopore embedded in an amphipathic or hydrophobic membrane separating a fluid filled chamber into at least two sides (e.g., chambers). In some embodiments, one side (e.g., a first side) of a fluid filled chamber can be a cis side and another side (e.g., a second side) of a fluid filled chamber can be a trans side. In some embodiments, the nanopore can be a conical shaped proteinaceous nanopore. In some embodiments, the nanopore can be a cylindrical shaped proteinaceous nanopore. In some embodiments, the nanopore can be a conical shaped proteinaceous nanopore having two openings (e.g., entrances). The nanopore may comprise an opening on a first side (e.g., a cis side) of a fluid filled chamber (e.g., a cis opening). The nanopore may comprise an opening on a second side (e.g., a trans side) of a fluid filled chamber (e.g., a trans opening).

A sensor system according to the invention is not taught or suggested in the art. Brauning et al. (Nature Communications Vol. 9, 1806 (2018)) disclosed the crystal structures of YaxA and YaxB, together with a cryo-electron microscopy map of the YaxAB complex. The structures revealed a pore predominantly composed of decamers of YaxA-YaxB heterodimers. Plotting of the pore diameter against the coordinate along the vertical axis of a fitted pore model of YaxA and B monomers revealed a narrowest construction of about 31 Å. Negative-stain TEM micrographs of YaxAB complexes in solutions distinguishes an upper, spoked rim from which density converges at a lower, cup-like funnel. Notably however, the cryo-EM images showed areas resembling an open and “leaky” basket structure. In no way these images could have predicted that YaxAB could be assembled into stable and functional, conductive conical nanopores for use in analyte sensing as disclosed in the present invention. Cryo-EM also showed that the N-terminus of YaxA and the N- and C-termini of YaxB point towards the interior of the nanopore. The first 40 residues of YaxA, however, are not observed in Cryo-EM images, strongly suggesting that they form an unstructured region of the nanopore. It was unknown, therefore, what effect these “polypeptide tails” might have on (protein) analytes lodged inside the nanopore.

According to the invention, a sensor system comprises a conical shaped proteinaceous nanopore embedded in an amphipathic or hydrophobic membrane. In some aspects, the present disclosure provides a sensor system comprising a pore. In some embodiments, the pore can be a nanopore. The nanopore can be conical shaped. The nanopore can be cylindrical shaped. The term “membrane” used herein in its conventional sense can refer to a thin, film-like structure that separates the chamber of the system into a first side (e.g., a cis side or cis compartment) and a second side (e.g., a trans side or trans compartment). The membrane separating the first and second sides can comprise at least one pore (e.g., a biological nanopore). The pore may be a nanopore. The nanopore may be conical shaped. Membranes can be generally classified into synthetic membranes and biological membranes. Any membrane may be used in accordance with the invention. Multiple nanopores may be present in one membrane. In some embodiments, a membrane of a nanopore system described herein may comprise at least about, at most about, or about 1, 2, 3, 4, 5, 6, 7, 8, 9, 10, 20, 30, 40, 50, 60, 70, 80, 90, 100, 200, 300, 400, 500, 600, 700, 800, 900, 1000, 5000, 10000 nanopores, or any number of nanopores between two of these values.

The membrane can be an amphiphilic layer. An amphiphilic layer can refer to a layer formed from amphiphilic molecules, such as phospholipids, which have both at least one hydrophilic portion and at least one lipophilic or hydrophobic portion. The amphiphilic layer may be a monolayer or a bilayer. The amphiphilic molecules may be synthetic or naturally occurring. In some embodiments, the membrane may comprise multiple layers. In some embodiments, the membrane may be functionalized. In some embodiments, the membrane may be functionalized with a thiol group, a peptide, a nucleic acid, a biomolecule, or combinations thereof. Non-naturally occurring amphiphiles which form a monolayer are known in the art and include, for example, block copolymers (Gonzalez-Perez et al., Langmuir, 2009, 25, 10447-10450). The block copolymers can comprise decane and show low ionic conductance and increased longevity of use.

In some embodiments, a membrane of a system described herein may comprise a thickness. In some embodiments, a membrane may be at least about 0.5 nm, 1 nm, 2 nm, 3 nm, 4 nm, 5 nm, 6 nm, 7 nm, 8 nm, 9 nm, 10 nm, 12 nm, 14 nm, 16 nm, 18 nm, 20 nm, 25 nm, 30 nm, 40 nm, 50 nm, 60 nm, 70 nm, 80 nm, 90 nm, 100 nm, 110 nm, 120 nm, 130 nm, 140 nm, 150 nm, or greater than about 150 nm thick. In some embodiments, a membrane comprise a thickness from about 0.5 nm to about 100 nm. In some embodiments, a membrane comprise a thickness from about 0.5 nm to about 1 nm, about 0.5 nm to about 2 nm, about 0.5 nm to about 3 nm, about 0.5 nm to about 4 nm, about 0.5 nm to about 5 nm, about 0.5 nm to about 10 nm, about 0.5 nm to about 20 nm, about 0.5 nm to about 30 nm, about 0.5 nm to about 40 nm, about 0.5 nm to about 50 nm, about 0.5 nm to about 100 nm, about 1 nm to about 2 nm, about 1 nm to about 3 nm, about 1 nm to about 4 nm, about 1 nm to about 5 nm, about 1 nm to about 10 nm, about 1 nm to about 20 nm, about 1 nm to about 30 nm, about 1 nm to about 40 nm, about 1 nm to about 50 nm, about 1 nm to about 100 nm, about 2 nm to about 3 nm, about 2 nm to about 4 nm, about 2 nm to about 5 nm, about 2 nm to about 10 nm, about 2 nm to about 20 nm, about 2 nm to about 30 nm, about 2 nm to about 40 nm, about 2 nm to about 50 nm, about 2 nm to about 100 nm, about 3 nm to about 4 nm, about 3 nm to about 5 nm, about 3 nm to about 10 nm, about 3 nm to about 20 nm, about 3 nm to about 30 nm, about 3 nm to about 40 nm, about 3 nm to about 50 nm, about 3 nm to about 100 nm, about 4 nm to about 5 nm, about 4 nm to about 10 nm, about 4 nm to about 20 nm, about 4 nm to about 30 nm, about 4 nm to about 40 nm, about 4 nm to about 50 nm, about 4 nm to about 100 nm, about 5 nm to about 10 nm, about 5 nm to about 20 nm, about 5 nm to about 30 nm, about 5 nm to about 40 nm, about 5 nm to about 50 nm, about 5 nm to about 100 nm, about 10 nm to about 20 nm, about 10 nm to about 30 nm, about 10 nm to about 40 nm, about 10 nm to about 50 nm, about 10 nm to about 100 nm, about 20 nm to about 30 nm, about 20 nm to about 40 nm, about 20 nm to about 50 nm, about 20 nm to about 100 nm, about 30 nm to about 40 nm, about 30 nm to about 50 nm, about 30 nm to about 100 nm, about 40 nm to about 50 nm, about 40 nm to about 100 nm, or about 50 nm to about 100 nm.

The nanopore system typically comprises a first side (e.g., cis side) comprising a first conductive liquid medium in liquid communication with a second side (e.g., trans side) comprising a second conductive liquid medium. The conductive liquid medium in the chambers of the nanopore system can have a wide range of ionic contents well known in the art, typically from 0.05 M to >3 M. A wide range of salts can be used, such as NaCl and KCl. Suitable solutions include 150 mM NaCl, 50 mM Tris-HCl, pH 7.5. In some embodiments, a salt, ion, osmolyte, or electrolyte concentration on the cis side can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the cis side can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the cis side can be from about 0.01 M to about 5 M. In some embodiments, a salt, ion, osmolyte, or electrolyte concentration on the cis side can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the cis side can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

The solution or solutions may have a pH of at least about 1, at least about 2, at least about 3, at least about 3.8, at least about 4, at least about 4.5, at least about 6, at least about 7, at least about 8, at least about 9, at least about 10, at least about 10.5 at least about 11, at least about 12, at least about 13, or greater than about 13 that can be employed. The solution or solutions may have a pH of at most about 13, at most about 12, at most about 11, at most about 10.5, at most about 10, at most about 9, at most about 8, at most about 7, at most about 6, at most about 4.5, at most about 4, at most about 3.8, at most about 3, at most about 2, at most about 1, or less than about 1 that can be employed.

The solution or solutions may have a pH from about 1 to about 13 that can be employed. The solution or solutions may have a pH from about 1 to about 2, about 1 to about 3, about 1 to about 4, about 1 to about 6, about 1 to about 7, about 1 to about 8, about 1 to about 9, about 1 to about 10, about 1 to about 11, about 1 to about 12, about 1 to about 13, about 2 to about 3, about 2 to about 4, about 2 to about 6, about 2 to about 7, about 2 to about 8, about 2 to about 9, about 2 to about 10, about 2 to about 11, about 2 to about 12, about 2 to about 13, about 3 to about 4, about 3 to about 6, about 3 to about 7, about 3 to about 8, about 3 to about 9, about 3 to about 10, about 3 to about 11, about 3 to about 12, about 3 to about 13, about 4 to about 6, about 4 to about 7, about 4 to about 8, about 4 to about 9, about 4 to about 10, about 4 to about 11, about 4 to about 12, about 4 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13 that can be employed.

The solution or solutions may have a pH of about 1, about 2, about 3, about 3.8, about 4, about 4.5, about 6, about 7, about 8, about 9, about 10, about 10.5 about 11, about 12, or about 13 that can be employed.

The first side and second side may be symmetric or asymmetric. A wide range of pH and temperature conditions can be used, for example in the range of pH 3-11, 10-80° C., for example at about room temperature or at about 37° C. In some embodiments, a cis chamber and/or a trans chamber may have a pH of at least about 1, at least about 2, at least about 3, at least about 3.8, at least about 4, at least about 4.5, at least about 6, at least about 7, at least about 8, at least about 9, at least about 10, at least about 10.5 at least about 11, at least about 12, at least about 13, or greater than about 13. In some embodiments, a first side and/or second side may have a pH of at most about 13, at most about 12, at most about 11, at most about 10.5, at most about 10, at most about 9, at most about 8, at most about 7, at most about 6, at most about 4.5, at most about 4, at most about 3.8, at most about 3, at most about 2, at most about 1, or less than about 1. In some embodiments, a cis chamber and/or a trans chamber may have a pH from about 1 to about 13 that can be employed. In some embodiments, a first side and/or second side may have a pH from about 1 to about 2, about 1 to about 3, about 1 to about 4, about 1 to about 6, about 1 to about 7, about 1 to about 8, about 1 to about 9, about 1 to about 10, about 1 to about 11, about 1 to about 12, about 1 to about 13, about 2 to about 3, about 2 to about 4, about 2 to about 6, about 2 to about 7, about 2 to about 8, about 2 to about 9, about 2 to about 10, about 2 to about 11, about 2 to about 12, about 2 to about 13, about 3 to about 4, about 3 to about 6, about 3 to about 7, about 3 to about 8, about 3 to about 9, about 3 to about 10, about 3 to about 11, about 3 to about 12, about 3 to about 13, about 4 to about 6, about 4 to about 7, about 4 to about 8, about 4 to about 9, about 4 to about 10, about 4 to about 11, about 4 to about 12, about 4 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13 that can be employed.

In some embodiments, a first side and/or second side may have a temperature of at least about 5° C., at least about 10° C., at least about 15° C., at least about 20° C., at least about 25° C., at least about 30° C., at least about 35° C., at least about 40° C., at least about 45° C., at least about 50° C., at least about 60° C., at least about 70° C., at least about 80° C., or greater than about 80° C. In some embodiments, a first side and/or second side may have a temperature of at most about 80° C., at most about 70° C., at most about 60° C., at most about 50° C., at most about 45° C., at most about 40° C., at most about 35° C., at most about 30° C., at most about 25° C., at most about 20° C., at most about 15° C., at most about 10° C., at most about 5° C., or less than about 5° C. In some embodiments, a first side and/or second side may have a temperature from about 5° C. to about 80 C. In some embodiments, a first side and/or second side may have a temperature from about 5° C. to about 10° C., about 5° C. to about 15° C., about 5° C. to about 20° C., about 5° C. to about 25° C., about 5° C. to about 30° C., about 5° C. to about 35° C., about 5° C. to about 40° C., about 5° C. to about 50° C., about 5° C. to about 60° C., about 5° C. to about 70° C., about 5° C. to about 80° C., about 10° C. to about 15° C., about 10° C. to about 20° C., about 10° C. to about 25° C., about 10° C. to about 30° C., about 10° C. to about 35° C., about 10° C. to about 40° C., about 10° C. to about 50° C., about 10° C. to about 60° C., about 10° C. to about 70° C., about 10° C. to about 80° C., about 15° C. to about 20° C., about 15° C. to about 25° C., about 15° C. to about 30° C., about 15° C. to about 35° C., about 15° C. to about 40° C., about 15° C. to about 50° C., about 15° C. to about 60° C., about 15° C. to about 70° C., about 15° C. to about 80° C., about 20° C. to about 25° C., about 20° C. to about 30° C., about 20° C. to about 35° C., about 20° C. to about 40° C., about 20° C. to about 50° C., about 20° C. to about 60° C., about 20° C. to about 70° C., about 20° C. to about 80° C., about 25° C. to about 30° C., about 25° C. to about 35° C., about 25° C. to about 40° C., about 25° C. to about 50° C., about 25° C. to about 60° C., about 25° C. to about 70° C., about 25° C. to about 80° C., about 30° C. to about 35° C., about 30° C. to about 40° C., about 30° C. to about 50° C., about 30° C. to about 60° C., about 30° C. to about 70° C., about 30° C. to about 80° C., about 35° C. to about 40° C., about 35° C. to about 50° C., about 35° C. to about 60° C., about 35° C. to about 70° C., about 35° C. to about 80° C., about 40° C. to about 50° C., about 40° C. to about 60° C., about 40° C. to about 70° C., about 40° C. to about 80° C., about 50° C. to about 60° C., about 50° C. to about 70° C., about 50° C. to about 80° C., about 60° C. to about 70° C., about 60° C. to about 80° C., or about 70° C. to about 80 C.

Suitably, the first side (e.g., cis side) may comprise a crowding or blocking agent that reduces unwanted nonspecific protein adsorption. The blocking agent may comprise a soluble globular protein. For example, the blocking agent may comprise bovine serum albumin (BSA) and/or transferrin. The blocking agent may comprise a microbead, nanobead, or any combination thereof. For example, the blocking agent can comprise a polymeric bead, an organic bead, or any combination thereof. The blocking agent can comprise polymers (e.g., linear and/or dendrimer forms). For example, a blocking agent described herein may comprise polyethylene glycol (PEG), fycol, dextran, polyacrylamides, or any combination thereof.

The system may comprise a circuit that can both apply the voltage and measure the current. Alternatively, it comprises one circuit to apply the voltage difference and another to measure the current. It is also possible to create the voltage difference with an asymmetric salt across the membrane. For example, one of the chambers may contain a solution of high ionic strength. Exemplary means for detecting the current between the cis and trans chambers were described in WO 00/79257 Patent Application No. 6,46,594, 6,673, 6,673,615, 6,627,067, 6,464,842, 6,362,002, 6,267,872, 6,015,714, 6,428,959, 6,617,113 and 5,795,782 and US Publications Nos. 2004/0121525, 2003/0104428 and 2003/0104428. They may include electrodes directly associated with the channel or pore at or near the porous opening, electrodes placed within the cis and trans chambers, and insulated glass microelectrodes. Electrodes may be capable of, detecting differences in ionic current around two chambers or tunneling electrical current around the porous opening. In another configuration, the transport property can be the flow of electrons around the diameter of the aperture which can be monitored by electrodes placed adjacent to or touching the circumference of the nanopore. Said electrodes can be attached to an Axopatch 200B amplifier to amplify a signal.

The sensor systems described herein may not be limited and that other systems for acquiring or measuring nanopore signals can be employed, including optical sensing. Optical sensing can be used to measure the wavelength output from nanopore sequencing, using fluorophores or other light-focused markers. Alternative electrical schemes can also be employed, on arrayed chip platforms for example, to achieve an equivalent voltage drop across the nanopore and/or membrane.

The sensor system can be advantageously integrated in a portable device comprising a plurality of sensor systems. In some embodiments, a portable device may comprise 1, 2, 3, 4, 5, 6, 7, 8, 9, 10, 15, 20, or more of the sensor systems described herein. Each sensor system may be configured to characterize the same analyte. Sensor systems within a portable device may be configured to characterize different analytes. Analytes may differ on size, length, weight, pH, charge, chemical composition, or any combination thereof. For example, the system may be comprised in a point-of-care diagnostic medical devices, which are in vitro diagnostics used by health care professionals to obtain results rapidly near or at the site of a patient. These products can be useful to quickly determine a marker responsible for a certain disease, e.g., at a doctor's office or clinic. The device can be designed for performing an analytical method as herein disclosed. The device can be a portable device, a medical device, implant, single use device, or a disposable device. In one aspect, the device can be configured to allow for real-time detection of at least one analyte, for example a clinically relevant analyte. Real-time detection may comprise detecting an analyte within 2000, 1000, 750, 500, 250, 100, 90, 80, 70, 60, 50, 40, 30, 20, 10, or less than 10 ms from applying the analyte to the device (e.g., applying the analyte to the sensor system). A clinically relevant analyte may comprise a protein or peptide sample from any disease or condition (e.g., an infectious disease, a cancer, or an autoimmune disease). A clinically relevant analyte may comprise a protein or peptide sample from a viral or bacterial pathogen.

The nanopore systems described herein may characterize a number of analytes. In some embodiments, a nanopore system described herein may characterize at least about 2 analytes, at least about 3 analytes, at least about 4 analytes, at least about 5 analytes, at least about 6 analytes, at least about 7 analytes, at least about 8 analytes, at least about 9 analytes, at least about 10 analytes, at least about 20 analytes, at least about 30 analytes, at least about 40 analytes, at least about 50 analytes, at least about 100 analytes, at least about 200 analytes, at least about 300 analytes, at least about 400 analytes, at least about 500 analytes, at least about 600 analytes, at least about 700 analytes, at least about 800 analytes, at least about 900 analytes, at least about 1000 analytes, at least about 1500 analytes, at least about 2000 analytes, at least about 2500 analytes, at least about 3000 analytes, at least about 3500 analytes, at least about 4000 analytes, at least about 4500 analytes, at least about 5000 analytes, at least about 5500 analytes, at least about 6000 analytes, at least about 6500 analytes, at least about 7000 analytes, at least about 7500 analytes, at least about 8000 analytes, at least about 8500 analytes, at least about 9000 analytes, at least about 9500 analytes, at least about 10000 analytes, or greater than about 10000 analytes. In some embodiments, a nanopore system described herein may characterize at most about 10000 analytes, at most about 9500 analytes, at most about 9000 analytes, at most about 8500 analytes, at most about 8000 analytes, at most about 7500 analytes, at most about 7000 analytes, at most about 6500 analytes, at most about 6000 analytes, at most about 5500 analytes, at most about 5000 analytes, at most about 4500 analytes, at most about 4000 analytes, at most about 3500 analytes, at most about 3000 analytes, at most about 2500 analytes, at most about 2000 analytes, at most about 1500 analytes, at most about 1000 analytes, at most about 900 analytes, at most about 800 analytes, at most about 700 analytes, at most about 600 analytes, at most about 500 analytes, at most about 400 analytes, at most about 300 analytes, at most about 200 analytes, at most about 100 analytes, at most about 90 analytes, at most about 80 analytes, at most about 70 analytes, at most about 60 analytes, at most about 50 analytes, at most about 40 analytes, at most about 30 analytes, at most about 20 analytes, at most about 10 analytes, at most about 9 analytes, at most about 8 analytes, at most about 7 analytes, at most about 6 analytes, at most about 5 analytes, at most about 4 analytes, at most about 3 analytes, at most about 2 analytes, or less than about 2 analytes.

In some embodiments, a nanopore system described herein may characterize from about 2 analytes to about 100 analytes may be characterized. In some embodiments, from about 2 analytes to about 5 analytes, about 2 analytes to about 10 analytes, about 2 analytes to about 20 analytes, about 2 analytes to about 30 analytes, about 2 analytes to about 40 analytes, about 2 analytes to about 50 analytes, about 2 analytes to about 60 analytes, about 2 analytes to about 70 analytes, about 2 analytes to about 80 analytes, about 2 analytes to about 90 analytes, about 2 analytes to about 100 analytes, about 5 analytes to about 10 analytes, about 5 analytes to about 20 analytes, about 5 analytes to about 30 analytes, about 5 analytes to about 40 analytes, about 5 analytes to about 50 analytes, about 5 analytes to about 60 analytes, about 5 analytes to about 70 analytes, about 5 analytes to about 80 analytes, about 5 analytes to about 90 analytes, about 5 analytes to about 100 analytes, about 10 analytes to about 20 analytes, about 10 analytes to about 30 analytes, about 10 analytes to about 40 analytes, about 10 analytes to about 50 analytes, about 10 analytes to about 60 analytes, about 10 analytes to about 70 analytes, about 10 analytes to about 80 analytes, about 10 analytes to about 90 analytes, about 10 analytes to about 100 analytes, about 20 analytes to about 30 analytes, about 20 analytes to about 40 analytes, about 20 analytes to about 50 analytes, about 20 analytes to about 60 analytes, about 20 analytes to about 70 analytes, about 20 analytes to about 80 analytes, about 20 analytes to about 90 analytes, about 20 analytes to about 100 analytes, about 30 analytes to about 40 analytes, about 30 analytes to about 50 analytes, about 30 analytes to about 60 analytes, about 30 analytes to about 70 analytes, about 30 analytes to about 80 analytes, about 30 analytes to about 90 analytes, about 30 analytes to about 100 analytes, about 40 analytes to about 50 analytes, about 40 analytes to about 60 analytes, about 40 analytes to about 70 analytes, about 40 analytes to about 80 analytes, about 40 analytes to about 90 analytes, about 40 analytes to about 100 analytes, about 50 analytes to about 60 analytes, about 50 analytes to about 70 analytes, about 50 analytes to about 80 analytes, about 50 analytes to about 90 analytes, about 50 analytes to about 100 analytes, about 60 analytes to about 70 analytes, about 60 analytes to about 80 analytes, about 60 analytes to about 90 analytes, about 60 analytes to about 100 analytes, about 70 analytes to about 80 analytes, about 70 analytes to about 90 analytes, about 70 analytes to about 100 analytes, about 80 analytes to about 90 analytes, about 80 analytes to about 100 analytes, or about 90 analytes to about 100 analytes.

In some embodiments, a nanopore system described herein may characterize from about 100 analytes to about 10,000 analytes. In some embodiments, a nanopore system described herein may characterize from about 100 analytes to about 200 analytes, about 100 analytes to about 300 analytes, about 100 analytes to about 400 analytes, about 100 analytes to about 500 analytes, about 100 analytes to about 750 analytes, about 100 analytes to about 1,000 analytes, about 100 analytes to about 2,500 analytes, about 100 analytes to about 5,000 analytes, about 100 analytes to about 7,500 analytes, about 100 analytes to about 10,000 analytes, about 200 analytes to about 300 analytes, about 200 analytes to about 400 analytes, about 200 analytes to about 500 analytes, about 200 analytes to about 750 analytes, about 200 analytes to about 1,000 analytes, about 200 analytes to about 2,500 analytes, about 200 analytes to about 5,000 analytes, about 200 analytes to about 7,500 analytes, about 200 analytes to about 10,000 analytes, about 300 analytes to about 400 analytes, about 300 analytes to about 500 analytes, about 300 analytes to about 750 analytes, about 300 analytes to about 1,000 analytes, about 300 analytes to about 2,500 analytes, about 300 analytes to about 5,000 analytes, about 300 analytes to about 7,500 analytes, about 300 analytes to about 10,000 analytes, about 400 analytes to about 500 analytes, about 400 analytes to about 750 analytes, about 400 analytes to about 1,000 analytes, about 400 analytes to about 2,500 analytes, about 400 analytes to about 5,000 analytes, about 400 analytes to about 7,500 analytes, about 400 analytes to about 10,000 analytes, about 500 analytes to about 750 analytes, about 500 analytes to about 1,000 analytes, about 500 analytes to about 2,500 analytes, about 500 analytes to about 5,000 analytes, about 500 analytes to about 7,500 analytes, about 500 analytes to about 10,000 analytes, about 750 analytes to about 1,000 analytes, about 750 analytes to about 2,500 analytes, about 750 analytes to about 5,000 analytes, about 750 analytes to about 7,500 analytes, about 750 analytes to about 10,000 analytes, about 1,000 analytes to about 2,500 analytes, about 1,000 analytes to about 5,000 analytes, about 1,000 analytes to about 7,500 analytes, about 1,000 analytes to about 10,000 analytes, about 2,500 analytes to about 5,000 analytes, about 2,500 analytes to about 7,500 analytes, about 2,500 analytes to about 10,000 analytes, about 5,000 analytes to about 7,500 analytes, about 5,000 analytes to about 10,000 analytes, or about 7,500 analytes to about 10,000 analytes.

In some embodiments, a nanopore system described herein may characterize about 2 analytes, about 3 analytes, about 4 analytes, about 5 analytes, about 6 analytes, about 7 analytes, about 8 analytes, about 9 analytes, about 10 analytes, about 20 analytes, about 30 analytes, about 40 analytes, about 50 analytes, about 100 analytes, about 200 analytes, about 300 analytes, about 400 analytes, about 500 analytes, about 600 analytes, about 700 analytes, about 800 analytes, about 900 analytes, about 1000 analytes, about 1500 analytes, about 2000 analytes, about 2500 analytes, about 3000 analytes, about 3500 analytes, about 4000 analytes, about 4500 analytes, about 5000 analytes, about 5500 analytes, about 6000 analytes, about 6500 analytes, about 7000 analytes, about 7500 analytes, about 8000 analytes, about 8500 analytes, about 9000 analytes, about 9500 analytes, or about 10000 analytes.

The system described herein can comprise a sensor or an array of sensors. The system can comprise an electrical energy source and two or more electrodes. In some embodiments, the system comprises a pair of electrodes. In some embodiments, an electrode may be disposed on a first side (e.g., a cis side) of the membrane of a sensor, and another electrode (e.g., a second electrode) may be disposed on a second side (e.g., a trans side). The electrical energy source can apply a potential between the two electrodes, which can cause ions in an electrolyte to conduct through the fluid, and through the pore of the sensor. The applied potential can also cause an analyte, if charged, to translocate to the pore and reside in the pore. The applied potential can create an electrophoretic force (EPF), which can provide a driving force for an analyte to translocate to the pore in order to generate a change in signal. The sensor system may further comprise two or more additional electrodes. For example, these electrodes can be configured to measure the electrical potential across the nanopore and/or membrane that changes when an analyte translocates to a pore. These electrodes can be configured to measure the current across a membrane as an analyte translocates to a pore and reside in a pore (e.g., in a constriction region of a pore). The sensor system can be in electrical communication with a recording device to record measured signals. The system can be in electrical communication with a computer or a processor (e.g., a circuit, an integrated circuit, etc.), which can receive a signal from the sensor or the array of sensors, store the signals in digital form, and/or process the signal.

In some embodiments, the EOF can result from a net ionic current flow from a first side (e.g., a cis side) to a second side (e.g., a trans side). In some embodiments, an EOF can result from a net ionic current flow from a first side (e.g., a cis side) to a second side (e.g., a trans side) over a total ionic current flow, (e.g., relative net current flow). In some embodiments, an electro-osmotic flow net EOF can form from a first side (e.g., a cis side) to a second side (e.g., a trans side). In some embodiments, an electro-osmotic flow net EOF can form from a second side (e.g., a trans side) to a first side (e.g., a cis side). The EOF can be either cation biased or anion biased. In some embodiments, the direction of the net electro-osmotic force can determine to which side an analyte is added.

In some embodiments, an electro-osmotic force may be generated by a chemical gradient of an ion between two sides of a pore. In some embodiments, an EOF can be generated with a strong asymmetric ion flow (e.g., an asymmetric ion concentration between two sides of a pore). A low salt concentration conditions can be used in the compartment from which it is desired to have low ionic transfer, relative to higher salt concentration in the compartment from which high ionic flux is desired. In some embodiments, a salt or electrolyte concentration on a first side (e.g., a cis side) can be greater than about 0.01, 0.05, 0.10, 0.20, 0.30, 0.40, 0.50, 0.60, 0.70, 0.80, 0.90, 1.00, 1.10, 1.25, 1.50, 1.75, 2, 2.5, 3, 3.5, 4, 4.5 or about 5 M. In some embodiments, a difference in salt or electrolyte concentrations between the first side (e.g., the cis side) and the second side (e.g., the trans side) is greater than about 0.01, 0.05, 0.10, 0.20, 0.30, 0.40, 0.50, 0.60, 0.70, 0.80, 0.90, 1.00, 1.10, 1.25, 1.50, 1.75, 2, 2.5, 3, 3.5, 4, 4.5 or about 5 M. In some embodiments, a salt or electrolyte comprises sodium chloride, potassium chloride, guanidinium chloride, guanidine hydrochloride, potassium glutamate, an alkali metal salt, a halide salt, an ionic liquid, or an organic salt. In some embodiments, when there is a salt concentration asymmetry of the sensor system both cations and anions may flow from first side to second side at moderate to low applied voltages. Thus, salt applied electro-osmosis may be highly an advantageous means for creating or enhancing EOF under lower voltages. In some embodiments, higher asymmetric salt conditions may be used in combination with pores with enhanced ion-selectivity.

In some embodiments, a system described herein comprises at least one solution. In some embodiments, the first side of the system can comprise a first solution and the second side of the system can comprise a second solution. In some embodiments, the first solution and second solution may be the same. In some embodiments, the first solution and second solution may be different. In some embodiments, the first solution comprises a first concentration of a solute. The second solution can comprise a second concentration of the solute. In some embodiments, the first solution and second solution comprise the same solute. In some embodiments, the first solution and second solution comprise different solutes. The first solution can comprise a concentration of a first solute and the second solution can comprise a concentration of a second solute.

In some embodiments, the solute can comprise an ion. In some embodiments, the solute can comprise an osmolyte. In some embodiments, an osmolyte can comprise a non-ionic or a zwitterionic solute, (e.g., glycine betaine, glucose, sucrose, glycerol, PEGs, dextrans, or any combination thereof). In some embodiments, different ionic concentrations between the first and second sides of a system may result in high mobility ions and low mobility ions. Without wishing to be bound by theory, high mobility ions can be used on one side of a membrane and low mobility and/or sterically inhibited counterions on the other side of a membrane to generate an EOF of the sensor system.

Salt imbalances on two sides of a pore can create strong osmotic gradients. Specific osmolytes can be selected and balanced based on their osmolarity and their concentrations. In some embodiments, osmolytes can be added either to symmetrical salt concentration or asymmetric salt concentration systems to create an osmotic gradient that acts in the same direction as the EOF to enhance the capture and/or translocation of an analyte to the pore. In some embodiments, osmolytes can be added either to symmetrical salt concentration or asymmetric salt concentration systems to create an osmotic gradient that acts in a different direction as the EOF to enhance the capture and/or translocation of an analyte to the pore.

In some embodiments, a salt, ion, osmolyte, or electrolyte concentration on the first side (e.g., cis side) can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the first side (e.g., cis side) can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the first side (e.g., cis side) can be from about 0.01 M to about 5 M. In some embodiments, a salt, ion, osmolyte, or electrolyte concentration on the first side (e.g., cis side) can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the first side (e.g., cis side) can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some embodiments, a salt, ion, osmolyte, or electrolyte concentration on the second side (e.g., trans side) can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the second side (e.g., trans side) can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the second side (e.g., trans side) can be from about 0.01 M to about 5 M. In some embodiments, a salt, ion, osmolyte, or electrolyte concentration on the second side (e.g., trans side) can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the second side (e.g., trans side) can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

### Methods and Applications

A nanopore system as described herein may find its use in various applications, ranging from analytical detection methods in a research setting, high throughput drug development to real-time diagnostic applications.

In some aspects, the nanopores, methods, and system provided herein comprise detecting and/or characterizing one or more characteristics of an analyte. Characteristics of the analyte (e.g., the non-nucleic acid based polymer analyte) comprise of the non-nucleic acid based polymer analyte comprise a shape of the non-nucleic acid based polymer analyte, a structure of the non-nucleic acid based polymer analyte, one or more mutations of the non-nucleic acid based polymer analyte, a sequence of the non-nucleic acid polymer analyte, a surface charge of the non-nucleic acid based polymer analyte, one or more post-translation modifications of the non-nucleic acid based polymer analyte, one or more ligands coupled to the non-nucleic acid based polymer analyte, or any combination thereof.

A method may comprises providing a nanopore system. In some embodiments, the nanopore system comprises a system described herein. In some embodiments, a method comprises translocating an analyte (e.g., a non-nucleic acid based polymer analyte) in a sensor system comprising a pore. The analyte may be translocated to a pore, captured by a pore, and retained in a pore. In some embodiments, the pore comprises a nanopore as described herein. In some embodiments, the pore comprises a functionalized nanopore described herein. In some embodiments, the pore is comprised in a membrane. In some embodiments, the membrane separates a fluidic chamber into a first side (e.g., a cis side) and a second side (e.g., a trans side). In some embodiments, the membrane insulates a first side (e.g., a cis side) and a second side (e.g., a trans side). In some embodiments, a sample can be added to the first side. In some embodiments, a sample can be added to the first side in a solution of one or more electrolytes. The solution can comprise an ion or osmolyte. A system can generate an EOF from a first side (e.g., a cis side) to a second side (e.g., a trans side) to shift an analyte in a solution. In some embodiments, a sample can be added to the second side. In some embodiments, a sample can be added to a trans side in a solution of one or more electrolytes. A system can comprise an EOF from a second side (e.g., a trans side) to a first side (e.g., a cis side).

The methods provided herein may comprise contacting an analyte (e.g., a non-nucleic acid based polymer analyte) with a nanopore. An analyte may contact a nanopore at any location of the nanopore. An analyte may contact a nanopore at a first opening (e.g., cis opening) or a second opening (e.g., a trans opening). An analyte may contact a nanopore within the pore (e.g., channel) of the nanopore. The analyte may contact and/or interact with amino acid residues within the channel. The analyte may contact a constriction region of the nanopore.

A method provided herein comprises measuring a signal generated by the translocation of the analyte (e.g., the non-nucleic acid based polymer analyte) to the pore (e.g., biological nanopore) and reside in the pore. One method can be to measure the ionic current from one side of the membrane to the other side. Another method can be to measure electric potential from one side to the other side. The impedance and/or conductivity can also be measured. In some embodiments, current rectification can be measured. In some embodiments, fluorescence probes for reporting ionic flux or field effect transistor systems can be used to measure properties of a translocation and/or capture event. In some embodiments, changes in the system's ionic concentrations can be measured without an applied electric potential. Instead, the changes may be measured by a chemical gradient of ions and/or analytes can provide the driving force for translocation of analytes to a pore and create measurable signals. In some embodiments, the applied potential can be a chemical potential or applied electric potential. A system described herein can comprise electrodes, spectroscopy tools, microscopes, etc. to measure the signals.

An applied electric potential can be maintained at a constant or fluctuating voltage for a fixed period (milliseconds, seconds, minutes, hours). In some embodiments, the voltage can be changed in discrete steps to alter the sensing conditions and/or obtain different information from the analytes. The voltage can be constantly changing, such as periodic waveforms (e.g. square wave, triangular wave, sinusoidal, etc.). Waveforms of different amplitudes, frequencies, and shapes can be used to translocate analytes, which can produce different signals from the same analytes.

In some embodiments, the absolute relative net electro-osmotic flow over applied voltage (IrelV), can be at least about 0.01 pA/mV, at least about 0.02 pA/mV, at least about 0.03 pA/mV, at least about 0.04 pA/mV, at least about 0.05 pA/mV, at least about 0.06 pA/mV, at least about 0.07 pA/mV, at least about 0.08 pA/mV, at least about 0.09 pA/mV, at least about 0.10 pA/mV, at least about 0.15 pA/mV, at least about 0.2 pA/mV, at least about 0.3 pA/mV, at least about 0.4 pA/mV, at least about 0.5 pA/mV, at least about 0.6 pA/mV, at least about 0.7 pA/mV, at least about 0.8 pA/mV, at least about 0.9 pA/mV, at least about 1 pA/mV, or greater than about 1 pA/mV. In some embodiments, the absolute relative net electro-osmotic flow over applied voltage (IrelV), can be at most about 1 pA/mV, at most about 0.9, at most about 0.8, at most about 0.7, at most about 0.6, at most about 0.5, at most about 0.4, at most about 0.3, at most about 0.2, at most about 0.15, at most about 0.10, at most about 0.09, at most about 0.08, at most about 0.07, at most about 0.06, at most about 0.05, at most about 0.04, at most about 0.03, at most about 0.02, at most about 0.01, or less than about 0.1 pA/mV.

In some embodiments, the absolute relative net electro-osmotic flow over applied voltage (IrelV), can be from about 0.01 pA/mV to about 1 pA/mV. In some embodiments, the absolute relative net electro-osmotic flow over applied voltage (IrelV), can be from about 0.01 pA/mV to about 0.02 pA/mV, about 0.01 pA/mV to about 0.04 pA/mV, about 0.01 pA/mV to about 0.06 pA/mV, about 0.01 pA/mV to about 0.08 pA/mV, about 0.01 pA/mV to about 0.1 pA/mV, about 0.01 pA/mV to about 0.15 pA/mV, about 0.01 pA/mV to about 0.2 pA/mV, about 0.01 pA/mV to about 0.4 pA/mV, about 0.01 pA/mV to about 0.6 pA/mV, about 0.01 pA/mV to about 0.8 pA/mV, about 0.01 pA/mV to about 1 pA/mV, about 0.02 pA/mV to about 0.04 pA/mV, about 0.02 pA/mV to about 0.06 pA/mV, about 0.02 pA/mV to about 0.08 pA/mV, about 0.02 pA/mV to about 0.1 pA/mV, about 0.02 pA/mV to about 0.15 pA/mV, about 0.02 pA/mV to about 0.2 pA/mV, about 0.02 pA/mV to about 0.4 pA/mV, about 0.02 pA/mV to about 0.6 pA/mV, about 0.02 pA/mV to about 0.8 pA/mV, about 0.02 pA/mV to about 1 pA/mV, about 0.04 pA/mV to about 0.06 pA/mV, about 0.04 pA/mV to about 0.08 pA/mV, about 0.04 pA/mV to about 0.1 pA/mV, about 0.04 pA/mV to about 0.15 pA/mV, about 0.04 pA/mV to about 0.2 pA/mV, about 0.04 pA/mV to about 0.4 pA/mV, about 0.04 pA/mV to about 0.6 pA/mV, about 0.04 pA/mV to about 0.8 pA/mV, about 0.04 pA/mV to about 1 pA/mV, about 0.06 pA/mV to about 0.08 pA/mV, about 0.06 pA/mV to about 0.1 pA/mV, about 0.06 pA/mV to about 0.15 pA/mV, about 0.06 pA/mV to about 0.2 pA/mV, about 0.06 pA/mV to about 0.4 pA/mV, about 0.06 pA/mV to about 0.6 pA/mV, about 0.06 pA/mV to about 0.8 pA/mV, about 0.06 pA/mV to about 1 pA/mV, about 0.08 pA/mV to about 0.1 pA/mV, about 0.08 pA/mV to about 0.15 pA/mV, about 0.08 pA/mV to about 0.2 pA/mV, about 0.08 pA/mV to about 0.4 pA/mV, about 0.08 pA/mV to about 0.6 pA/mV, about 0.08 pA/mV to about 0.8 pA/mV, about 0.08 pA/mV to about 1 pA/mV, about 0.1 pA/mV to about 0.15 pA/mV, about 0.1 pA/mV to about 0.2 pA/mV, about 0.1 pA/mV to about 0.4 pA/mV, about 0.1 pA/mV to about 0.6 pA/mV, about 0.1 pA/mV to about 0.8 pA/mV, about 0.1 pA/mV to about 1 pA/mV, about 0.15 pA/mV to about 0.2 pA/mV, about 0.15 pA/mV to about 0.4 pA/mV, about 0.15 pA/mV to about 0.6 pA/mV, about 0.15 pA/mV to about 0.8 pA/mV, about 0.15 pA/mV to about 1 pA/mV, about 0.2 pA/mV to about 0.4 pA/mV, about 0.2 pA/mV to about 0.6 pA/mV, about 0.2 pA/mV to about 0.8 pA/mV, about 0.2 pA/mV to about 1 pA/mV, about 0.4 pA/mV to about 0.6 pA/mV, about 0.4 pA/mV to about 0.8 pA/mV, about 0.4 pA/mV to about 1 pA/mV, about 0.6 pA/mV to about 0.8 pA/mV, about 0.6 pA/mV to about 1 pA/mV, or about 0.8 pA/mV to about 1 pA/mV.

In some embodiments, the absolute relative net electro-osmotic flow over applied voltage (IrelV), can be about 0.01 pA/mV, about 0.02 pA/mV, about 0.03 pA/mV, about 0.04 pA/mV, about 0.05 pA/mV, about 0.06 pA/mV, about 0.07 pA/mV, about 0.08 pA/mV, about 0.09 pA/mV, about 0.10 pA/mV, about 0.15 pA/mV, about 0.2 pA/mV, about 0.3 pA/mV, about 0.4 pA/mV, about 0.5 pA/mV, about 0.6 pA/mV, about 0.7 pA/mV, about 0.8 pA/mV, about 0.9 pA/mV, or about 1 pA/mV.

In some embodiments, electrodes of a sensor system described herein can provide an applied voltage. The applied voltage may generate the electro-osmotic force (EOF) which may assist in translocating the analyte to the pore. In some embodiments, the applied voltage may be a negative voltage on a first side of a fluid chamber of the system. In some embodiments, the applied voltage may be a positive voltage on a first side of a fluid chamber of the system. In some embodiments, the applied voltage may be a negative voltage on a second side of a fluid chamber of the system. In some embodiments, the applied voltage may be a positive voltage on a second side of a fluid chamber of the system. In some embodiments, the applied voltage across the membrane can be at least about 1 mV, at least about 5 mV, at least about 10 mV, at least about 20 mV, at least about 30 mV, at least about 40 mV, at least about 50 mV, at least about 60 mV, at least about 70 mV, at least about 80 mV, at least about 90 mV, at least about 100 mV, at least about 150 mV, at least about 200 mV, at least about 250 mV, at least about 300 mV, at least about 350 mV, at least about 400 mV, at least about 450 mV, at least about 500 mV, at least about 600 mV, at least about 700 mV, at least about 800 mV, at least about 900 mV, at least about 1000 mV, or greater than about 1000 mV in magnitude. In some embodiments, the applied voltage across the membrane can be at least about 1000 mV, at most about 900 mV, at most about 800 mV, at most about 700 mV, at most about 600 mV, at most about 500 mV, at most about 450 mV, at most about 400 mV, at most about 350 mV, at most about 300 mV, at most about 250 mV, at most about 200 mV, at most about 150 mV, at most about 100 mV, at most about 90 mV, at most about 80 mV, at most about 70 mV, at most about 60 mV, at most about 50 mV, at most about 40 mV, at most about 30 mV, at most about 20 mV, at most about 10 mV, at most about 5 mV, at most about 1 mV, or less than about 1 mV in magnitude.

In some embodiments, the applied voltage across the membrane can be from about 1 mV to about 100 mV in magnitude. In some embodiments, the applied voltage across the membrane can be from about 1 mV to about 5 mV, about 1 mV to about 10 mV, about 1 mV to about 20 mV, about 1 mV to about 30 mV, about 1 mV to about 40 mV, about 1 mV to about 50 mV, about 1 mV to about 60 mV, about 1 mV to about 70 mV, about 1 mV to about 80 mV, about 1 mV to about 90 mV, about 1 mV to about 100 mV, about 5 mV to about 10 mV, about 5 mV to about 20 mV, about 5 mV to about 30 mV, about 5 mV to about 40 mV, about 5 mV to about 50 mV, about 5 mV to about 60 mV, about 5 mV to about 70 mV, about 5 mV to about 80 mV, about 5 mV to about 90 mV, about 5 mV to about 100 mV, about 10 mV to about 20 mV, about 10 mV to about 30 mV, about 10 mV to about 40 mV, about 10 mV to about 50 mV, about 10 mV to about 60 mV, about 10 mV to about 70 mV, about 10 mV to about 80 mV, about 10 mV to about 90 mV, about 10 mV to about 100 mV, about 20 mV to about 30 mV, about 20 mV to about 40 mV, about 20 mV to about 50 mV, about 20 mV to about 60 mV, about 20 mV to about 70 mV, about 20 mV to about 80 mV, about 20 mV to about 90 mV, about 20 mV to about 100 mV, about 30 mV to about 40 mV, about 30 mV to about 50 mV, about 30 mV to about 60 mV, about 30 mV to about 70 mV, about 30 mV to about 80 mV, about 30 mV to about 90 mV, about 30 mV to about 100 mV, about 40 mV to about 50 mV, about 40 mV to about 60 mV, about 40 mV to about 70 mV, about 40 mV to about 80 mV, about 40 mV to about 90 mV, about 40 mV to about 100 mV, about 50 mV to about 60 mV, about 50 mV to about 70 mV, about 50 mV to about 80 mV, about 50 mV to about 90 mV, about 50 mV to about 100 mV, about 60 mV to about 70 mV, about 60 mV to about 80 mV, about 60 mV to about 90 mV, about 60 mV to about 100 mV, about 70 mV to about 80 mV, about 70 mV to about 90 mV, about 70 mV to about 100 mV, about 80 mV to about 90 mV, about 80 mV to about 100 mV, or about 90 mV to about 100 mV in magnitude.

In some embodiments, the applied voltage across the membrane can be from about 100 mV to about 1,000 mV in magnitude. In some embodiments, the applied voltage across the membrane can be from about 100 mV to about 150 mV, about 100 mV to about 200 mV, about 100 mV to about 250 mV, about 100 mV to about 300 mV, about 100 mV to about 400 mV, about 100 mV to about 500 mV, about 100 mV to about 600 mV, about 100 mV to about 700 mV, about 100 mV to about 800 mV, about 100 mV to about 900 mV, about 100 mV to about 1,000 mV, about 150 mV to about 200 mV, about 150 mV to about 250 mV, about 150 mV to about 300 mV, about 150 mV to about 400 mV, about 150 mV to about 500 mV, about 150 mV to about 600 mV, about 150 mV to about 700 mV, about 150 mV to about 800 mV, about 150 mV to about 900 mV, about 150 mV to about 1,000 mV, about 200 mV to about 250 mV, about 200 mV to about 300 mV, about 200 mV to about 400 mV, about 200 mV to about 500 mV, about 200 mV to about 600 mV, about 200 mV to about 700 mV, about 200 mV to about 800 mV, about 200 mV to about 900 mV, about 200 mV to about 1,000 mV, about 250 mV to about 300 mV, about 250 mV to about 400 mV, about 250 mV to about 500 mV, about 250 mV to about 600 mV, about 250 mV to about 700 mV, about 250 mV to about 800 mV, about 250 mV to about 900 mV, about 250 mV to about 1,000 mV, about 300 mV to about 400 mV, about 300 mV to about 500 mV, about 300 mV to about 600 mV, about 300 mV to about 700 mV, about 300 mV to about 800 mV, about 300 mV to about 900 mV, about 300 mV to about 1,000 mV, about 400 mV to about 500 mV, about 400 mV to about 600 mV, about 400 mV to about 700 mV, about 400 mV to about 800 mV, about 400 mV to about 900 mV, about 400 mV to about 1,000 mV, about 500 mV to about 600 mV, about 500 mV to about 700 mV, about 500 mV to about 800 mV, about 500 mV to about 900 mV, about 500 mV to about 1,000 mV, about 600 mV to about 700 mV, about 600 mV to about 800 mV, about 600 mV to about 900 mV, about 600 mV to about 1,000 mV, about 700 mV to about 800 mV, about 700 mV to about 900 mV, about 700 mV to about 1,000 mV, about 800 mV to about 900 mV, about 800 mV to about 1,000 mV, or about 900 mV to about 1,000 mV in magnitude.

In some embodiments, the applied voltage across the membrane can be about 1 mV, about 5 mV, about 10 mV, about 20 mV, about 30 mV, about 40 mV, about 50 mV, about 60 mV, about 70 mV, about 80 mV, about 90 mV, about 100 mV, about 150 mV, about 200 mV, about 250 mV, about 300 mV, about 350 mV, about 400 mV, about 450 mV, about 500 mV, about 600 mV, about 700 mV, about 800 mV, about 900 mV, or about 1000 mV in magnitude.

In one aspect, the invention provides a method for detecting and/or characterizing at least one target analyte using a nanopore system according to the invention, comprising: (a) allowing capture of the target analyte by the nanopore so that the analyte temporarily lodges into the conical vestibule of the nanopore; (b) optionally applying an electrical potential across the nanopore; and (c) measuring ionic current passing through the nanopore, wherein a change in the frequency and/or magnitude of ionic current indicates the presence, concentration, identity and/or other characteristics of the target analyte in the sample. In some aspects, the present disclosure provides a method for detecting and/or characterizing at least one target analyte. In some embodiments, the analyte may be characterized using a sensor system comprising a pore. In some embodiments, the pore can be a nanopore. In some embodiments, the pore can be a biological nanopore. In some embodiments, the nanopore can be a cylindrical nanopore. In some embodiments, the nanopore can be a conical nanopore. In some embodiments, the analyte may be characterized by lodging in the nanopore (e.g., getting captured or residing in the nanopore). An electrical potential can be applied across the nanopore when the analyte is residing in the nanopore. An electrical potential can be applied across the nanopore when the analyte is not residing in the nanopore. An ionic current passing through the nanopore may be measured. An analyte may be characterized by measuring a frequency change in the ionic current. An analyte may be characterized by measuring a magnitude change in the ionic current. An analyte may be characterized by measuring a frequency and magnitude change in the ionic current. Characterization of the analyte may comprise determining a presence of an analyte, a concentration of an analyte, a sequence of an analyte, a chemical composition of an analyte, a size of an analyte, and/or other characteristics of the analyte in a sample.

In some embodiments, the EOF and/or electrophoretic potential generated by the applied electrical potential may translocate an analyte (e.g., the non-nucleic acid based polymer analyte) to the pore. In some embodiments, the EOF and/or electrophoretic potential generated by the chemical gradient potential may translocate an analyte (e.g., the non-nucleic acid based polymer analyte) to the pore. The analyte can enter the nanopore through a first opening or a second opening. The analyte may exit the nanopore through a first opening or a second opening.

Characterization of an analyte may comprise identifying a bound moiety to the analyte. For example, an analyte may occur in various different forms e.g. structure, shape, free or ligand bound, post-translationally modification, charge, or any combination thereof. The analyte and/or bound moiety may comprise any other type or level of heterogeneity. In some embodiments, the nanopore (e.g., a conical nanopore described herein) can distinguish between different forms of a moiety bound to an analyte. In some embodiments, the bound moiety may have a distinct signal from the analyte. In some embodiments, a first bound moiety may have a signal that is distinct from the analyte and/or a second bound moiety. For example, a bound moiety may change a current when the bound moiety and analyte reside in a nanopore and/or system described herein. The current may change relative to a reference signal (e.g., a current signal without the moiety bound to the analyte). The change in signal may be a change in the magnitude of the ionic current, the noise of the current signal, the frequency components of the current changes, the dwell time of any changes in current, or any combination thereof.

In some embodiments, a sample to be analyzed for the presence of a target analyte that can be captured and trapped by the pore (e.g., nanopore or conical nanopore) can be added to the first side (e.g., cis side) of the nanopore system. A sample to be analyzed for presence of a target analyte may be added to the second side (e.g., trans side) of a nanopore system. A sample to be analyzed for presence of a target analyte may be added to the cis chamber and the trans chamber of a nanopore system.

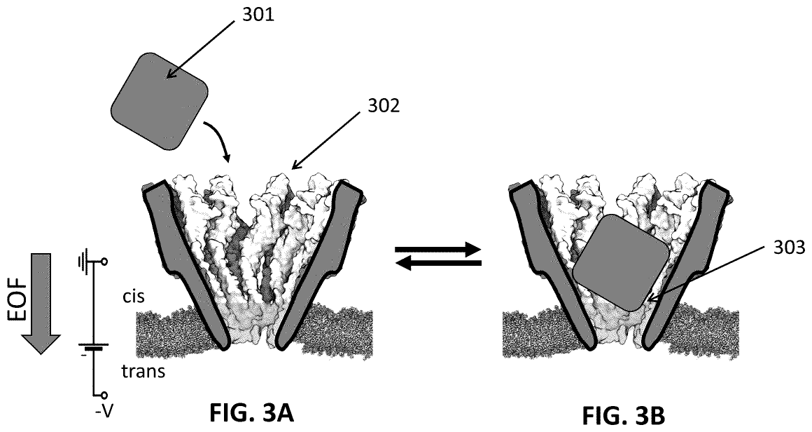

In some embodiments, a membrane can provide an electro-osmotic force (EOF). The EOF may assist in the capture and/or characterization of an analyte using the nanopores, systems, and/or methods described herein. As shown in FIG. 3A, an analyte (301) may translocate to a first opening (302) of a nanopore due to an applied EOF. The EOF may originate from a chemical gradient, an applied electrical potential, or any combination thereof. For example, an electrode may apply a charged potential to a nanopore system, allowing an analyte to translocate to a nanopore and contact the nanopore. The analyte can enter a nanopore through an opening (302). The charge gradient (e.g., EOF) may be in direction of a first side (e.g., a cis side) to a second side (e.g., a trans side). The analyte (301) in FIG. 3B has been captured by the nanopore and resides in a constriction region (303). The constriction region can have a dimension (e.g., length, width, diameter, circumference, widest dimension or any combination thereof) that is smaller than a first opening of the nanopore. As the arrows of FIGS. 3A and 3B indicate, the analyte can both enter and reside in the nanopore, as well as leave the nanopore, providing a measure of an open-pore and an occupied (e.g., blocked) pore.

Upon entering the nanopore under influence of an electrophoretic and/or electroosmotic force, an analyte's interactions with the nanopore can depend on its chemical composition. These interactions can give rise to specific residence times of the analyte within the nanopore, (e.g., dwell times). The analyte's presence in the nanopore also can block the flow of ions through the pore, which can be detected as a change in current. This residual blockage current (Ires %) can depends on the chemical composition, size, shape, or pH of the analyte, or any analyte characteristic described herein. Without wishing to be bound by theory, chemically distinct analytes (e.g., mixed analytes of a complex sample) may be distinguished using a nanopore as described herein based on a measure of dwell time, blockade noise (σ_{blockade}), blockage current, or any combination thereof.

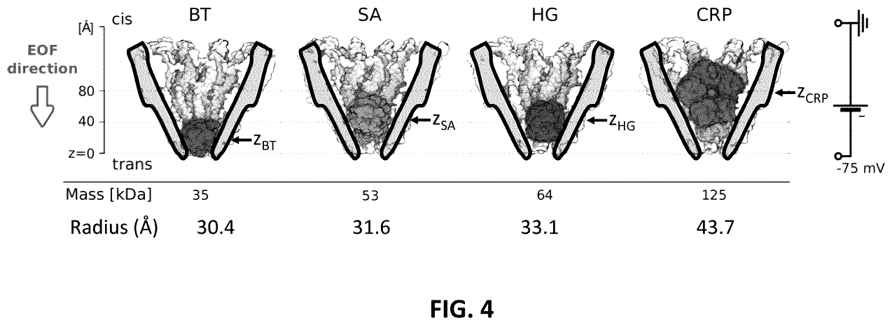

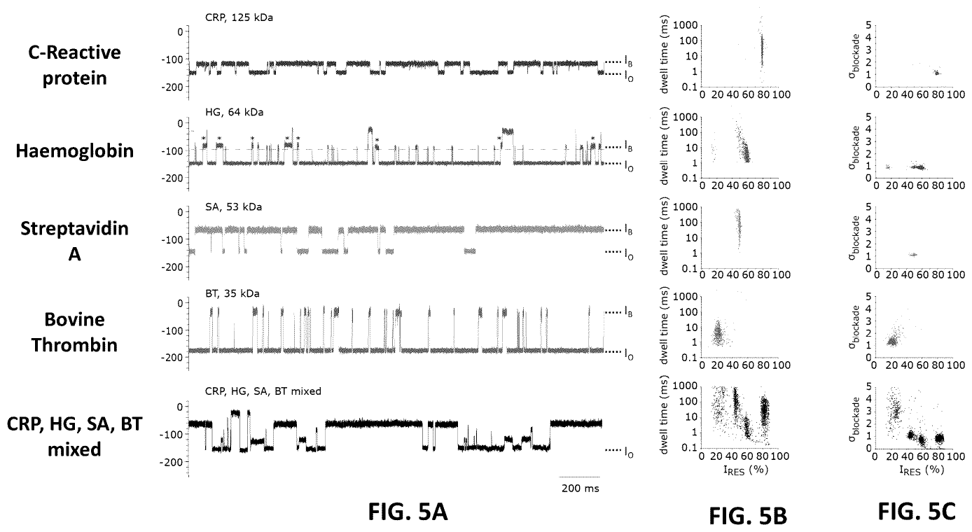

As shown in FIG. 4 and FIGS. 5A-5C, an analytes size can impact a position of an analyte in a nanopore and measures of dwell time, blockade noise (σ_{blockade}), and blockage current. In FIG. 4, increasingly smaller analytes, as denoted by mass (e.g., kDa) and radius (Å), are captured at deeper positions in a nanopore (as shown by z position). Electrodes of the nanopore system can comprise a first electrode on a first side (e.g., a cis side) and a second electrode on a second side (e.g., a trans side). The electrodes can provide a charge to create an electro-osmotic flow (EOF). The EOF can apply a potential from a first side to a second side (e.g., a cis to trans direction), which allows an analyte to translocate and be captured by the nanopore. The position of each analyte can be represented as the z-position. As the smaller analyte comprises a smaller mass and radius, it can travel farther into the analyte and may be captured closer to the second opening (e.g., the opening by the trans side).

When measuring the current of FIG. 5A, the lowest current level can be the open-pore current of the pore (I_{O}), and the step-like upwards events can be the result of captured analytes occluding a portion of the ionic current flowing through the nanopore (event blockades, I_{B}). Residual current (Iᵣₑₛ) can be shown as Iᵣₑₛ (%)=(I_{B}/I_{O})×100, providing a summary of the open-pore current and event blockades. As in FIG. 5A, each analyte of the complex sample can be characterized by a different current pattern. When the complex sample is analyzed, the individual analytes may be identified by the different peaks in the current signal for example. The y-axis of FIG. 5A denotes current (in pA) and is measured across time on the x-axis. Furthermore, the analytes may be detected and identified through analysis of dwell time (e.g. the time period of the analyte in the nanopore) and/or blockade noise (e.g., the variability in current blockade signal). The blockade noise (σ_{blockade}) provides a measure of variability in pA on the y-axis for the blockade events of the analyte. As shown in the bottom row of FIGS. 5A-5C, these three measurements can provide a detailed analysis of the composition of a complex sample as the Iᵣₑₛ%, dwell time, and blockade noise can be distinct for each analyte of the sample.

The methods provided herein may be used to detect any type of analyte. In some aspects, the method comprises detecting and/or characterizing a protein, protein assembly or protein complex. In one aspect, the method comprises detecting and/or characterizing a protein, protein assembly or protein complex. In some embodiments, the analyte may comprise a folded protein, folded peptide, folded polypeptide, or any combination thereof. For example, an analyte may comprise (i) a protein (e.g., folded protein) having a molecular weight of at least 80 kDa (e.g., at least about 100 kDa); (ii) a size between about 2-20 nm (e.g., between about 3-15 nm) in at least two dimensions; (iii) a hydrodynamic radius of at least about 20 Å (e.g., at least 28 Å or 30 Å); or (iv) any combination thereof. The hydrodynamic radius can comprise a structural measurement of the analyte's shape and/or size and may be indicative of the radius of ion in solution as the analyte moves through the solution. In the case of proteins, the hydrodynamic value that can be experimentally derived may be the Stokes radius (Rs), which can refer to the radius of a sphere with the same hydrodynamic properties (i.e., frictional coefficient) as the biomolecule. A hydrodynamic radius may be measured using dynamic light scattering, size exclusion chromatography, or a combination thereof. The analyte may comprise a size between 1-50 nm in a length and width, a length and diameter, a length and circumference, a width and diameter, a width and circumference, or any combination thereof.

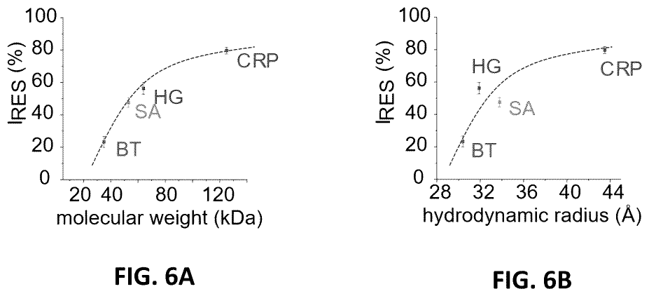

FIGS. 6A-6B demonstrates the relationship of an analyte's characteristic to a level of residual current (e.g., Iᵣₑₛ%). A larger analyte can have a larger residual current (e.g., Iᵣₑₛ%). The larger analyte can have a larger molecule weight (kDa). In some cases, the larger analyte can have a larger hydrodynamic radius (Å). A large analyte may comprise of greater than about 50 kDa, greater than about 75 kDa, greater than 100 kDa, or greater than 150 kDa. Using the relationship between an analyte's size and residual current, it can be possible to estimate a residual current of an analyte in a nanopore described herein. Using the relationship between an analyte's size and residual current, it can be possible to estimate an analyte's size (e.g., weight, hydrodynamic radius, volume, shape, or any combination thereof) from a residual current of an analyte in a nanopore described herein.

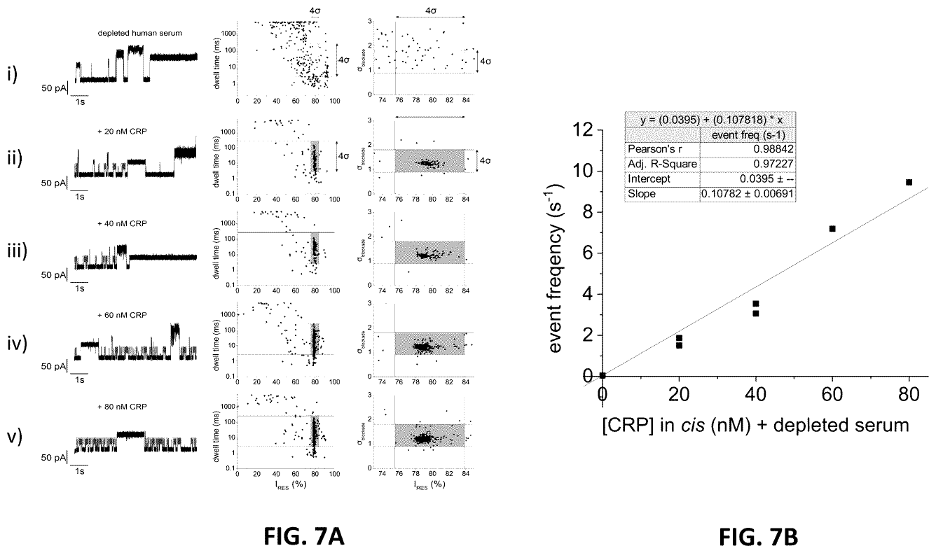

A concentration of an analyte may also affect an average number of blockade events of a nanopore over time. As shown in FIG. 7A, increasing concentrations of analyte C-reactive protein (CRP) can lead to increasing blockade of a nanopore described herein. The higher concentrations may increase the average amount of time in blockade due to a higher frequency of blockades. FIG. 7A shows measures of current signal, measured in picoAmps (pA) (y-axis) over time (x-axis). The dwell time and blockade noise (σ_{blockade}) are also plotted for the different concentrations of CRP. The increasing concentrations of an analyte may lead to increasing dwell time in the nanopore. Without wishing to be bound by theory, a higher concentration of an analyte may provide more of the analyte (e.g., peptide, protein, polypeptide, or any combination thereof) to occupy the nanopore once the pore is open. The increased concentration may increase a number of blockade events over a time period. The increased concentration of an analyte may provide for a greater average number of blockade events of multiple nanopores of a system. In some embodiments, an increased concentration may change a dwell time. In some embodiments, an increased concentration may not change a dwell time. Increasing the concentration of an analyte may increase a dwell time, a level of blockade noise, or any combination thereof, in a nanopore system described herein. FIG. 7B provides an example of the relationship between a concentration of an analyte (e.g., CRP) and a frequency of blockade events. In some embodiments, a higher concentration of an analyte may increase a frequency of blockade events in the nanopores, nanopore systems, or methods described herein.

In some aspects, the present disclosure provides a method of capturing a target analyte. In some embodiments, the method comprises using a pore. In some embodiments, the method can comprise using a pore of a sensor system. The sensor system may comprise multiple pores. The pores may be nanopores. The nanopores may be cylindrical. The nanopores may be conical-shaped. In some embodiments, the method may comprise filtering a target analyte in a sample. In some embodiments, the method may comprise filtering a target analyte using a pore-derived sensor system. The pore-derived sensor system may comprise at least one pore (e.g., a nanopore, e.g., a conical-shaped nanopore) in a membrane. In some embodiments, the methods may comprise capturing an analyte in a pore and filtering the target analyte from different (e.g., non-target) analytes. In some embodiments, the different (e.g., non-target) analytes may comprise different shape, structure, mutations, sequence, surface charge, post-translation modifications, or any combination thereof. The pore may capture a target analyte based on an ionic current. The pore may capture the target analyte based on a size, shape, and/or configuration of the target analyte. The pore may capture a target analyte that has been disposed in a cis compartment of a fluid filled chamber. The pore may capture a target analyte that has been disposed in a trans compartment of a fluid filled chamber.

The present disclosure provides methods using a nanopore (e.g., a biological nanopore) for selectively capturing an analyte. In some embodiments, the nanopore may filter a target analyte from different (e.g., non-target) analytes. The analytes may be filtered by the nanopore in a first solution (e.g., cis solution). The analytes may be filtered by the nanopore in a second solution (e.g., trans solution). In some embodiments, the invention provides an analytical method using functionalized conical shaped nanopores for selectively capturing and/or filtering different analytes in the cis solution. The method suitably comprises the use of a nanopore system comprising a conical shaped biological nanopore that can be functionalized to selectively enhance the capture of a target analyte from a complex mixture of components, such as biomolecules. The functionalized nanopore partially or fully traps the analyte in the vestibule of the nanopore.

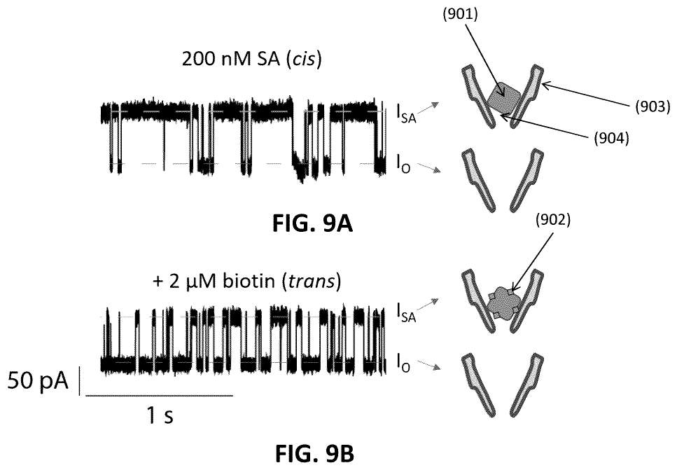

FIGS. A-9B9 show examples of recordings from a nanopore capturing an analyte (e.g., Streptavidin A (SA)). In FIG. 9A, the analyte (901) is captured at a constriction region (904) of the nanopore (903). The analyte may reside in the constriction region and the current is recorded from the nanopore system. The presence of the analyte in the nanopore can shift the current from the open-pore current to the blockage current. Analysis of the current blockade from SA (I_{SA}) compared to the open-pore current (I_{O}) can characterize a captured analyte. Conjugated elements to an analyte may be detected and/or identified by measured differences in current output. For example, in FIG. 9B, biotin (902) may be conjugated to the analyte. Biotin can affect the current blockade from the analyte and lead to changes in the output current measurement. Analysis of the new I_{SA} current and comparison to the non-conjugated SA in FIG. 9A can lead to detection of the conjugated element (e.g., biotin). For example, conjugation of biotin may reduce a dwell time of SA in the nanopore and lead to less blockade events as shown by the decrease in I_{SA} current.

In some aspects, the present disclosure provides methods comprising providing a mixture containing or suspected of containing an analyte (e.g., a peptide, protein, polypeptide, or any combination thereof). A nanopore disclosed herein may be used to measure a concentration or relative amount of an analyte (e.g., a peptide, protein, polypeptide, or any combination thereof) in the mixture. In some cases, a nanopore disclosed herein may be used to determine an identity, a concentration, a presence, or any combination thereof, of an analyte in the mixture.

A measure of the concentration or relative amount of an analyte may be generated at an accuracy of at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%. A measure of the concentration or relative amount of an analyte may be generated at an accuracy of at most about 100%, at most about 99%, at most about 98%, at most about 97%, at most about 96%, at most about 95%, at most about 94%, at most about 93%, at most about 92%, at most about 91%, at most about 90%, at most about 85%, at most about 80%, at most about 70%, at most about 60%, at most about 50%, or less than about 50%. A measure of the concentration or relative amount of an analyte may be generated at an accuracy from about 50% to about 100%. A measure of the concentration or relative amount of an analyte may be generated at an accuracy from about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%.

A determination of the identity, concentration, presence, or any combination thereof, of an analyte may be generated at an accuracy of at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%. A determination of the identity, concentration, presence, or any combination thereof, of an analyte may be generated at an accuracy of at most about 100%, at most about 99%, at most about 98%, at most about 97%, at most about 96%, at most about 95%, at most about 94%, at most about 93%, at most about 92%, at most about 91%, at most about 90%, at most about 85%, at most about 80%, at most about 70%, at most about 60%, at most about 50%, or less than about 50%.

A determination of the identity, concentration, presence, or any combination thereof, of an analyte may be generated at an accuracy from about 50% to about 100%. A determination of the identity, concentration, presence, or any combination thereof, of an analyte may be generated at an accuracy from about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%.

Using this strategy, it may also be possible to filter for desired target analyte(s) from unwanted target analytes in (complex) mixtures such as clinical samples. A ‘capture and filtering’ method as described herein may employ a nanopore system comprising nanopores that are functionalized at the cis end with a recognition element as defined herein above. In a specific aspect, the method relies on YaxAB nanopores that are functionalized with a polymeric (e.g. peptide) extension containing a recognition element that is specific to a target analyte enables binding of the target analyte, and subsequently promotes capture of the analyte into the nanopore vestibule.

For samples to be analyzed for the presence of a very small target analyte that cannot be captured and trapped by the pore (e.g., nanopore or conical nanopore), the target analyte can be advantageously detected as part of a complex formed with a binding protein (e.g., a large cognate binding protein). The protein-analyte complex can be captured and/or characterized by the pore (e.g., nanopore or conical nanopore). The binding protein may be a protein, oligopeptide, or polypeptide. The protein-analyte complex may form by direct coupling. The protein-analyte complex may form by indirect coupling. In some embodiments, the binding protein may comprise an enzyme. For example, the binding protein may be a NTP binding enzyme. Non-limiting examples of binding proteins herein can include glucose binding proteins, actin-binding proteins, TATA-binding proteins, calcium binding proteins, CREB-binding proteins, nanobodies or a fragment thereof, antibodies or a fragment thereof, monobodies or a fragment thereof, designed ankyrin repeat proteins (DARPins), or any combination thereof.

In another aspect, the target analyte may be too small to be captured in the nanopore vestibule since its size and geometry of the allow entry and exit at both the wide end and the constriction region of the pore. For example, it has a geometry of less than 5 nm (e.g., 1-4 nm), in at least two dimensions. In these cases, the analyte can be suitably detected by using a large binder to trap the small target analyte that would otherwise be too small to be detected by the nanopore. A protein-analyte complex may form inside a nanopore. For example, a binding protein may enter a nanopore prior to an analyte. The analyte may then enter the nanopore and complex with the binding protein. As another example, an analyte may enter a nanopore prior to a binding protein. The binding protein may then enter the nanopore and complex with the analyte. As another example, a binding protein may enter a nanopore on a first side (e.g., through a first opening) and the analyte may enter the nanopore through a second side (e.g., through a second opening). The analyte and binding protein may then complex in the nanopore. The nanopore may then characterize the protein-analyte complex.

A protein-analyte complex may form outside of a pore (e.g., on a first side or a second side of a sensor system described herein). For example, a binding protein and analyte may both be present on a first side of a nanopore and bind on the first side of the nanopore. The bound protein-analyte complex may then enter the nanopore and be characterized. As another example, a binding protein and analyte may both be present on a second side of a nanopore and bind on the second side of the nanopore. The bound protein-analyte complex may then enter the nanopore and be characterized.

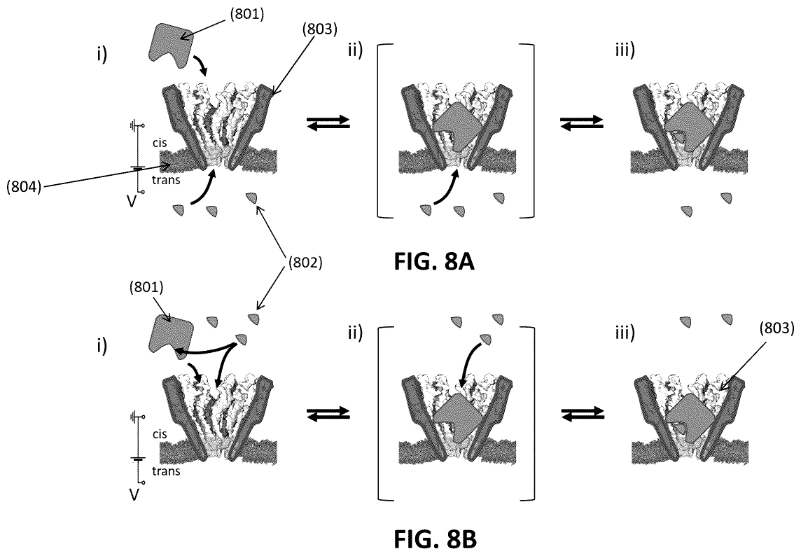

FIGS. 8A-8B provides an example of how a binding protein may assist in capture and/or characterization of an analyte. In FIG. 8A, panel (i) shows the binding protein (801) can be present on a first side (e.g., cis side) of a membrane (804) with a nanopore (803) disposed in the membrane. The analyte (802) is present on a second side of the membrane. The analyte (802) may be small enough to travel through the nanopore without capture. In panel (ii) of FIG. 8A, the binding protein (801) enters the nanopore through a first opening and can be captured in the nanopore. The binding protein may be characterized by the nanopore system. In panel (iii) of FIG. 8A, the analyte enters the second opening of the nanopore and interacts (e.g., binds) to the binding protein. The analyte coupled to the binding protein may be characterized by the nanopore system to determine one or more properties of the analyte (e.g., shape, size, charge, amino acid mutation(s), post-translational modification(s), or any combination thereof). In FIG. 8B, panel (i) shows the binding protein (801) and analyte (802) present on the same side of the membrane (e.g., the first side of the membrane). The analyte (802) may be of a size wherein the analyte may not be captured by the nanopore (803). As shown in panel (ii), the binding protein (801) may enter the nanopore (803) through the first opening and reside in the nanopore (e.g., the binding protein may be captured in a constriction region of the nanopore). In panel (iii), the analyte (802) may enter the nanopore through the same opening (e.g., the first opening) and interact (e.g., bind) to the binding protein (801), providing capture of the analyte and/or characterization of the analyte (802).

In some aspects, the present disclosure provides a method for detecting and/or characterizing at least one analyte (A). In some embodiments, the analyte may be detected by the nanopore following binding with a substrate (e.g., a binding protein or cognate binding protein). The analyte may be in a cis and/or a trans compartment of a pore (e.g., nanopore or conical nanopore). The binding protein may be in a cis and/or a trans compartment of a pore (e.g., nanopore or conical nanopore). The binding protein and analyte may bind to form a protein-analyte (BP-A) complex. The BP-A complex may form in the pore (e.g., nanopore or conical nanopore). The BP-A complex may form out of the pore (e.g., nanopore or conical nanopore). The binding protein and target analyte may both be present on a cis side of a pore (e.g., nanopore or conical nanopore). The binding protein and target analyte may both be present on a trans side of a pore (e.g., nanopore or conical nanopore). In some embodiments, a binding protein may enter a pore (e.g., nanopore or conical nanopore) first and a target analyte may bind to the binding protein in the pore. In some embodiments, a target analyte may enter a pore (e.g., nanopore or conical nanopore) first and a binding protein may bind to the target analyte in the pore.

In some aspects, the present disclosure provides a method for detecting and/or characterizing at least one analyte (A), wherein A can be detected as part of a complex formed with a cognate binding protein (BP), comprising adding to the cis side of the conical nanopore a BP that cannot pass a trans constriction of the nanopore, and allowing for capture of the binding protein-analyte (BP-A) complex by the nanopore. The binding protein-analyte (BP-A) complex may form in solution before capture of the complex by the nanopore and/or BP can be first captured in the nanopore and subsequently bind A to form a BP-A complex. See FIGS. 8A-8B for a schematic representation. FIGS. 8A-8B show a schematic model of the capture of binder protein-analyte (BP-A) complexes in YaxAB nanopores. FIG. 8A shows the capture of a binder entity BP (801) from the cis compartment, which in turn binds its cognate target analyte A (802) initially present in the trans compartment, so that a binder-analyte (BP-A) complex can be captured in the nanopore vestibule (panel iii). The capture of the binder-analyte complex may proceed by direct capture of the binder-analyte complex in a pre-bound state (i.e. the target bound to the binder outside of the nanopore), or via an intermediate state where the binder is captured first (bracketed state in panel ii), which then subsequently binds a target analyte while resident inside the vestibule of the nanopore. FIG. 8B shows a similar scheme to that shown in FIG. 8A, but where the target analyte (802) can be initially present on the same (cis) side as the binder.

To ensure that binding protein (BP) cannot pass from a first side (e.g., cis side) to a second side (e.g., trans side) of the nanopore system, it can be advantageous that BP has a size of 2-20 nm, for example greater than 3 nm and less than 15 nm, in at least two dimensions and/or a hydrodynamic radius (rₕ) of at least 20 Å, for example at least 25 Å, or at least 28 Å or at least 30 Å. In one aspect, it has a rₕ in the range of about 25 to 50 Å, for example 28 to 50 Å.

In some embodiments, a binding protein (BP) has at least one dimension (e.g., length, width, height, diameter, and/or circumference) that is at least about 2 nm, at least about 3 nm, at least about 4 nm, at least about 5 nm, at least about 6 nm, at least about 7 nm, at least about 8 nm, at least about 9 nm, at least about 10 nm, at least about 12 nm, at least about 15 nm, at least about 18 nm, at least about 20 nm, at least about 25 nm, or greater than about 25 nm in length. In some embodiments, a binding protein (BP) has at least one dimension (e.g., length, width, height, diameter, and/or circumference) that is at most about 25 nm, at most about 20 nm, at most about 18 nm, at most about 15 nm, at most about 12 nm, at most about 10 nm, at most about 9 nm, at most about 8 nm, at most about 7 nm, at most about 6 nm, at most about 5 nm, at most about 4 nm, at most about 3 nm, at most about 2 nm, or less than about 2 nm in length. In some embodiments, a binding protein (BP) has at least one dimension (e.g., length, width, height, diameter, and/or circumference) from about 3 nm to about 20 nm in length. In some embodiments, a binding protein (BP) has at least one dimension (e.g., length, width, height, diameter, and/or circumference) from about 3 nm to about 4 nm, about 3 nm to about 5 nm, about 3 nm to about 6 nm, about 3 nm to about 7 nm, about 3 nm to about 8 nm, about 3 nm to about 9 nm, about 3 nm to about 10 nm, about 3 nm to about 12 nm, about 3 nm to about 15 nm, about 3 nm to about 18 nm, about 3 nm to about 20 nm, about 4 nm to about 5 nm, about 4 nm to about 6 nm, about 4 nm to about 7 nm, about 4 nm to about 8 nm, about 4 nm to about 9 nm, about 4 nm to about 10 nm, about 4 nm to about 12 nm, about 4 nm to about 15 nm, about 4 nm to about 18 nm, about 4 nm to about 20 nm, about 5 nm to about 6 nm, about 5 nm to about 7 nm, about 5 nm to about 8 nm, about 5 nm to about 9 nm, about 5 nm to about 10 nm, about 5 nm to about 12 nm, about 5 nm to about 15 nm, about 5 nm to about 18 nm, about 5 nm to about 20 nm, about 6 nm to about 7 nm, about 6 nm to about 8 nm, about 6 nm to about 9 nm, about 6 nm to about 10 nm, about 6 nm to about 12 nm, about 6 nm to about 15 nm, about 6 nm to about 18 nm, about 6 nm to about 20 nm, about 7 nm to about 8 nm, about 7 nm to about 9 nm, about 7 nm to about 10 nm, about 7 nm to about 12 nm, about 7 nm to about 15 nm, about 7 nm to about 18 nm, about 7 nm to about 20 nm, about 8 nm to about 9 nm, about 8 nm to about 10 nm, about 8 nm to about 12 nm, about 8 nm to about 15 nm, about 8 nm to about 18 nm, about 8 nm to about 20 nm, about 9 nm to about 10 nm, about 9 nm to about 12 nm, about 9 nm to about 15 nm, about 9 nm to about 18 nm, about 9 nm to about 20 nm, about 10 nm to about 12 nm, about 10 nm to about 15 nm, about 10 nm to about 18 nm, about 10 nm to about 20 nm, about 12 nm to about 15 nm, about 12 nm to about 18 nm, about 12 nm to about 20 nm, about 15 nm to about 18 nm, about 15 nm to about 20 nm, or about 18 nm to about 20 nm in length.

In some embodiments, a binding protein (BP) may comprise a hydrodynamic radius in the range of about 25 to 50 Å, for example 28 to 50 Å. In some embodiments, a binding protein (BP) described herein may have a hydrodynamic radius of at least about 10 Å, at least about 15 Å, at least about 20 Å, at least about 21 Å, at least about 22 Å, at least about 23 Å, at least about 24 Å, at least about 25 Å, at least about 26 Å, at least about 27 Å, at least about 28 Å, at least about 29 Å, at least about 30 Å, at least about 35 Å, at least about 40 Å, at least about 45 Å, at least about 50 Å, or greater than about 50 Å. In some embodiments, an analyte described herein may have a hydrodynamic radius of at most about 50 Å, at most about 45 Å, at most about 40 Å, at most about 35 Å, at most about 30 Å, at most about 29 Å, at most about 28 Å, at most about 27 Å, at most about 26 Å, at most about 25 Å, at most about 24 Å, at most about 23 Å, at most about 22 Å, at most about 21 Å, at most about 20 Å, at most about 15 Å, at most about 10 Å, or less than about 10 Å. In some embodiments, a binding protein (BP) described herein may have a hydrodynamic radius from about 10 Å to about 50 Å. In some embodiments, a binding protein (BP) described herein may have a hydrodynamic radius from about 10 Å to about 15 Å, about 10 Å to about 20 Å, about 10 Å to about 22 Å, about 10 Å to about 24 Å, about 10 Å to about 26 Å, about 10 Å to about 28 Å, about 10 Å to about 30 Å, about 10 Å to about 35 Å, about 10 Å to about 40 Å, about 10 Å to about 45 Å, about 10 Å to about 50 Å, about 15 Å to about 20 Å, about 15 Å to about 22 Å, about 15 Å to about 24 Å, about 15 Å to about 26 Å, about 15 Å to about 28 Å, about 15 Å to about 30 Å, about 15 Å to about 35 Å, about 15 Å to about 40 Å, about 15 Å to about 45 Å, about 15 Å to about 50 Å, about 20 Å to about 22 Å, about 20 Å to about 24 Å, about 20 Å to about 26 Å, about 20 Å to about 28 Å, about 20 Å to about 30 Å, about 20 Å to about 35 Å, about 20 Å to about 40 Å, about 20 Å to about 45 Å, about 20 Å to about 50 Å, about 22 Å to about 24 Å, about 22 Å to about 26 Å, about 22 Å to about 28 Å, about 22 Å to about 30 Å, about 22 Å to about 35 Å, about 22 Å to about 40 Å, about 22 Å to about 45 Å, about 22 Å to about 50 Å, about 24 Å to about 26 Å, about 24 Å to about 28 Å, about 24 Å to about 30 Å, about 24 Å to about 35 Å, about 24 Å to about 40 Å, about 24 Å to about 45 Å, about 24 Å to about 50 Å, about 26 Å to about 28 Å, about 26 Å to about 30 Å, about 26 Å to about 35 Å, about 26 Å to about 40 Å, about 26 Å to about 45 Å, about 26 Å to about 50 Å, about 28 Å to about 30 Å, about 28 Å to about 35 Å, about 28 Å to about 40 Å, about 28 Å to about 45 Å, about 28 Å to about 50 Å, about 30 Å to about 35 Å, about 30 Å to about 40 Å, about 30 Å to about 45 Å, about 30 Å to about 50 Å, about 35 Å to about 40 Å, about 35 Å to about 45 Å, about 35 Å to about 50 Å, about 40 Å to about 45 Å, about 40 Å to about 50 Å, or about 45 Å to about 50 Å. In one embodiment, this “complexation” method comprises detecting and/or characterizing at least one target analyte (A) capable of passing the trans constriction of the conical nanopore, wherein said target analyte (e.g., small analyte) can be captured in the nanopore by complexing to a large cognate binding protein (BP) that cannot pass the trans constriction of the nanopore. In some cases, the analyte may be added to the cis and/or trans side of the nanopore system, whereas BP can be added to a first side (e.g., a cis side). In some cases, the analyte may be added to a first side (e.g., a cis side) and/or a second side (e.g., a trans side) of the nanopore system, whereas BP can be added to the second side (e.g., trans side). The “complexation” approach may not be restricted to detection of small analytes; it can be used for protein-analyte complexes comprises two or more large entities, e.g. large protein-protein complexes or oligomers. The protein-complex (or indeed any complex) method can be used to detect both small and large radius by virtue of the difference between the unbound binder state and the bound-binder state. For example, an analyte and a binder protein may comprise the same mass. For example, an analyte and a binder protein may comprise the same length and/or width. This approach can be used to detect protein-ligand complexes, e.g. protein-drug binding, protein-cofactor binding, enzyme-substrate binding, or any combinations thereof. For example, the nanopores, methods, and/or systems described herein may detect and/or characterize an antibody-drug conjugate, a transcription factor complex, a protein export complex, a protein transport complex, or any combination thereof. In some embodiments, the nanopores, methods, and/or systems described herein may detect and/or characterize a drug bound to albumin, al-acid glycoprotein, lipoprotein, or any combination thereof. The drug may be for any disease or condition, including but not limited to, a cancer, bacterial infection, viral infection, immunological condition, autoimmune disease, infectious disease, or any coinfection.

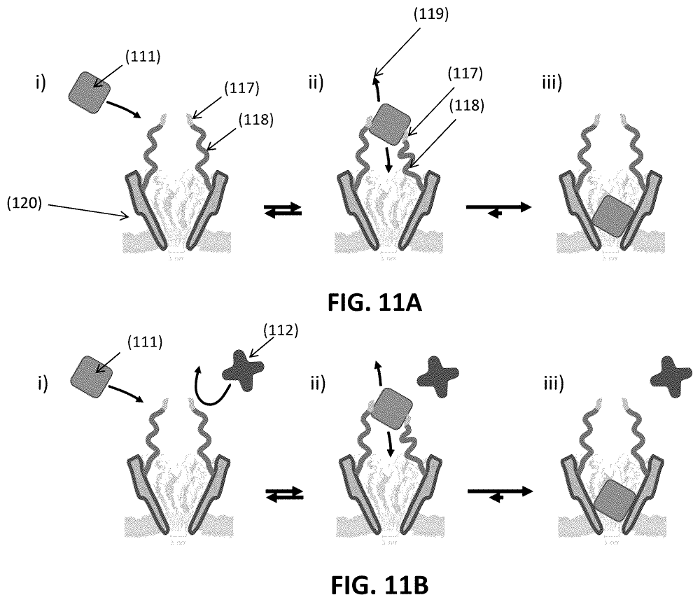

In some embodiments, a binding protein (BP) can bind to a target analyte of a sample and may not bind to a different (e.g., non-target) analyte of the sample. In some embodiments, the sample may be a complex biological sample, and the complexation methods described herein provide a means of trapping and/or filtering the analyte in a nanopore. For example, FIGS. 11A-11B show a schematic model of these selective “capture and filtering” strategies. In some embodiments, a recognition element may contact at least one location of a target analyte. In some embodiments, the recognition element may contact the target analyte and capture the analyte within the pore. The schematic model in FIGS. 11A-11B show a strategy of employing functionalized nanopores (e.g., YaxAB nanopores) for selectively capturing and/or filtering different analytes in the cis solution. In FIG. 11A, the functionalized nanopores (e.g., YaxAB nanopores) with a polymeric (e.g. peptide, protein, or polypeptide) extension (118) contains a recognition element (117) that is specific to a target analyte (111), can enable binding of the target analyte (panel ii), and subsequently can promote capture of the analyte (111) into the nanopore vestibule (panel iii). The functionalized nanopore partially or fully traps the analyte in the vestibule. As shown in Panel (ii), once bound to the recognition element, the analyte may exit the recognition element (119) or become captured by the nanopore for characterization (as shown in (iii)). In FIG. 11B, the figure shows a strategy for filtering for desired target analytes (111) from unwanted target analytes (112) in mixtures using functionalized nanopores (e.g., YaxAB nanopores). In (i) the recognition element can be specific for the target analyte (111). The non-target analyte (112) may not bind to the recognition element and therefore may not be captured by the nanopore for characterization. Once the recognition element binds to the analyte (111) in (ii), the analyte can be captured and can reside in the nanopore as shown in panel (iii) for further characterization.

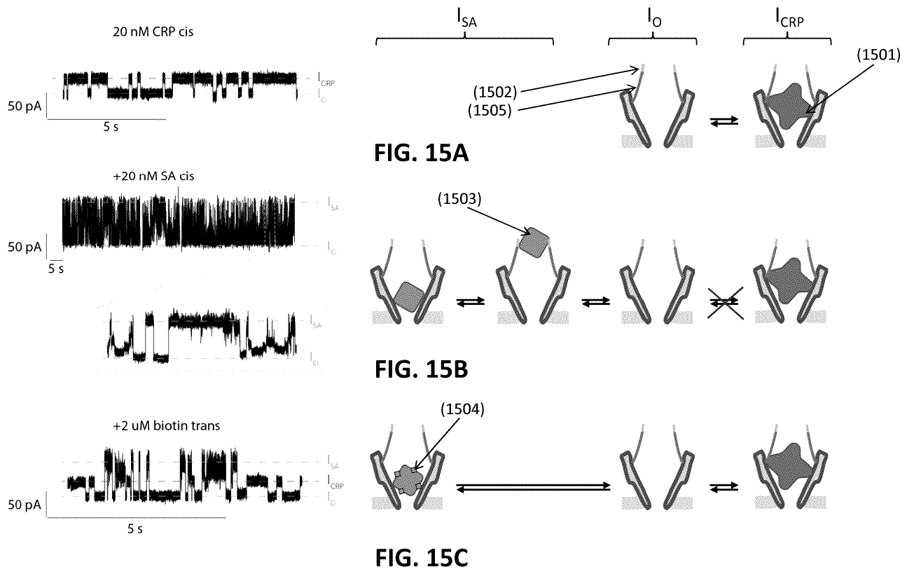

FIGS. 15A-15C shows an example of capturing and filtering target analytes. FIG. 15A shows addition of 20 nM CRP (1501) to a nanopore and/or nanopore system. The current output characterizes the open-pore (I_{O}) and the blockage current for CRP (I_{CRP}). The nanopore captures the CRP protein (1501), providing a reading of the blockage current I_{CRP} current and demonstrating the analyte resides in the nanopore. As shown by the two-way arrows, the capture of the analyte is reversible and the current output may return to an open-pore level if the analyte leaves the pore. FIG. 15B shows the capture of a second analyte, streptavidin (SA; 1503), by a recognition element. The recognition element (1502) can be conjugated to the nanopore via a linker (1505) The recognition element can have an affinity for the SA analyte, allowing for the capture of the analyte and an increased dwell time in the nanopore. The current output can now be seen as comprising the blockage current for SA (I_{SA}). The recognition element can be selective for SA, so it further inhibits the capture of the CRP analyte, allowing for filtering of analytes in a complex sample. In FIG. 15C, the streptavidin can comprise conjugated biotin (1504) which may inhabit the binding sites targeted by the recognition element. The biotin may allow for the SA to be captured by the nanopore, but it can reduce the effect of the recognition element y competing for the same binding sites on SA, and may decrease the dwell time of the analyte in the pore. The CRP analyte can now also be captured by the nanopore as a result of the reduced effectiveness of the recognition element, and the current output displays the open-pore current signal and blockage currents of both analytes (I_{SA} and I_{CRP}). Furthermore, the change from FIG. 15B to FIG. 15C demonstrates that the small biotin analyte can be detected by proxy due to the change in residence time of the SA and CRP.

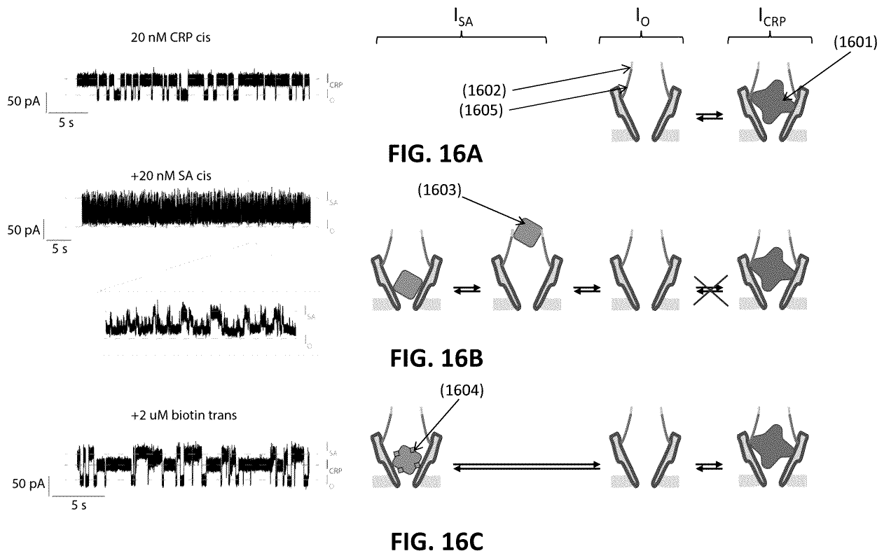

In FIG. 16A, the CRP protein (1601) can reside in the nanopore, which may result in characteristic blockage current, I_{CRP}. Upon addition of 20 nM SA in FIG. 16B, the SA (1603) is bound by the recognition element (1602) which is attached to the nanopore via a linker (1605). The recognition element captures the SA and can increase a dwell time in the nanopore. The increased dwell time can result in a dominating SA blockage current, I_{SA}. In FIG. 16C, biotin (1604) may be attached to the analyte. The biotin can interfere with the binding of the recognition element (1602) which decreases the dwell time of SA in the pore. The decreased SA dwell time can lead to CRP capture in the pore and CRP blockade events in the current output.

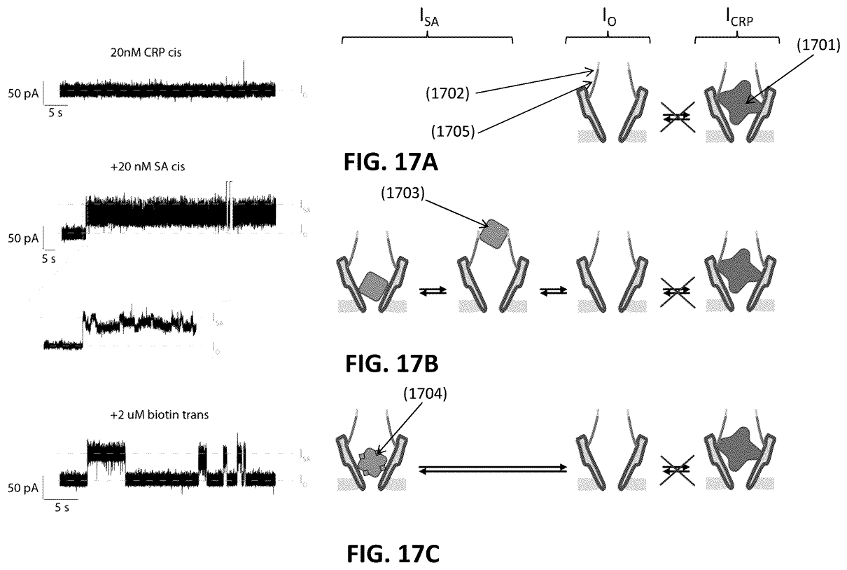

In FIG. 17A, the CRP (1701) may be unable to reside in the nanopore, which may result in a dominant open-pore current, I_{O}. As SA is added in FIG. 17B, the SA analyte (1703) binds to the recognition element (1702), the capture of the analyte can produce a dominant SA blockage current (I_{SA}). The CRP (1701) may not be able to dwell in the nanopore and may show no blockade events. The conjugation of biotin (1704) to the SA can reduce the dwell time of SA in the pore, which may reduce the frequency of SA blockage current in the output.

A method of the invention is suitably used for the real-time identification of at least one target (proteinaceous) analyte in a sample. In some embodiments, real-time identification of an analyte using the nanopores, methods, and/or system described herein may comprise a time period of less than 10 seconds, 9 seconds, 8 seconds, 7 seconds, 6 seconds, 5 seconds, 4 seconds, 3 seconds, 2 seconds, 1 seconds, 500 milliseconds, 400 ms, 300 ms, 200 ms, 100 ms, 50 ms, 10 ms, 5 ms, or less than 5 ms. The method of the invention is suitably used to characterize a mixture of analytes in a mixed sample. The method of the invention in suitably used to determine unique characteristics of at least one target analyte in a sample, including but not limited to the identity, concentration, shape, structure, mutations, surface charge, post-translational modifications or bound ligands. The unique characteristic can refer to a characteristic distinct to an analyte in a complex sample (e.g., a characteristic not shared by at least one other analyte in the sample).

### Other Embodiments

In particular, a goal of the invention can be to provide a uniformly sized nanopore with a sufficiently large diameter and an appropriate selectivity to allow capture of >80 kDa, preferably >100 kDa folded proteins. The nanopore system can be readily adapted to enhance selective capture of (unlabeled) analytes from a complex mixture of components, e.g. biomolecules such as proteins. Preferably, the nanopore can be sufficiently stable under conditions used for electrophysiological sensing experiments. Furthermore, the nanopore can enable reliable real-time identification of various size proteins in complex biological samples.

The inventors surprisingly found that at least some of these goals can be met by the provision of a conical shaped nanopore, such as the YaxAB nanopore having a large (about 15 nm for the hetero-dodecameric species) cis opening and a much smaller (about 3.5 nm) trans constriction region. This unique pore geometry can allow for the characterisation of an unprecedented wide range of (protein) analyte sizes and may make it the largest proteinaceous nanopore for molecular analysis characterized thus far. Molecular dynamics and electrical recording showed that the resistance of the nanopore may be dominated by the trans constriction region. In turn, the charge of nanopore, particularly at the constriction, can generate a strong electroosmotic flow (EOF) that promotes the capture of proteins with a wide range of net electrostatic charges. Conveniently, proteins in at least the 33-120 kDa range can be trapped within the conical shape of the nanopore for a time that can be tuned by the external bias. Interestingly, and contrary to the currently used cylindrical nanopores, the current blockage can decrease with the size of the trapped protein, as smaller proteins penetrate deeper into the constriction region than larger proteins. This characteristic can be especially useful for characterising large proteins, as exemplified for pentameric C-reactive protein (CRP), a widely used health indicator of around 120 kDa, which shows a unique signal that could be identified in real-time in the presence of depleted blood.

Accordingly, the invention provides a sensor system comprising a proteinaceous nanopore embedded in an amphipathic or hydrophobic membrane separating a fluid filled chamber into a cis side and a trans side, wherein the nanopore can be a conical shaped proteinaceous nanopore having a cis entrance of at least 11 nm and a trans constriction of less than 5 nm. The conical nanopore may have a cis entrance of about 12 to 20 nm, and/or a trans constriction of 2 to 4 nm. A sensor system of the invention can allow for capture of proteins, multi-protein assemblies and protein-ligand complexes within the conical shape (vestibule) of the nanopore for a time period that can be tuned by an external bias.

In one embodiment, the nanopore system comprises a two-component, or bipartite, heterooligomeric pore, such as the alpha-xenorhabdolysin family of binary toxin or an ortholog thereof toxin like the YaxAB toxin of Yersinia enterocolitica or XaxAB of Xenorhabdus nematophila. The Yersinia YaxAB system represents a family of binary α-PFTs with orthologues in human, insect, and plant pathogens.

A nanopore system of the invention may comprise members of the Alpha-xenorhabdolysin family of binary toxins, for example the toxin of Yersinia enterocolitica (YaxA, YaxB), Providencia alcalifaciens (PaYaxA, PaYaxB), Pseudomonas syringae (PsYaxA, PsYaxB), Proteus mirabilis (PmYaxA, PmYaxB), Morganella morganii (MmYaxA, MmYaxB), Photorhabdus luminescens (PaxA, PaxB), or Xenorhabdus nematophila (XaxA, XaxB).

Table 5 herein below provides the amino acid sequences of Alpha-xenorhabdolysin family binary toxin orthologues. Also encompassed are full-length, wild-type, mutant, functional and/or truncated variants of these toxin forming proteins.

Preferably, the nanopore comprises an oligomeric assembly of YaxA and YaxB subunits, or variant(s) thereof. The nanopore may comprise different number of monomeric units. For example, the conical nanopores can be formed by an oligomeric assembly of 7 to 13, preferably 8 to 12, heterodimers of YaxA and YaxB subunits, or orthologs thereof. In one embodiment, the nanopore comprises a nanopore in a decamer of YaxAB heterodimer arrangement, herein also referred to as 20-mer or hetero-eicosameric nanopore.

In one embodiment, at least one of the YaxA and/or YaxB subunits or orthologs thereof can be N-truncated variants. For example, provided is a sensor system comprising a truncated variant of YaxA, in particular YaxA lacking at least partially the unstructured N-terminal region. For example, YaxA or its ortholog may lack residues 1-20, or 1-30, preferably 1-40, more preferably 1-41, as found in ProteinID YE1984 or the corresponding N-truncated ortholog thereof. Alternatively, the full-length version of YaxA or its ortholog can be suitably used. YaxB may also be used as full-length protein or as truncated variant. In a specific aspect, the nanopore system comprises N-terminally truncated YaxA subunits or orthologs thereof in combination with full-length YaxB subunits or orthologs thereof.

The optionally truncated YaxA subunit or ortholog thereof may comprise one or more of the mutations. For example, one or more amino acid substitution can be made on the basis of a sequence comparison with orthologues of YaxA, such as PaxA, Mm YaxA and/or XaxA. See Figure S12 of Brauning et al. for multiple sequence alignment of YaxA and YaxB orthologues. Preferably, conserved amino acids or regions, such as the hydrophobic foot, conserved residues facing the lipid milieu as part of the transmembrane segment and/or residues engaged in YaxB-YaxB contacts, can be maintained in a construct for use in the present invention.

Exemplary variable amino acid positions can include R150, K250 and S282 of the YaxA sequence. For example, PaxA and XaxA can have G at position R150, MmYaxA can have R at position K250, and six YaxA orthologues can have G at position S282. Accordingly, in one embodiment the optionally truncated YaxA subunit may comprise one or more of the mutations R150G, K250R and S282G with respect to the sequence of ProteinID YE1984. A further optional mutation is N17S.

The nanopore system may comprise one or more variant YaxB subunit(s) or ortholog(s) thereof comprise mutation V284I, wherein the residue numbering corresponds to ProteinID YE1985.

In a specific aspect, the invention can involve the use of a so-called EOF mutant wherein one or more negatively charged lumen facing residue(s) in the constriction region can be mutated to a neutral residue to remove the EOF, or to a positive residue to reverse the EOF. In one embodiment, a nanopore system comprises a EOF mutant of YaxB, preferably YaxB comprising mutations E208N, E212N and/or D214N or YaxB comprising E208R, E212R and/or D214R.

In certain analyte sensing applications, it may be desirable to tune the residence time of a target analyte in the vestibule of the conical nanopore. Whereas it is often sufficient, or even preferred, to have short residence of >10 ms, e.g., 10 ms to 1 sec, for basic analyte detection, it can be in some cases advantageous to have a much longer residence time of >1 sec. Depending on the analyte and/or the nanopore characteristics, if needed, the trapping time may be increased by the functionalization of the proteinaceous conical nanopore. Herewith, the functionalized nanopore can enhance capture frequency of the target analyte from solution into the nanopore vestibule and/or reduces the unbinding (release) of the target analyte from the nanopore. Suitably, the conical nanopore can be functionalized at, or near to, the top of its cis entrance with one or more polymeric extensions, optionally also comprising one or more recognition element(s) capable of specifically binding to a target analyte. The recognition element R can but does not need to be of proteinaceous nature; it can be a small-molecule (e.g., a ligand to a target protein), a protein (folded or unfolded), DNA, RNA, etc. The molecular weight or size of the (proteinaceous) recognition element can vary. In one aspect, it can be small e.g. below 5 kDa.

R can be conjugated to a nanopore subunit by any known means in the art, including chemical conjugation (e.g. using cysteine coupling chemistries, click chemistries, etc.) or biological attachment e.g. by genetic fusion. For example, a nanopore comprising YaxAB subunits or orthologs thereof can be functionalized by modification of one or more A and/or B subunits. Individual nanopore subunits can be functionalized with the same or with different recognition elements. Herewith, nanopores with different functionalities in various stoichiometries can be obtained when the subunits may be mixed. Alternatively, two or more different recognition elements can be added to one subunit by concatenating the different recognition elements together (with or without intervening section of linker). By building multiple different recognition elements into a single oligomeric nanopore (whether formed of differentially modified subunits or formed from a single species of subunit that contains multiple different recognition elements) it can be possible to control capture and binding of multiple different target analytes to a single nanopore sensor. Alternatively, the multiple recognition elements on a single nanopore might bind to different regions of the same target analyte to increase the specificity for detecting the given target analyte over binding to unwanted analytes in a mixture.

The recognition element(s) can be preferably attached to the nanopore via a flexible (unstructured) linker moiety. The linker moiety can consist or comprise proteinaceous, DNA, other unstructured polymeric moietie(s) such as PEG etc., or any combination thereof.

The linker length can vary according to needs. For example, the linker can be at least 1 nm, or at least 3 nm, or at least 6 nm, or at least 10 nm or at least 20 nm. Longer linkers of 25 nm or more, 30 nm or more, or 50 nm or more may also be envisaged. In one aspect, the linker can have a length in the range of 1-30 nm, 1-25 nm, 6-25 nm, 1-10 nm, or 10 to 30 nm.

In one embodiment, the at least one recognition element can be attached to the nanopore via a peptide linker sequence. Good results can be obtained with a nanopore system comprising an oligomeric assembly of subunits, wherein at least one subunit can be functionalized with a recognition element via an N- and/or C-terminal peptide extension comprising a linker sequence and recognition element. Suitably, the peptide linker sequence comprises at least 3 amino acids, preferably 3 to 100 amino acids, more preferably 10 to 70 amino acids.

Protein linkers may be known in the art, and can include three major types of linkers: flexible, rigid, and in vivo cleavable. Flexible linkers may consist (mainly) of many small glycine residues, giving them the ability curl into a dynamic, adaptable shape. Rigid linkers may be formed of large, cyclic proline residues, which can be helpful when highly specific spacing between domains must be maintained.

Preferred amino acids constituting a linker sequence for use in the present invention can include a wide range of amino acids, including hydrophilic and aromatic amino acids. The linker can be preferably mostly unstructured, but can also have rigid elements and/or a-helical elements. Exemplary amino acid sequence motifs include Ala-Pro (rigid AP motif), the EAAAK motif (alpha helical rigid) and FG-motif. In a specific aspect, a peptide linker can be mainly composed of G, S, T, and very few A and N. Charged linkers may contain R and K (positively charged), or D and E (negatively charged).

The peptide extension may be attached to the full-length nanopore-forming subunit, or it may attach to one or more truncated nanopore subunits from which at least part of the N-/or C-terminal (unstructured) region may have been removed. For example, an extension peptide comprising a recognition element and a flexible linker sequence may be used to replace at least part, preferably all, of an unstructured terminal region of YaxA or an ortholog thereof. In one embodiment, an extension peptide comprising a recognition element and a flexible linker sequence can be fused to the N- or C-terminus of YaxB or an ortholog thereof. In a specific aspect, the invention provides a nanopore system comprising YaxAB nanopores wherein at least the YaxB subunits can be functionalized. For example, good results are obtained wherein YaxB monomers are N- or C-terminally fused to an extension peptide comprising at its “free” terminus a proteinaceous recognition element.

See Table 2 for exemplary functionalized YaxB subunits.

The invention also provides a functionalized YaxA or YaxB polypeptide or ortholog thereof that may be capable of forming a conical shaped nanopore, the functionalized polypeptide comprising a recognition element R capable of specifically binding to a target analyte of interest. As described herein above, R can be of proteinaceous or non-proteinaceous nature, for example R can be a small-molecule, a protein (folded or unfolded), DNA, RNA, etc. Preferably, R can be a proteinaceous moiety. The functionalized YaxA or YaxB polypeptide or ortholog may comprise a variant, mutant and/or truncated version of YaxA, YaxB or ortholog thereof as described herein above.

In one aspect, R can be attached to the variant polypeptide via a flexible linker L, preferably wherein L can be a polypeptide, a polynucleotide or any other type of unstructured polymer, such as PEG. Preferably, the flexible linker can be a polypeptide linker, e.g. a polypeptide linker comprising at least 3 amino acids, preferably 3 to 100 amino acids, more preferably 10 to 70 amino acids, e.g. 12, 15, 20, 25, 30, 35, 40, 50, 60 or 65 amino acids.

Suitably, a proteinaceous recognition element R can be genetically fused to the N- and/or C-terminus of an optionally truncated YaxA or YaxB polypeptide or ortholog thereof. Preferably, a proteinaceous R can be fused to said YaxA, YaxB or ortholog thereof via a peptide linker L. In one aspect, the invention provides an optionally (N-) truncated YaxA polypeptide that can be functionalized. The YaxA polypeptide may comprise one or more of the mutations N17S, R150G, K250R and S282G. In another aspect, the invention provides a YaxB polypeptide that can be functionalized with an additional sequence at the N-terminus or C-terminus, composed of a recognition element and a linker. The YaxB polypeptide may be full-length. It may comprise mutation V284I.

The functionalized nanopore-forming subunit can advantageously comprise one or more additional sequences (motifs) known in the art that can aid in the (recombinant) production and/or purification of the variant polypeptide. These include protein purification tags, e.g. His6-tag, Strep-tag, SUMO tag, MBP tag, etc. and protease cleavage sites, such as tobacco etch virus (TEV) protease cleavage site. The additional motifs can be separated by a spacer.

In a specific aspect, the nanopore subunit, e.g. an YaxA, YaxB or ortholog thereof, can include an additional 24 amino acids at its N-terminus: MSYY, followed by FH-4HHHHH (6×His tag), DYDIPTT (a spacer region), ENLYFQG or ENLYFQS (TEV protease cleavage site).

Also provided herein is a conically shaped nanopore comprising at least one variant YaxA or YaxB polypeptide or ortholog thereof to which a recognition element R capable of specifically binding to a target analyte can be attached, e.g. by chemical attachment or by genetic fusion.

Still further, the invention relates to an isolated nucleic acid molecule encoding a functionalized and/or mutated YaxA or YaxB polypeptide or ortholog thereof as described herein above.

Also provided is an expression vector comprising the nucleic acid molecule, and a host cell comprising such expression vector.

According to the invention, a sensor system comprises a conical shaped proteinaceous nanopore embedded in an amphipathic or hydrophobic membrane. The term “membrane” can be used herein in its conventional sense to refer to a thin, film-like structure that separates the chamber of the system into a cis side (or cis compartment) and a trans side (trans compartment). The membrane separating the cis and trans compartments comprises at least one conical shaped proteinaceous nanopore. Membranes can be generally classified into synthetic membranes and biological membranes. Any membrane may be used in accordance with the invention. Multiple nanopores may be present in one membrane.

The membrane can be preferably an amphiphilic layer. An amphiphilic layer can be a layer formed from amphiphilic molecules, such as phospholipids, which have both at least one hydrophilic portion and at least one lipophilic or hydrophobic portion. The amphiphilic layer may be a monolayer or a bilayer. The amphiphilic molecules may be synthetic or naturally occurring. Non-naturally occurring amphiphiles which form a monolayer are known in the art and include, for example, block copolymers (Gonzalez-Perez et al., Langmuir, 2009, 25, 10447-10450).

The nanopore system typically comprises a cis chamber comprising a first conductive liquid medium in liquid communication with a trans chamber comprising a second conductive liquid medium. The conductive liquid medium in the chambers of the nanopore system can have a wide range of ionic contents well known in the art, typically from 0.05 M to >3 M. A wide range of salts can be used, such as NaCl and KCl. Suitable solutions include 150 mM NaCl, 50 mM Tris-HCl, pH 7.5. The cis and trans chamber may be symmetric or asymmetric. A wide range of pH and temperature conditions can be used, for example in the range of pH 3-11, 10-80° C., preferably at about room temperature or at about 37° C. Suitably, the cis chamber may comprise a crowding or blocking agent that reduces unwanted nonspecific protein adsorption. In one embodiment, the blocking agent can be BSA.

The system may comprise a circuit that can both apply the voltage and measure the current. Alternatively, it comprises one circuit to apply the voltage difference and another to measure the current. It can also be possible to create the voltage difference with an asymmetric salt across the membrane. For example, one of the chambers may contain a solution of high ionic strength. Exemplary means for detecting the current between the cis and trans chambers were described in WO 00/79257 Patent Application No. 6,46,594, 6,673, 6,673,615, 6,627,067, 6,464,842, 6,362,002, 6,267,872, 6,015,714, 6,428,959, 6,617,113 and 5,795,782 and US Publications Nos. 2004/0121525, 2003/0104428 and 2003/0104428. They may include electrodes directly associated with the channel or pore at or near the porous opening, electrodes placed within the cis and trans chambers, and insulated glass microelectrodes. Electrodes may be capable of, detecting differences in ionic current around two chambers or tunneling electrical current around the porous opening. In another configuration, the transport property can be the flow of electrons around the diameter of the aperture which can be monitored by electrodes placed adjacent to or touching the circumference of the nanopore. Said electrodes can be attached to an Axopatch 200B amplifier to amplify a signal.

It can be understood that acquisition systems described herein is not limited and that other systems for acquiring or measuring nanopore signals can be employed, including optical sensing e.g. essentially as described by Huang et al. (Nature Nanotechnology, Vol. 10, pg. 986-991 (2015). Alternative electrical schemes can also be employed, on arrayed chip platforms for example, to achieve an equivalent voltage drop across the nanopore and/or membrane.

The sensor system can be advantageously integrated in a portable device comprising a plurality of sensor systems. For example, it can be comprised in a point-of-care diagnostic medical devices, which are in vitro diagnostics used by health care professionals to obtain results rapidly near or at the site of a patient. These products can be useful to quickly determine a marker responsible for a certain disease, e.g., at a doctor's office or clinic. The device can be preferably designed for performing an analytical method as herein disclosed. The device can be a portable device, a medical device, implant, single use device, disposable device, In one aspect, the device can be configured to allow for real-time detection of at least one analyte, preferably a clinically relevant analyte.

As will be appreciated by a person skilled in the art, a method or sensor system of the invention can be readily designed to detect any target analyte (or multiple target analytes) of interest. The invention can be advantageously used to detect a label-free target analyte. The conical shaped nanopore system captures a wide range of particles in a similar size range. Examples include inorganic particles (e.g. gold beads), polymeric particles such as plastics/beads/dendrimers, oligomeric particles e.g. micelles, liposomes and other fatty droplets.

In one embodiment, the invention provides a method for detecting a target analyte/antigen selected from the group consisting of a protein, polypeptide, a protein assembly, a protein/DNA assembly, polysaccharide, lipid, lipid membrane, lipid particle, bacterium, virus capsid, virus particle, dendrimer, polymer, or any combination thereof.

In one embodiment, the target analyte can be a clinically relevant analyte, preferably a clinically relevant protein or fragment thereof. In a specific embodiment, the target analyte can be a cytokine, an inflammation marker (e.g. C-reactive protein) or a cell metabolite.

Preferably, the target analyte can be a protein, for example selected from the group consisting of a folded/native protein, a protein biomarker, a pathogenic protein, a cell surface protein.

The present invention can be particularly suitable for detecting protein targets covering a very wide range of masses and dimensions, from very small proteins and peptides to very large proteins and complexes.

As explained herein above, the present invention can be particularly suitable for detecting (folded) protein analytes or protein complexes that are larger than 80 kDa, preferably larger than 100 kDa, most preferably larger than 150 kDa.

In one aspect, the size and geometry of the target analyte only allow entry and exit at the wide cis end into the vestibule of the conical nanopore, while it cannot pass the narrow constriction region of the pore to prevent translocation. For detecting an analyte, one dimension (length or width) larger than the constriction can be enough. For analyte trapping, it may be preferred that both are larger than the constriction. For example, the analyte has a geometry of 2-20 nm, preferably >3 nm and <15 nm, in at least one, preferably at least two dimensions. The analyte may have a hydrodynamic radius of at least 20 Å, preferably at least 25 Å, more preferably at least 28 Å or at least 30 Å. In one aspect, it has a hydrodynamic radius in the range of about 25 to 50 Å, preferably 28 to 50 Å

In another aspect, the target analyte can be too small to be captured in the nanopore vestibule since its size and geometry of the allow entry and exit at both the wide end and the constriction region of the pore. For example, it has a geometry of less than 5 nm, preferably 1-4 nm, in at least two dimensions. In these cases, the analyte can be suitably detected by using a large binder to trap the small target analyte that would otherwise be too small to be detected by the nanopore. See further herein below for “complexation” methods and systems involving the capture and analysis of small analytes using a cognate binder.

As will be recognized and appreciated by a person skilled in the art, a nanopore system as herein disclosed finds its use in various applications, ranging from analytical detection methods in a research setting, high throughput drug development to real-time diagnostic applications.

In one aspect, the invention provides a method for detecting and/or characterizing at least one target analyte using a nanopore system according to the invention, comprising: (a) allowing capture of the target analyte by the nanopore so that the analyte temporarily lodges into the conical vestibule of the nanopore; (b) optionally applying an electrical potential across the nanopore; and (c) measuring ionic current passing through the nanopore, wherein a change in the frequency and/or magnitude of ionic current indicates the presence, concentration, identity and/or other characteristics of the target analyte in the sample. For example, an analyte of interest may occur in various different forms e.g. structure, shape, free or ligand bound, post-translationally modified etc. or it may have any other type or level of heterogeneity. In one aspect of the invention, the conical nanopore can distinguish between such different forms.

Typically, a sample to be analyzed for the presence of a target analyte that can be captured and trapped by the conical nanopore can be added to the cis chamber of the nanopore system.

As described herein above, a method of the invention can be suitably used to detect any type of analyte. In one aspect, the method comprises detecting and/or characterizing a protein, protein assembly or protein complex, preferably wherein the target analyte comprises a folded protein. For example, the target analyte a) comprises or consists of a (folded) protein having a molecular weight of at least 80 kDa, most preferably at least 100 kDa; b) has a size of 2-20 nm, preferably >3 nm and <15 nm, in at least two dimensions; and/or c) has a hydrodynamic radius of at least 20 Å, preferably at least 25 Å, more preferably at least 28 Å or at least 30 Å.

A method according to the invention can be very suitable for the analysis of a complex sample, e.g. a solution comprising a mixture of components including one or more target analytes and one or more unwanted analytes. For example, the sample can be a complex sample comprising a mixture of proteins, preferably wherein the sample comprises a (diluted) clinical sample, more preferably a bodily fluid or sample, such as whole blood, plasma, blood serum, urine, feces, saliva, cerebrospinal fluid, nasopharyngeal swab, breast milk or sputum. In another aspect, the sample comprises (diluted) complex media.

In one embodiment, the invention provides an analytical method using functionalized conical shaped nanopores for selectively capturing and/or filtering different analytes in the cis solution. The method suitably comprises the use of a nanopore system comprising a conical shaped biological nanopore that can be functionalized to selectively enhance the capture of a target analyte from a complex mixture of components, such as biomolecules. The functionalized nanopore partially or fully traps the analyte in the vestibule of the nanopore. Using this strategy, it can also be possible to filter for desired target analyte(s) from unwanted target analytes in (complex) mixtures such as clinical samples. See for example FIGS. 12A-12C showing a schematic model of these selective “capture and filtering” strategies.

Preferably, a ‘capture and filtering’ method as herein disclosed employs a nanopore system comprising nanopores that are functionalized at the cis end with an R element as defined herein above. In a specific aspect, the method relies on YaxAB nanopores that are functionalized with a polymeric (e.g. peptide) extension containing a recognition element that can be specific to a target analyte enables binding of the target analyte, and subsequently promotes capture of the analyte into the nanopore vestibule. See FIGS. 12A-12C for a schematic representation.

For samples to be analyzed for the presence of a very small target analyte that cannot be captured and trapped by the conical nanopore, the target analyte can be advantageously detected as part of a complex formed with a large cognate binding protein, which protein-analyte complex can be captured by the conical nanopore.

Hence, the invention provides a method for detecting and/or characterizing at least one analyte (A), wherein A can be detected as part of a complex formed with a cognate binding protein (BP), comprising adding to the cis side of the conical nanopore a BP that cannot pass the trans constriction of the nanopore, and allowing for capture of the binding protein-analyte (BP-A) complex by the nanopore. The binding protein-analyte (BP-A) complex may form in solution before capture of the complex by the nanopore and/or BP can be first captured in the nanopore and subsequently binds A to form a BP-A complex. See FIGS. 8A-8B for a schematic representation.

To ensure that BP cannot pass from the cis to the trans side of the nanopore system, it can be advantageous that BP has a size of 2-20 nm, preferably >3 nm and <15 nm, in at least two dimensions and/or a hydrodynamic radius (rh) of at least 20 Å, preferably at least 25 Å, more preferably at least 28 Å or at least 30 Å. In one aspect, it has a rh in the range of about 25 to 50 Å, preferably 28 to 50 Å.

In one embodiment, this “complexation” method comprises detecting and/or characterizing at least one small target analyte (Å) capable of passing the trans constriction of the conical nanopore, wherein said small analyte can be captured in the nanopore by complexing to a large cognate binding protein (BP) that cannot pass the trans constriction of the nanopore. In these cases, the analyte may be added to the cis and/or trans side of the nanopore system, whereas BP can be added to the cis side.

Importantly, this “complexation” approach may not be restricted to detection of small analytes; it can also be suitably used for protein-analyte complexes comprises two or more large entities, e.g. large protein-protein complexes or oligomers. The protein-complex (or indeed any complex) method can be used to detect both small and large radius by virtue of the difference between the unbound binder state and the bound-binder state. This approach can be suitably used to detect protein-ligand complexes, e.g. protein-drug binding, protein-cofactor binding, enzyme-substrate binding, and the like.

In a preferred embodiment, for example when the sample can be a complex biological sample, the invention provides a detection method wherein the complexation approach can be combined with a trapping and filtering strategy. See Example 6.

A method of the invention can be suitably used for the real-time identification of at least one target (proteinaceous) analyte in a sample. The method of the invention can be suitably used to characterize a mixture of analytes in a mixed sample. The method of the invention in suitably used to determine unique characteristics of at least one target analyte in a sample, including but not limited to the identity, concentration, shape, structure, mutations, surface charge, post-translational modifications or bound ligands.

### Computer Systems

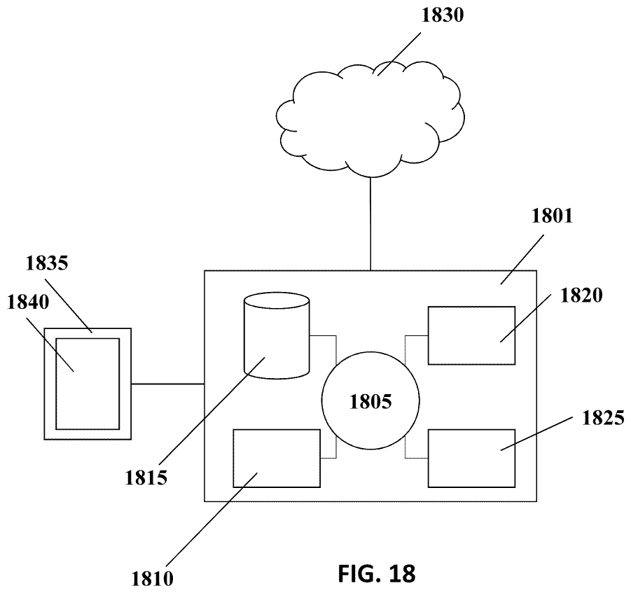

The present disclosure provides computer systems that are programmed to implement methods of determining one or more characteristics of an analyte. FIG. 18 shows a computer system 1801 that is programmed or otherwise configured to determine one or more characteristics of an analyte. The computer system 1801 can regulate various aspects of detecting presence or absence of one or more characteristics of the analyte, such as, for example, determining the sequence of the analyte. The computer system 1801 can be an electronic device of a user or a computer system that is remotely located with respect to the electronic device. The electronic device can be a mobile electronic device.

The computer system 1801 includes a central processing unit (CPU, also “processor” and “computer processor” herein) 1805, which can be a single core or multi core processor, or a plurality of processors for parallel processing. The computer system 1801 also includes memory or memory location 1810 (e.g., random-access memory, read-only memory, flash memory), electronic storage unit 1815 (e.g., hard disk), communication interface 1820 (e.g., network adapter) for communicating with one or more other systems, and peripheral devices 1825, such as cache, other memory, data storage and/or electronic display adapters. The memory 1810, storage unit 1815, interface 1820 and peripheral devices 1825 are in communication with the CPU 1805 through a communication bus (solid lines), such as a motherboard. The storage unit 1815 can be a data storage unit (or data repository) for storing data. The computer system 1801 can be operatively coupled to a computer network (“network”) 1830 with the aid of the communication interface 1820. The network 1830 can be the Internet, an internet and/or extranet, or an intranet and/or extranet that is in communication with the Internet. The network 1830 in some cases is a telecommunication and/or data network. The network 1830 can include one or more computer servers, which can enable distributed computing, such as cloud computing. The network 1830, in some cases with the aid of the computer system 1801, can implement a peer-to-peer network, which may enable devices coupled to the computer system 1801 to behave as a client or a server.

The CPU 1805 can execute a sequence of machine-readable instructions, which can be embodied in a program or software. The instructions may be stored in a memory location, such as the memory 1810. The instructions can be directed to the CPU 1805, which can subsequently program or otherwise configure the CPU 1805 to implement methods of the present disclosure. Examples of operations performed by the CPU 1805 can include fetch, decode, execute, and writeback.

The CPU 1805 can be part of a circuit, such as an integrated circuit. One or more other components of the system 1801 can be included in the circuit. In some cases, the circuit is an application specific integrated circuit (ASIC).

The storage unit 1815 can store files, such as drivers, libraries and saved programs. The storage unit 1815 can store user data, e.g., user preferences and user programs. The computer system 1801 in some cases can include one or more additional data storage units that are external to the computer system 1801, such as located on a remote server that is in communication with the computer system 1801 through an intranet or the Internet.

The computer system 1801 can communicate with one or more remote computer systems through the network 1830. For instance, the computer system 1801 can communicate with a remote computer system of a user (e.g., a personal computer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1801 via the network 1830.

Methods as described herein can be implemented by way of machine (e.g., computer processor) executable code stored on an electronic storage location of the computer system 1801, such as, for example, on the memory 1810 or electronic storage unit 1815. The machine executable or machine readable code can be provided in the form of software. During use, the code can be executed by the processor 1805. In some cases, the code can be retrieved from the storage unit 1815 and stored on the memory 1810 for ready access by the processor 1805. In some situations, the electronic storage unit 1815 can be precluded, and machine-executable instructions are stored on memory 1810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1801 can include or be in communication with an electronic display 1835 that comprises a user interface (UI) 1840 for providing, for example, the identification of the target nucleic acid sequence. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1805.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and the computer memory coupled thereto. The computer memory comprises machine executable code that, upon execution by the one or more computer processors, implements any of the methods above or elsewhere herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Examples

### Example 1: Development of YaxA/YaxB Nanopores and Systems

### Materials

C-reactive protein (CRP, AG723), Thrombin (CAS #9002-04-4), Haemoglobin (CAS #54651-57-9), δ-Cyclohexylhexyl β-D-maltoside (Cymal-6, CAS #228579-27-9), Diphytanoyl-sn-glycero-3-phosphocholine (DPhPC, CAS #207131-40-6) and Hexadecane 99% (CA S #544-76-3) were obtained from Sigma/Merck. Phusion Polymerase (M0530) was ordered from NEB. Streptavidin (21122), GeneJET Plasmid Miniprep Kit (K0503) GeneJET PCR Purification Kit (K0701), Phire Hot Start II DNA polymerase (F1225), T4 DNA Ligase (EL0011), DpnI (ER1701), NdeI (FD0583) and HindIII (ER0502) restriction enzymes, and High Select™ Top 14 Abundant Protein Depletion mini spin columns (A36369) were ordered form Fisher Scientific. Other chemicals used were purchased from Carl-Roth. Sequencing was done by Macrogen and primers were acquired from Integrated DNA Technologies (IDT).

### Cloning of YaxA and YaxB Variants

YaxAB was prepared using pRSET-A plasmids encoding YaxA and YaxB genes. The YaxA construct (PDB: 6EL1) contained additional mutations N17S, R150G, K250R and S282G from the wild-type gene (YE1984). The YaxB construct (PDB: 6EL1) contained additional mutation V284I from the wild-type gene (YE1985). The pRSET-A plasmids encoding the YaxB-linker constructs were prepared with USER-cloning. The YaxA_{Δ40} variant was prepared by deleting the first 40 amino acids (and the N-terminal Methionine) from the unstructured part of the WT sequence by ultramer PCR. The gene was amplified with Phire Hot Start II DNA polymerase (100 μL final volume, pre-denaturation at 98° C. for 30 sec, 30 cycles of: Denaturation at 98° C. for 5 sec, Annealing at 54° C. for 15 sec and Extension at 72° C. for 30 sec) and purified using the GeneJET PCR Purification Kit. The YaxA_{Δ40} PCR product was digested with DpnI (to remove circular template DNA of YaxA) for 2 hours at 37° C., and restricted-digested using the NdeI and HindIII restriction sites. The PCR purified product was ligated into NdeI and HindIII pre-digested pRSET-A vector with T4 ligase, and transformed into electrocompetent E. cloni cells (Lucigen). Plasmids were purified from acquired transformants with the GeneJET Plasmid Miniprep Kit and sent for sequencing (Macrogen). Successful clones were selected for further protein purification. See Table 4 herein below for amino acid sequences of the various YaxA and YaxB constructs used.

### Expression and Purification of YaxA and YaxB Monomers

The pRSET-A plasmids containing the YaxA or YaxB gene variants were transformed into E. cloni® EXPRESS BL21(DE3) cells (Lucigen) by electroporation. The transformed cells were plated out on LB agar plates supplemented with 100 g/mL ampicillin and grown overnight at 37° C. The acquired colonies were inoculated into 2×YT medium supplemented with 100 g/mL ampicillin. The expression culture was grown at 37° C. while shaking at 200 rpm, until the optical density at 600 nm reached an OD₆₀₀ of ˜0.8. To induce protein expression, 0.5 mM Isopropyl 0-D-thiogalactopyranoside (IPTG) was added to the culture, which was then grown overnight at 25° C. while shaking at 200 rpm. The bacterial cells were harvested by centrifugation at 8000×g for 15 minutes and stored at −80° C.

The cell pellets were subjected to three freeze-thaw cycles to make the cells more susceptible to cell lysis. Each cell pellet, from 50 mL cell culture, was resuspended in 20 mL lysis buffer (50 mM Tris-HCl pH 8.0, 300 mM NaCl, 1 mM MgCl₂, 10 g/mL lysozyme, 0.2 U/mL DNase, with an additional 2 M urea for YaxA variants and one tablet of protease inhibitor EDTA-free per pellet of YaxB monomers) and incubated for 30 minutes at room temperature while shaking. The bacterial cells were disrupted by probe sonication at 30% output power for 3×60 seconds. Cell debris was removed by centrifugation at 4400×g for 30 min at 4° C. The supernatant was incubated for 15 minutes with 200 μL of Ni-NTA resin (Qiagen) at 4° C. while rotating at 10 rpm. The incubated resin was loaded onto a gravity flow column (Bio-Rad) and washed with 10 mL of wash buffer (50 mM Tris-HCl pH 8.0, 300 mM NaCl, 10 mM imidazole). The protein was eluted from the Ni-NTA resin with 3× times 200 μL elution buffer (50 mM Tris-HCl pH 8.0, 300 mM NaCl, 100 mM Imidazole). The protein concentration was measured by Bradford assay and the monomers were stored at 4° C. until oligomerization.

### Oligomerization and Purification of YaxAB

For oligomerization, the protein concentrations of both the YaxA and YaxB monomers were diluted to 1 mg/mL. Oligomerization was triggered by incubation of both YaxA and YaxB in a 1:1 ratio for 30 minutes at room temperature. It was shown that after oligomerization inactive oligodimers might be formed where the narrower trans sides of two pores stick to each other forming an hourglass shaped oligomer. To separate such oligomers, 1.5% w/v 6-Cyclohexylhexyl β-D-maltoside (Cymal-6) detergent was added to the solution for 30 minutes at 4° C. (longer incubation with Cymal-6 results in the dismantling of the pores back into monomers). Next, the incubation mixture was loaded on size exclusion chromatography (SEC) column to lower the concentration of Cymal-6 and to separate the unreacted monomers from single YaxAB pores. SEC was performed on the Akta pure chromatography system (Cytiva). YaxAB samples (500 μL) were loaded onto a Superose® 6 10/300 GL SEC-column (Cytiva) pre-equilibrated with buffer A (25 mM HEPES pH 7.0, 150 mM NaCl, 0.05% w/v Cymal-6). Protein elution was monitored by measuring absorbance at 280 nm wavelength. The first peak corresponded to the YaxAB oligodimers, the second peak corresponded to different oligomeric forms of YaxAB pores and the third peak corresponds to the YaxA and YaxB monomers. Fractions of both sides of the main peak corresponding to YaxAB nanopores were collected separately, as they correspond to different oligomeric forms. The three fractions were sampled into 20 μL aliquots and flash frozen in liquid nitrogen and stored at −80° C. for further use.

### Electrical Recordings in Planar Lipid Bilayers

The measuring setup consists of a chamber containing two 500 μL compartments (cis and trans) separated by a 20 m PTFE film with a central aperture of ˜100 m diameter. A lipid bilayer was formed on the aperture by adding a drop of hexadecane [4% (v/v) in pentane] on to the trans side of the PTFE film directly above the aperture. Next, each compartment was filled with 400 μL SDEX buffer (150 mM NaCl, 15 mM TrisHCl, pH 7.5) and then two drops of 5 mg/mL DPhPC lipids. Ag/AgCl electrodes were inserted to each compartment: trans was the connecting electrode, cis was the ground electrode. By lowering and raising the buffer level in one compartment, a lipid bilayer was formed over the aperture. After letting the bilayer stabilize for 5 min, a pipet tip was dipped into the YaxAB solution and dipped afterward into the buffer of the cis compartment. Upon pore insertion, −35 mV potential was applied, resulting usually in a current between −60 to −120 pA. For protein capture experiments, pores with a current of −80 pA±10% were used. All proteins were added to the cis, experiments were executed in triplicate. All measurements were conducted with a 50 kHz sampling frequency and a 10 kHz Bessel filter. All experiments were recorded with 150 mM NaCl, 15 mM Tris.HCl pH 7.5 in both cis and trans compartments, at room temperature and at −75 mV unless otherwise specified.

### Electrophysiological Data Recording and Analysis

All experimental nanopore data were recorded under a negative applied potential (−35 to −100 mV), using an Axopatch 200B patch clamp amplifier connected to a DigiData 1440 Å/D converter (Axon Instruments), using Clampex 10.7 software (Molecular Devices). I/V-curves were taken from −100 mV to +100 mV with increment of 10 mV. Data recordings were made in gap-free setting, or in a sweep protocol (−35 mV for 50 ms, +100 mV for 180 ms, and a measuring potential for 6 s). Recordings were analyzed with Clampfit 10.7 software (Molecular Devices). Only the experiments with depleted serum were additionally filtered with a Gaussian low-filter with 5 kHz cut-off prior to analysis. Open pore (I_{O}) current was determined from Gaussian fits to all-point histograms with a bin width of 0.5 pA. Protein block currents (I_{B}) were detected by the Single-Channel Search function in Event Detection. Residual current (I_{RES} (%)) was calculated as (I_{B}/I_{O})*100% for all events using in-house Matlab script. Dwell time and standard deviation of I_{B} (σ_{blockade}) per blockade event were also determined in the Single-Channel Search function. The σ_{blockade} was normalized by dividing it over the standard deviation of the open pore current (i.e. σ_{blockade}/σₒₚₑₙₚₒᵣₑ). The I_{O} and σₒₚₑₙₚₒᵣₑ were determined from a recording prior to adding analyte to cis (blank).

### Ion Selectivity Determination by Reverse Potential Experiments

Reverse potentials were obtained using similar electrophysiology set-up as described above, with exception of the following. Each compartment was filled with 400 μL 300 mM NaCl, 15 mM TrisHCl, pH 7.5. Ag/AgCl electrodes were separated from the compartments by 1% agarose bridges containing a 3M KCl solution. Upon pore insertion, the solution in trans was washed to 75 mM NaCl in six steps of 100 μL. I/V curves from −100 mV to +100 mV were collected before and after buffer replacement. The resulting voltage at zero current is the reversal potential (Vᵣ). The ion selectivity (P_{Na}_{⁺}/P_{Cl}_{^{−}}) was then calculated using the Goldman-Hodgkin-Katz equation (S1), where [a_{Na}_{⁺}_{/Cl}_{^{−}}]_{cis/trans} is the activity of the Na⁺ or Cl⁻ in the cis or trans compartment, R the gas constant (8.3145 J/mol·K), T the temperature (298 K) and F the Faraday's constant (96 485 C/mol).

$$\frac{{P}_{N {a}^{+}}}{{P}_{{Cl}^{-}}} = \frac{{[ {a}_{{Cl}^{-}} ]}_{trans} - {[ {a}_{{Cl}^{-}} ]}_{cis} \times {e}^{{V}_{r} F / RT}}{{[ {a}_{N {a}^{+}} ]}_{trans} \times {e}^{{V}_{r} F / RT} - {[ {a}_{N {a}^{+}} ]}_{c i s}}$$

The activity of ions was calculated by multiplying the molar concentration of the ion with the mean ion activity coefficients (0.7889 for 75 mM NaCl and 0.7224 for 300 mM NaCl, computed with linear regression; Lide et al., CRC handbook of chemistry and physics, 84th edition, 2003-2004. Handb. Chem. Phys. 53, 2616 (2003).

### Depleted Serum Experiments & CRP Titration

Human serum (100 μL; Sigma-Aldrich, H4522) was depleted using High Select™ Top 14 Abundant Protein Depletion mini spin columns (ThermoFisher Scientific, A36369). The final volume was 300 μL, hence the serum proteins were diluted 3× during depletion. YaxA_{Δ40}B*80 was used to detect serum proteins. Depleted human serum (2.5 μL) was added to a cis side (400 μL total volume), diluting the serum proteins 160×. For the titration experiment, YaxAB capturing depleted serum proteins was measured for ˜2 minutes, and then 20-80 nM CRP (final concentration) was titrated to the cis chamber and again measured for ˜2 minutes.

### Example 2—Characterisation of YaxAB Nanopores

CryoEM and X-Ray crystallographic analysis revealed that YaxAB form a transmembrane nanopore (PDB:6EL1) composed of YaxAB heterodimers, where YaxA occupy the exterior and YaxB the interior and transmembrane region of the nanopore rings. CryoEM analysis also revealed that the nanopore assembled in various stoichiometry, ranging from about 8 to about 12 heterodimers, with the decamer of dimers arrangement (composed of 10 units of YaxA-YaxB dimers; 20-mer) being the most frequently observed. The first 40 amino acids of the monomer YaxA are not resolved in the crystal structure, suggesting they form flexible random coils. Two variants of YaxA, the full-length (YaxA, FIG. 1A) and a truncated one, were prepared where the 40 amino acids of the N-terminal tail was deleted (YaxA_{Δ40}, FIG. 1B). Each of the two YaxA variants was mixed with full-length YaxB subunits. Both full-length YaxAB and YaxAΔ₄₀B variants assembled into lipid membranes forming conductive nanopores. When inserted into membranes, the YaxAB and YaxA_{Δ40}B nanopores produced a distribution of unitary conductance values (FIG. 2A), indicating that YaxAB nanopores assembled into different nanopore stoichiometries in lipid membranes. At −35 mV, the currents showed a distribution of −93±31 pA for YaxAB and −87±36 pA YaxA_{Δ40}B mutated nanopores, indicating that multiple pore sizes are present in the SEC-purified protein fractions. Further experiments primarily made use of the most prevalent size (−80 pA±10% at −35 mV in 150 mM NaCl): termed YaxAB*80 and YaxAΔ₄₀B*80, respectively. These pores likely correspond to the decameric nanopores.

Despite the presence of the N-terminus tails in the full-length YaxA, the YaxAB and YaxA_{Δ40}B nanopores were found to exhibit very similar conductance at both positive and negative applied voltages (FIG. 2B). Nanopores with different unitary conductance showed similar conductance properties and similar I/V curve shape relative to their smaller or larger relative conductance to the primary *80 population, further suggesting that nanopores with different oligomeric forms might be used for electrical recordings.

Non-equilibrium MD simulations of the decameric version of the YaxAB and YaxAΔ₄₀B nanopores was then performed. Both pores exhibited statistically indistinguishable currents at +125 mV, in agreement with the experimental observations. This is likely because the N-terminal tails fluctuated at the top of the nanopore where the resistance is lower. Quasi-1D approximation of the decameric YaxAB and the YaxA_{Δ40}B revealed that about 50% of the total resistance of the nanopore is in the first 3.5 nm from the trans entry of the nanopore, which may herein be referred to as the “constriction region”. By using the average electrolyte conductivity of NaCl 0.15 M (1.9 S/m) the estimated conductance of the YaxAΔ₄₀B and YaxAB nanopores are 2.55±0.2 nS and 2.85±0.2 nS, respectively, corresponding to 90±10 pA and 100±10 pA at +35 mV, which is close to the experimentally determined values.

### Example 2—Ion Transport Across YaxAB Nanopores

To characterise the nanofluidic properties of YaxAB nanopores, the reverse potential was measured under asymmetric salt conditions and used the Goldman-Hodgkin-Katz equation (see Methods) to calculate the ion selectivity and electro-osmotic properties. For YaxAB*80 and YaxA_{Δ40}B*80 the ion selectivity (P_{Na+}/P_{Cl-}) was 2.60±0.09 and 2.46±0.22, respectively (FIG. 2B). This indicates that both pores are highly cation selective. MD simulations showed that the nanopore constriction (region of about 0<z<35 Å) is predominantly negatively charged, which promotes the accumulation of cations. Under an applied bias, the electric field exerts a net volume force on the charged regions of the electrolyte, generating an electroosmotic flow (EOF) in the direction of the cation flow. The total electrical force acting on the electrolyte solution (i.e. the Coulombian force pushing the free moving ions) and the EOF at ΔV=±125 mV was estimated by non-equilibrium MD simulations, resulting in a net force for the YaxA_{Δ40}B pore of −119±92 pN at −125 mV, corresponding to an EOF of −61.3±3 molecules/ns. YaxAB comprising full-length YaxA showed slightly higher values, likely due to the negatively charged unstructured N-tails, which attract additional cations inside the pore.

In YaxAB three negatively charged rings were identified, facing towards the pore lumen and lying in the constriction region: E208, E212 and D214 of YaxB. These residues might govern the ionic selectivity of the pore and therefore the EOF. Additional MD simulations and experiments on two different mutated pores were performed, substituting the three acidic residues with either neutral or positively charged residues. MD results showed that by neutralising the charge of the three acidic rings (i.e. mutating them into neutral asparagines, E208N E212N D214N; NNN system) the EOF goes to approximately zero. By mutating the three rings into positively charged arginines (E208R E212R D214R; RRR system) the EOF was completely reversed with respect to the YaxA_{Δ40}B. In agreement with the MD simulations, in electrophysiology experiments a weakening and/or reversal of ion selectivity was observed: 0.80±0.04 and 0.71±0.01 for the NNN and the RRR system, respectively.

The maximum velocity field inside the constriction region for the YaxA_{Δ40}B pore is about 0.3 m/s, which is larger with respect to other commonly employed smaller nanopores, such as ClyA (<0.21 m/s at 100 mV, bi-cylindrical nanopore, 5.5-3.5 nm cis-trans diameters), FraC (<0.23 m/s at 100 mV, conical nanopore, 5.5 nm cis-diameter 1.5 nm trans-diameter, extrapolated values) and the α-Hemolysin (0.05 m/s at 125 mV for pH 7, 0.1 m/s at +125 mV at pH 2.8, cylindrical transmembrane region with 2 nm diameter). A similar average EOF velocity was also reported for artificial DNA-origami nanopores (0.3 m/s at 100 mV for a cylindrical nanopore of ˜2 nm diameter).

### Example 4—Capture and Detection of Protein Analytes

Biological nanopores with a large diameter can be used to detect and study folded proteins. Furthermore, YaxAB nanopores were found to have a strong cis-to-trans EOF arising from ion selective current flow, which can be very advantageous for driving capturing and trapping a wide range of analytes with diverse charge (FIGS. 3A-3B). FIGS. 3A-3B show a schematic model showing example YaxAB nanopores in a membrane for capture and characterisation of large analyte species (i) from the cis solution into the conical vestibule of the nanopore. The figure shows the reversible capture of large molecular analytes that can be characterized, for example by ionic current through the nanopore by application of an applied potential across the membrane.

To test the ability of YaxA_{Δ40}B*80 nanopores to capture and detect proteins, capture of four neutral or weakly charged proteins of different sizes were tested (FIG. 4): C-Reactive Protein (CRP, 125 kDa, hydrodynamic radius rₕ=43.5 Å, pI 7.4), Streptavidin (SA, 53 kDa, rₕ=33.8 Å, pI 7.00), Haemoglobin (HG, 64 kDa, rₕ=31.9 Å, pI 7.88) and Bovine Thrombin (BT, 35 kDa, rₕ=30.4 Å, pI 7.63). Ionic current blockades were only observed when each of the four proteins was added on the cis side and when a negative applied potential was applied (FIG. 5A), indicating that the protein entered the nanopore from cis to trans as a result of the strong cis-to-trans electroosmotic flow. Blockades were characterised by measuring the duration of the event (dwell time, ms), the residual current percent (Iᵣₑₛ (%)), as calculated from the ratio percent of the blocked (I_{B}) and open pore (I_{O}) currents (Iᵣₑₛ (%)=(I_{B}/I_{O})×100), and the blockade noise (σ_{blockade}, pA). See Table 1.

TABLE 1

| Protein | Mass (kDa) | rₕ (Å) | I_{RES} + σ (%) | σ_{blockade} | kₒₙ (μM⁻¹ s⁻¹) | k_{off} (s⁻¹) |
|---|---|---|---|---|---|---|
| CRP | 125 | 43.74 | 79.7 ± 2.1 | 1.4 ± 0.2 | 308.9 ± 9.7 | 27.0 ± 0.7 |
| buffer | (pentamer) | | | | | |
| CRP + | | | 78.9 ± 0.5 | 1.2 ± 0.1 | ND* | 36.8 ± 6.1 |
| serum | | | | | | |
| HG | 64 | 33.10 | 56.3 ± 3.6 | 0.95 ± 0.15 | 208.6 ± 8.9 | 189.1 ± 3.9 |
| | (tetramer) | | | | | |
| SA | 53 | 31.62 | 47.6 ± 2.9 | 1.1 ± 0.2 | 473.2 ± 16.6 | 13.3 ± 0.3 |
| | (tetramer) | | | | | |
| BT | 35 | 30.38 | 23.3 ± 3.4 | 1.3 ± 0.1 | 150.9 ± 4.4 | 121 ± 8.7 |
| | (dimer) | | | | | |

At −75 mV, CRP and SA showed well-defined narrow distribution clusters (FIGS. 5B-5C). HG and BT presented broader Iᵣₑₛ (%) and dwell time distributions, suggesting that the proteins might have more than one interaction site within the nanopore lumen.

FIG. 4 shows a schematic model showing use of exemplary YaxAB nanopores for characterization of different protein analytes. Snapshots were taken from the last frame of 25 ns SMD simulations. The figure shows models of the binding of four different folded proteins to YaxA_{Δ40}B*80 nanopores: C-Reactive Protein (CRP, 125 kDa, hydrodynamic radius=43.5 Å, pI 7.4), Streptavidin (SA, 53 kDa, hydrodynamic radius=33.8 Å, pI 7.00), Haemoglobin (HG, 64 kDa, hydrodynamic radius=31.9 Å, pI 7.88) and Bovine Thrombin (BT, 35 kDa, hydrodynamic radius=30.4 Å, pI 7.63). The four different protein analytes have different mass and hydrodynamic radius (as computed using HullRad software), and as a result sit at different z-locations (vertical axis) relative to bottom of the nanopore when captured into the vestibule. For modelling the z-axis was aligned vertical to the nanopore orientation, and perpendicular to the plane of the membrane, with axis origin the lowest features of the nanopore structure.

The voltage dependency of the escape times revealed that increasing the external bias caused the blockades to become longer with reduced Iᵣₑₛ (%), suggesting that increasing the EOF strength traps the proteins deeper in the conical structure of the nanopore and for longer. For all the four proteins, increasing the external bias increased the capture frequency and the blockade dwell time, which is consistent with EOF-driven capture.

FIGS. 6A-6B depict graphs showing the experimentally determined average residual current blockade level (Iᵣₑₛ (%)=I_{B}/I_{O}×100%) for CRP, SA, HG and BT versus their molecular weight (FIG. 6A) and hydrodynamic radius (FIG. 6B). Interestingly, the Iᵣₑₛ (%) of each protein at a given voltage was found to be inversely proportional to the analyte size (FIG. 6), with the largest molecule CRP showing the largest Iᵣₑₛ (%) (Iᵣₑₛ (%) [CRP]=77.68±0.5%, −75 mV) and the smallest BT the lowest Iᵣₑₛ (%) (Iᵣₑₛ (%) [BT]=38.6±2.5%, −75 mV). This is in contrast to the nanopore resistive pulse behaviour as observed for other large nanopores, such as ClyA and PlyAB nanopores, where larger proteins give a larger current blockade. This observation can be explained by the conical shape of the nanopore; large proteins sit higher up in the vestibule (where they block less of the ionic current due to a proportionality lower effect on the current flow near the constriction region), while smaller proteins sit lower/deeper into the vestibule (where they block more of the ionic current due to a proportionality larger effect on the current flow near the constriction region). It follows that, even if the CRP protein has a larger volume, it may not effectively occupy the constriction and it may block the current to a lesser extent. This hypothesis was tested by Steered-Molecular Dynamics (MD) simulations, where each protein was pulled from the cis to the trans side of the nanopore. The average steady state vertical position within the nanopore (z, relative to bottom most features of the nanopore) reached by each protein is inversely proportional to the volume of the protein, with the CRP protein lying at the centre of the pore (z_{CRP}˜80 Å) and BT lying deepest inside the constriction (z_{BT}<20 Å) (FIG. 4). Translocation events across the pore constriction were not observed within the MD simulation time. The theoretical computed blockades obtained from the last frames of the simulations were in good agreement with respect to the experimental values, suggesting that the SMD captured the essence of the hindrance mechanism of the four proteins inside the nanopore.

To test the ability of YaxAB nanopores to differentiate between different protein analytes in a complex mixture, YaxA_{Δ40}B*80 nanopores were tested with a mixture containing CRP (50 nM), SA (20 nM), HG (50 nM) and BT (50 nM) at −75 mV (FIGS. 5A-5C bottom panels). The proteins can be differentiated, enabling identification and quantification for example, by differences in the ionic current blockade signals, for example by differences in the Iᵣₑₛ (%), dwell time, σ_{blockade} and noise. FIG. 5A illustrates electrophysiology data for four protein analytes CRP, SA, HG and BT using YaxAΔ₄₀B*80 nanopores. FIG. 5A shows electrophysiology current-time traces showing representative ionic current data of the protein analytes measured as individual proteins in separate nanopore experiments (120 nM each analyte, added to a cis compartment), and when mixed together in a single experiment (50 nM BT, 50 nM CRP, 50 nM HG, 20 nM SA mixed in cis). All experiments were performed at −75 mV. The current-time traces show the characteristic changes in open-pore current (I_{O}) to a blockade current levels (I_{B}) upon capture of the proteins. Each protein demonstrated different Is current levels which can assist in detecting different analytes of a mixed (e.g., complex) sample.

FIGS. 5B-5C shows scatter plots of blockade events of data described in A), showing the dwell-time vs. residual current of each blockade event (FIG. 5B) and the blockade noise vs. residual current of each blockade event (FIG. 5C), for each of the protein analytes when measured individually and when mixed together. The figure shows that the different proteins can be discriminated from each other by both the magnitude of the ionic current blockades and the duration of the blockade events. For example, FIG. 5B shows that some of the analytes have markedly different dwell time as well as being separated by Iᵣₑₛ (e.g., CRP and Streptavidin have much longer dwell times). Similarly, noise (σ_{blockade}) can also be used to separate and identity, as FIG. 5C shows that BSA has a much higher noise.

### Example 5—CRP Detection in Serum

In the clinic, CRP can be measured to monitor patients at risk for infections, cardiovascular events and autoimmune diseases. Furthermore, CRP can be an important parameter when prescribing antibiotics. The reference CRP concentration in human serum is <0.2-10.5 mg/L for healthy adults. CRP can increase to 40-350 mg/L during inflammation, with typical values for e.g. viral infections (10-40 mg/L), bacterial infection (>200 mg/L), and cardiovascular risk (systemic>3 mg/L), with 3 mg/L corresponding to 24 nM of CRP.

We tested whether YaxAB nanopores could also detect CRP in presence of other proteins in a biologically complex sample: human serum. Human serum was depleted for the top 14 most abundant proteins such as IgG, albumin, and transferrin. Typically, high concentrations of serum reduce the stability of the lipid bilayer comprising the nanopore. Therefore, serum was added in a diluted for (2.5 μL depleted human serum added to a cis side of the nanopore to a final dilution of 160-fold) to allow for electrical recordings for a few minutes.

FIGS. 7A-7B shows electrophysiology data showing the detection of C-Reactive Protein (CRP) in depleted human serum (depleted of the top 14 most abundant proteins such as IgG, albumin, and transferrin). FIG. 7A shows recordings of depleted human serum samples using YaxA_{Δ40}B nanopores, doped with increasing amounts of additional CRP. The traces show representative current blockades (left), the dwell time vs. residual current Iᵣₑₛ (%) scatterplots (middle) and the normalized σ_{blockade} vs residual current Iᵣₑₛ (%) scatterplots (right) for increasing amounts of CRP added to the cis compartment. Data in panel (i) was recorded for 10 minutes, and data for panels (ii-v) were recorded for 2 minutes. Experiments were performed in 150 mM NaCl, 15 mM TrisHCl pH 7.5, −75 mV, with 50 kHz sampling rate and 10 kHz low-pass Bessel filter. FIG. 7B shows a graph showing the event frequency of the CRP blockade events vs. the concentration of CRP added to the cis compartment for the same data shown in FIG. 7A. Under an applied potential of −75 mV, the protein from depleted serum was found to induce a variety of blockades with an Iᵣₑₛ(%) of 20-80% when added to an electrophysiology system containing YaxAΔ₄₀B*80 nanopores (FIG. 7A i). Occasionally, serum proteins remained trapped inside the nanopore for several seconds. In these instances, the potential was reversed to release the protein. Among the blockades recorded in 627 seconds, 6 events could be assigned to CRP (events within 2G of the Iᵣₑₛ (%), σ_{blockade} and dwell time), most likely as a result of the basal levels of CRP in the baseline sample. Doping an additional 20-80 nM (2.5-10 mg/L) of CRP to the cis compartment in the background of 2.5 μL of depleted serum resulted in a clear increase in the number of blockades with parameters matching those of CRP (FIG. 7A ii-v), which were highly similar to CRP events in buffered solution in the control experiments. This indicated that depleted serum did not significantly alter the signature blockade of CRP or significantly interfere with its detection. Further, the event frequency was determined to be directly proportional to the concentration of CRP added (FIG. 7B), indicating that the method is suitably used to quantify the concentration of CRP in complex (clinical) samples.

### Example 6—Detection of Small Analytes Via Protein Complexes

This example demonstrates that YaxAB nanopores can be used to measure analytes complexed to a secondary dedicated binding partner, such as co-factors or ligands (e.g. proteins complexed to ligands or drugs), where the co-factor/ligands would otherwise in isolation be too small to be captured in YaxAB nanopores i.e. the dimensions of the molecules are smaller than the constriction. To that end, YaxAΔ₄₀B*80 nanopores were tested with Streptavidin SA (added to a cis side) in the presence of biotin (added to either the cis or the trans), as shown schematically in FIGS. 8A-8B.

Addition of 200 nM SA to the cis side of YaxAΔ₄₀B*80 nanopores resulted in characteristic SA blockades (I_{SA}=55%, dwell time=99 ms) (FIG. 9A). After adding 2 μM of biotin to the trans side of the pore, the residual current of the SA blockade changed to I_{SA}=60%, with reduced dwell time=17 ms (FIG. 9B). Biotin alone could not be detected. This demonstrated that biotin can be detected through complexation with streptavidin by the change in the I_{RES} (%) and the change in the average dwell time of the SA blockades. Similar 5-10 pA increases in residual current and reductions in dwell time of the SA binding level were observed when biotin was added to the cis of YaxA_{Δ40}B*80 nanopores with SA, or when biotin was added to either the cis or trans of functionalised YaxAB nanopores with SA (e.g. FIGS. 12A-12C, FIGS. 13A-13C, FIGS. 14A-14B, FIGS. 15A-15C, FIGS. 16A-16C, FIGS. 17A-17C, Table 3). FIGS. 9A-9B shows YaxAΔ₄₀B*80: truncated and otherwise unmodified pore, recorded at −70 mV. 200 nM Streptavidin (SA) was added to a cis side, resulting in characteristic SA blockades (I_{SA}) from the open-pore current level (I_{O}), with I_{RES} (%)=55%, dwell time=99 ms. After adding 2 μM of biotin to trans the SA blockade changes to I_{RES} (%)=60%, dwell time=17 ms. The binding of biotin to streptavidin complex can be detected by the change in the I_{RES} (%) and the change in the average dwell time of the SA level. By determining the proportion of events corresponding to analyte vs. analyte-ligand complex, the system can be used to detect and quantify the amount of ligand in the system and/or the complexation properties of the analyte-ligand complex.

### Example 7—Nanopore Functionalization

This example describes functionalizing YaxAB nanopores near the cis entrance of the pore with polymeric extensions in order to modulate nanopore capture and retention of target and non-target analytes that are present in the cis solution. The polymer extensions also comprise a recognition element/motif R for binding to specific target analyte(s) to further control the capture of the target analyte(s) in a system containing a mixture of analytes.

From structural models, the N- and C-termini of the YaxB subunit of YaxAB were determined to have an optimal location for creating polymeric extensions near the cis entrance of the nanopores. YaxB subunits of YaxA_{Δ40}B nanopores were therefore modified at either the N- or C-termini by genetic fusion with additional amino acid residues. The YaxB subunits were functionalised with StrepII tag motifs as recognition element, to specifically bind Streptavidin (SA) as a target analyte. The recognition element was connected to the nanopore via a wide range of different peptide linker lengths and composition (Tables 2 and 4). Linkers from 3 to 70 amino acids in length were designed to test the effect of binding and steric crowding at the cis entrance of the conical nanopore. FIGS. 10A-10F schematically illustrates the length of the linkers to approximate scale (in a semi stretched form). The schematic model of FIGS. 10A-10F shows the strategy of functionalizing the cis entrance of YaxAB nanopores with a recognition element using peptide extensions of varying length. The figure shows a schematic of nanopores with approximately to scale peptide extensions (in a semi-stretched state) comprised of linker sections (ii) of varying lengths from 3 amino acids long to 70 amino acids long, also comprising a recognition element (i), which is connected to the nanopore near to the top of the large cis entrance of the nanopore (iii). For image clarity only two peptide extensions are shown from opposing sides of the nanopores. It shows that the shortest lengths only partially affect the entrance to the nanopore, while the longer linkers create a substantial barrier to the cis entrance of the nanopores.

To test the utility of StrepII recognition elements on YaxAB nanopores, various functionalised YaxAB nanopores were tested against the ability to capture and retain the target analyte, SA, both in isolation (FIG. 11A) and in the presence of a non-target protein analyte, CRP (FIG. 11B).

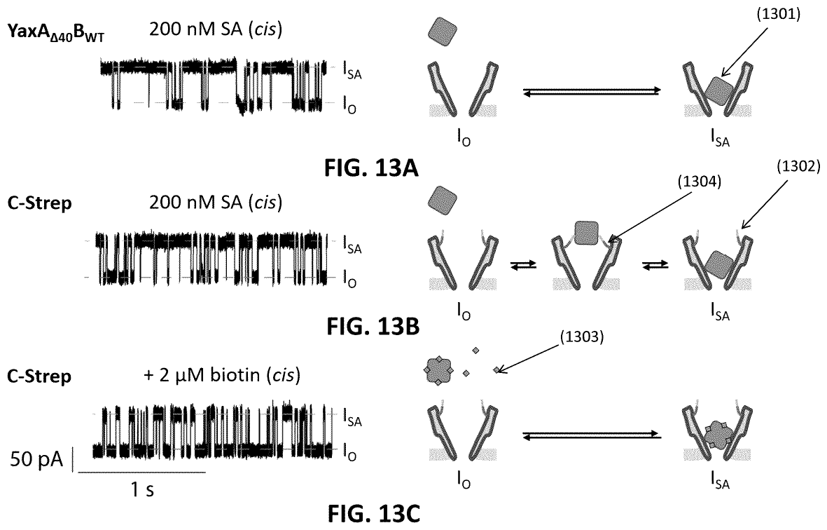

N- and C-terminally functionalised YaxAB nanopores with 3 amino acid linkers were tested with SA to determine the effect of the linkers on the behaviour of the nanopores. The linkers were found to have a minimal effect on the formation and behaviour of the nanopores (FIGS. 12A-12C and FIGS. 13A-13C), showing similar electrical behaviour in electrophysiology experiments, and the ability to capture and detect SA from the cis compartment. Further, similar to the unmodified nanopores, addition of biotin (added to the cis compartment) resulted in a significant reduction in dwell time and a 4-10% increase in residual current for the SA binding level. FIGS. 12A-12C shows N-terminally functionalized YaxAB nanopores for capturing proteins and proteins complexes. FIG. 12A shows representative example of unmodified YaxAΔ₄₀B*80 nanopores showing clear capture of streptavidin (SA) added to the cis side of the system (SA blockade levels of I_{RES} (%)=˜55%). FIG. 12B shows a representative example of functionalized YaxA_{Δ40}B_{N-strepII-3aa} (with N-terminal StrepII-tag (3aa linker) on YaxB), with 200 nM SA added to a cis side (SA I_{RES} (%)=˜55%). C) Addition of 2 μM of biotin to cis, SA blockade level changed to I_{RES} (%)=˜60%. Data were recorded at −70 mV. FIGS. 13A-13C shows C-terminally functionalized YaxAB nanopores for capturing proteins and proteins complexes. FIG. 13A shows representative example of unmodified YaxAΔ₄₀B*80 nanopores showing clear capture of streptavidin (SA) added to the cis side (SA blockade levels of I_{RES} (%)=˜55%). The current output is similar to that of FIG. 12A, in which there is no recognition element attached to the nanopore and the Streptavidin analyte was reversibly captured by the nanopore. FIG. 13B shows representative example of functionalized YaxA_{Δ40}B_{C-strepII-3aa} (with C-terminal StrepII-tag (3aa linker) on YaxB), with 200 nM SA added to a cis side (I_{RES} (%)=˜53%). The C-terminal StrepII-tag (3aa linker) on YaxB showed affinity for the streptavidin analyte, as shown by the I_{SA} current designating capture of the analyte in the nanopore. FIG. 13C shows the addition of 2 μM of biotin to cis, SA blockade changed to I_{RES} (%)=˜58%. Data were recorded at −70 mV. As the biotin was competing with the StrepII tags for the same binding sites on SA, the recognition element was not able to bind and capture the analyte. The current output shows an increased amount of time at the open-pore current (e.g., I_{O}) compared to that from FIG. 13A.

### Filtering Effect: Adding CRP to Different YaxA_{Δ40}B-Peptide Nanopores

To test the capture of large analytes, 20 nM CRP was added to YaxAB nanopores functionalised with linkers from 3 to 70 amino acids in length and a terminal StrepII tag as recognition element (see Table 2 for different YaxB constructs and nanopore design). CRP is a large protein (125 kDa, heptamer) that enters unmodified YaxAΔ₄₀B*80 (see FIG. 4, FIGS. 5A-5C, and FIGS. 12A-12C). The presence of the peptide linkers altered the entry of CRP inside the nanopore (Table 3 and FIGS. 12A-12C, FIGS. 13A-13C, FIGS. 14A-14B, and FIGS. 15A-15C). With the unmodified YaxAΔ₄₀B, the frequency of CRP capture was ˜310 events μM⁻¹ s⁻¹, and it was only slightly reduced when using functionalised YaxAB nanopores with polypeptide linker lengths of up to 40 amino acids (FIGS. 15A-15C and Table 3). FIGS. 15A-15C shows N-termini functionalized YaxA_{Δ40}B_{N-strepII-30aa-flex} with N-terminal StrepII-tag separated with 30 amino acids from YaxB. FIG. 15A shows 20 nM CRP added to a cis side is captured by the nanopore producing characteristic blockade level I_{CRP}. FIG. 15B shows additional 20 nM SA added to a cis side, producing long SA blockades with multiple levels show (I_{RES} (%)˜44-95%), where the top level corresponds to SA (I_{RES} (%)=˜44%). After addition of SA no CRP events are observed due to SA being captured in the pore for long periods. FIG. 15C shows addition of 2 μM biotin to a trans side blocks the binding of the linker extensions, thus reducing the dwell time of SA blockades, and individual CRP events can be distinguished again.

The capture frequency, however, was reduced to ˜60 events (μM⁻¹ s⁻¹) using the 50 amino acid linker YaxA_{Δ40}B_{N-strepII-50aa-flex} (FIGS. 16A-16C and Table 3). FIGS. 16A-16C shows N-termini Functionalized YaxA_{Δ40}B_{N-strepII-50aa-flex} with N-terminal StrepII-tag separated with 50 amino acids from YaxB. FIG. 16A shows 20 nM CRP added to a cis side is captured by the nanopore producing characteristic blockade level I_{CRP}. FIG. 16B shows additional 20 nM SA added to a cis side, producing very long SA blockades. No CRP blockades are observed. FIG. 16C shows addition of 2 μM biotin to trans reduces the dwell time of SA blockades, and individual CRP events can be distinguished. No CRP events were observed when using the 70 amino acid linker YaxA_{Δ40}B_{N-strepII-70aa-flex} (over an observation period of about 5 minutes, N=4 different nanopores) (FIGS. 17A-17C and Table 3). FIGS. 17A-17C shows N-termini Functionalized YaxA_{Δ40}B_{N-strepII-70aa-flex} with N-terminal StrepII-tag separated with 70 amino acids from YaxB. FIG. 17A shows 20 nM CRP added to a cis side produces no capture or characteristic blockades. FIG. 17B shows additional 20 nM SA added to a cis side, produces very long SA blockades. No CRP events are observed. FIG. 17C shows addition of 2 μM biotin to trans results in infrequent SA blockade levels and reduces the dwell time of SA blockades, no individual CRP events are observed. This demonstrates that it is possible to tune the length of the polymer extensions at the cis entrance of the YaxAB nanopores to reduce or entirely prevent the capture of large analytes from the cis solution.

Residence Time of SA within Different YaxA_{Δ40}B-Peptide Nanopores

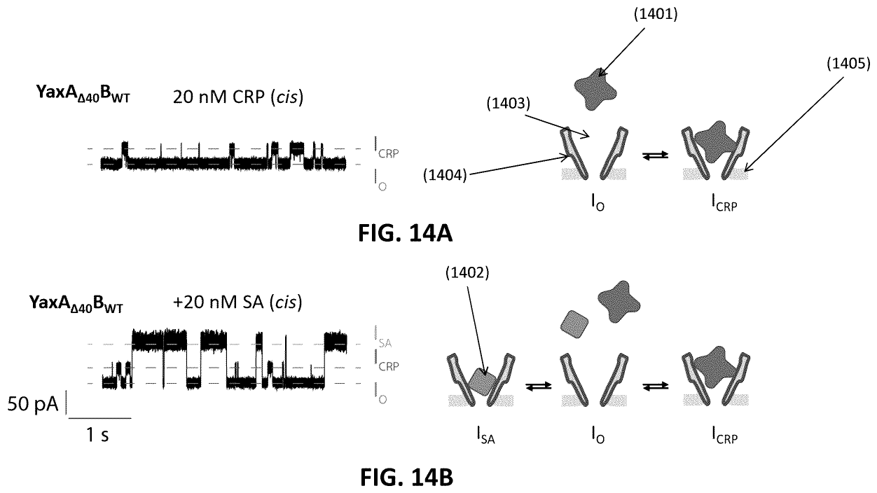

In the same experiments described with CRP, a further 20 nM SA was subsequently added to the cis compartment of the functionalized YaxAB nanopores to test the effect of the linkers (comprising the terminal StrepII tag that binds streptavidin) on the capture of SA. The functionalized YaxA_{Δ40}B nanopores increased the residence time of SA capture (FIGS. 15A-15C, FIGS. 16A-16C, and FIGS. 17A-17C and Table 3) depending on the length of the peptide linker and independently of the presence of CRP vs. unmodified nanopores (FIGS. 14A-14B and Table 3). The SA capture dwell (residence) time increased from ˜200 ms (YaxA_{Δ40}B) to about 400 ms for YaxA_{Δ40}B_{N-strepII-10aa-neg} and YaxA_{Δ40}B_{N-strepII-10aa-flex}. For a linker of 20 amino acids or longer, the residence time increased to more than 60 seconds. Accurate statistics of the residence time were difficult given the long residence times observed. Interestingly, using YaxA_{Δ40}B_{N-strepII-70aa-flex} nanopores, when both CRP (20 nM) and SA (20 nM) were added to the cis solution, only SA blockades could be observed (observation time 5 minutes, N=4 different nanopores). FIGS. 14A-14B shows YaxA_{Δ40}B*80: truncated and otherwise unmodified pore. FIG. 14A shows 20 nM CRP (1401) added to a cis side is captured by the pore (1404) and produces characteristic blockades I_{CRP}. The nanopore (1404) disposed in a membrane (1405) contained a first opening (1403) by which the analyte could enter. Capture of the CRP analyte by the nanopore produced a change in current from open-pore level (I_{O}) to CRP blockade level (I_{CRP}). The peaks of the current signal showed the reversible capture of the analyte. FIG. 14B shows adding additional 20 nM SA (1402) to a cis side of the same nanopore produces a new SA blockade level I_{SA}. When adding biotin to cis or trans, the I_{RES} (%) of SA increases and dwell time is decreased, both CRP and SA are still captured. As shown in FIG. 14B, it is possible to differentiate the analytes of the sample based on the magnitude of the peaks in the current output.

Addition of biotin (to the trans compartment), which binds very strongly to SA, resulted in large reductions in the capture dwell (residence) time for SA (FIGS. 15A-15C, FIGS. 16A-16C, and FIGS. 17A-17 and Table 3). This indicates that biotin is competing with the StrepII tags for the same binding sites on SA, thus partially negating the trapping properties of the functionalised nanopores. For the shorter linker nanopores able to capture CRP, the dwell time of CRP was unaffected by the addition of biotin. This proves that the recognition motif of the functionalised nanopores is specific for controlling and enhancing the capture of the target analyte.

TABLE 2
YaxB design and linker sequences

| | | Peptide Length |
|---|---|---|
| construct name | YaxB design | (Linker + tag) |
| YaxA_{Δ40}B wt | YaxB | N/A |
| YaxA_{Δ40}B_{N-strepII-N}_3 aa | NstrepII-GSS-YaxB | 4.0 nm |
| YaxA_{Δ40}B_{C-strepII-C}_3 aa | YaxB-GSS-CstrepII | 4.0 nm |
| YaxA_{Δ40}BN_{-strepII-10 aa-flex} | NstrepII-GSSGSAGSAG-YaxB | 6.6 nm |
| YaxA_{Δ40}B_{N-strepII-10 aa-neg} | NstrepII-GDSGDEGSEG-YaxB (−4) | 6.6 nm |
| YaxA_{Δ40}B_{N-strepII-20 aa-neg} | NstrepII-GSSDSDGSSGEAGDEG-YaxB (−5) | 10.2 nm |
| YaxA_{Δ40}B_{N-strepII-30 aa-flex} | NstrepII-GSSGSTSNASAGTGSATGSNSTAGASSGNS-YaxB | 13.9 nm |
| YaxA_{Δ40}B_{N-strepII-40 aa-FG} | NstrepII- | |
| | TPQQNKTPFSFGTANNNSNTTNQNSSTGAGAFGTGQSTFG | |
| YaxA_{Δ40}B_{N-strepII-50 aa-flex} | NstrepII- | 21.2 nm |
| | GSSGSTSNASAGTGSATGSNSTAGASSGNSAGTGTSSGSTGSA | |
| | NSSAGTG-YaxB | |
| YaxA_{Δ40}B_{N-strepII-70 aa-flex} | NstrepII- | 28.5 nm |
| | GSSGSTSNASAGTGSATGSNSTAGASSGNSAGTGTSSGSTGSA | |
| | NSSAGTGSGSATASAGNSGTSTAGNST-YaxB | |

TABLE 3
Functionalized YaxAB constructs properties. Results are
the average of >3 experiments for each nanopore type.

| | CRP capture | SA | SA | SA + biotin | SA + biotin |
|---|---|---|---|---|---|
| | frequency | Iᵣₑₛ | Dwell time | Iᵣₑₛ | Dwell time |
| construct name | (μM⁻¹ s⁻¹) | (%) | (ms) | (%) | (ms) |
| YaxA_{Δ40}B wt | 308.9 ± 9.7 | 50.3 ± 6.2 | 187.5 ± 103.4 | 60.6 ± 0.2 | 24.0 ± 15.9 |
| YaxA_{Δ40}B_{N-strepII-10aa-flex} | 211 | 53.3 ± 0.7 | 351.5 ± 209.5 | 53.3 ± 4.5 | 31.0 ± 28.8 |
| YaxA_{Δ40}B_{N-strepII-10aa-neg} | 345 | 48.9 ± 6.0 | 396.0 ± 288.0 | 52.5 ± 5.3 | 86.6 ± 75.1 |
| YaxA_{Δ40}B_{N-strepII-20aa-neg} | 187 | 44.7-96.7* | >10,000 | 53.4 ± 5.1 | 55.0 ± 19.4 |
| YaxA_{Δ40}B_{N-strepII-30aa-flex} | 216 | 44.4-94.4* | >60,000 | 51.1 ± 1.4 | 79.2 ± 29.3 |
| YaxA_{Δ40}B_{N-strepII-40aa-FG} | 243 | 41.0-88.0* | >60,000 | 49.5-80.0* | 339.9 ± 99.1 |
| YaxA_{Δ40}B_{N-strepII-50aa-flex} | 59 | 49.6-83.8* | >60,000 | 62.1 ± 1 | 1210 + 640 |
| YaxA_{Δ40}B_{N-strepII-70aa-flex} | Not determined | 47.8-76.2* | >60,000 | 57.1 ± 2.2 | 3730 ± 484 |

*multiple discrete current levels were observed in the range indicated

TABLE 4
Protein Constructs and Sequences

| | SEQ ID | | |
|---|---|---|---|
| Construct | NO. | Component | Sequence |
| His6-TEV- | 1 | Full Sequence | MSYYHHHHHHDYDIPTTENLTRSSENLYFQSGGTQTQLAI |
| YaxA | | | DNVLASAESTIQLNELPKVVLDFITGEQTSVARSGGIFTKE |
| | | | DLINLKLYVRKGLSLPTRQDEVEAYLGYKKIDVAGLEPKD |
| | | | IKLLFDEIHNHALNWNDVEQAVLQQSLDLDIAAKNIISTGN |
| | | | EIINLINQMPITLRVKTLLGDITDKQLENITYESADHEVASA |
| | | | LKDILDDMKGDINRHQTTTENVRKKVSDYRITLTGGELSS |
| | | | GDKVNGLEPQVKTKYDLMEKSNMRKSIKELDEKIKEKRQ |
| | | | RIEQLKKDYDKFVGLSFTGAIGGIIAMAITGGIFGAKAENA |
| | | | RKEKNALISEVAELESKVSSQRALQTALEALSLSFSDIGIRM |
| | | | VDAESALNHLDFMWLSVLNQITESQIQFAMINNALRLTSF |
| | | | VNKFQQVITPWQSVGDSARQLVDIFDEAIKEYKKVYG* |
| | 2 | His-tag | HHHHHH |
| | 3 | TEV Cleavage | ENLYFQS |
| | | Site | |
| | 4 | Linker | TQTQLAIDNVLASAESTIQLNELPKVVLDFITGEQTSVAR |
| His6-TEV- | 5 | Full Sequence | MHHHHHHENLYFQSSGGIFTKEDLINLKLYVRKGLSLPTR |
| YaxAΔ40 | | | QDEVEAYLGYKKIDVAGLEPKDIKLLFDEIHNHALNWND |
| | | | VEQAVLQQSLDLDIAAKNIISTGNEIINLINQMPITLRVKTL |
| | | | LGDITDKQLENITYESADHEVASALKDILDDMKGDINRHQ |
| | | | TTTENVRKKVSDYRITLTGGELSSGDKVNGLEPQVKTKYD |
| | | | LMEKSNMRKSIKELDEKIKEKRQRIEQLKKDYDKFVGLSF |
| | | | TGAIGGIIAMAITGGIFGAKAENARKEKNALISEVAELESK |
| | | | VSSQRALQTALEALSLSFSDIGIRMVDAESALNHLDFMWL |
| | | | SVLNQITESQIQFAMINNALRLTSFVNKFQQVITPWQSVGD |
| | | | SARQLVDIFDEAIKEYKKVYG* |
| | 2 | His-tag | HHHHHH |
| | 3 | TEV Cleavage | ENLYFQS |
| | | Site | |
| His6-TEV- | 6 | Full Sequence | MSYYHHHHHHDYDIPTTENLYFQGAEISTFPHSGLSYPDIN |
| YaxB | | | FKIFSQGVKNISHLAQFKTTGVEVLQEKALRVSLYSQRLD |
| | | | VIVRESLSSLQVKLENTLALTYFTTLEEIDEALISQDIDEESK |
| | | | SEMRKERINIIKNLSNDITQLKQLFIEKTELLDKSSSDLHNV |
| | | | VIIEGTDKVLQAEQLRQKQLTEDIATKELERKEIEKKRDKII |
| | | | EALDVIREHNLVDAFKDLIPTGENLSELDLAKPEIELLKQSL |
| | | | EITKKLLGQFSEGLKYIDLTDARKKLDNQIDTASTRLTELN |
| | | | RQLEQSEKLIAGVNAIIKIDQEKSAVVVEAEKLSRAWHIFI |
| | | | HEITALQGTSLNEVELSKPLIKQQIYLESLIKQLI* |
| | 2 | His-tag | HHHHHH |
| | 7 | TEV Cleavage | ENLYFQG |
| | | Site | |
| His6- | 8 | Full Sequence | MSYYHHHHHHDYDIPTTENLYFQGWSHPQFEKGSSAEIST |
| TEV_StrepII_ | | | FPHSGLSYPDINFKIFSQGVKNISHLAQFKTTGVEVLQEKA |
| GSS-YaxB | | | LRVSLYSQRLDVIVRESLSSLQVKLENTLALTYFTTLEEIDE |
| | | | ALISQDIDEESKSEMRKERINIIKNLSNDITQLKQLFIEKTEL |
| | | | LDKSSSDLHNVVIIEGTDKVLQAEQLRQKQLTEDIATKELE |
| | | | RKEIEKKRDKIIEALDVIREHNLVDAFKDLIPTGENLSELDL |
| | | | AKPEIELLKQSLEITKKLLGQFSEGLKYIDLTDARKKLDNQI |
| | | | DTASTRLTELNRQLEQSEKLIAGVNAIIKIDQEKSAVVVEA |
| | | | EKLSRAWHIFIHEITALQGTSLNEVELSKPLIKQQIYLESLIK |
| | | | QLI* |
| | 2 | His-tag | HHHHHH |
| | 7 | TEV Cleavage | ENLYFQG |
| | | Site | |
| | 39 | StrepII-tag | WSHPQFEK |
| | 9 | Linker | GSS |
| His6- | 10 | Full Sequence | MSYYHHHHHHDYDIPTTENLYFQGAEISTFPHSGLSYPDIN |
| TEV_YaxB_ | | | FKIFSQGVKNISHLAQFKTTGVEVLQEKALRVSLYSQRLD |
| GSS_Strep | | | VIVRESLSSLQVKLENTLALTYFTTLEEIDEALISQDIDEESK |
| II | | | SEMRKERINIIKNLSNDITQLKQLFIEKTELLDKSSSDLHNV |
| | | | VIIEGTDKVLQAEQLRQKQLTEDIATKELERKEIEKKRDKII |
| | | | EALDVIREHNLVDAFKDLIPTGENLSELDLAKPEIELLKQSL |
| | | | EITKKLLGQFSEGLKYIDLTDARKKLDNQIDTASTRLTELN |
| | | | RQLEQSEKLIAGVNAIIKIDQEKSAVVVEAEKLSRAWHIFI |
| | | | HEITALQGTSLNEVELSKPLIKQQIYLESLIKQLIGSSWSHP |
| | | | QFEK* |
| | 2 | His-tag | HHHHHH |
| | 7 | TEV Cleavage | ENLYFQG |
| | | Site | |
| | 9 | Linker | GSS |
| | 39 | StrepII-tag | WSHPQFEK |
| His6- | 11 | Full Sequence | MSYYHHHHHHDYDIPTTENLYFQGWSHPQFEKGSSGSAG |
| TEV_StrepII_ | | | SAGAEISTFPHSGLSYPDINFKIFSQGVKNISHLAQFKTTGV |
| 10 aa- | | | EVLQEKALRVSLYSQRLDVIVRESLSSLQVKLENTLALTYF |
| neut_YaxB | | | TTLEEIDEALISQDIDEESKSEMRKERINIIKNLSNDITQLKQ |
| | | | LFIEKTELLDKSSSDLHNVVIIEGTDKVLQAEQLRQKQLTE |
| | | | DIATKELERKEIEKKRDKIIEALDVIREHNLVDAFKDLIPTG |
| | | | ENLSELDLAKPEIELLKQSLEITKKLLGQFSEGLKYIDLTDA |
| | | | RKKLDNQIDTASTRLTELNRQLEQSEKLIAGVNAIIKIDQE |
| | | | KSAVVVEAEKLSRAWHIFIHEITALQGTSLNEVELSKPLIK |
| | | | QQIYLESLIKQLI* |
| | 2 | His-tag | HHHHHH |
| | 7 | TEV Cleavage | ENLYFQG |
| | | Site | |
| | 39 | StrepII-tag | WSHPQFEK |
| | 12 | Linker | GSSGSAGSAG |
| His6- | 13 | Full Sequence | MSYYHHHHHHDYDIPTTENLYFQGWSHPQFEKGSDGDEG |
| TEV_StrepII_ | | | SEGAEISTFPHSGLSYPDINFKIFSQGVKNISHLAQFKTTGV |
| 10 aa-neg- | | | EVLQEKALRVSLYSQRLDVIVRESLSSLQVKLENTLALTYF |
| link YaxB | | | TTLEEIDEALISQDIDEESKSEMRKERINIIKNLSNDITQLKQ |
| | | | LFIEKTELLDKSSSDLHNVVIIEGTDKVLQAEQLRQKQLTE |
| | | | DIATKELERKEIEKKRDKIIEALDVIREHNLVDAFKDLIPTG |
| | | | ENLSELDLAKPEIELLKQSLEITKKLLGQFSEGLKYIDLTDA |
| | | | RKKLDNQIDTASTRLTELNRQLEQSEKLIAGVNAIIKIDQE |
| | | | KSAVVVEAEKLSRAWHIFIHEITALQGTSLNEVELSKPLIK |
| | | | QQIYLESLIKQLI* |
| | 2 | His-tag | HHHHHH |
| | 7 | TEV Cleavage | ENLYFQG |
| | | Site | |
| | 39 | StrepII-tag | WSHPQFEK |
| | 14 | Linker | GSDGDEGSEG |
| His6- | 15 | Full Sequence | MSYYHHHHHHDYDIPTTENLYFQGWSHPQFEKGSRKSRG |
| TEV_StrepII_ | | | SKGAEISTFPHSGLSYPDINFKIFSQGVKNISHLAQFKTTGV |
| 10 aa-pos- | | | EVLQEKALRVSLYSQRLDVIVRESLSSLQVKLENTLALTYF |
| link_YaxB | | | TTLEEIDEALISQDIDEESKSEMRKERINIIKNLSNDITQLKQ |
| | | | LFIEKTELLDKSSSDLHNVVIIEGTDKVLQAEQLRQKQLTE |
| | | | DIATKELERKEIEKKRDKIIEALDVIREHNLVDAFKDLIPTG |
| | | | ENLSELDLAKPEIELLKQSLEITKKLLGQFSEGLKYIDLTDA |
| | | | RKKLDNQIDTASTRLTELNRQLEQSEKLIAGVNAIIKIDQE |
| | | | KSAVVVEAEKLSRAWHIFIHEITALQGTSLNEVELSKPLIK |
| | | | QQIYLESLIKQLI* |
| | 2 | His-tag | HHHHHH |
| | 7 | TEV Cleavage | ENLYFQG |
| | | Site | |
| | 39 | StrepII-tag | WSHPQFEK |
| | 16 | Linker | GSRKSRGSKG |
| His6- | 17 | Full Sequence | MSYYHHHHHHDYDIPTTENLYFQGWSHPQFEKGSSDSDG |
| TEV_StrepII_ | | | SSGEAGDEGAEISTFPHSGLSYPDINFKIFSQGVKNISHLAQ |
| 20 aa-neg- | | | FKTTGVEVLQEKALRVSLYSQRLDVIVRESLSSLQVKLENT |
| link_YaxB | | | LALTYFTTLEEIDEALISQDIDEESKSEMRKERINIIKNLSND |
| | | | ITQLKQLFIEKTELLDKSSSDLHNVVIIEGTDKVLQAEQLRQ |
| | | | KOLTEDIATKELERKEIEKKRDKIIEALDVIREHNLVDAFK |
| | | | DLIPTGENLSELDLAKPEIELLKQSLEITKKLLGQFSEGLKYI |
| | | | DLTDARKKLDNQIDTASTRLTELNRQLEQSEKLIAGVNAII |
| | | | KIDQEKSAVVVEAEKLSRAWHIFIHEITALQGTSLNEVELS |
| | | | KPLIKQQIYLESLIKQLI* |
| | 2 | His-tag | HHHHHH |
| | 7 | TEV Cleavage | ENLYFQG |
| | | Site | |
| | 39 | StrepII-tag | WSHPQFEK |
| | 18 | Linker | GSSDSDGSSGEAGDEG |
| His6- | 19 | Full Sequence | MSYYHHHHHHDYDIPTTENLYFQGWSHPQFEKGSSGSTSN |
| TEV_StrepII_ | | | ASAGTGSATGSNSTAGASSGNSAEISTFPHSGLSYPDINFKI |
| 30 aa-flex- | | | FSQGVKNISHLAQFKTTGVEVLQEKALRVSLYSQRLDVIV |
| link_YaxB | | | RESLSSLQVKLENTLALTYFTTLEEIDEALISQDIDEESKSE |
| | | | MRKERINIIKNLSNDITQLKQLFIEKTELLDKSSSDLHNVVII |
| | | | EGTDKVLQAEQLRQKQLTEDIATKELERKEIEKKRDKIIEA |
| | | | LDVIREHNLVDAFKDLIPTGENLSELDLAKPEIELLKQSLEI |
| | | | TKKLLGQFSEGLKYIDLTDARKKLDNQIDTASTRLTELNR |
| | | | QLEQSEKLIAGVNAIIKIDQEKSAVVVEAEKLSRAWHIFIHE |
| | | | ITALQGTSLNEVELSKPLIKQQIYLESLIKQLI* |
| | 2 | His-tag | HHHHHH |
| | 7 | TEV Cleavage | ENLYFQG |
| | | Site | |
| | 39 | StrepII-tag | WSHPQFEK |
| | 20 | Linker | GSSGSTSNASAGTGSATGSNSTAGASSGNS |
| His6- | 21 | Full Sequence | MSYYHHHHHHDYDIPTTENLYFQGWSHPQFEKGSSGSTSN |
| TEV_StrepII_ | | | ASAGTGSATGSNSTAGASSGNSAGTGTSSGSTGSANSSAG |
| 50 aa-flex- | | | TGAEISTFPHSGLSYPDINFKIFSQGVKNISHLAQFKTTGVE |
| link_YaxB | | | VLQEKALRVSLYSQRLDVIVRESLSSLQVKLENTLALTYFT |
| | | | TLEEIDEALISQDIDEESKSEMRKERINIIKNLSNDITQLKQL |
| | | | FIEKTELLDKSSSDLHNVVIIEGTDKVLQAEQLRQKQLTEDI |
| | | | ATKELERKEIEKKRDKIIEALDVIREHNLVDAFKDLIPTGEN |
| | | | LSELDLAKPEIELLKQSLEITKKLLGQFSEGLKYIDLTDARK |
| | | | KLDNQIDTASTRLTELNRQLEQSEKLIAGVNAIIKIDQEKSA |
| | | | VVVEAEKLSRAWHIFIHEITALQGTSLNEVELSKPLIKQQIY |
| | | | LESLIKQLI* |
| | 2 | His-tag | HHHHHH |
| | 7 | TEV Cleavage | ENLYFQG |
| | | Site | |
| | 39 | StrepII-tag | WSHPQFEK |
| | 22 | Linker | GSSGSTSNASAGTGSATGSNSTAGASSGNSAGTGTSSGSTG |
| | | | SANSSAGTG |
| His6- | 23 | Full Sequence | MSYYHHHHHHDYDIPTTENLYFQGWSHPQFEKGSSGSTSN |
| TEV_StrepII_ | | | ASAGTGSATGSNSTAGASSGNSAGTGTSSGSTGSANSSAG |
| 70 aa-flex- | | | TGSGSATASAGNSGTSTAGNSTAEISTFPHSGLSYPDINFKI |
| link_YaxB | | | FSQGVKNISHLAQFKTTGVEVLQEKALRVSLYSQRLDVIV |
| | | | RESLSSLQVKLENTLALTYFTTLEEIDEALISQDIDEESKSE |
| | | | MRKERINIIKNLSNDITQLKQLFIEKTELLDKSSSDLHNVVII |
| | | | EGTDKVLQAEQLRQKQLTEDIATKELERKEIEKKRDKIIEA |
| | | | LDVIREHNLVDAFKDLIPTGENLSELDLAKPEIELLKQSLEI |
| | | | TKKLLGQFSEGLKYIDLTDARKKLDNQIDTASTRLTELNR |
| | | | QLEQSEKLIAGVNAIIKIDQEKSAVVVEAEKLSRAWHIFIHE |
| | | | ITALQGTSLNEVELSKPLIKQQIYLESLIKQLI* |
| | 2 | His-tag | HHHHHH |
| | 7 | TEV Cleavage | ENLYFQG |
| | | Site | |
| | 39 | StrepII-tag | WSHPQFEK |
| | 24 | Linker | GSSGSTSNASAGTGSATGSNSTAGASSGNSAGTGTSSGSTG |
| | | | SANSSAGTGSGSATASAGNSGTSTAGNST |

YaxA full-length starts at: TQTQ
YaxA_{Δ40} starts at: SGGIFT
YaxB starts at: AEIST
M = methionine, start codon at beginning of sequence.
* = stop codon, end of sequence.
Remaining amino acids are spacers.

TABLE 5
Alpha-xenorhabdolysin family binary toxin orthologues

| | | GenBank | UniProt | SEQ ID | |
|---|---|---|---|---|---|
| Organism | Species | ID | ID | NO. | Sequence |
| Yersinia | YaxA | CAL12063. | YE1984 | 25 | MTQTQLAIDNVLASAENTIQLNELPKVV |
| enterocolitica | | 1 | | | LDFITGEQTSVARSGGIFTKEDLINLKLY |
| | | | | | VRKGLSLPTRQDEVEAYLGYKKIDVAG |
| | | | | | LEPKDIKLLFDEIHNHALNWNDVEQAVL |
| | | | | | QQSLDLDIAAKNIISTGNEIINLINQMPIT |
| | | | | | LRVKTLLRDITDKQLENITYESADHEVA |
| | | | | | SALKDILDDMKGDINRHQTTTENVRKK |
| | | | | | VSDYRITLTGGELSSGDKVNGLEPQVKT |
| | | | | | KYDLMEKSNMRKSIKELDEKIKEKKORI |
| | | | | | EQLKKDYDKFVGLSFTGAIGGIIAMAITS |
| | | | | | GIFGAKAENARKEKNALISEVAELESKV |
| | | | | | SSQRALQTALEALSLSFSDIGIRMVDAES |
| | | | | | ALNHLDFMWLSVLNQITESQIQFAMINN |
| | | | | | ALRLTSFVNKFQQVITPWQSVGDSARQL |
| | | | | | VDIFDEAIKEYKKVYG* |
| | YaxB | CAL12064. | YE1985 | 26 | MAEISTFPHSGLSYPDINFKIFSQGVKNIS |
| | | 1 | | | HLAQFKTTGVEVLQEKALRVSLYSQRL |
| | | | | | DVIVRESLSSLQVKLENTLALTYFTTLEE |
| | | | | | IDEALISQDIDEESKSEMRKERINIIKNLS |
| | | | | | NDITQLKQLFIEKTELLDKSSSDLHNVVII |
| | | | | | EGTDKVLQAEQLRQKQLTEDIATKELER |
| | | | | | KEIEKKRDKIIEALDVIREHNLVDAFKDL |
| | | | | | IPTGENLSELDLAKPEIELLKQSLEITKKL |
| | | | | | LGQFSEGLKYIDLTDARKKLDNQIDTAS |
| | | | | | TRLTELNRQLEQSEKLIAGVNAVIKIDQE |
| | | | | | KSAVVVEAEKLSRAWHIFIHEITALQGTS |
| | | | | | LNEVELSKPLIKQQIYLESLIKQLI* |
| Providencia | PaYaxA | MTC35923 | | 27 | MNTQFNYDRIIEKESIGDATLALLTNQDS |
| alcalifaciens | | 1 | | | LSARSAGIFTLEDLISIHRHVQFALALPV |
| (Pa) | | | | | KDSDILKWFGINDENSASLPAIELMDIIID |
| | | | | | IRKHANSWNNVELKVKEQSVDLSLTSR |
| | | | | | NIMQTGGQVLEYINHMPIIKKISDTLEDL |
| | | | | | SEESLSQITYQNDDQQIATELLSILHLIKG |
| | | | | | DIKQQSIKTIHIKNTVSDFRAYITGGYLS |
| | | | | | DYTHVESLLFTLKGLYQQLESKEDPSNN |
| | | | | | ESFLKELITFKKNELHQLEQEYSHFVKLC |
| | | | | | FTGLAGGIIGLIITGSIFGSKAENIRHRKN |
| | | | | | MLIKEIEEINHKLHQEQLLQKTIFDIQINL |
| | | | | | QKIDGLFKDARLAIDHLDYMWLVILTEI |
| | | | | | KQSIDIFARINNAEKLIQFITQFKRIIMSW |
| | | | | | SAIQDYSIHLIKLFDELQISEKNSQ* |
| | PaYaxB | EUD10256. | | 28 | MHDNLDLPMLPSIDIYQLRCISNSISSFN |
| | | 1 | | | KNILNYDYIIYGRIQKIAAKVERLNELIR |
| | | | | | NSVPILKIKLNYIIKNDAYELLTLEDNND |
| | | | | | DYSDIRRKIVMTGFIDDLLDIKSELEKLIA |
| | | | | | KTRYALESLLVFHLNEPNKLKHSQYLKQ |
| | | | | | KEFLVNSKNGKQSKINELNNHLSIILAAE |
| | | | | | EIILKHRITDFFDKYFQGKELIDSIDIQPN |
| | | | | | KKDILKAAISYIRNLLTMVDDGLEFSQL |
| | | | | | VDVRIYVSDQIIEAREEINTIDNNIFRISQII |
| | | | | | SYANDISQIESHKQTIITQTGALKSYWES |
| | | | | | WCGFMSEKISEECLNLEHIKTMSQMFMT |
| | | | | | YLNDIEYQYQRQLPD* |
| Pseudomonas | PsYaxA | WP_198709422 | | 29 | MLTPAALLENEDIARVTLKPVEYLNVIF |
| syringae | | | | | DDSGGRSAGLVLTKEDILSLKRYERHAL |
| (Ps) | | | | | NIPTSLSRVEQQGFTKSGIPGLEPEDMLK |
| | | | | | TYKAINSHGKSWSGVEDGIKRTGFNIDL |
| | | | | | FAAQFSVQGQQIIDRIEKMDFARQLDLT |
| | | | | | VADLIIEEVRNTPPVLEKNDQRVCSTLAE |
| | | | | | FLKKTASQIKNHQHAAETLEQHIDTFSS |
| | | | | | VLSVTLIPGINDKVKLAARSDLDQQIKEL |
| | | | | | EKDIEQLTTNIEQKNKEYKTALNNIAWG |
| | | | | | GFGGPIGVAITGGIFGAKAEKIRKEKNR |
| | | | | | MVASKAQKVQLLKEKVPLTAAVRSLQL |
| | | | | | MFDDMYIRMLDAHKGATHLKDLWLLL |
| | | | | | ATYIERSAHELAAIKDDQALLIFAMQFQ |
| | | | | | GVVMPWLEIKGKTTELLKIFDSALDQFK |
| | | | | | REQQPAIGIGR* |
| | PsYaxB | WP 03265 | | 30 | MTMSTETLGKPMVLTPPDTEIMNAASQ |
| | | 4465 | | | NILAQVNTLKLDFLPAMKEKMLSLQNA |
| | | | | | LTRADNAYREALADITVQLNNVNLQPID |
| | | | | | LRQQHIEADARLSDKQKKQAISLINGER |
| | | | | | IRLLSNLTAVLRSSAHALAERSDDMAQIN |
| | | | | | LTQEDNRLQDILQQQIDGMTQRSAALES |
| | | | | | AMSVIAEDRRLLDTTIKTYEKYNLADLF |
| | | | | | KDMLPTQEELQIVAMPSPELALITAGIAR |
| | | | | | LGKLLDKISSALTYLDLTEERDRLRSRY |
| | | | | | NALLAESRTVAQEAKSINGRLDELTGLA |
| | | | | | DISKSKALWVKEARKVYHSLYKFLDQIT |
| | | | | | SQDDASAPISTPVEKLKTYIKSFYDIRRL |
| | | | | | V* |
| Proteus | PmYaxA | QGM69233.1 | | 31 | MDTTTLDHIDKVIEQYQLPEVTLSLILGK |
| mirabilis | | | | | NIDHGRSAGIFTPYDLNKIKSYIDKVLKL |
| (Pm) | | | | | PYQKDTIIELLGYEDINISQISAETIQTLFL |
| | | | | | NMRTHAFEWQKIEYSVKQQAIDLEITGR |
| | | | | | EITSTGEQLLAFINKMPIIKKINRKIEDITP |
| | | | | | EELSSIKYSTQDKKVSKQLVAILESMKN |
| | | | | | DINEQYNNTLKIKNMIYDFRIEIVGGENS |
| | | | | | KGNICHSIYYDIINKKKYINDFFDNSDDS |
| | | | | | LNEQRILLENEIEILKKEYQHFVKLAFTG |
| | | | | | LAFGIIGVIITGGIFGSKAEKIRKKRNETIE |
| | | | | | KLHNINKGIKQQSTISECLQRFQVDLDEI |
| | | | | | EQYIEDADMSVEHLLYMWQTILTEINAS |
| | | | | | LINFKKIDNAMELIRFSIYLEKIIAPWYM |
| | | | | | VVGYSKEMMAVFDEALSSFYSSK* |
| | PmYaxB | QKV33119. | | 32 | MNSVTSQLQEFDCNQFFHTIKSIRKITSE |
| | | 1 | | | LFEHDNILNHNLITISINNNKVYDEIIIFSI |
| | | | | | LLKSRLNQKLLTGIFKNINEINHAMADR |
| | | | | | ELTQQLSEKLLQEKMFIEQLFKDELTESI |
| | | | | | EIIEKHISFINDKKQLLTTTTIISEIDKLIRN |
| | | | | | KEEISIEINEKIAKQKLSINEIKEQLDIITKS |
| | | | | | EDIIYNKNILDFFTSHLPSKQTISKLQLAS |
| | | | | | SEKDILSVFIDLLKQFFTHLEKGFSYQKL |
| | | | | | VETRHTLIDNYLIQIKNLTKLEQKKQALL |
| | | | | | FTLSRYYAVTEINQYIDMLNQQLSLLEQ |
| | | | | | YWHTLIEQLIQLKRNLSEAKTVLSPHLV |
| | | | | | FLNHFSHHYQ* |
| Morganella | MmYaxA | QQU39789. | | 33 | MHTQTMPADRLITADEIGPATLKLLLNE |
| morganii | | 1 | | | EEDTFRAAGIFTHNDLVHIKRYINYGMT |
| (Mm) | | | | | LPREKEDVIEFIGYRDTELPGFEPHNIQSL |
| | | | | | FIHVHQHALSWDHVESMTKQQAIDLEIA |
| | | | | | GSAITATGNYILDAINEMPVIRRAEISLQ |
| | | | | | DATADKLRDITYRRQDNIISGELTKILSA |
| | | | | | MKDDIAEERRKTQKVKNIISRFRLEMIG |
| | | | | | GADESGNEIPSLIYDVKRKQKILLKNQDS |
| | | | | | DATNDLLSEIRVRNEEIEILKREYNQFVK |
| | | | | | LSFSGLIGGIIGLIITGGIFGYRAEQVRRR |
| | | | | | KNKLLEEVAELEEQIISRQAIQQLIIRLDK |
| | | | | | ELSTLDGYFTDAHVAVDHLDFMWQVM |
| | | | | | LTEITESLNTFMQINDAYSLLQFSLHLKK |
| | | | | | ITLPWQRVRGYAKELVAVFDTANLAA* |
| | MmYaxB | AWC94797.1 | | 34 | MTHSDITPEHITLAHLRAKYHDISNDAEI |
| | | | | | LSLPAADLHFQIRDIKKWQTKILNMISAE |
| | | | | | SAIIYSHLHNFDLENLLGTFSPDTGMNLF |
| | | | | | SLPHEKQIYEFLTSQMNTIYHSVNSINTL |
| | | | | | FNTEFDATAIPLLQGGLLHHQSYLDKAIS |
| | | | | | NARPDIDKFKCDKLYWEEKLAVIIQSEEI |
| | | | | | IHQRGFQSLFGTTTLPTTEQLKNVEMSSS |
| | | | | | ERFIMDELHRIISAVINTLTEGLSYVQLV |
| | | | | | ETRTTLSQRIYDLSKLIRNLKKDLKQLQ |
| | | | | | DHMQEISSALTLLPKLREFNNRISAVLLF |
| | | | | | WLQSVRQYEPYFSQNVPLPGLDALILAQ |
| | | | | | RRYFSAFIGMA* |
| Photorhabdus | PaxA | WP_105398085 | | 35 | MEKYMLLAQLPAEKTLNETDIPSATLQL |
| luminescens | | | | | LTGKQEGVARPGGIFTKEDLINIKLYVK |
| | | | | | KGLSLPFNLEEVKSYLGYQKIDIAGLEPE |
| | | | | | DIHSLFEEIRTHSFSWSDVENDILQQSIDL |
| | | | | | EVVGKQITETGENIISIINEMPIITRIKRRL |
| | | | | | GEISHRQLANITYTHEDREVSSALGEILD |
| | | | | | TMKNDINKQREKTEKVKTEVSDFKLKLI |
| | | | | | GGRLSNGSIALGLQPQVENKRKLMRDN |
| | | | | | KISVSIGDLDDKITEKKEEITQLKKDYDR |
| | | | | | FVGLAFSGIAGGLIGLAITGGIFGSRAEE |
| | | | | | VRKRKNMLIEEVRSLEESIKGKRALQKSI |
| | | | | | TSLSLDFSDIDTRLLDAEVALNHLDYMW |
| | | | | | QSMLTQISASKDKFTQINDALNLTSFITK |
| | | | | | FQQVINPWKEVEGSAKQLVKVFDEALK |
| | | | | | EYKHRYN* |
| | PaxB | UJD76268. | | 36 | MSEIITFPQQTVVYPEINVKTLSQAVKNI |
| | | 1 | | | WRLSHQQKSGIEIIQEKTLRISLYSRDLD |
| | | | | | EAARASVPQLQTVLRQLPPQDYFLTLTEI |
| | | | | | DTELEDPELDDETRNTLLEARSEHIRNLK |
| | | | | | KDVKGVIRSLRKEANLMASRIADVSNV |
| | | | | | VILERLESSLKEEQERKAEIQADIAQQEK |
| | | | | | NKAKLVVDRNKIIESQDVIRQYNLADMF |
| | | | | | KDYIPNISDLDKLDLANPKKELIKQAIKQ |
| | | | | | GVEIAKKILGNISKGLKYIELADARAKLD |
| | | | | | ERINQINKDCDDLKIQLKGVEQRIAGIED |
| | | | | | VHQIDKERTTLLLQAAKLEQAWNIFAK |
| | | | | | QLQNTIDGKIDQQDLTKIIHKQLDFLDDL |
| | | | | | ALQYNSMLLS* |
| Xenorhabdus | XaxA | ABB526431 | | 37 | MENDMSSNQTLAEKKIPVSEVPSATLK |
| nematophila | | | | | MLTSQAEGVARPGGIFTKGDLINIKLYV |
| | | | | | KHSLELPFTLEGVKEYIGYNDIDIDGLKP |
| | | | | | AKMATLFKEIHDHALSWSGVESKVQQQ |
| | | | | | SIDLENAGKQITLTGDEIISVIDQMPIIERV |
| | | | | | KNKLGDLTDKQLAEITYTNDDKEIAVEL |
| | | | | | GNILESMKKDIKKQQENTQKVKTAVSD |
| | | | | | FKLKLIGGELSDGTIAQGLQPQISSKKKL |
| | | | | | MDDNNLSTTIKDLQSKIDEKNKEIDQLQ |
| | | | | | KDYNKYVGLAFSGMVGGIISWAITGGIF |
| | | | | | GDKAEKARKQKNKLIDEVKDLQSQVKD |
| | | | | | KSALQTSVQNLSLSFAGIHTSMVDAEEA |
| | | | | | LNHLDFMWNTMLTQITTSRDKFDDIND |
| | | | | | ALKLTSFVIAFKQVIEPWRDVQGSAAQL |
| | | | | | IQTFDEALAEYKKLYH* |
| | XaxB | ABB526441 | | 38 | MSDNTLSQKENMYPEINIKAMNQAVNTI |
| | | | | | WLLAQRQTSGIEIINDKVKRISLYSREFD |
| | | | | | EMMRDSLAQLAPVLKQLTSDAAFQTIA |
| | | | | | QIDEALADPSLSKDDREALTLERNNLLQ |
| | | | | | NLSKDIDNVIVSFTGRTNKLTNKISDISD |
| | | | | | MVIAERLQDLVTQAESQKTELQSDIDPK |
| | | | | | TEKRNKLDADREKIIESQDVIRQNNIAD |
| | | | | | MFKDFIPSAKDIDGLDFTQPKKEAIKQAI |
| | | | | | KQGAEIARKILGKVSEGLKYIDLADARM |
| | | | | | KLSDQIDQLITETDELKAKIREVELRLSG |
| | | | | | LKDVMQIDTERTTLLTEAVKIEQVWISF |
| | | | | | AEQLHKLSNDEINQQDLSNLINGQLDFL |
| | | | | | NNLTLQYNKLK* |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-132. (canceled)

133. A method comprising:
(a) providing a nanopore system, wherein the nanopore system comprises (1) a fluid chamber and (2) a membrane comprising a nanopore, wherein the membrane separates the fluid chamber into a first side and a second side, wherein the nanopore comprises (i) a first opening of at least 11 nanometers (nm) and (ii) a second opening of less than 11 nm; and
(b) contacting the nanopore with a non-nucleic acid based polymer analyte.

134. The method of claim 133, wherein the first opening comprises at least 15 nm.

135. The method of claim 133, wherein the second opening comprises less than 5 nm.

136. The method of claim 133, wherein the nanopore comprises at least a portion of an alpha-helical pore forming protein or peptide thereof, or at least a portion of a beta-barrel pore forming protein or peptide thereof.

137. The method of claim 133, wherein the non-nucleic acid based polymer analyte comprises a protein, a polypeptide, a peptide, a protein assembly, a protein DNA assembly, saccharides, lipids, a bacterium, a virus capsid, a virus particle, a dendrimer, a polymer, inorganic particles, oligomeric particles, or any combination thereof.

138. The method of claim 133, wherein the non-nucleic acid based polymer analyte comprises a mass of at least about 5 kilodaltons (kDa).

139. The method of claim 133, wherein the non-nucleic acid based polymer analyte is at most 2 nanometers (nm) in length.

140. The method of claim 133, wherein the nanopore comprises one or more monomers of an Aerolysin (Aer) pore, Cytolysin K (CytK) pore, MspA pore, alpha-hemolysin (aHL) pore, CsgG pore, Fragaceatoxin C (FraC) pore, Lysenin pore, phage derived portal proteins (Phi29, G20c) pore, pleurotolysin (PlyA or PlyB) pore, ClyA pore, or homolog thereof, or paralog thereof, or ortholog thereof.

141. The method of claim 133, wherein the nanopore comprises a pore-forming toxin derived from Xenorhabdus, Yersinia, Providencia, Pseudomonas, Proteus, Morganella, or Photorhabdus.

142. The method of claim 133, wherein the nanopore comprises one or more subunits from an alpha-xenorhabdolysin family of binary toxins.

143. The method of claim 142, wherein a subunit of the one or more subunits comprises one or more proteins from the alpha-xenorhabdolysin family of binary toxins.

144. The method of claim 143, wherein the one or more proteins of the subunit from the alpha-xenorhabdolysin family of binary toxins are derived from Yesinia enterocolitica (Yax), Providencia alcalifaciens (Pa), Pseudomonas syringae (Ps), Proteus mirabilis (Pm), Morganella morganii (Mm), Photorhabdus luminescens (Pax), Xenorhabdus nematophila (Xax), or any combination thereof.

145. The method of claim 143, wherein the one or more proteins of the subunit from the alpha-xenorhabdolysin family is YaxA, YaxB, PaYaxA, PaYaxB, PsYaxA, PsYaxB, PmYaxA, PmPaxB, MmYaxA, MmYaxB, PaxA, PaxB, XaxA, XaxB, functional homolog thereof, functional ortholog thereof, functional paralog thereof, or any combination thereof.

146. The method of claim 133, wherein the nanopore comprises a monomeric unit comprising a heterodimer.

147. The method of claim 133, wherein the non-nucleic acid based polymer analyte is coupled to a binder protein.

148. The method of claim 147, wherein the binder protein is larger than 2 nm in size.

149. The method of claim 133, wherein the nanopore is coupled to one or more recognition elements.

150. The method of claim 149, wherein the one or more recognition elements comprises a protein, peptide, small molecule, nucleic acid, or any combination thereof.

151. The method of claim 133, further comprising measuring a signal generated by contacting the non-nucleic acid based polymer analyte with the nanopore.

152. The method of claim 151, wherein the signal comprises an ionic current, a change in ionic current, or derivations thereof.
